(19) United States
(12) Patent Application Publication
(10) Pub. No.: US 2002/0198216 A1
(43) Pub. Date: Dec. 26, 2002

(54) Novel farnesyl protein transferase inhibitors as antitumor agents

(76) Inventors: F. George Njoroge, Warren, NJ (US); Bancha Vibulbhan, Kenilworth, NJ (US); Alan B. Cooper, West Caldwell, NJ (US); Timothy Guzi, Chatam, NJ (US); Dinanath F. Rane, Morganville, NJ (US); Keith P. Minor, Sayreville, NJ (US); Ronald J. Doll, Maplewood, NJ (US); Viyyoor M. Girijavallabhan, Parisppany, NJ (US); Bama Santhanam, Bridgewater, NJ (US); Patrick A. Pinto, Morris Plains, NJ (US); Hugh Y. Zhu, Scotch Plains, NJ (US); Kartik M. Keertikar, Rahway, NJ (US); Carmen S. Alvarez, Roselle Park, NJ (US); John J. Baldwin, Gwynedd Valley, NJ (US); Ge Li, Shangi Hai (CN); Chia-Yu Huang, Plainsboro, NJ (US); Ray A. James, Plainsboro, NJ (US); W. Robert Bishop, Pompton Plains, NJ (US); James J-S Wang, Westfield, NJ (US); Jagdish A. Desai, Monroe Township, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/940,811

(22) Filed: Aug. 28, 2001

Related U.S. Application Data
(60) Provisional application No. 60/229,183, filed on Aug. 30, 2000.

(51) Int. Cl. A61K 31/496; A61K 31/473; C 07D 4 3/14

(52) U.S. Cl. 514/253.03; 514/290; 544/361; 546/93

(57) ABSTRACT

The present invention discloses novel tricyclic compounds represented by the formula (1.0):

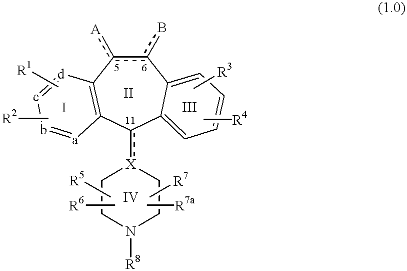

a prodrug thereof, or a pharmaceutically acceptable salt or solvate of the compound or of said prodrug useful for inhibiting farnesyl protein transferase. Also disclosed are pharmaceutical compositions comprising such compounds their preparation as well as methods of using them to treat proliferative diseases such as cancer.

This application claims the benefit of U.S. Provisional Application No. 60/229,183 filed Aug. 30, 2000.

### BACKGROUND

WO 95/10516, published Apr. 20, 1995 and WO 97/23478, published Jul. 3, 1997 disclose tricyclic compounds useful for inhibiting farnesyl protein transferase.

In view of the current interest in inhibitors of farnesyl protein transferase, a welcome contribution to the art would be compounds useful for the inhibition of farnesyl protein transferase. Such a contribution is provided by this invention.

### SUMMARY OF THE INVENTION

This invention provides compounds useful for the inhibition of farnesyl protein transferase (FPT). The compounds of this invention are represented by the formula:

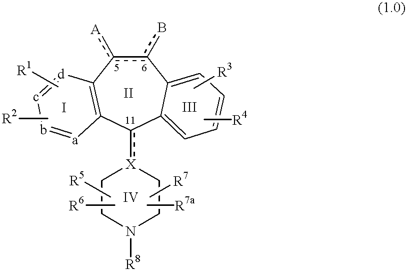

or a pharmaceutically acceptable salt or solvate thereof, wherein:

one of a, b, c and d represents N or N⁺O⁻, and the remaining a, b, c, and d groups represent carbon, wherein each carbon has an R¹ or R² group bound to said carbon; or each of a, b, c, and d is carbon, wherein each carbon has an R¹ or R² group bound to said carbon;

the dotted line (———) represents optional bonds;

X represents N or CH when the optional bond (to C11) is absent, and represents C when the optional bond (to C11) is present;

When the optional bond is present between carbon atom 5 (i.e., C-5) and carbon atom 6 (i.e., C-6) (i.e., there is a double bond between C-5 and C-6) then there is only one A substituent bound to C-5 and there is only one B substituent bound to C-6 and A or B is other than H;

When the optional bond is not present between carbon atom 5 and carbon atom 6 (i.e., there is a single bond between C-5 and C-6) then there are two A substituents bound to C-5, wherein each A substituent is independently selected and two B substituents bound to C-6, wherein each B substituent is independently selected, i.e.,

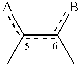

In formula 1.0 represents

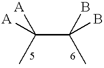

when there is a single bond between C-5 and C-6 and each A and each B are independently selected, and wherein at least one of the two A substituents or one of the two B substituents are H, and wherein at least one of the two A substituents or one of the two B substituants is other than H, (i.e., when there is a single bond between C-5 and C-6 one of the four substituents (A, A, B, and B) is H and one is other than H);

A and B is independently selected from:

(1) —H;

(2) —R⁹;

(3) —R⁹—C(O)—R⁹;

(4) —R⁹—CO₂—R^{9a};

(5) —(CH₂)pR²⁶;

(6) —C(O)N(R⁹)₂, wherein each R⁹ is the same or different;

(7) —C(O)NHR⁹;

(8) —C(O)NH—CH₂—C(O)—NH₂;

(9) —C(O)NHR²⁶;

(10) —(CH₂)pC(R⁹)—O—R^{9a};

(11) —(CH₂)p(R⁹)₂, wherein each R⁹ is the same or different;

(12) —(CH₂)pC(O)R⁹;

(13) —(CH₂)pC(O)R^{27a};

(14) —(CH₂)pC(O)N(R⁹)₂, wherein each R⁹ is the same or different;

(15) —(CH₂)pC(O)NH(R⁹);

(16) —(CH₂)pC(O)N(R²⁶)₂, wherein each R²⁶ is the same or different;

(17) —(CH₂)pN(R⁹)—R^{9a}, (e.g. —CH₂—N(CH2-pyridine)-CH₂-imidazole);

(18) —(CH₂)pN(R²⁶)₂, wherein R²⁶ is the same or different (e.g., —(CH₂)ₚ—NH—CH₂—CH₃);

(19) —(CH₂)pNHC(O)R⁵⁰;

(20) —(CH₂)pNHC(O)₂R⁵⁰;

(21) —(CH₂)pN(C(O)R^{27a})₂ wherein each R^{27a} is the same or different;

(22) —(CH₂)pNR⁵¹C(O)R²⁷, or R⁵¹ and R²⁷ taken together with the atoms to which they are bound form a heterocycloalkyl ring consisting of 5 or 6 members, provided that when R⁵¹ and R²⁷ form a ring, R⁵¹ is not H;

(23) —(CH₂)pNR⁵¹C(O)NR²⁷, or R⁵¹ and R²⁷ taken together with the atoms to which they are bound form a heterocycloalkyl ring consisting or 5 or 6 members, provided that when R⁵¹ and R²⁷ form a ring, R⁵¹ is not H;

(24) —(CH₂)pNR⁵¹C(O)N(R^{27a})₂, wherein each R^{27a} is the same or different;

(25) —(CH₂)pNHSO₂N(R⁵¹)₂, wherein each R⁵¹ is the same or different;

(26) —(CH₂)pNHCO₂R⁵⁰;

(27) —(CH₂)pNC(O)NHR⁵¹;

(28) —(CH₂)pCO₂R⁵¹;

(29) —NHR⁹;

(30)

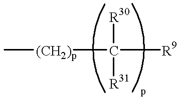

wherein R³⁰ and R³¹ are the same or different;

(31)

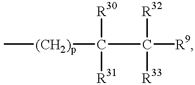

wherein R³⁰, R³¹ R³² and R³³ are the same or different;

(32)-alkenyl-CO₂R^{9a};

(33)-alkenyl-C(O)R

(34)-alkenyl-CO₂R⁵¹;

(35)-alkenyl-C(O)—R^{27a};

(36) (CH₂)ₚ-alkenyl-CO₂—R⁵¹;

(37) —(CH₂)pC═NOR⁵¹ or

(38) —(CH₂)ₚ-Phthalimid;

p is 0, 1, 2, 3 or 4;

each R¹ and R² is independently selected from H, Halo, —CF₃, —OR¹⁰, COR¹⁰, —SR¹⁰, —S(O)ₜR¹⁵ wherein t is 0, 1 or 2, —N(R¹⁰)₂, —NO₂, —OC(O)R¹⁰, CO₂R¹⁰, —OCO₂R¹⁵, —CN, —NR¹⁰COOR¹⁵, —SR¹⁵C(O)OR¹⁵, —SR¹⁵N(R¹³)₂ provided that R¹⁵ in —SR¹⁵N(R¹³)₂ is not —CH₂ and wherein each R¹³ is independently selected from H or —C(O)OR¹⁵, benzotriazol-1-yloxy, tetrazol-5-ylthio, or substituted tetrazol-5-ylthio, alkynyl, alkenyl or alkyl, said alkyl or alkenyl group optionally being substituted with halogen, —OR¹⁰ or —CO2R¹⁰;

R³ and R⁴ are the same or different and each independently represent H, and any of the substituents of R¹ and R²;

R⁵, R⁶, R⁷ and R^{7a} each independently represent H, —CF₃, —COR¹⁰, alkyl or aryl, said alkyl or aryl optionally being substituted with —OR¹⁰, —SR¹⁰, —S(O)ₜR¹⁵, —NR¹⁰COOR¹⁵, —N(R¹⁰)₂, —NO₂, —C(O)R¹⁰, —OCOR¹⁰, —OCO₂R¹⁰, —CO₂R¹⁰, OPO₃R¹⁰, or R⁵ is combined with R⁶to represent ═O or ═S;

R⁸ is selected from:

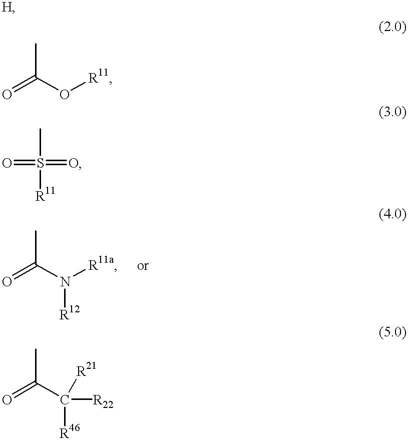

R⁹ is selected from:

(1) heteroaryl;

(2) substituted heteroaryl;

(3) arylalkoxy;

(4) substituted arylalkoxy;

(5) heterocycloalkyl;

(6) substituted heterocycloalkyl;

(7) heterocycloalkylalkyl;

(8) substituted heterocycloalkylalkyl;

(9) heteroarylalkyl;

(10) substituted heteroarylalkyl;

(11) heteroarylalkenyl;

(12) substituted heteroarylalkenyl;

(13) heteroarylalkynyl and

(14) substituted heteroarylalkynyl;

wherein said substituted R⁹ groups are substituted with one or more (e.g. 1, 2 or 3) substituents selected from:

(1) —OH;

(2) —CO₂R¹⁴;

(3) —CH₂OR¹⁴,

(4) halogen (e.g. Br, Cl or F),

(5) alkyl (e.g. methyl, ethyl, propyl, butyl or t-butyl);

(6) amino;

(7) trityl;

(8) heterocycloalkyl;

(9) cycloalkyl, (e.g. cyclopropyl or cyclohexyl);

(10) arylalkyl;

(11) heteroaryl;

(12) heteroarylalkyl and

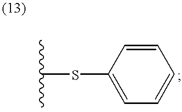

wherein R¹⁴ is independently selected from: H; alkyl; aryl, arylalkyl, heteroaryl and heteroarylalkyl;

R^{9a} is selected from: alky or arylalkyl;

R¹⁰ is selected from: H; alkyl; aryl or arylalkyl;

R¹¹ is selected from:

(1) alkyl;

(2) substituted alkyl;

(3) aryl;

(4) substituted aryl;

(5) cycloalkyl;

(6) substituted cycloalkyl;

(7) heteroaryl;

(8) substituted heteroaryl;

(9) heterocycloalkyl; and

(10) substituted heterocycloalkyl;

wherein said substituted R¹¹ groups have one or more (e.g. 1, 2 or 3) substituents selected from:

(1) —OH;

(2) halogen (e.g. Br, Cl or F) and

(3) alkyl;

R^{11a} is selected from:

(1) H;

(2) OH;

(3) alkyl;

(4) substituted alkyl;

(5) aryl;

(6) substituted aryl;

(7) cycloalkyl;

(8) substituted cycloalkyl;

(9) heteroaryl;

(10) substituted heteroaryl;

(11) heterocycloalkyl; and

(12) substituted heterocycloalkyl;

wherein said substituted R^{11a} groups have one or more (e.g. 1, 2 or 3) substituents selected from:

(1) —OH;

(2) —CN;

(3) —CF₃;

(4) halogen (e.g Br, Cl or F);

(5) alkyl;

(6) cycloalkyl;

(7) heterocycloalkyl;

(8) arylalkyl;

(9) heteroarylalkyl;

(10) alkenyl and

(11) heteroalkenyl;

R¹² is selected from: H, or alkyl;

R¹⁵ is selected from: alkyl or aryl;

R²¹, R²² and R⁴⁶ are independently selected from:

(1) —H;

(2) alkyl (e.g., methyl, ethyl, propyl, butyl or t-butyl);

(3) aryl, (e.g. phenyl);

(4) substituted aryl, optionally substituted with one or more substituents selected from: alkyl, halogen, CF₃ or OH;

(5) cycloalkyl, (e.g. cyclohexyl);

(6) substituted cycloalkyl; optionally substituted with one or more substituents selected from: alkyl, halogen, CF₃ or OH;

(7) heteroaryl of the formula,

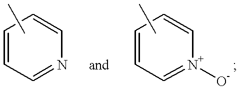

(8) heterocycloalkyl of the formula:

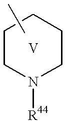

wherein R⁴⁴ is selected from:

(1) —H,

(2) alkyl, (e.g., methyl, ethyl, propyl, butyl or t-butyl);

(3) alkylcarbonyl (e.g., CH₃C(O)—);

(4) alkyloxy carbonyl (e.g., —C(O)O—t—C₄H₉, —C(O)OC₂H₅, and —C(O)OCH₃);

(5) haloalkyl (e.g., trifluoromethyl) and

(6) —C(O)NH(R⁵¹);

when R²¹, R²² or R⁴⁶ is the heterocycloalkyl of the formula above (i.e. Ring V), Ring V includes:

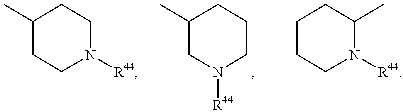

Examples of Ring V include:

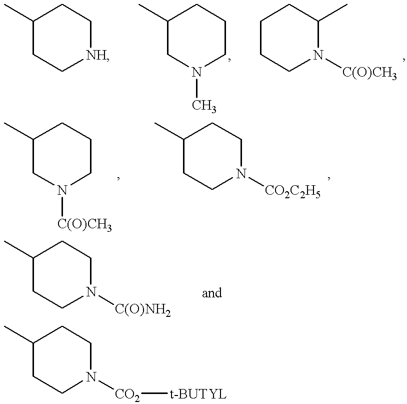

R²⁶ is selected from:

(1) —H;

(2) alkyl (e.g. methyl, ethyl, propyl, butyl or t-butyl);

(3) alkoxyl (e.g. methoxy, ethoxy, propoxy);

(4) —CH₂—CN;

(5) R⁹;

(6) —CH₂CO₂H;

(7) —C(O)alkyl and

(8) CH₂CO₂alkyl;

R²⁷ is selected from:

(1) —H;

(2) —OH;

(3) alkyl (e.g. methyl, ethyl, propyl, or butyl), and

(4) alkoxy;

R^{27a} is selected from:

(1) alkyl (e.g. methyl, ethyl, propyl, or butyl), and

(2) alkoxy;

R³⁰, R³¹, R³² and R³³ is independently selected from:

(1) —H;

(2) —OH;

(3) ═O;

(4) alkyl;

(5) aryl (e.g. phenyl) and

(6) arylalkyl (e.g. benzyl);

R⁵⁰ is selected from:

(1) alkyl;

(2) heteroaryl;

(3) substituted heteroaryl and

(4) amino;

wherein said substituents on said substituted R⁵⁰ groups are independently selected from: alkyl (e.g. methyl, ethyl, propyl, or butyl); halogen (e.g. Br, Cl, or F); and —OH;

R^{50a} is selected from:

(1) heteroaryl;

(2) substituted heteroaryl and

(3) amino;

R⁵¹ is selected from: —H, or alkyl (e.g.;methyl, ethyl, propyl, butyl or t-butyl);

The compounds of this invention: (i) potently inhibit farnesyl protein transferase, but not geranylgeranyl protein transferase I, in vitro; (ii) block the phenotypic change induced by a form of transforming Ras which is a farnesyl acceptor but not by a form of transforming Ras engineered to be a geranylgeranyl acceptor; (iii) block intracellular processing of Ras which is a farnesyl acceptor but not of Ras engineered to be a geranylgeranyl acceptor; and (iv) block abnormal cell growth in culture induced by transforming Ras.

The compounds of this invention inhibit farnesyl protein transferase and the farnesylation of the oncogene protein Ras. Thus, this invention further provides a method of inhibiting farnesyl protein transferase, (e.g., ras farnesyl protein transferase) in mammals, especially humans, by the administration of an effective amount (e.g. a therapeutically effective amount) of the tricyclic compounds described above. The administration of the compounds of this invention to patients, to inhibit farnesyl protein transferase, is useful in the treatment of the cancers described below.

This invention provides a method for inhibiting or treating the abnormal growth of cells, including transformed cells, by administering an effective amount (e.g. a therapeutically effective amount) of a compound of this invention. Abnormal growth of cells refers to cell growth independent of normal regulatory mechanisms (e.g., loss of contact inhibition). This includes the abnormal growth of: (1) tumor cells (tumors) expressing an activated Ras oncogene; (2) tumor cells in which the Ras protein is activated as a result of oncogenic mutation in another gene; and (3) benign and malignant cells of other proliferative diseases in which aberrant Ras activation occurs.

This invention also provides a method for inhibiting or treating tumor growth by administering an effective amount (e.g., a therapeutically effective amount) of the tricyclic compounds, described herein, to a mammal (e.g., a human) in need of such treatment. In particular, this invention provides a method for inhibiting or treating the growth of tumors expressing an activated Ras oncogene by the administration of an effective amount (e.g. a therapeutically effective amount) of the above described compounds.

The present invention also provides a method of treating proliferative diseases, especially cancers (tumors), comprising administering an effective amount (e.g., a therapeutically effective amount) of a compound of the invention, described herein, to a mammal (e.g., a human) in need of such treatment in combination with (2) an effective amount of at least one anti-cancer agent i.e. a chemotherapeutic agent and/or radiation).

The present invention also provides a method of treating proliferative diseases, especially cancers (tumors), comprising administering an effective amount (e.g., a therapeutically effective amount) of a compound of the invention, described herein, to a mammal (e.g., a human) in need of such treatment in combination with (2) an effective amount of at least one signal transduction inhibitor.

Examples of proliferative diseases (tumors) which may be inhibited or treated include, but are not limited to, lung cancer (e.g., lung adenocarcinoma), pancreatic cancers (e.g., pancreatic carcinoma such as, for example, exocrine pancreatic carcinoma), colon cancers (e.g., colorectal carcinomas, such as, for example, colon adenocarcinoma and colon adenoma), myeloid leukemias (for example, acute myelogenous leukemia (AML)), thyroid follicular cancer, myelodysplastic syndrome (MDS), bladder carcinoma, epidermal carcinoma, melanoma, breast cancer and prostate cancer.

It is believed that this invention also provides a method for inhibiting or treating proliferative diseases, both benign and malignant, wherein Ras proteins are aberrantly activated as a result of oncogenic mutation in other genes—i.e., the Ras gene itself is not activated by mutation to an oncogenic form—with said inhibition or treatment being accomplished by the administration of an effective amount (e.g. a therapeutically effective amount) of the tricyclic compounds described herein, to a mammal (e.g., a human) in need of such treatment. For example, the benign proliferative disorder neurofibromatosis, or tumors in which Ras is activated due to mutation or overexpression of tyrosine kinase oncogenes (e.g., neu, src, abl, Ick, and fyn), may be inhibited or treated by the tricyclic compounds described herein.

The tricyclic compounds useful in the methods of this invention inhibit or treat the abnormal growth of cells. Without wishing to be bound by theory, it is believed that these compounds may function through the inhibition of G-protein function, such as Ras p21, by blocking G-protein isoprenylation, thus making them useful in the treatment of proliferative diseases such as tumor growth and cancer. Without wishing to be bound by theory, it is believed that these compounds inhibit ras farnesyl protein transferase, and thus show antiproliferative activity against ras transformed cells.

### DETAILED DESCRIPTION OF THE INVENTION

As used herein, the following terms are used as defined below unless otherwise indicated:

MH⁺—represents the molecular ion plus hydrogen of the molecule in the mass spectrum;

BOC—represents tert-butyloxycarbonyl;

CBZ—represents —C(O)OCH₂C₆H₅ (i.e., benzyloxycarbonyl);

CH₂Cl₂—represents dichloromethane;

CIMS—represents chemical ionization mass spectrum;

DBU—represents 1,8-Diazabicyclo[5.4.0]undec-7-ene;

DEAD—represents diethylazodicarboxylate;

DEC—represents EDCI which represents 1-(3-dimethyl-aminopropyl)-3-ethylcarbodiimide hydrochloride;

DMF—represents N,N-dimethylformamide;

Et—represents ethyl;

EtOAc—represents ethyl acetate;

EtOH—represents ethanol;

HOBT—represents 1-hydroxybenzotriazole hydrate;

IPA—represents isopropanol;

i-PrOH—represents isopropanol;

Me—represents methyl;

MeOH—represents methanol;

MS—represents mass spectroscopy;

FAB—represents FABMS which represents fast atom bombardment mass spectroscopy;

HRMS—represents high resolution mass spectroscopy;

NMM—represents N-methylmorpholine;

PPh₃—represents triphenyl phosphine;

Ph—represents phenyl;

Pr—represents propyl;

EM—represents 2,2-(Trimethylsilyl)ethoxymethyl;

TBDMS—represents tert-butyidimethylsilyl;

Et₃N—represents TEA which represents triethylamine;

t-BUTYL—represents -C—(CH₃)₃;

TFA—represents trifluoroacetic acid;

THF—represents tetrahydrofuran;

Tr—represents trityl;

Tf—represents SO₂CF₃;

at least one—represents one or more-(e.g. 1-6), more preferrably 1-4 with 1, 2 or 3 being most preferred;

alkyl—represents straight and branched carbon chains and contains from one to twenty carbon atoms, preferably one to six carbon atoms, more preferably one to four carbon atoms; even more preferably one to two carbon atoms.

arylalkyl—represents an alkyl group, as defined above, substituted with an aryl group, as defined below, such that the bond from another substituent is to the alkyl moiety;

alkoxy—represents an alkyl moiety, alkyl as defined above, covalently bonded to an adjacent structural element through an oxygen atom, for example, methoxy, ethoxy, propoxy, butoxy and the like;

phenoxy represents an alkoxy moiety, as defined above, wherein the covalently bonded moiety is an aryl group, as defined below, for example, —O-phenyl;

alkenyl represents straight and branched carbon chains having at least one carbon to carbon double bond and containing from 2-12 carbon atoms, preferably from 2 to 6 carbon atoms and most preferably from 3 to 6 carbon atoms;

alkynyl represents straight and branched carbon chains having at least one carbon to carbon triple bond and containing from 2-12 carbon atoms, preferably from 2 to 6 carbon atoms and most preferably from 2 to 4 carbon atoms;

amino represents an —NH₂ moiety;

aryl-(including the aryl portion of arylalkyl and heteroarylalkyl)-represents a carbocyclic group containing from 6 to 15 carbon atoms and having at least one aromatic ring (e.g., aryl is a phenyl ring), with all available substitutable carbon atoms of the carbocyclic group being intended as possible points of attachment, said carbocyclic group being optionally substituted with one or more (e.g., 1 to 3) of halo, alkyl, hydroxy, alkoxy, phenoxy, CF₃, —C(O)N(R¹⁸)₂, —SO₂R¹⁸, —SO₂N(R¹⁸)₂, amino, alkylamino, dialkylamino, —COOR²³ or —NO wherein R¹⁸ represents H, alkyl, aryl, arylalkyl, heteroaryl or cycloalkyl and R²³ represents alkyl or aryl;

cycloalkyl—represents saturated carbocyclic rings of from 3 to 20 carbon atoms, preferably 3 to 7 carbon atoms, said cycloalkyl ring being optionally substituted with one or more (e.g., 1, 2 or 3) of the same or different alkyl groups (e.g., methyl or ethyl);

cycloalkylalkyl—represents an alkyl group, as defined above, substituted with a cyclo group, as defined above, such that the bond from another substituent is to the alkyl moiety;

heterocycloalkylalkyl—represents an alkyl group, as defined above, substituted with a heterocycloalkyl group, as defined below, such that the bond from another substituent is to the alkyl moiety;

halo—represents halogen i.e. fluoro, chloro, bromo and iodo;

haloalkyl—represents an alkyl group, as defined above, substituted with a halo group, as defined above, such that the bond from another substituent is to the alkyl moiety;

heteroarylalkyl—represents an alkyl group, as defined above, substituted with a heteroaryl group, as defined below, such that the bond from another substituent is to the alkyl moiety;

heteroarylalkenyl—represents an alkenyl group, as defined above, substituted with a heteroaryl group, as defined below, such that the bond from another substituent is to the alkyl moiety;

heteroalkyl—represents straight and branched carbon chains containing from one to twenty carbon atoms, preferably one to six carbon atoms interrupted by 1 to 3 heteroatoms selected from —O—, —S— and —N—;

heteroalkenyl—represents straight and branched carbon chains having at least one carbon to carbon double bond and containing from one to twenty carbon atoms, preferably one to six carbon atoms interrupted by 1 to 3 heteroatoms selected from —O—, —S— and —N—;

heteroalkynyl—represents straight and branched carbon chains having at least one carbon to carbon triple bond and containing from one to twenty carbon atoms, preferably one to six carbon atoms interrupted by 1 to 3 heteroatoms selected from —O—, —S— and —N—;

arylheteroalkyl—represents a heteroalkyl group, as defined above, substituted with an aryl group, as defined above, such that the bond from another substituent is to the alkyl moiety;

alkylcarbonyl—represents an alkyl group, as defined above, covalently bonded to a carbonyl moiety (—CO—), for example, —COCH₃;

alkyloxycarbonyl—represents an alkyl group, as defined above, covalently bonded to a carbonyl moiety (—CO—) through an oxygen atom, for example, —C(O)—OC₂H₅;

heteroaryl—represents cyclic groups, optionally substituted with R³ and R⁴, having at least one heteroatom selected from O, S or N, said heteroatom interrupting a carbocyclic ring structure and having a sufficient number of delocalized pi electrons to provide aromatic character, with the aromatic heterocyclic groups preferably containing from 2 to 14 carbon atoms, e.g., 2- or 3-furyl, 2- or 3-thienyl, 2-, 4- or 5-thiazolyl, 2-, 4- or 5-imidazolyl, 2-, 4- or 5-pyrimidinyl, 2-pyrazinyl, 3- or 4-pyridazinyl, 3-, 5- or 6-[1 ,2,4-triazinyl], 3- or 5-[1 ,2,4-thiadizolyl], 2-, 3-, 4-, 5-, 6- or 7-benzofuranyl, 2-, 3-, 4-, 5-, 6- or 7-indolyl, 3-, 4- or 5-pyrazolyl, 2-, 4- or 5-oxazolyl, triazolyl, 2-, 3- or 4-pyridyl, or 2-, 3- or 4-pyridyl N-oxide, wherein pyridyl N-oxide can be represented as:

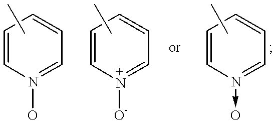

and

heterocycloalkyl- represents a saturated, branched or unbranched carbocylic ring containing from 3 to 15 carbon atoms, preferably from 4 to 6 carbon atoms, which carbocyclic ring is interrupted by 1 to 3 hetero groups selected from —O—, —S— or —NR²⁴, (e.g., —NC(O)—NH₂) wherein R²⁴ represents alkyl, aryl, —C(O)N(R¹⁸)₂ wherein R¹⁸ is as above defined, suitable heterocycloalkyl groups include 2- or 3-tetrahydrofuranyl, 2- or 3- tetrahydrothienyl, 2-, 3- or 4-piperidinyl, 2- or 3-pyrrolidinyl, 1-, 2-, 3-, or 4-piperizinyl, 2- or 4-dioxanyl, morpholinyl, and

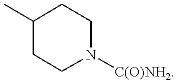

The positions in the tricyclic ring system are:

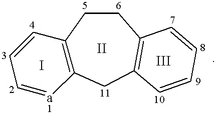

The compounds of formula 1.0 include the preferred R isomer:

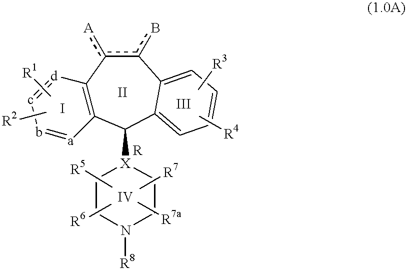

X=N or CH

a=N or C

wherein the optional bond between C-5 and C-6 is present, and B is H, or the optional bond between C-5 and C-6 is absent and each B is H; and the preferred S isomer:

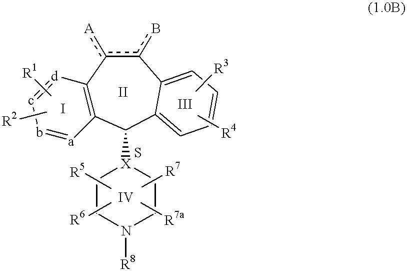

X=N or CH

a=N or C

wherein the optional bond between C-5 and C-6 is present and A is H, or the optional bond between C-5 and C-6 is absent and each A is H.

Preferably, R¹, R², R³, and R⁴ are independently selected from H or halo, more preferably H, Br, F or Cl, and even more preferably H, or Cl. Representative compounds of formula 1.0 include dihalo (e.g., 3,8-dihalo) and monohalo (e.g., 8-halo) substituted compounds, such as, for example: (3-bromo, 8-chloro), (3,8-dichloro), (3-bromo) and (3-chloro).

Substituent a is preferably C or N with N being most preferred.

Preferably, R⁸ is selected from:

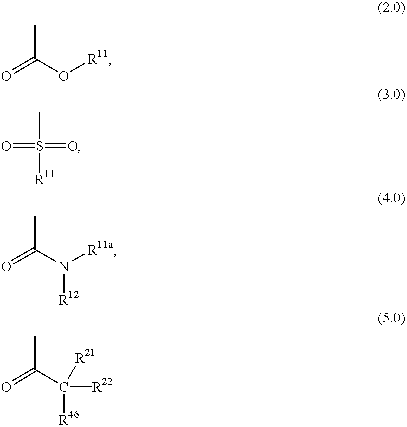

More preferably R⁸ is 2.0 or 4.0; and most preferably R⁸ is 4.0.

Preferably, R^{11a} is selected from: alkyl, substituted alkyl, aryl, substituted aryl, heteroaryl, substituted heteroaryl, cyloalkyl or substituted cycloalkyl; wherein, said substituted aryl, heteroary, and cycloalkyl, R^{11a} groups are substituted with substituents independently selected from: halo (preferably F or Cl), cyano, —CF₃, or alkyl; and wherein said substituted alkyl R^{11a} groups substituted with substituents selected from halo, (preferably F or Cl), cyano or CF₃. Most preferably, R^{11a} is selected from: alkyl, aryl, substituted aryl, cyloalkyl, or substituted cycloalkyl, wherein, said substituted aryl and substituted cycloalkyl groups are substituted with substituents independently selected from: halo, (preferably F or Cl), CN or CF₃. More preferably, R^{11a} is selected from methyl, t-butyl, phenyl, cyanophenyl, chlorophenyl, fluorophenyl, or cyclohexyl. Still more preferably, R^{11a} is selected from: t-butyl, cyanophenyl, chlorophenyl, fluorophenyl or cyclohexyl. Even more preferably, R^{11a} is selected from cyanophenyl, with p-cyanophenyl being even still more preferred.

Preferably, R¹¹, is selected from alkyl, cycloalkyl, or substituted cycloalkyl, wherein said substituted cycloalkyl group is substituted with 1, 2 or 3 substituents independently selected from: halo (preferably chloro or fluoro), or alkyl,(preferably methyl or t-butyl). Examples of R¹¹ groups include: methyl, ethyl, propyl, t-butyl, cyclohexyl or substituted cyclohexyl. More preferably, R¹¹ is selected from methyl, t-butyl, cyclohexyl, chlorocyclohexyl, (preferably p-chlorocyclohexyl) or fluorocyclohexyl, (preferably p-fluorocyclohexyl). Most preferably, R¹¹ is selected from: methyl, t-butyl, or cyclohexyl, with t-butyl or cyclohexyl being still more preferred.

Preferably, R¹² is selected from H or methyl. Most preferably, R¹² is H. R⁵, R⁶, R⁷ and R^{7a} are preferably H.

Preferably, R⁹ is selected from:

(1) heteroaryl;

(2) substituted heteroaryl;

(3) arylalkoxy;

(4) substituted arylalkoxy;

(5) heterocycloalkyl;

(6) substituted heterocycloalkyl;

(7) heterocycloalkylalkyl;

(8) substituted heterocycloalkylalkyl;

(9) heteroarylalkyl;

(10) substituted heteroarylalkyl;

(11) heteroarylalkenyl and

(12) substituted heteroarylalkenyl;

wherein said substituted R⁹ groups are substituted with one or more substituents (e.g., 1, 2, or 3) independently selected from:

(1) —OH;

(2) —CO₂R;

wherein, R¹⁴ is selected from: H or alkyl (e.g., methyl or ethyl), preferably alkyl,most preferably methyl or ethyl;

(3) alkyl, substituted with one or more —OH groups (e.g., 1, 2, or 3, preferably 1), for example —(CH₂)qOH wherein, q is 1-4, with q=1 being preferred.

(4) halo (e.g., Br, F, I, or Cl);

(5) alkyl, usually C1-C6 alkyl, preferably C1-C4 alkyl (e.g. methyl, ethyl, propyl, or butyl (preferably isopropyl, or t-butyl));

(6) amino;

(7) trityl;

(8) heterocycloalkyl;

(9) arylalkyl (e.g. benzyl);

(10) heteroaryl (e.g. pyridyl) and

(11) heteroarylalkyl (piperidine-CH₃);

Most preferably, R⁹ is selected from:

(1) heterocycloalkyl;

(2) substituted heterocycloalkyl;

(3) heterocycloalkylalkyl;

(4) substituted heterocycloalkylalkyl;

(5) heteroarylalkyl;

(6) substituted heteroarylalkyl;

(7) heteroarylalkenyl and

(8) substituted heteroarylalkenyl;

wherein said substituted R⁹ groups are substituted with substituents independently selected from:

(1) —OH;

(2) —CO₂R¹⁴;

wherein, R¹⁴ is selected from: H or alkyl (e.g., methyl or ethyl), preferably alkyl, and most preferably methyl or ethyl;

(3) alkyl, substituted with one or more —OH groups (e.g.,1, 2, or 3, preferably 1), for example —(CH₂)qOH wherein, q is 1-4, with q=1 being preferred.

(4) halo (e.g., Br or Cl);

(5) alkyl, usually C1-C6 alkyl, preferably C1-C4 alkyl (e.g. methyl, ethyl, propyl, isopropyl, butyl or t-butyl, most preferably t-butyl);

(6) amino;

(7) trityl;

(8) heterocycloalkyl;

(9) arylalkyl;

(10) heteroaryl and

(11) heteroarylalkyl;

More preferably, R⁹ is selected from:

(1) heterocycloalkyl;

(2) substituted heterocycloalkyl;

(3) heterocycloalkylalkyl;

(4) substituted heterocycloalkylalkyl;

(5) heteroarylalkyl;

(6) substituted heteroarylalkyl;

(7) heteroarylalkenyl and

(8) substituted heteroarylalkenyl;

wherein substituents for said substituted R⁹ groups are each independently selected from:

(1) halo (e.g., Br, or Cl);

(2) alkyl, usually C1-C6 alkyl, preferably C1-C4 alkyl (e.g. methyl, ethyl, propyl, isopropyl, butyl or t-butyl, most preferably t-butyl);

(3) alkyl, substituted with one or more (i.e. 1, 2, or 3, preferably 1) —OH groups, (e.g. —(CH₂)qOH wherein q is 1-4, with q=1 being preferred).

(4) amino;

(5) trityl;

(6) arylalkyl, and

(7) heteroarylalkyl.

Even more preferably, R⁹ is selected from:

(1) heterocycloalkylalkyl;

(2) substituted heterocycloalkylalkyl;

(3) heteroarylalkyl and

(4) substituted heteroarylalkyl;

wherein substituents for said substituted R⁹ groups are each independently selected from:

(1) halo (e.g., Br, or Cl);

(2) alkyl, usually C1-C6 alkyl, preferably C1-C4 alkyl (e.g. methyl, ethyl, propyl, isopropyl, butyl or t-butyl, most preferably t-butyl);

(3) amino and

(4) trityl.

Still more preferably, R⁹ is selected from:

(1) heterocycloalkylalkyl;

(2) substituted heterocycloalkylalkyl;

(3) heteroarylalkyl and

(4) substituted heteroarylalkyl;

wherein substituents for said substituted R⁹ groups are each independently selected from:

(1) halo (e.g., Br,or Cl) and

(2) alkyl, usually C1-C6 alkyl, preferably C1-C4 alkyl (e.g. methyl, ethyl, propyl, isopropyl, butyl or t-butyl, most preferably t-butyl).

Yet even more preferably, R⁹ is selected from:

(1) piperidinyl;

(2) piperizinyl;

(3) —(CH2)p-piperidinyl;

(4) —(CH2)p-piperizinyl;

(5) —(CH2)p-morpholinyl and

(6) —(CH2)p-imidazolyl;

wherein p is 0 to 1, and wherein the ring moiety of each R⁹ group is optionally substituted with one, two or three substituents independently selected from:

(1) halo (e.g., Br,or Cl) and

(2) alkyl, usually C1-C6 alkyl, preferably C1-C4 alkyl (e.g. methyl, ethyl, propyl, isopropyl, butyl or t-butyl, most preferably t-butyl).

Still more preferably, R⁹ is selected from:

(1) -piperizinyl;

(2) —(CH2)p-piperidinyl;

(3) —(CH2)p-imidazolyl; and

(4) —(CH2)p-morpholinyl,

wherein p is 1 to 4, and the ring moiety of each R⁹ group is optionally substituted with one, two or three substituents independently selected from: methyl, ethyl, and isopropyl.

Yet even more preferably, R⁹ is selected from —(CH₂)-Imidazolyl, wherein said imidazolyl ring is optionally substituted with 1, 2, or 3 substituants, preferably 1, independently selected from methyl or ethyl.

Still even more preferably, R⁹ is selected from —(CH₂)—(2-methyl)-imidazole.

Preferably, at least one of R²¹, R²² and R⁴⁶ is other than H or alkyl. More preferably, R²¹ and R²² is H and R⁴⁶ is other than H or alkyl. Most preferably, R²¹ and R²² is H and R⁴⁶ is selected from heteroaryl or heterocycloalkyl.

Preferably, said heteroaryl groups for said R²¹, R²² or R⁴⁶ is 3-pyridyl, 4-pyridyl, 3-pyridyl-N-Oxide or 4-pyridyl- N-Oxide; more preferably 4-pyridyl or 4-pyridyl- N-Oxide; most preferably 4-pyridyl- N-Oxide.

Preferably, said heterocycloalkyl groups for said R²¹, R²², or R⁴⁶ is piperidine Ring V:

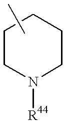

wherein R⁴⁴ is —C(O)NHR⁵¹, and preferably R⁵¹ is —C(O)NH₂. More preferably, piperidine Ring V is:

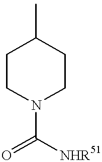

and most preferred Ring V is:

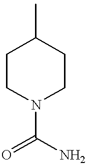

Thus, R²¹, R²² and R⁴⁶ are preferably independently selected from:

(1) H;

(2) aryl (most preferably phenyl);

(3) heteroaryl and

(4) heterocycloalkyl (i.e., Piperidine Ring V)

wherein at least one or R²¹, R²², or R⁴⁶ is other than H, and most preferably R²¹ and R²² are H and R⁴⁶ is other than H, and more preferably R²¹ and R²² are H and R⁴⁶ is selected from heteroaryl or heterocycloalkyl, and still more preferably R²¹ and R²² are H and R⁴⁶ is Piperidine Ring V; wherein the preferred definitions of heteroaryl and Piperidine Ring V are as described above.

Preferably, A and B are independently selected from:

(1) —H;

(2) —R⁹;

(3) —R⁹—C(O)—R⁹;

(4) —R⁹—CO₂—R^{9a};

(5) —C(O)NHR⁹;

(6) —C(O)NH—CH₂—C(O)—NH₂;

(7) —C(O)NHR²⁶;

(8) —(CH₂)p(R⁹)₂, wherein each R⁹ is the same or different;

(9) —(CH₂)pC(O)R⁹;

(10) —(CH2)pC(O)R^{27a};

(11) —(CH₂)pC(O)N(R⁹)₂, wherein each R⁹ is the same or different;

(12) —(CH₂)pC(O)NH(R⁹);

(13) —(CH₂)pNHC(O)R⁵⁰;

(14) —(CH₂)pNHC(O)₂R⁵⁰;

(15) —(CH₂)pN(C(O)R^{27a})₂ wherein R^{27a} is the same or different;

(16) —(CH₂)pNR⁵¹C(O)R²⁷, optionally, R⁵¹ and R²⁷, taken together with the atoms to which they are bound, form a heterocycloalkyl ring consisting of 5 or 6 members, provided that when R⁵¹ and R²⁷ form a ring, R⁵¹ is not H;

(17) —(CH₂)pNR⁵¹C(O)NR²⁷, optionally, R⁵¹ and R²⁷, taken together with the atoms to which they are bound, form a heterocycloalkyl ring consisting or 5 or 6 members, provided that when R⁵¹ and R²⁷ form a ring, R⁵¹ is not H;

(18) —(CH₂)pNR⁵¹C(O)N(R^{27a})₂, wherein each R^{27a} is the same or different;

(19) —(CH₂)pNHSO₂N(R⁵¹)₂, wherein each R⁵¹ is the same or different;

(20) —(CH₂)pNHCO₂R⁵⁰;

(21) —(CH₂)pCO₂R⁵¹;

(22) —NHR⁹;

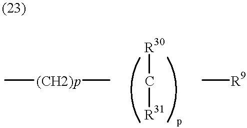

wherein R³⁰ and R³¹ are the same or different and

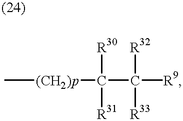

wherein R³⁰, R³¹, R³² and R³³ are the same or different.

Most preferably, A and B are independently selected from:

(1) —H;

(2) —R⁹;

(3) —R⁹—C(O)—R⁹;

(4) —R⁹—CO₂—R^{9a};

(5) —C(O)NHR⁹;

(6) —(CH₂)p(R⁹)₂, wherein each R⁹ is the same or different;

(7) —(CH₂)pC(O)R⁹;

(8) —(CH₂)pC(O)N(R⁹)₂, wherein each R⁹ is the same or different;

(9) —(CH₂)pC(O)NH(R⁹);

(10) —(CH₂)pNR⁵¹C(O)R²⁷, optionally, R⁵¹ and R²⁷, taken together with the atoms to which they are bound, form a heterocycloalkyl ring consisting of 5 or 6 members, provided that when R⁵¹ and R²⁷ form a ring, R⁵¹ is not H;

(12) —(CH₂)pNR⁵¹C(O)NR²⁷, optionally, R⁵¹ and R²⁷, taken together with the atoms to which they are bound, form a heterocycloalkyl ring consisting of 5 or 6 members, provided that when R⁵¹ and R²⁷ form a ring, R⁵¹ is not H and

(13) —NHR⁹.

Examples of A and B include but are not limited to:

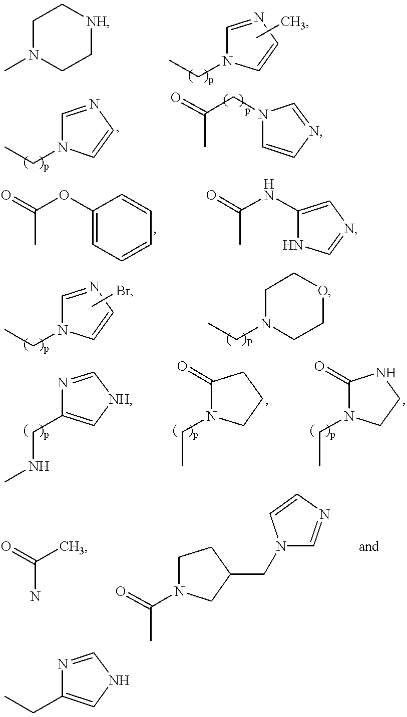

wherein p is 0, 1, 2, 3 or 4;

When the optional bond between C-5 and C-6 is present (i.e., there is a double bond between C-5 and C-6), then preferably one of A or B is H and the other is R⁹, and preferably, R⁹ is selected from:

(1) heteroaryl;

(2) substituted heteroaryl;

(3) arylalkyl;

(4) substituted arylalkyl;

(5) arylalkoxy;

(6) substituted arylalkoxy;

(7) heterocycloalkyl;

(8) substituted heterocycloalkyl;

(9) heterocycloalkylalkyl;

(10) substituted heterocycloalkylalkyl;

(11) heteroarylalkyl;

(12) substituted heteroarylalkyl;

(13) alkenyl;

(14) substituted alkenyl;

(15) heteroarylalkenyl and

(16) substituted heteroarylalkenyl,

wherein the substituents for said substituted R⁹ groups are each independently selected from:

(1) —OH;

(2) —CO₂R¹⁴;

(3) —CH₂OR¹⁴,

(4) halo,

(5) alkyl (e.g. methyl, ethyl, propyl, butyl or t-butyl);

(6) amino;

(7) trityl;

(8) heterocycloalkyl;

(9) arylalkyl;

(10) heteroaryl and

(11) heteroarylalkyl,

wherein R¹⁴ is independently selected from: H; or alkyl, preferably methyl or ethyl.

More preferably, when there is a double bond between C-5 and C-6, A is H and B is R⁹. Most preferably, when there is a double bond between C-5 and C-6, A is H and B is R⁹ wherein R⁹ is selected from:

(1) arylalkyl;

(2) substituted arylalkyl;

(3) arylalkoxy;

(4) substituted arylalkoxy;

(5) heterocycloalkyl;

(6) substituted heterocycloalkyl;

(7) heterocycloalkylalkyl;

(8) substituted heterocycloalkylalkyl;

(9) heteroarylalkyl;

(10) substituted heteroarylalkyl;

(11) alkenyl;

(12) substituted alkenyl;

(13) heteroarylalkenyl and

(14) substituted heteroarylalkenyl,

wherein the substituents for said substituted R⁹ groups are independently selected from:

(1) —OH;

(2) halo, (preferably Br);

(3) alkyl (e.g. methyl, ethyl, propyl, butyl, or t-butyl);

(4) amino and

(5) trityl.

Still more preferably, when there is a double bond between C-5 and C-6, A is H and B is R⁹ wherein R⁹ is selected from:

(1) heterocycloalkylalkyl;

(2) substituted heterocycloalkylalkyl;

(3) heteroarylalkyl and

(4) substituted heteroarylalkyl,

wherein said substituents for said substituted R⁹ groups are the same or different alkyl groups (e.g., C1-C4 alkyl).

Even more preferably, when there is a double bond between C-5 and C-6, A is H and B is R⁹ wherein R⁹ is selected from:

(1) heteroaryl(C1-C3)alkyl and

(2) substituted heteroaryl(C1-C3)alkyl,

wherein the substituents for said substituted R⁹ group are as defined above.

Yet still more preferably, when there is a double bond between C-5 and C-6, A is H and B is R⁹ wherein R⁹ is selected from:

(1) heteroaryl(C1-C3)alkyl, with heteroaryl-CH₂— being preferred and

(2) substituted heteroaryl(C1-C3)alkyl, with substituted heteroaryl-CH₂— being preferred,

wherein the substituents for said substituted R⁹ groups are selected from one or more (e.g. 1, 2 or 3) with one being preferred, of the same or different alkyl groups (e.g., —CH₃, —C₂H₅, —C₃H₄) with —CH₃ being preferred.

Even still more preferably, when there is a double bond between C-5 and C-6, A is H and B is R⁹ wherein R⁹ is selected from:

(1) —CH₂-imidazolyl;

(2) substituted imidazolyl-CH₂—;

(3) —(CH₂)₂-imidazolyl;

(4) substituted imidazolyl-(CH₂)₂—;

(5) —(CH₂)₃-imidazolyl;

(6) substituted imidazolyl-(CH₂)₃—;

(7) —CH₂-piperazinyl and

(8)-CH₂-morpholinyl;

wherein the substituents for said substituted R⁹ groups are selected from one or more (e.g. 1, 2 or 3), with one being preferred, of the same or different alkyl groups (e.g., —CH₃, —C₂H₅, —C₃H₄) with —CH₃ being preferred; and wherein, the substituted imidazolyl groups:

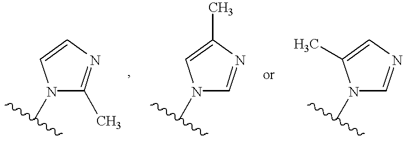

are preferred, with

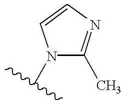

being most preferred.

Yet still more preferably, when there is a double bond between C-5 and C-6, A is H and B is R⁹ wherein R⁹ is substituted imidazolyl-CH₂—, with

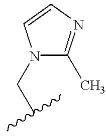

being preferred.

When B is H and A is R⁹, and there is a double bond between C-5 and C-6, the R⁹ groups for A are those described above for B.

When the optional bond between C-5 and C-6 is not present (i.e, there is a single bond between C-5 and C-6), each A and each B are independently selected and the definitions of A and B are the same as those described above when the optional bond is present, provided that when there is a single bond between C-5 and C-6 then one of the two A substituents or one of the two B substituents is H (i.e., when there is a single bond between C-5 and C-6 one of the four substituents (A, A, B, and B) has to be H).

Preferably, there is a double bond between C-5 and C-6.

Compounds of this invention having C-11 R- and S-stereochemistry include:

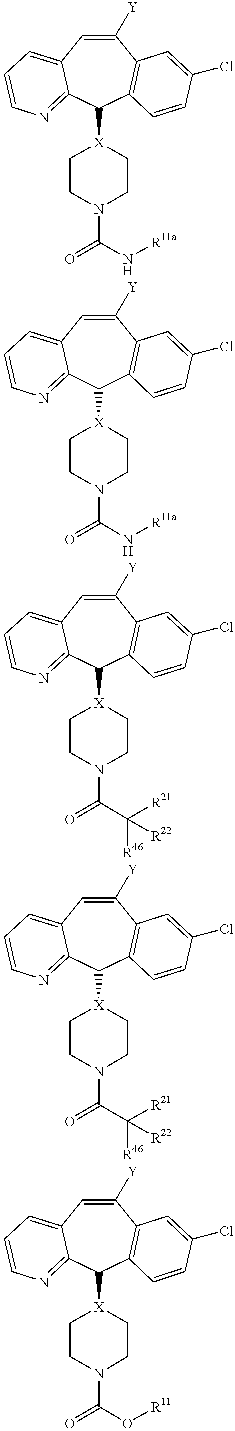

wherein X=N or C;

Q=Br or Cl;

Y=alkyl, arylalkyl, or heteroarylalkyl.

Preferred compounds of this invention are listed below:

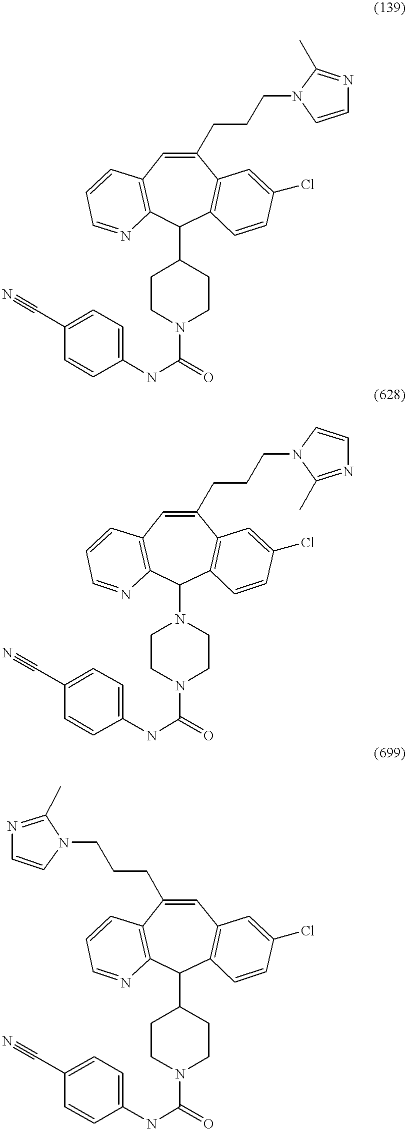

More preferred compounds of this invention are listed below:

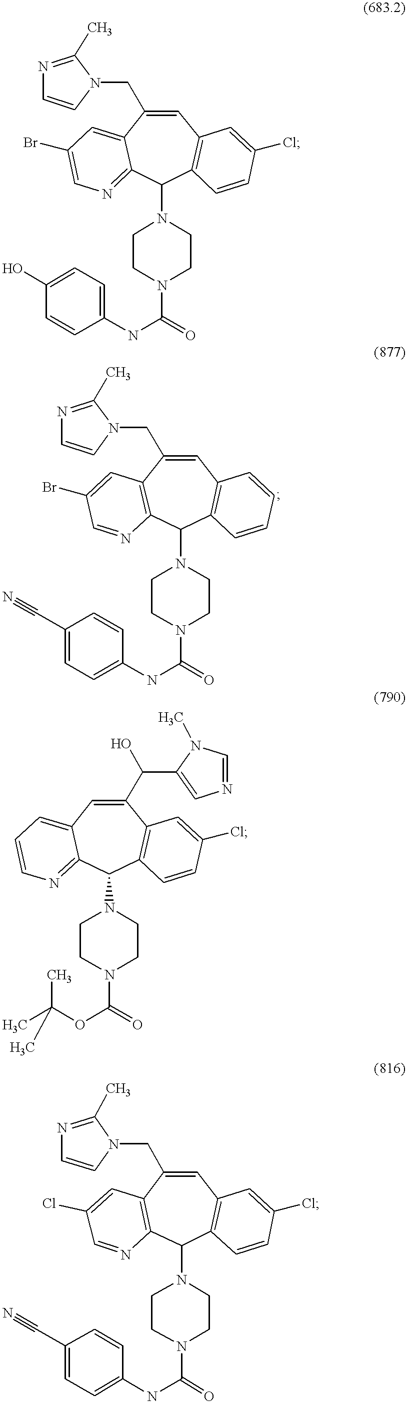

Most preferred compounds of this invention are listed below:

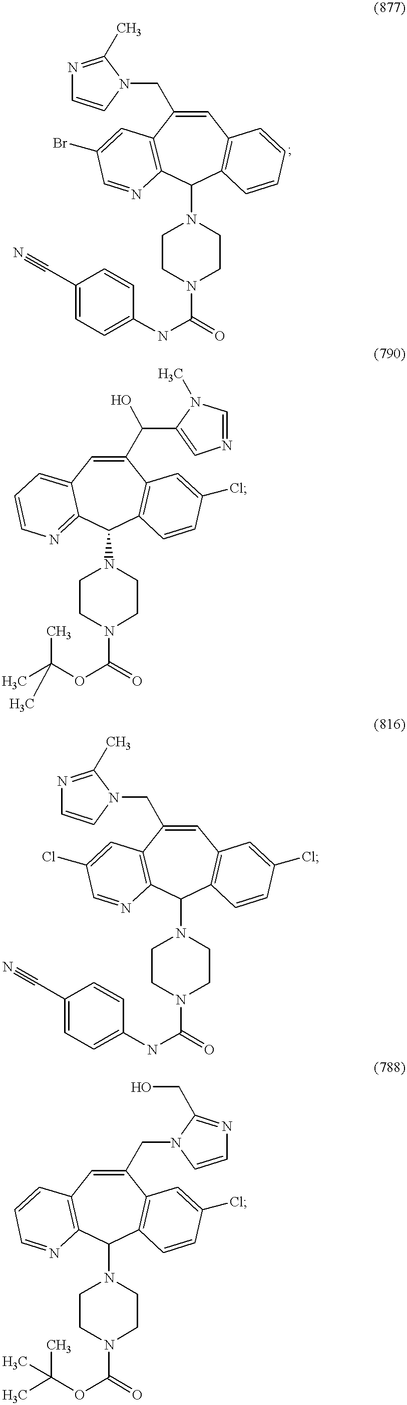

Lines drawn into the ring systems indicate that the indicated bond may be attached to any of the substitutable ring carbon atoms.

Certain compounds of the invention may exist in different isomeric (e.g., enantiomers, diastereoisomers, atropisomers) forms. The invention contemplates all such isomers both in pure form and in admixture, including racemic mixtures. Enol forms are also included.

Certain tricyclic compounds will be acidic in nature, e.g. those compounds which possess a carboxyl or phenolic hydroxyl group. These compounds may form pharmaceutically acceptable salts. Examples of such salts may include sodium, potassium, calcium, aluminum, gold and silver salts. Also contemplated are salts formed with pharmaceutically acceptable amines such as ammonia, alkyl amines, hydroxyalkylamines, N-methylglucamine and the like.

Certain basic tricyclic compounds also form pharmaceutically acceptable salts, e.g., acid addition salts. For example, the pyrido-nitrogen atoms may form salts with strong acid, while compounds having basic substituents such as amino groups also form salts with weaker acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous NaOH, potassium carbonate, ammonia and sodium bicarbonate. The free base forms differ from their respective salt forms somewhat in certain physical properties, such as solubility in polar solvents, but the acid and base salts are otherwise equivalent to their respective free base forms for purposes of the invention.

All such acid and base salts are intended to be pharmaceutically acceptable salts within the scope of the invention and all acid and base salts are considered equivalent to the free forms of the corresponding compounds for purposes of the invention.

The compounds of formula 1.0 can exist in unsolvated as well as solvated forms, including hydrated forms, e.g., hemi-hydrate. In general, the solvated forms, with pharmaceutically acceptable solvents such as water, ethanol and the like are equivalent to the unsolvated forms for purposes of the invention.

The method of treating proliferative diseases (cancer), according to this invention, includes a method for treating (inhibiting) the abnormal growth of cells, including transformed cells, in a patient in need of such treatment (e.g., a mammal such as a human), by administering, concurrently or sequentially, an effective amount of a compound of this invention and an effective amount of a chemotherapeutic agent and/or radiation. Abnormal growth of cells means cell growth independent of normal regulatory mechanisms (e.g., loss of contact inhibition), including the abnormal growth of: (1) tumor cells (tumors) expressing an activated ras oncogene; (2) tumor cells in which the ras protein is activated as a result of oncogenic mutation in another gene; and (3) benign and malignant cells of other proliferative diseases.

In preferred embodiments, the methods of the present invention include methods for treating or inhibiting tumor growth in a patient in need of such treatment (e.g., a mammal such as a human) by administering, concurrently or sequentially, (1) an effective amount of a compound of this invention and (2) an effective amount of at least one antineoplastic agent, microtubule affecting agent and/or radiation therapy. Examples of tumors which may be treated include, but are not limited to, epithelial cancers, e.g., prostate cancer, lung cancer (e.g., lung adenocarcinoma), pancreatic cancers (e.g., pancreatic carcinoma such as, for example, exocrine pancreatic carcinoma), breast cancers, colon cancers (e.g., colorectal carcinomas, such as, for example, colon adenocarcinoma and colon adenoma), ovarian cancer, and bladder carcinoma. Other cancers that can be treated include melanoma, myeloid leukemias (for example, acute myelogenous leukemia), sarcomas, thyroid follicular cancer, and myelodysplastic syndrome. In particular, the proliferative disease (tumor) that may be treated is selected from lung cancer, pancreatic cancer, prostate cancer and myeloid leukemia. Preferrably for the methods of the present invention, the disease (tumor) that may be treated is selected from lung cancer and myeloid leukemia.

The methods of treating proliferative diseases, according to this invention, also include a method for treating (inhibiting) proliferative diseases, both benign and malignant, wherein ras proteins are aberrantly activated as a result of oncogenic mutation in other genes—i.e., the ras gene itself is not activated by mutation to an oncogenic form. This method comprises administering, concurrently or sequentially, an effective amount of a compound of this invention and an effective amount of an antineoplastic agent and/or radiation therapy to a patient in need of such treatment (e.g., a mammal such as a human). Examples of such proliferative diseases which may be treated include: the benign proliferative disorder neurofibromatosis, or tumors in which ras is activated due to mutation or overexpression of tyrosine kinase oncogenes (e.g., neu, src, abl, Ick, lyn, fyn).

For radiation therapy, y-radiation is preferred.

The methods of treating proliferative diseases (cancer), according to this invention, also include a method for treating (inhibiting) the abnormal growth of cells, including transformed cells, in a patient in need of such treatment (e.g., a mammal such as a human), by administering, concurrently or sequentially, an effective amount of a compound of this invention and an effective amount of at least one signal transduction inhibitor.

Typical signal transduction inhibitors include but are not limited to:

(i) Bcr/abl kinase inhibitors such as, for example, STI 571 (Gleevec);

(ii) Epidermal growth factor (EGF) receptor inhibitor such as, for example, Kinase inhibitors (Iressa, SSI-774) and antibodies (Imclone: C225 [Goldstein et al. (1995), Clin Cancer Res. 1:1311-1318], and Abgenix: ABX-EGF) and

(iii) Her-2/neu receptor inhibitors such as, for example, Herceptin® (trastuzumab).

As used herein the following terms have the following meanings unless indicated otherwise:

antineoplastic agent—a chemotherapeutic agent effective against cancer;

concurrently—(1) simultaneously in time, or (2) at different times during the course of a common treatment schedule; and

sequentially—(1) administration of one component of the method ((a) compound of the invention, or (b) chemotherapeutic agent, signal transduction inhibitor and/or radiation therapy) followed by administration of the other component or components; after adminsitration of one component, the next component can be administered substantially immediately after the first component, or the next component can be administered after an effective time period after the first component; the effective time period is the amount of time given for realization of maximum benefit from the administration of the first component.

The term “in association with” as used herein in reference to the combination therapies of the invention means-the agents or components are adminstered concurrently or sequentially as defined above.

### CHEMOTHERAPEUTIC AGENTS

Classes of compounds that can be used as chemotherapeutic agents (antineoplastic agent/microtubule affecting agents) include but are not limited to: alkylating agents, antimetabolites, natural products and their derivatives, hormones and steroids (including synthetic analogs), and synthetics. Examples of compounds within these classes are given below.

Alkylating agents (including nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (including vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, paclitaxel (paclitaxel is commercially available as Taxol® and is described in more detail below in the subsection entitled “Microtubule Affecting Agents”), paclitaxel derivatives (e.g. taxotere), Mithramycin, Deoxyco-formycin, Mitomycin-C, L-Asparaginase, Interferons (especially IFN-a), Etoposide, and Teniposide.

Hormones and steroids (including synthetic analogs): 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex.

Synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Particularly preferred are the antineoplastic agents selected from Cyclophasphamide, 5-Fluorouracil, Temozolomide, Vincristine, Cisplatin, Carboplatin, and Gemcitabine. Most preferrably, the antineoplastic agent is selected from Gemcitabine, Cisplatin and Carboplatin.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

### MICROTUBULE AFFECTING AGENTS

As explained above, the present invention also provides methods of treating diseased cells by contacting the cells with an FPT inhibiting compound of the invention and a microtubule affecting agent (e.g., paclitaxel, a paclitaxel derivative or a paclitaxel-like compound). As used herein, a microtubule affecting agent is a compound that interferes with cellular mitosis, i.e., having an anti-mitotic effect, by affecting microtubule formation and/or action. Such agents can be, for instance, microtubule stabilizing agents or agents which disrupt microtubule formation.

Microtubule affecting agents useful in the invention are well known to those of skill in the art and include, but are not limited to allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), paclitaxel derivatives (e.g., Taxotere, NSC 608832), thiocolchicine (NSC 361792), trityl cysteine (NSC 83265), vinblastine sulfate (NSC 49842), vincristine sulfate (NSC 67574), epothilone A, epothilone, and discodermolide (see Service, (1996) Science, 274:2009) estramustine, nocodazole, MAP4, and the like. Examples of such agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

Particularly preferred agents are compounds with paclitaxel-like activity. These include, but are not limited to paclitaxel and paclitaxel derivatives (paclitaxel-like compounds) and analogues. Paclitaxel and its derivatives (e.g. Taxol and Taxotere) are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

In a preferred embodiment, compounds with possible tubulin polymerization activity are screened in vitro. In a preferred embodiment, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol., 89: 737-758. U.S. Patent No. 5,569,720 also provides in vitro and in vivo assays for compounds with paclitaxel-like activity.

Methods for the safe and effective administration of the above-mentioned microtubule affecting agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, NJ 07645-1742, USA); the disclosure of which is incorporated herein by reference thereto.

General Preparative Schemes

The following processes may be employed to produce compounds of the invention.

### Pyridyl Tricyclic Compounds

One skilled in the art will appreciate that the compounds of the invention represented by Formula 1, wherein one of a, b, c or d is N or N⁺—O⁻ can be prepared according to the following schemes:

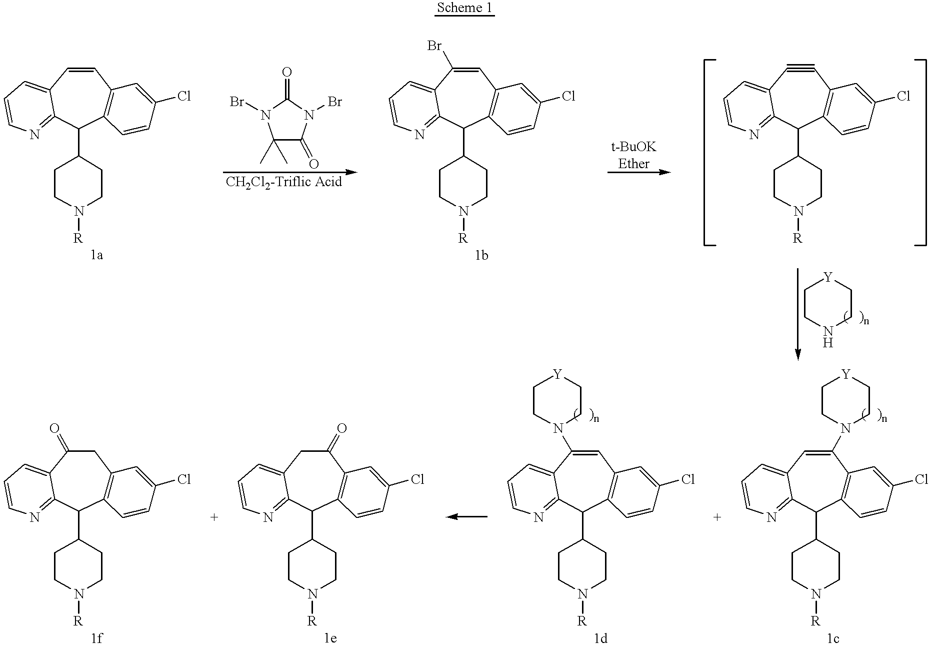

The synthesis of 5-bromo tricyclic compound 1b begins with bridgehead olefin 1a (J. Med Chem (1998), 41,1561-1567) which is treated with dibromo dimethylhydantoin in triflic acid media. Further treatment of the vinylbromide with potassium t-butoxide in the presence of the appropriate secondary amine gives the 5 and 6-substituted enamine adducts. When Y is NH (piperazine case), acylations, sulfonylations and amide formation can be carried out using standard procedures. Treatment of these amine adducts with HCl(aq) at the appropriate temperatures results in the formation of the 5 and 6 azaketones, 1f and 1e respectively.

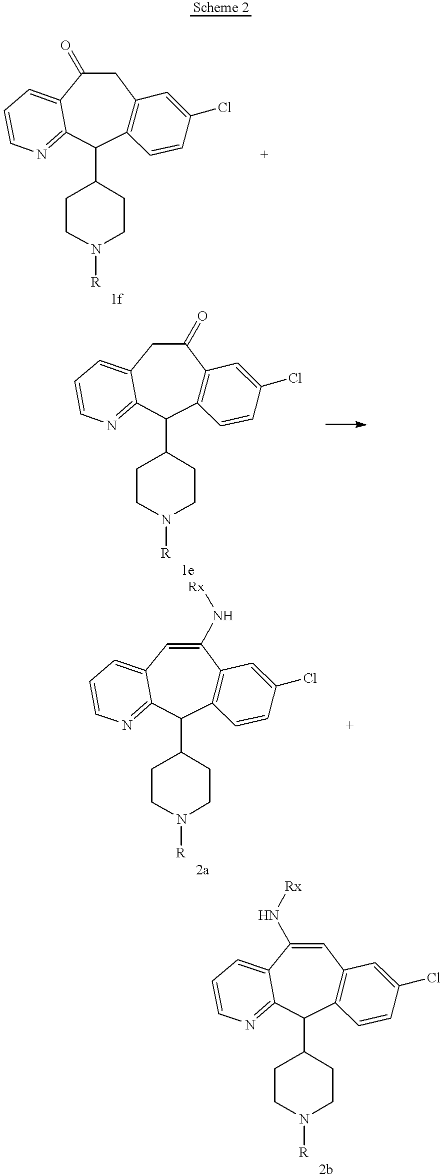

In cases where secondary enamines were required, synthesis from 1f and 1e-azaketones were utilized as outlined in scheme 2. Thus, the appropriate ketone and 5 amine was refluxed in toluene in the presence of p-toluene sulfonic acid in a Dean Stark apparatus.

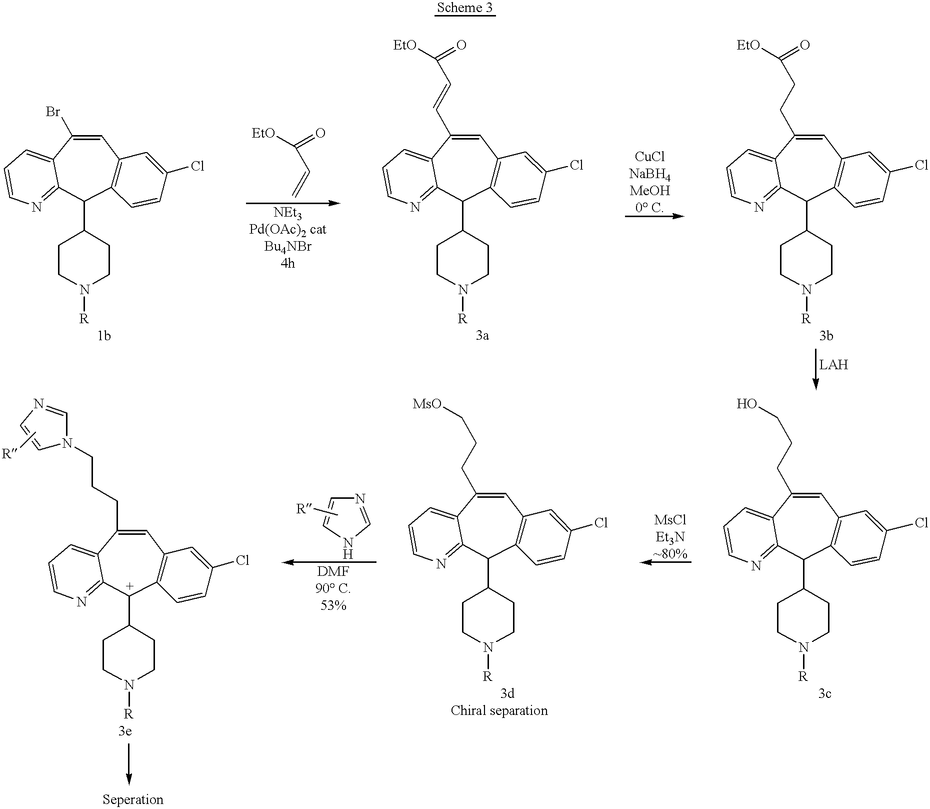

Synthesis of 3-carbon spaced analogs can be prepared as outlined in scheme 3. Thus, subjecting tricyclic vinyl bromide 1 b to a Heck type reaction using ethyl acrylate and catalyzed by Pd⁰ gives the α-β un-saturated ester 3a. Reduction of the conjugated double bond was carried out using copper chloride-sodium borohydride reducing reagent. The ester was further reduced to alcohol using lithium aluminum hydride. Treatment of the alcohol with methanesulfonyl chloride in an appropriate aprotic solvent, followed by displacement with an appropriate sodium salt resulted in the desired imidazole targets. In most cases, separation of isomers were effected at this point. Where the R group of 3e was a BOC group, deprotection using HCl-dioxane gave the hydrochloride salts of amines. Using standard chemistry, these amines were converted to ureas, carbamates, sulfonamides and amides.

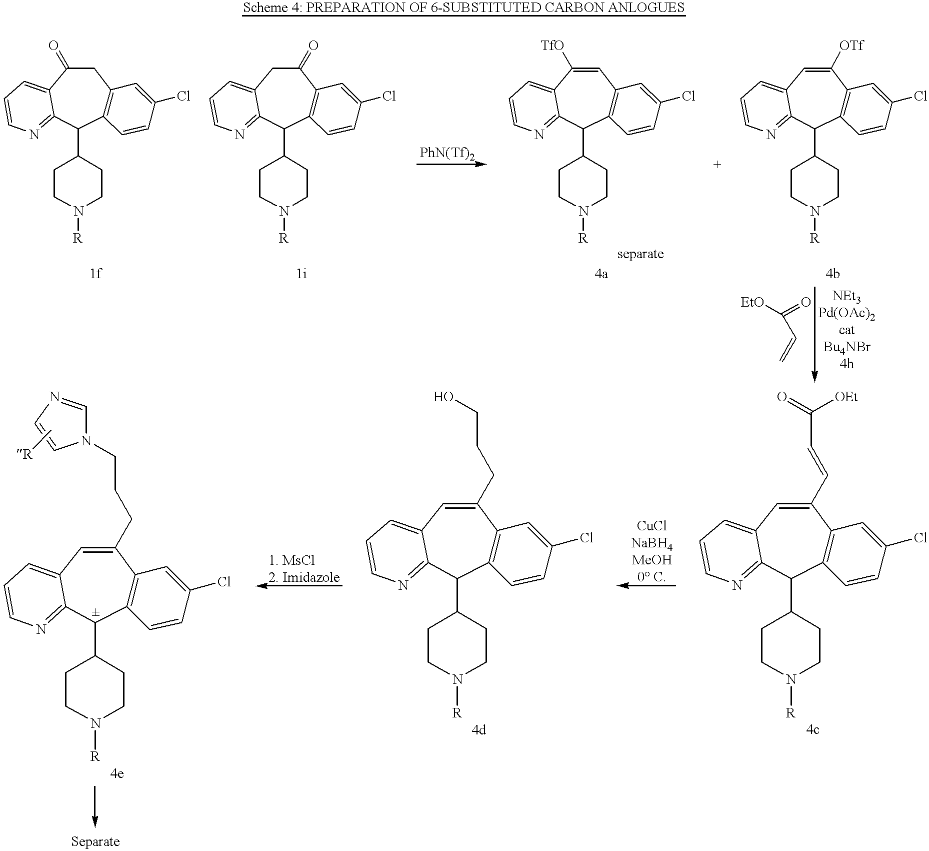

Preparation of 6-substituted 3-carbon spaced imidazole compounds was carried out as outlined in scheme 4. A mixture of ketones if and 1i were treated with N-phenytrifluoromethane sulfonimide to give a seperable mixture of 5 and 6-tricyclic triflate compounds. The 6-trilate adduct was converted to the desired 3-carbon spaced analogs using similar protocol as described for the 5-bromo tricyclic compounds outlined in scheme 3.

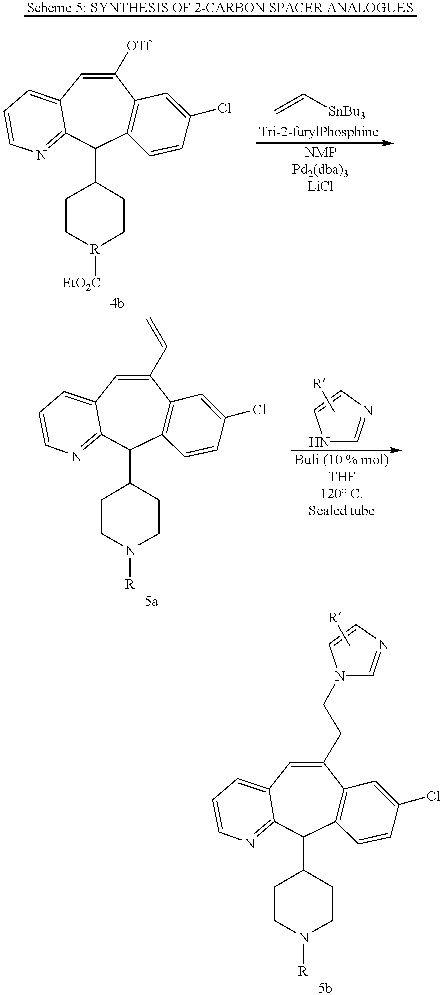

Two carbon spaced analogs were prepared as outlined in scheme 5. Thus, triflate 4b was subjected to Stille chemistry, by reacting with tributylvinyl stannate catalyzed by an appropriate Pd⁰ to afford the tricyclic vinyl compound 5b. The 2-carbon spaced compounds were obtained by treating the tricylic compound with the appropriate imidazole that had been previously treated with Buli-THF in a sealed tube and refluxed at 120° C. Further funtionalization was carried out as previously described. Suberane compounds were prepared in a similar way.

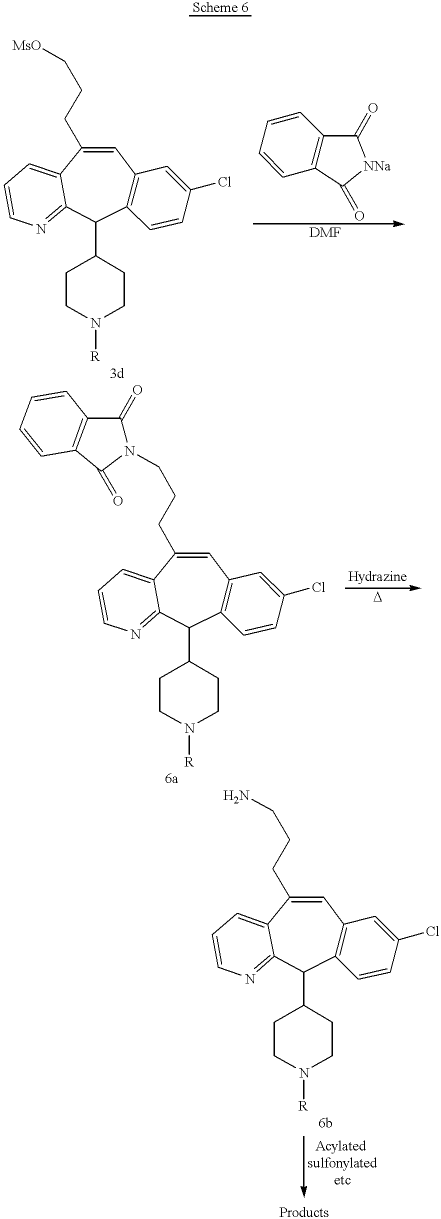

Scheme 6 illustrates method of making amine 6b through phthalimido displacement of a mesylate followed by hydazine hydrolysis of the phthalimido moiety. Amine 6b can be converted to targets that have acyl, sufonyl,

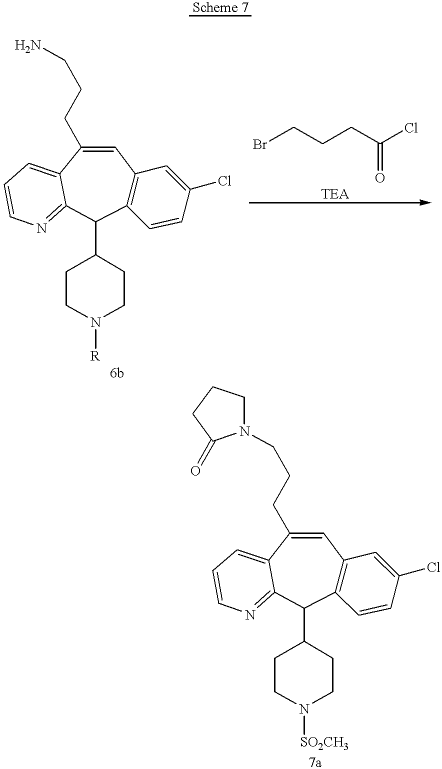

Lactams 7a can be prepared from amine 6b by reacting with bromo butanonyl acid chloride as outlined in scheme 7.

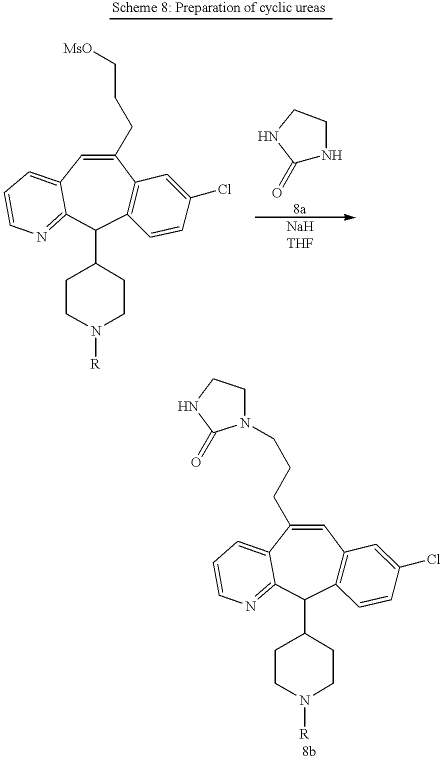

Cyclic urea can be prepared from the mesylate shown above by treating with the salt of the cyclic urea 8a as outlined in scheme 8.

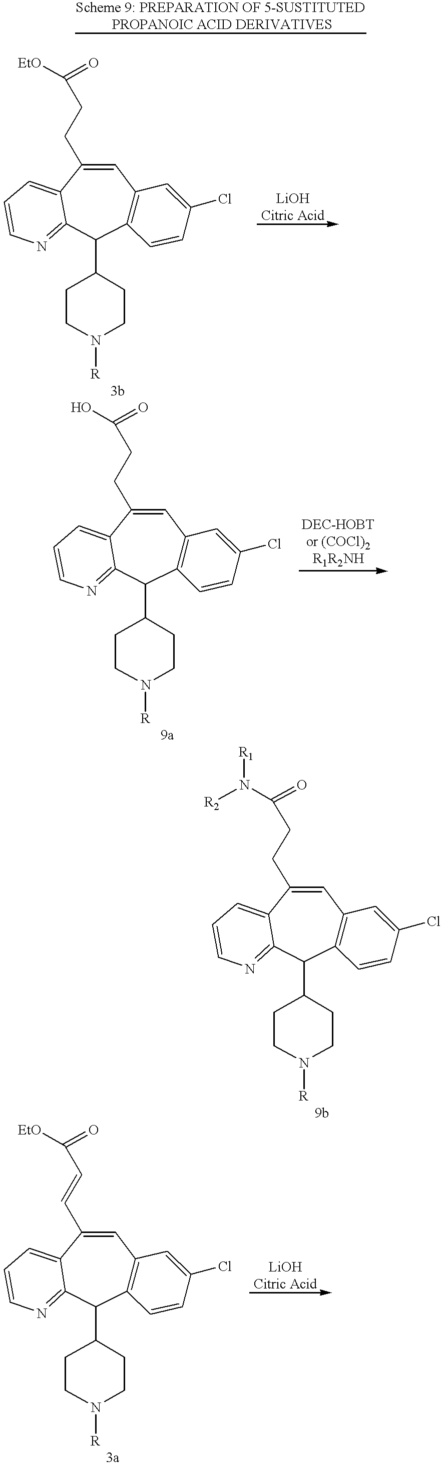

Amides from 3-carbon spaced carboxylic acid 9a and 9c can be prepared as outlined in scheme 10 using either DEC—HOBT mediated protocol or from the appropriate acid chloride.

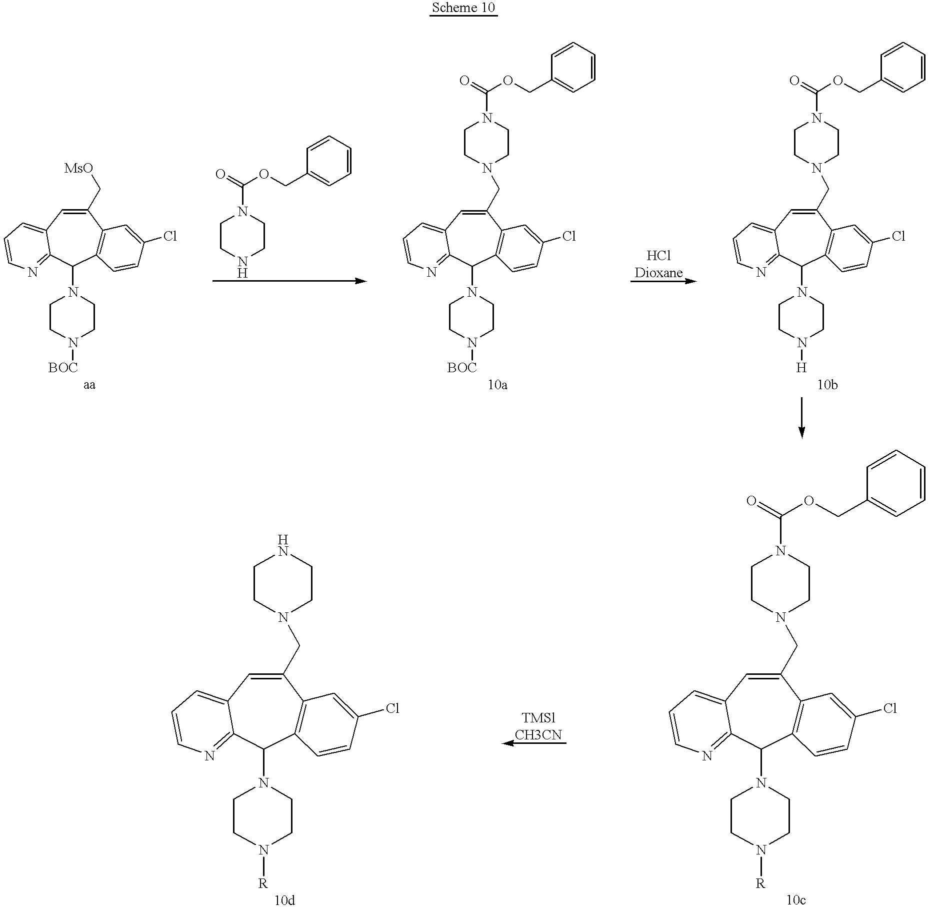

Preparation of piperazine compounds off the bridgehead starts from mesylate aa which is reacted with CBZ-protected piperazine. The BOC group is then removed and the resulting amine 10c is functionalized appropriately. Removal of CBZ group off the piperazine is effected with TMSI.

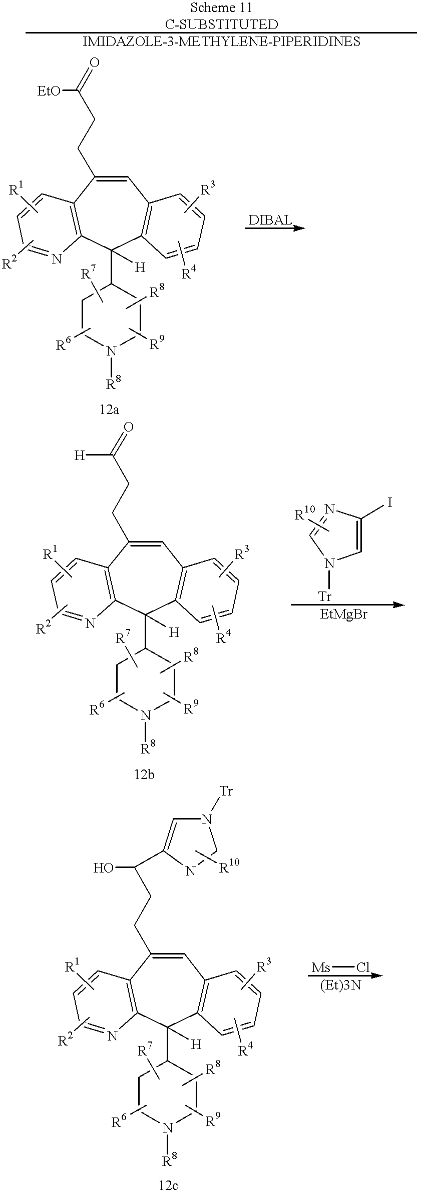

Compound 12a is reduced with DIBAL in an inert solvent such as toluene or tetrahydrofuran to give 12b after acidic workup. Treatment of 12b with an appropriately substituted and tritylated imidazole iodide in the presence of ethylmagnesium bromide in solvents such as dichloromethane at ambient temperature yields the adduct 12c. Elimination of the hydroxyl group by converting the hydroxyl group to an appropriate leaving group such as a mesylate, tosylate, or halide, using methanesulfonyl chloride, p-toluenesulfonyl chloride, or thionyl chloride, followed by elimination using an appropriate base such as triethylamine gives 12e. Removal of the trityl group with acid such as trifluoroacetic acid or hydrochloric acid gives the double bond compound 12f which is then hydrogenated using an appropriate catalyst such as platinum oxide under from 1 to 55 psi of hydrogen in an appropriate solvent such as ethanol gave the desired product 12g.

Alternatively the ester 12a can be saponified with an appropriate base such as lithium hydroxide to obtain the acid 12h. Converting the acid 12h to the “Weinreb amide” followed by reaction with an appropriately substituted and tritylated imidazole iodide in the presence of ethylmagnesium bromide in solvents such as dichloromethane at ambient temperature yields the adduct 12c (shown in Scheme 12 below).

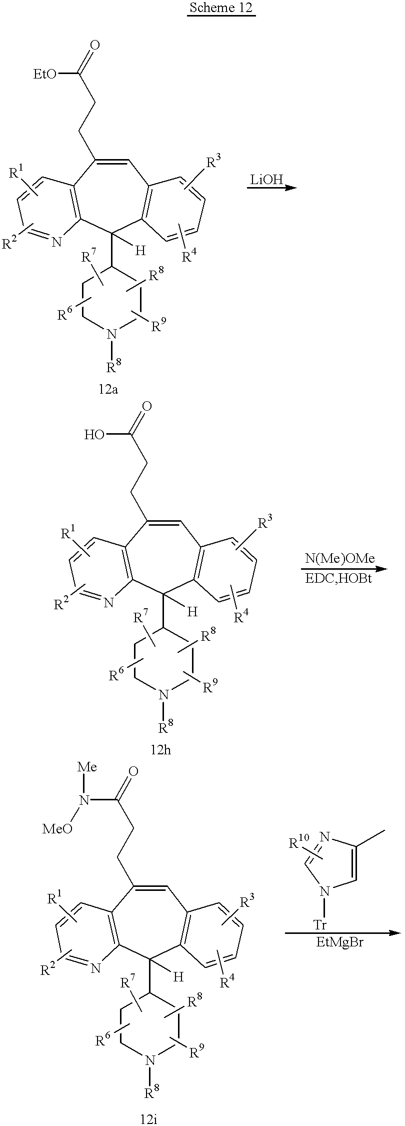

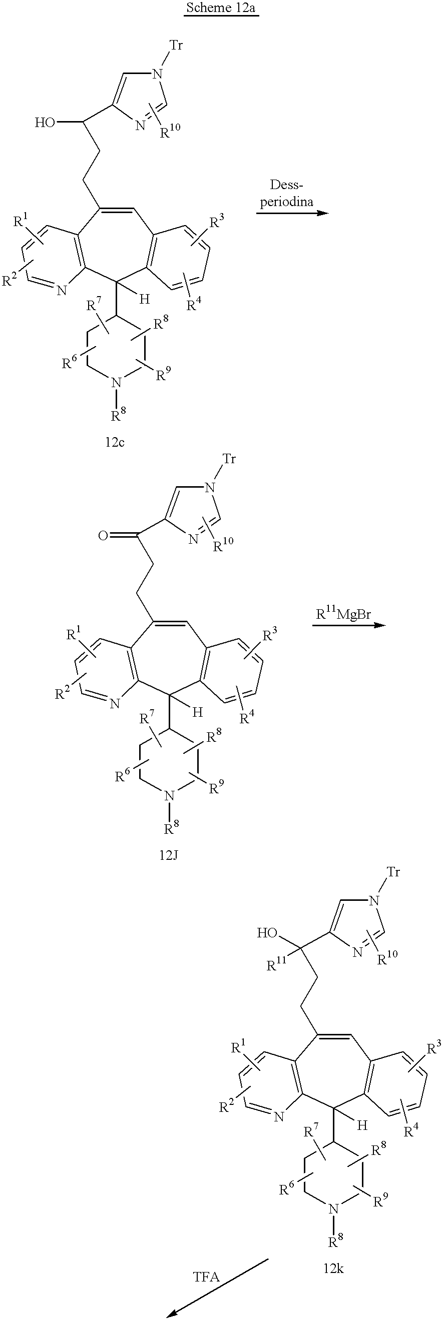

Compounds of type 12L were prepared as shown above. Oxidation of the hydroxyl compound 12c can be accomplished with the Dess Martin periodinane to obtain 12j. Reaction with a grignard reagent gave 12k. The trityl group is removed under standard conditions mentioned above to give the desired compound 12L.

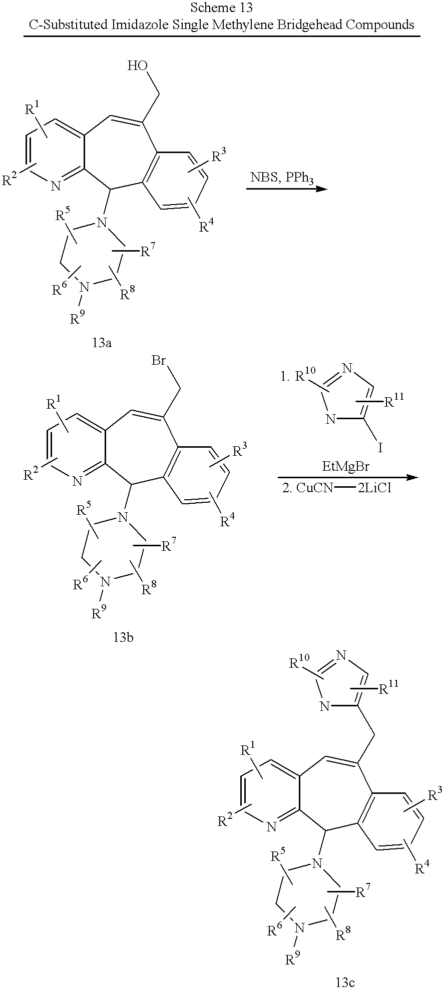

Single methylene bridgehead C-Imidazole derivatives (13c) were prepared as shown above. Compound 13a was first converted to bromide 13b. Treatment of compound 13b with C-imidazole cuprates (prepared from corresponding iodo imidazole) yielded the adduct 13c.

Scheme 14: Preparation of one-methylene piperazines

Ketone A is brominated with brominating reagents such as NBS, with a small amount of an activator such as benzoyl peroxide, in solvents such as dichloromethane at elevated temperature, such as 80-100° C. to give dibromo compound B.

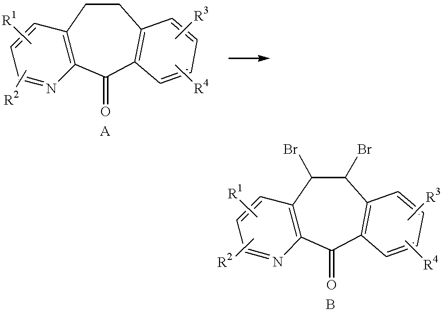

Dibromo compound B is reacted with a base such as DBU in a solvent such as dichloromethane at temperatures from 0° C. to room temperature to give vinylbromides C and D. These vinylbromides are separated by chromatography such as silica gel flash chromatography using solvents mixtures such as ethyl acetate and hexane. Alternatively, vinylbromides C and D can be separated by crystallization from solvents such as dichloromethane.

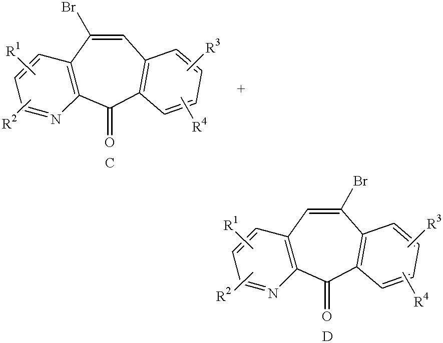

The ketone groups of separated vinylbromides C and D are reduced to the corresponding alcohols E and F with a reducing agent such as NaBH₄ in solvents such as methanol or ethanol at temperatures of 0° C. to room temperature.

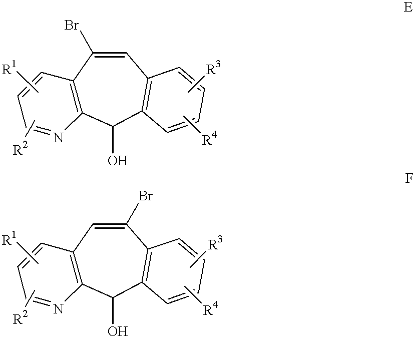

The resulting alcohols functions of E and F are converted to a leaving group, such as a halide, with reagents such as SOCl₂ in solvents such as dichloromethane containing a base such as 2,6-lutidine and running the reaction at 0° C. to room temperature. The resulting intermediate halides are reacted, without purification, with piperazine or a protected piperazine, such as BOC-piperazine in a solvent such as dichloromethane at room temperature giving intermediates G and H.

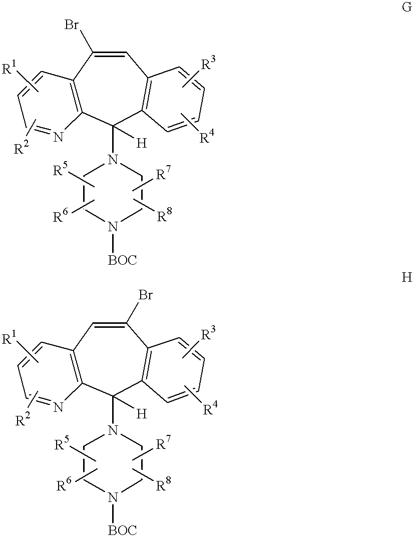

The vinylhalide intermediates are carbonylated with CO gas under a pressure of about 100 psi and a temperature of 80° C. to 100° C. using a palladium catalyst such as PdCl₂ and triphenyl phosphine in toluene and containing DBU and an alcohol such as methanol. If methanol is used, methyl esters I and J are obtained.

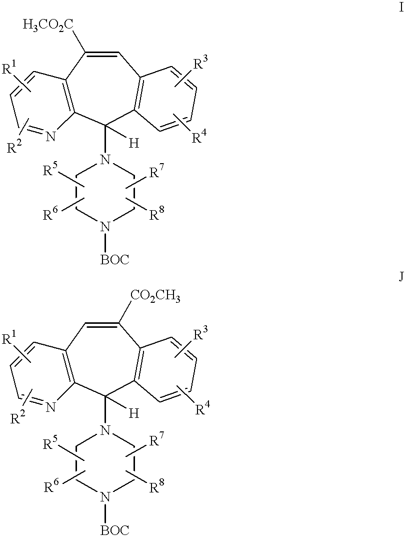

The ester functions are of I and J are reduced to hydroxymethyl functions of K and L. This can be done directly by first removing the protecting BOC group with TFA or HCl-dioxane and then reducing with a reducing agent such as DIBAL-H, followed by reintroduction of the BOC group with di-tert-butyl dicarbonate. Alternatively, the ester function is hydrolyzed with LiOH and water followed by neutralization with citric acid. The resulting carboxylic acids are then converted into a function that is easily reduced, such as a mixed anhydride or an acyl imidazole. This is done by reacting the resulting carbocylic acids with a chloroformate to form the mixed anhydride or with carbonydiimidazole to form the acyl imidazole (Synlett. (1995), 839). The resulting activated carboxylic acids are reduced with NaBH₄ in solvents such as methanol, ethanol or aqueous THF.

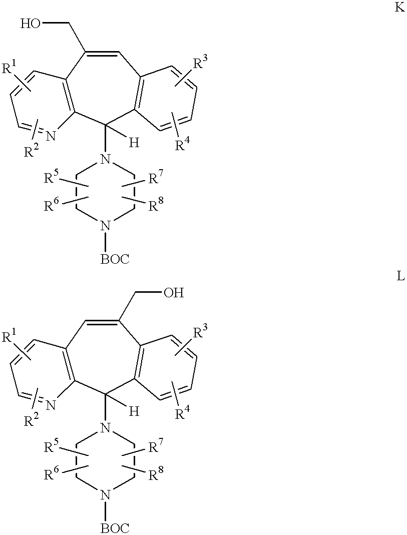

The hydroxy functions of K and L are converted into leaving groups such as a methanesulfonate or an arylsulfonate such as a tosylate, by reacting with the appropriate sulfonyl chloride in dichloromethane containing a base such as triethylamine. The sulfonate leaving groups can be displaced by nucleophiles such amines. The nucloephile can also be basic heterocycles such as imidazole or a substituted imidazole. In the case of an imidazole, the anion of the imidazole is first formed with NaH in DMF and then reacted with the above sulfonate. Displacement of the sulfonates with a nucleophile gives O and P, which can be converted to the compounds of this invention 1.0, by first removing the BOC protecting group and then forming the desired amide, urea, carbamate or sulfonamide on the resulting amine by methods well known in the art.

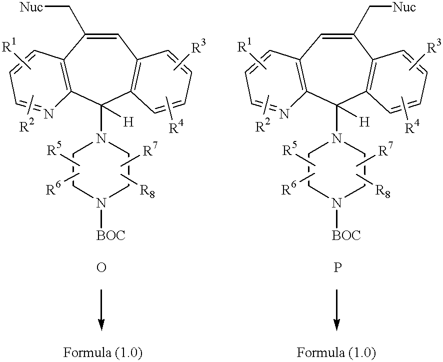

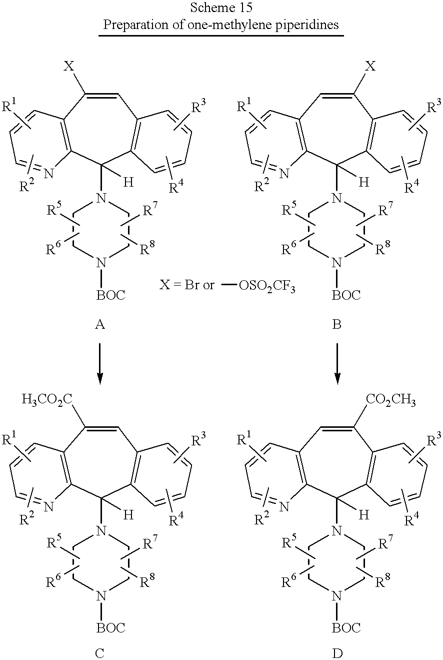

The vinylhalide or vinyltriflate intermediates A and B, (described in other general schemes) are carbonylated with CO gas under a pressure of about 100 psi and a temperature of 80° C. to 100° C. using a palladium catalyst such as PdCl₂ and triphenyl phosphine in toluene and containing DBU and an alcohol such as methanol. If methanol is used, methyl esters C and D are obtained. Intermediates C and D are reacted as are intermediates I and J in the general scheme for one methylene piperazines to yield compounds of Formula 1.0, of this invention.

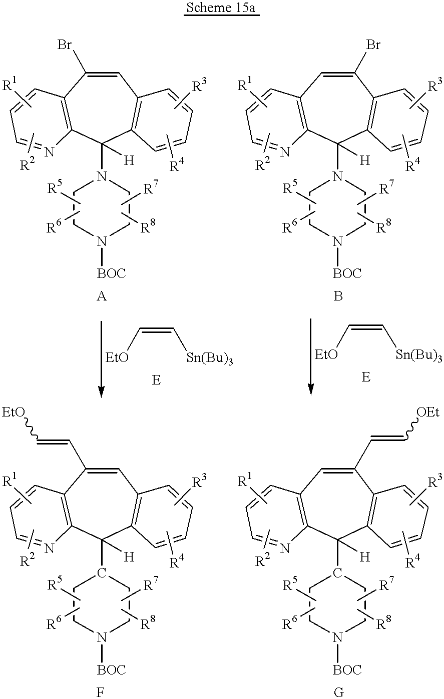

Alternatively, Intermediates A and B can be reacted with tin vinylether E, in the presence of PdCl₂, as described in Tetrahedron, (1991), 47, 1877, to yield vinylethers F and G (Scheme 15a). Allowing F and G to stand until aldehyde is visible by NMR (at least two weeks) and then reacting with Hg(OAc)₂, Kl followed by NaBH₄, as described in J. Chem. Soc., Perkin Trans., (1984), 1069 and Tet. Lett., (1988), 6331, yields mixtures H, I and J, K. Intermediates H and J are separated and reacted as are intermediates K and L in the general scheme for one methylene piperazines to yield compounds of Formula 1.0, of this invention.

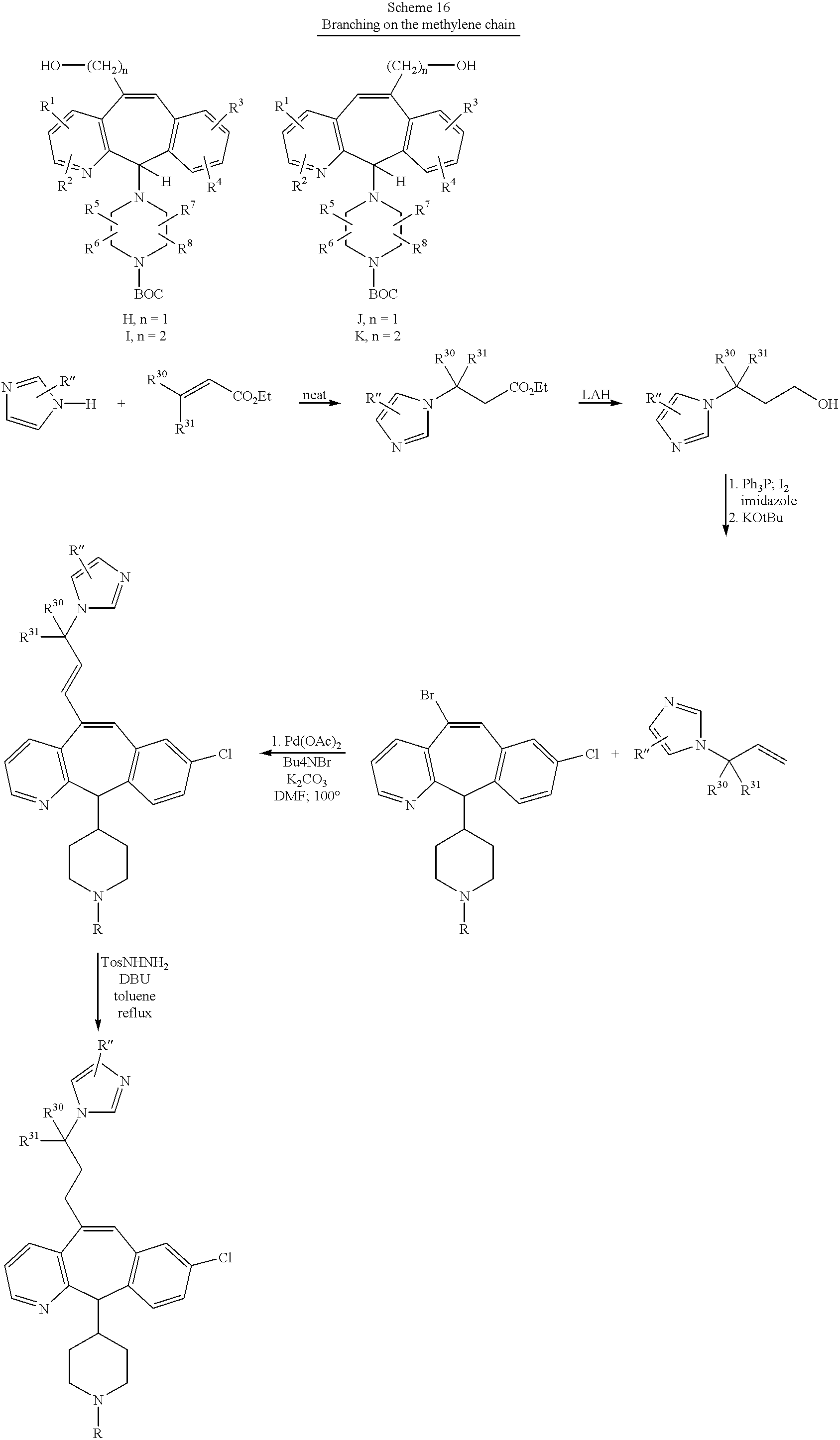

Compounds with substitution along the chain can be synthesized starting with a substituted ethyl acrylate derivative. Addition of imidazole across the olefin followed by reduction gives the terminal alkene, which can be added to the appropriately substituted vinyl bromide under Heck reaction conditions. Selective reduction of the di-substituted olefin gives the saturated derivative (Scheme 16).

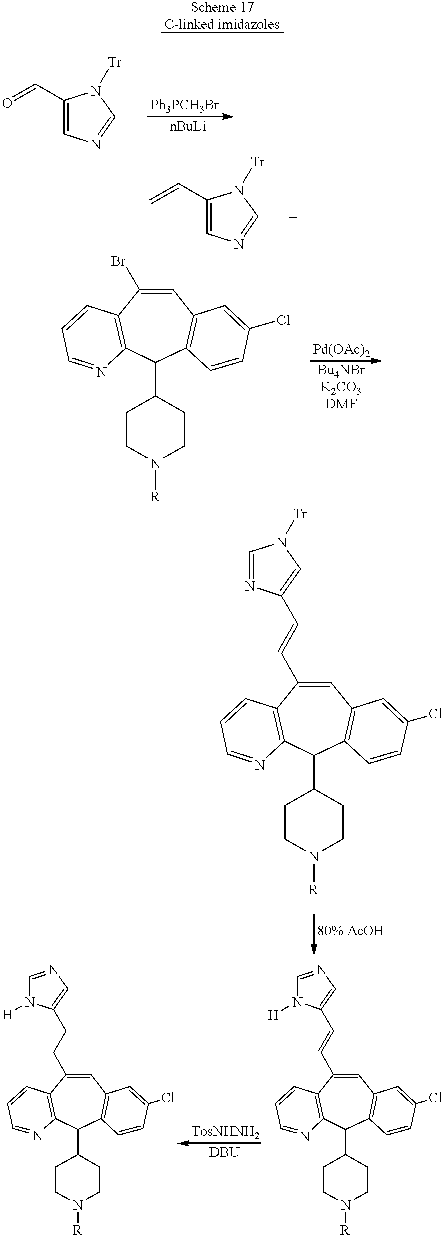

The synthesis of the C-linked imidazoles proceeds through the Heck reaction of the appropriately substituted vinyl imidazole with the appropriate vinyl bromide. Selective reduction of the resulting di-substituted olefin gives the target compound. A similar procedure can be carried out with differentially N-substituted imidazoles to give N-alkyl imidazole derivatives (Scheme 17).

### Suberyl Compounds

One skilled in the art will appreciate that the compounds of the invention represented by Formula 1.0, wherein a, b, c or d is C can be prepared according to the following schemes:

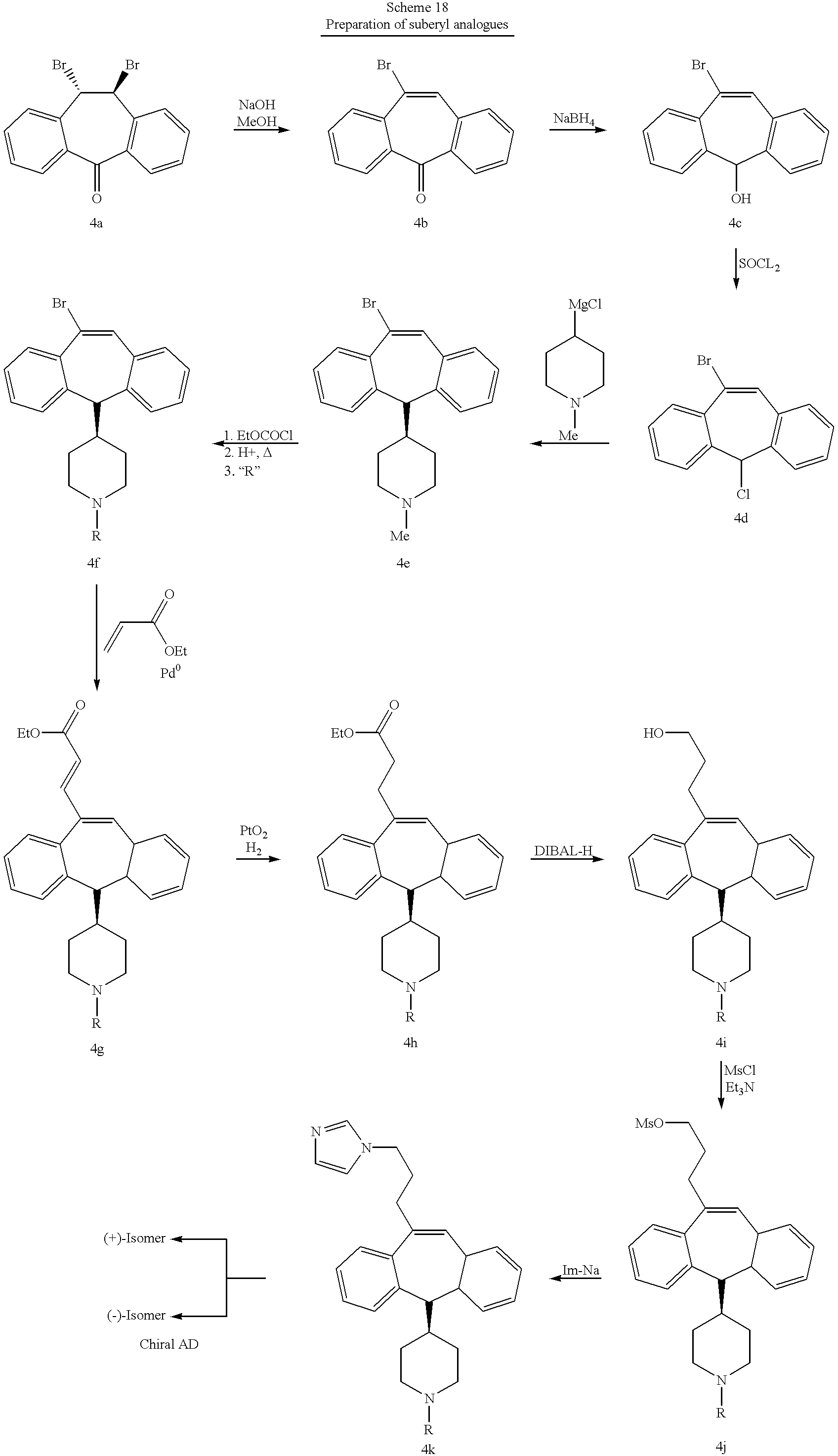

Tricyclic vinyl bromide azaketone 4b was prepared as described by Rupard et. al. (J. Med. Chem. 1989, 32, 2261-2268). Reduction of ketone to alcohol 4c was carried out with NaBH₄. The alcohol was converted to chloride 4d and then treated with N-methylpiperidine Grignard reagent to give piperidine derivative 4e. Demethylation was effected with ethyl chloroformate followed by acid hydrolysis and subsequent derivitization (i.e sulfonylation, acylation and carbomylation etc.). Preparation of compounds with 3-carbon substituted imidazole moieties on the suberane trycyclic bridgehead was carried out in a similar way as described in scheme 3.

### PREPARATION OF INTERMEDIATES AND EXAMPLES

### Preparative Example 1

Step A Preparation of Compound (2).

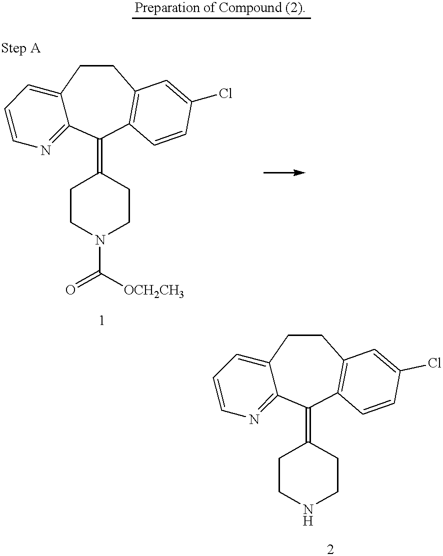

Loratadine® (448 g, 1.17 mol) was refuxed in 2 L of 70% aqueous HCl (1.4 L conc.HCl in 600 ml H₂O) for 12h. The reaction mixture was then cooled and poured into ice. It was then basified with 950 mL of 50% NaOH followed by extraction with CH2Cl₂ (1×4L, and 2×2.5L). The organic phase was washed with brine, dried over Na₂SO₄ and MgSO₄ and then filtered. All the volatiles were then removed to give 368 g of the title compound (2). MH+=311

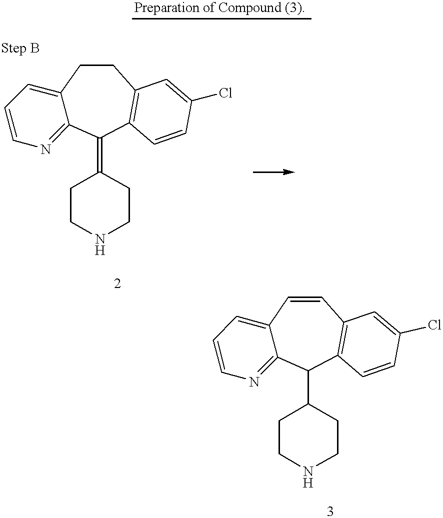

To the title compound from Preparative Example 1, Step A (363 g, 1.17 mol) was added trifuromethane sulfonic acid (1.8 Kg) under N₂. The reaction mixture was refluxed at 170° C. The progress of the reaction was monitored by ¹H NMR. After 4 days the reaction was only 63% complete. After 8 days the reaction was found to be 80% complete according to ¹H NMR; thus another 130 mL of CF₃SO₃H were added and refuxing continued for another 24 h. It was then poured into ice and basified with 800 mL of NaOH (50%) and extracted twice with CH2Cl₂(1×8L then 1×7L). The organic phase was combined, washed with H₂O and filtered through celite. It was then dried over MgSO₄ and Na₂SO₄ and again filtered through celite. The filtrate was concentrated to give a black brown semi-solid that was pre adsorbed on 600 g of silica gel and then chromatographed on 2.3 Kg of silica gel eluting first with 5% CH₃OH—CH₂Cl₂ (saturated with ammonia) and then with 10% CH₃OH—CH2Cl₂ (saturated with ammonia) to give 102 g of the title compound (3) as a solid. mp=73-75; MS (FAB) m/z 483 (MH+).

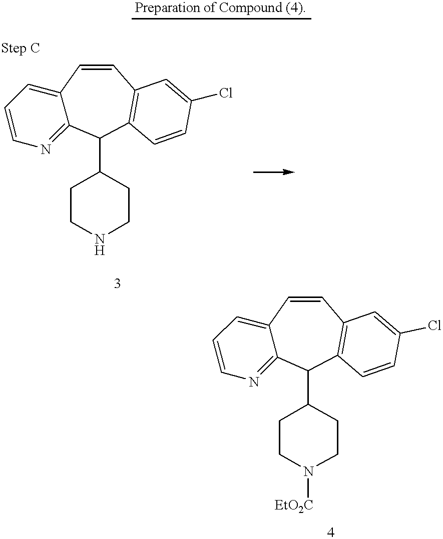

To a solution of the title compound of Preparative Example 1, Step B (145 g) in 1 L of CH₂Cl₂ at 0° C. was added ethylchloroformate (55 mL), dropwise. The reaction mixture was stirred at room temperature overnight. It was further diluted with 1 L CH₂Cl₂ and stirred with 2L of dilute NaHCO₃, pH ˜7-8. The organic layer was separated and dried over MgSO₄ and Na₂SO₄, filtered and concentrated to afford 174 g of a brown black gum. The crude compound was purified by silica gel column chromatography, eluting with 20-60% ethyl acetate-hexane to afford the title compound (4). MS (FAB) m/z 383 (MH+).

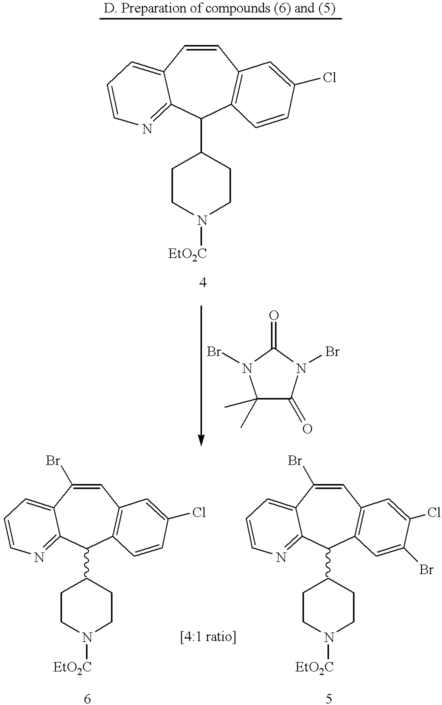

The title compound of Preparative Example 1, Step C (251 g, 0.65 mol) was dissolved in 1.65 L of CH2Cl₂ and dibromo dimethylhydantoin, (132 g, 0.462 mol) was then added. The solution was stirred until the system was homogeneous. The solution was cooled to 0° C. under N₂ atmosphere and 174 mL of CF₃SO₃H were added over 37 min. while keeping temperatures between ⁻1 to 1° C. The reaction mixture was stirred for 3 h, cooled to ⁻10° C. and basified with 50% NaOH (170 mL), keeping the temperature below 1° C. The aqueous phase was extracted with CH2Cl₂ and then dried over MgSO₄, dried and concentrated to give 354 g of yellow foam that was chromatographed on silica gel eluting with 10-50% of ethyl acetate-hexanes gradient to give 50 g of compound (5) (14% yield) and 147 grams of the desired title compound (6) (49% yield). Compound (6) MS m/z (rel intens) 462 (MH+); Compound (5) MS m/z (rel intens) 542 (MH+).

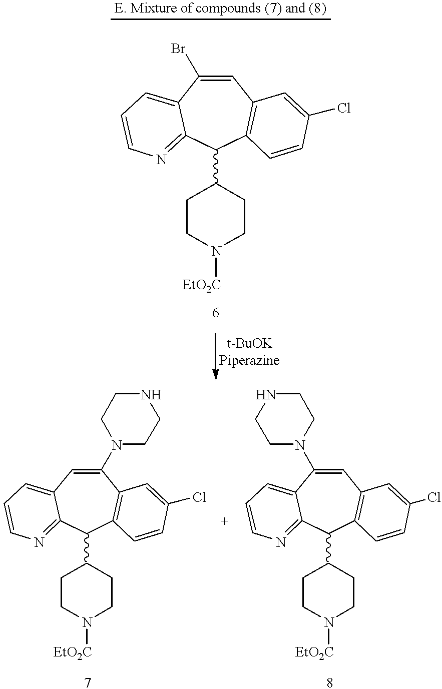

To a solution of piperazine 0.186 g (2.2 mmol, 5 equiv.) in 5 mL of THF was added 0.20 g (0.4 mmol) of compound 6 (from Preparative Example 1, Step D. The reactants stirred at room temperature until everything was in solution. To this mixture was added potassium t-butoxide (0.243 g, 2.1 mmol, 5 equivalents) in one portion. The reaction mixture was stirred at room temperature for 2 h. All of the THF was removed by rotary evaporation and the resulting crude product was purified by flash chromatography eluting with 3-4% (10% CH₃OH: saturated with NH₄OH)—CH₂Cl₂ to give a mixture of title compounds (7) and (8). FAB m/z 467 (MH+).

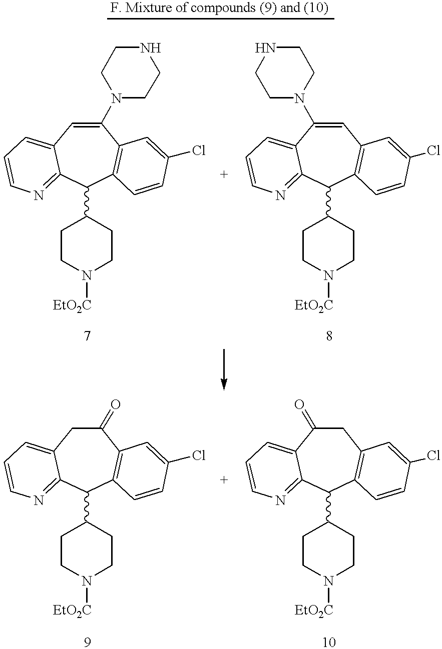

The mixture of compounds from Preparative Example 1, Step E (43.6 g) in 100 mL of conc. HCl was stirred at room temperature for 16 h. The reaction mixture was poured into ice and basified with conc. NH₄OH and then extracted with CH2Cl₂ to give a mixture of compounds (9) and (10). MS (FAB) m/z 399 (MH+).

### Preparative Example 2

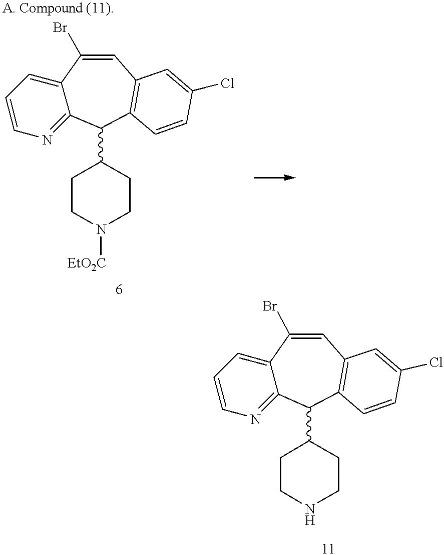

Compound 6 from Preparative Example 1, Step D (10 g, 21.7 mmol) was hydrolyzed in the same manner as described in Preparative Example 1, Step A, to give the title compound (11). MH+=389.

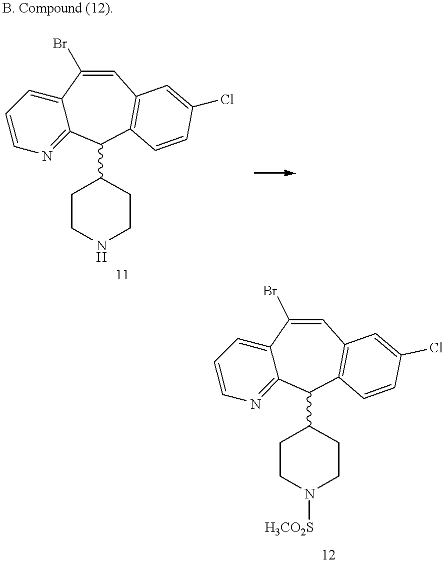

To the amine product from Preparative Example 2, Step A (20 g, 0.5 mol) and triethylamine (10.4 g, 14.4 mL, 1.02 mol) dissolved in anhydrous dichloromethane (100 mL) was added methanesulfonyl chloride (8.8 g, 6 mL, 0.77 mol). After stirring at room temperature overnight, the solution was diluted with dichloromethane, washed with saturated NaHCO₃ and dried over anhydrous magnesium sulfate. Filtration and concentration in vacuo afforded the crude product that was purified by flash chromatography on a silica gel column, eluting with 1% CH₃OH(saturated with ammonia)-CH₂Cl₂ to give the title compound (12). MS (FAB) m/z 469 (MH+).

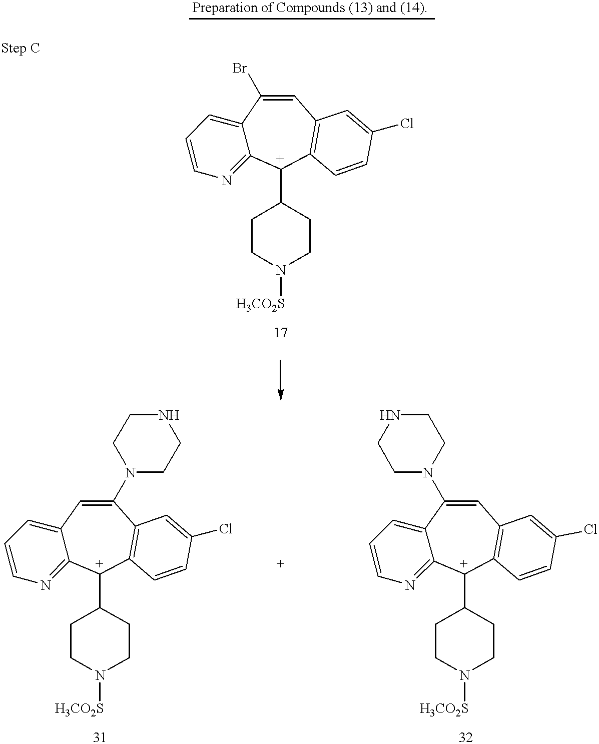

Product from Preparative Example 2, Step B (21.25 g, 45.3 mmol) was treated in the same manner as described in Preparative Example 1, Step E, to give 22.2 g of a mixture of compounds (13) and (14). MS (473) (MH+).

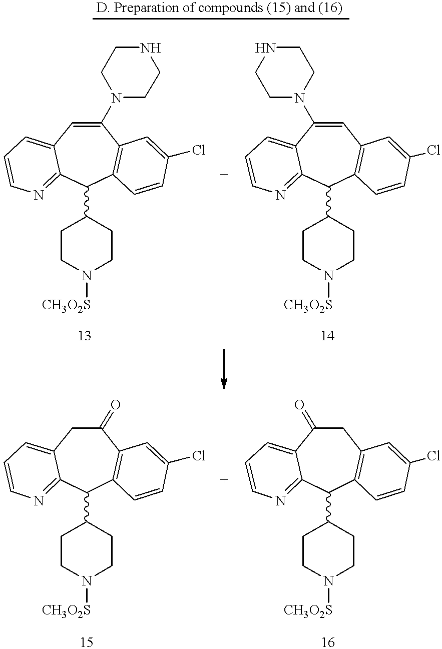

The products from Preparative Example 2, Step C (22.5 g) was dissolved in 150 mL of conc.HCl and stirred for 16 h. The reaction mixture was poured into ice, basified with conc. NH₄OH and then extracted with CH2Cl₂ to give a mixture of compounds (15) and (16), MS (FAB) m/z 405 (MH+).

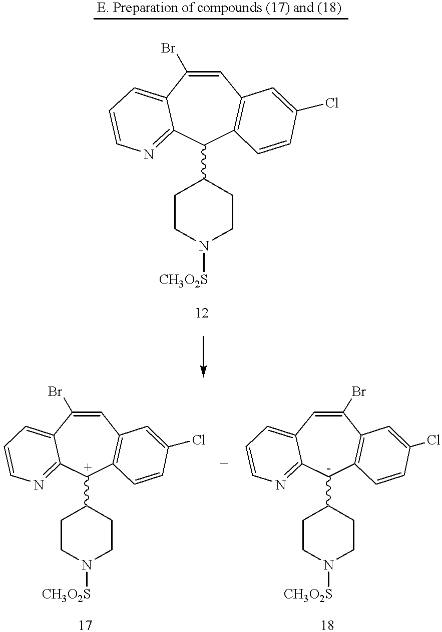

Separation of compound of Preparative Example 2 Step B by HPLC using a Chiralpack AD column eluting with 40-50% isopropanol:60-50% hexane-0.2% diethylamine gave enantiomeric amines (17) and (18).

Compound 17: mp=118-119; [α]_{D}²²=+136.9° (9.00 mg/2 mL, MeOH); MS (FAB) m/z 469 (MH+).

Compound 18: mp=119-120; [α]_{D}²²=−178.2° (9.90 mg/2 mL, MeOH); MS (FAB) m/z 469 (MH+).

### Preparative Example 3

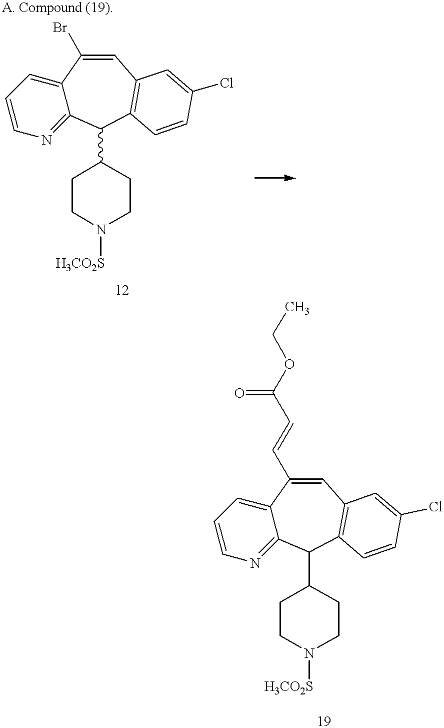

To a solution of the title compound from Preparative Example 2, Step B (2.0 g, 4.3 mmole) in DMF (50 ml) under nitrogen atmosphere, was added triethyl amine (17 ml), ethyl arcrylate (2.5 ml), potassium carbonate (3 g, 21.4 mmole), tetrabutylamonium bromide (2.8 g, 8.6 mmole) and palladium (II) acetate (0.1255 g, 0.56 mmol). The resulting mixture was heated to 100° C., and stirred for 4 h then it was cooled to room temperature and the solvent was removed. To the residue was added CH₂Cl₂ and water and the mixture was then extracted with CH₂Cl₂. The organic layer was dried over magnesium sulfate, filtered and concentrated to dryness. The crude product was purified using pre-adsorbed flash silica column chromatography eluting with 30-50% ethyl acetate-hexane gradient to give the title compound (19). MS 487 (MH⁺).

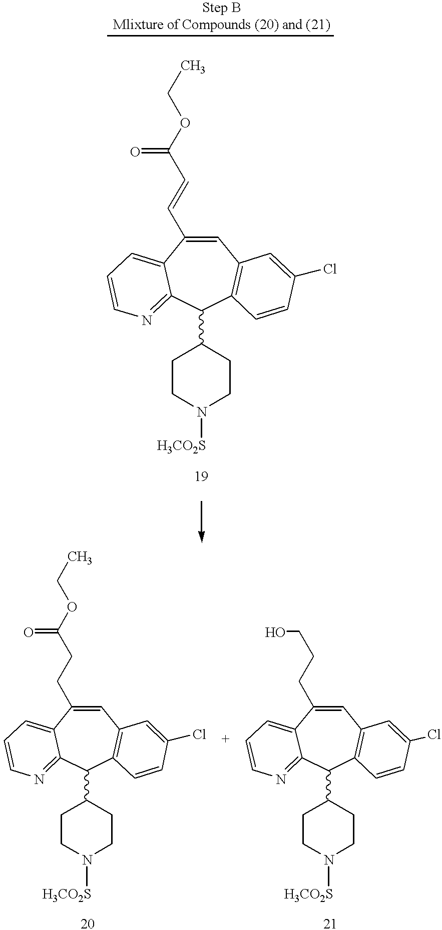

To a solution of the title compound from Preparative Example 3, Step A (6.4 g, 13 mmole) in ethanol (500 ml), was added copper chloride (0.96 g, 9.7 mmole). The reaction was cooled to 0° C. Portionwise, added sodium borohydride (4.97 g, 131 mmole). The reaction stirred overnight at room temperature. Another portion of sodium borohydride (2.46 g, 65 mmole) was added and the reaction stirred for 2 more hours, then the solvent was removed. To the residue was added saturated sodium

### Preparative Example 4

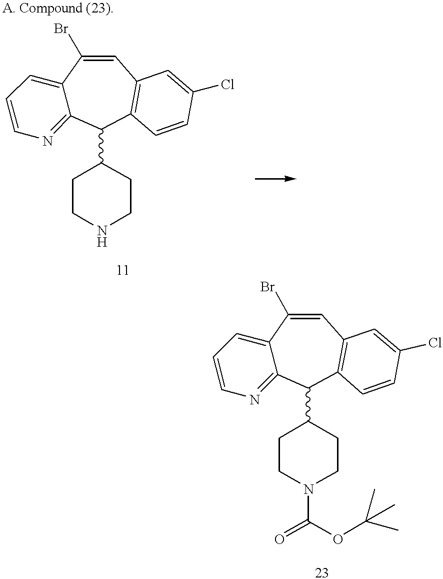

To a solution of title compound (11) from Preparative Example 2, Step A (20 g, 51.32 mmole) in CH₃OH/H₂O (400 ml, 50:1) was added di-tert-butyl dicarbonate (16.8 g, 77.0 mmole). The pH was adjusted to 9 and the mixture was stirred for 4 h. The solvent was removed, then water was added. The mixture was extracted with CH₂Cl₂. The organic layer was dried over magnesium sulfate, filtered and concentrated to dryness affording the title compound (23). MS 491 (MH+).

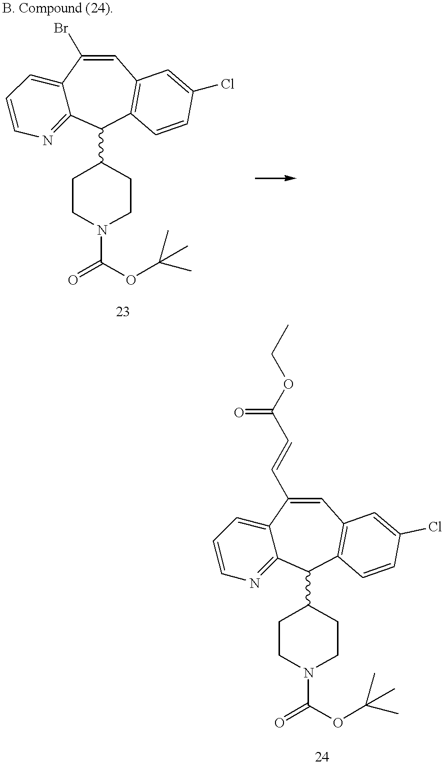

Following a similar procedure as in Preparative Example 3, Step A, the title compound (24) was prepared. MS 509 (MH+).

C. Compound (25).

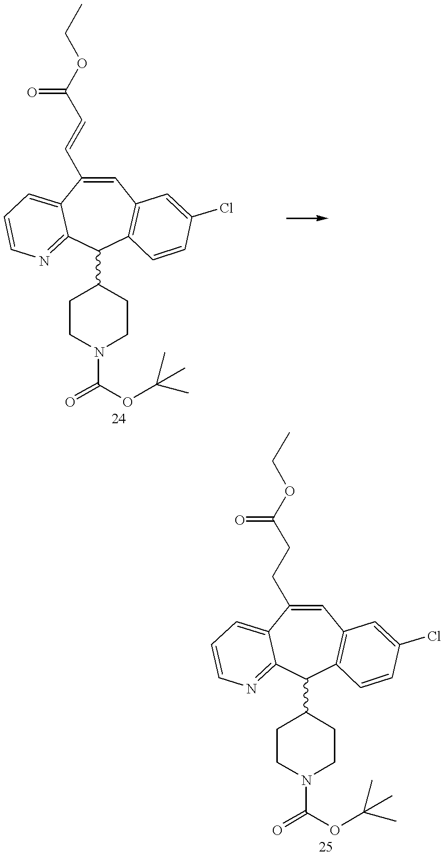

To a solution of the title compound from Preparative Example 3, Step B (19.62 g. 38.5 mmole) in ethanol (150 ml) was added platinum (IV) oxide (1.962 g). The reaction stirred over night at room temperature under H₂ balloon pressure atmosphere. After monitoring the reaction, an additional 2% (by weight) of platinum (IV) oxide was added and the reaction stirred for 6 more hours, under H₂ balloon pressure atmosphere. The mixture was filtered through celite and concentrated to dryness to afford the title compound (25) as a white solid. MS 511 (MH⁺).

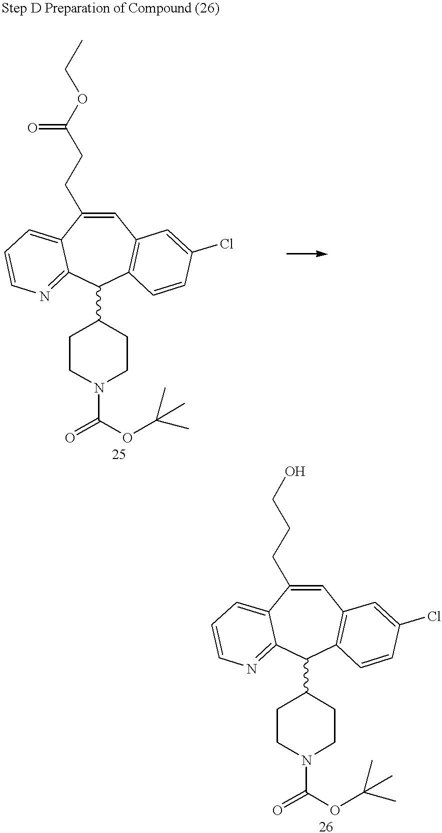

Dissolved product from Preparative Example 3, Step C (2.0 g, 3.9 mmole) in THF (30 ml) and cooled to 0° C. in an ice bath. To the reaction was added diisobutylaluminum hydride (7.8 ml, 7.8 mmole). The reaction was allowed to stir and come to room temperature over night. The reaction did not go to completion. The mixture was cooled in an ice bath (0° C.) and fresh diisobutylaluminum hydride/toluene (7.8 ml) was added. After the reaction stirred for 4 more hours, it was still not complete. The reaction mixture was cooled to 0° C., and an additional 3.9 ml of diisobutylaluminum hydride as added. The reaction stirred for 3 more hours. The crude reaction mixture was then extracted with ethyl acetate:10% citric acid, and 1.0 N NaOH. The organic layer was dried over magnesium sulfate, filtered and concentrated to dryness to afford the desired title compound (26). MS 471 (MH⁺).

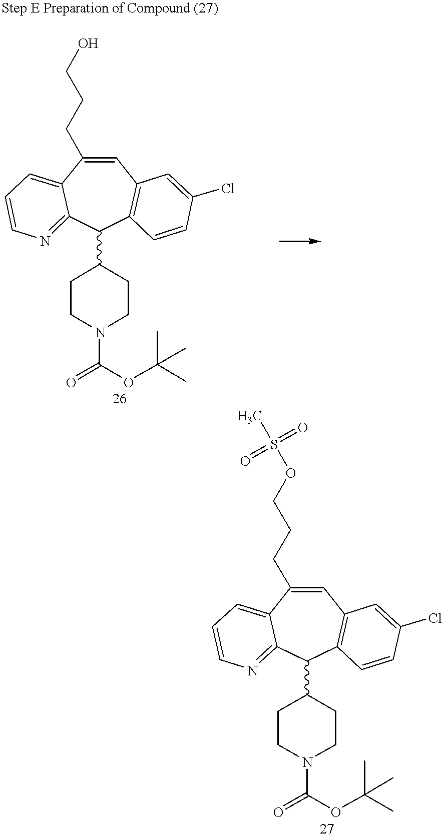

Following a similar procedure described in Preparative Example 3, Step C, the title compound (27) was prepared. MS 549 (MH⁺).

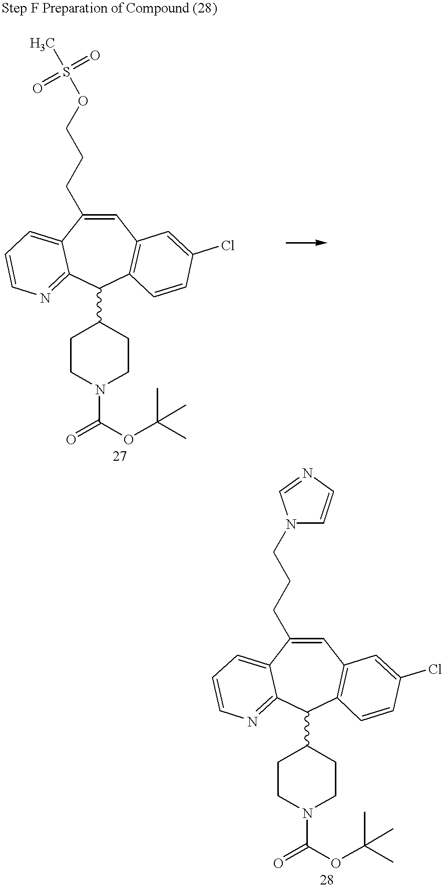

To a solution of the title compound from Preparative Example 4, Step E (1.6 g, 3.01 mmole) in DMF (50 ml) was added imidazolylsodium (Aldrich) (0.407 g, 4.52 mmole). The reaction mixture was heated to 90° C. for 2 h. The reaction was cooled and the DMF was removed. Saturated sodium bicarbonate was added and the mixture was extracted with CH₂Cl₂. The organic layer was dried over magnesium sulfate, filtered and concentrated to dryness. The crude product was purified by column chromatography eluting with 2% CH₃OH: saturated with ammonia-CH₂Cl₂, to afford the title compound (28). MS 519 (MH⁺).

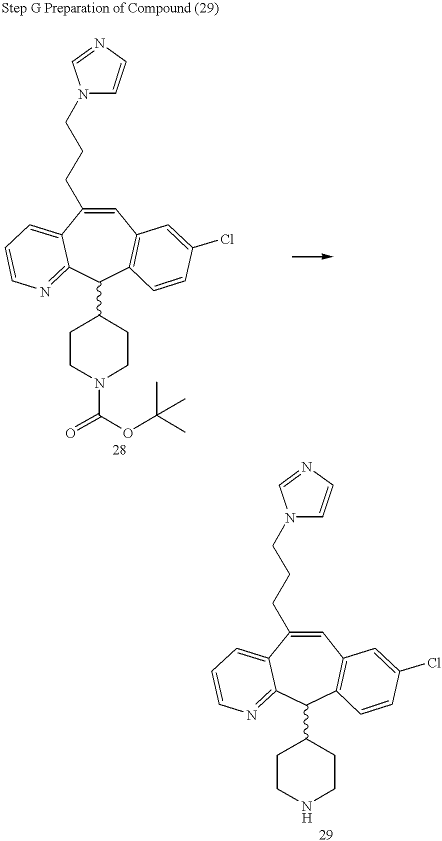

Dissolved the product from Preparative Example 4, Step F (0.55 g, 1.08 mmole) in 4 N dioxane/HCl (20 ml). The reaction mixture was stirred for 3 h at room temperature and then concentrated to dryness to afford the title compound (29) as a light yellow solid. HRMS 419 (MH⁺).

### Preparative Example 5

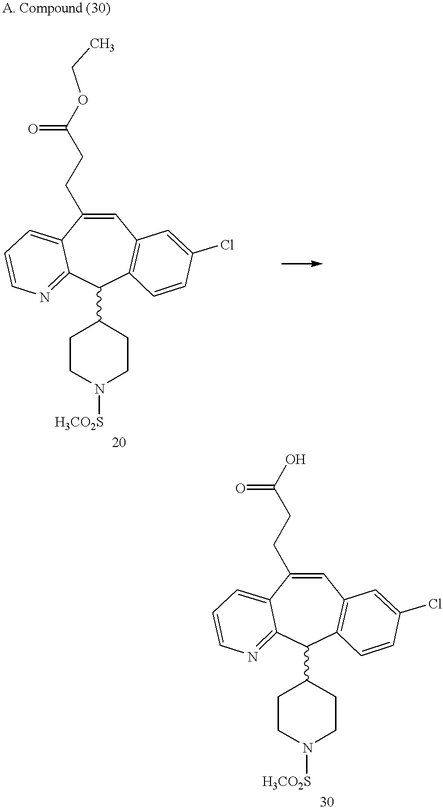

Compound (20) from Preparative Example 3, Step B (0.67 g, 1.37 mmole) was dissolved in THF (5 ml). To the mixutre was added 1N NaOH (6.9 ml) and the resulting solution stirred over night at room temperature. The reaction mixture was concentrated, acidified with 10% citric acid (w/v) and extracted with CH₂Cl₂. The organic layer was drived over magnesium sulfate, filtered and concentrated to dryness to afford the title compound (30) as a yellow solid. mp 122.7-123.4° C.; MS 461 (MH⁺).

### Example 1

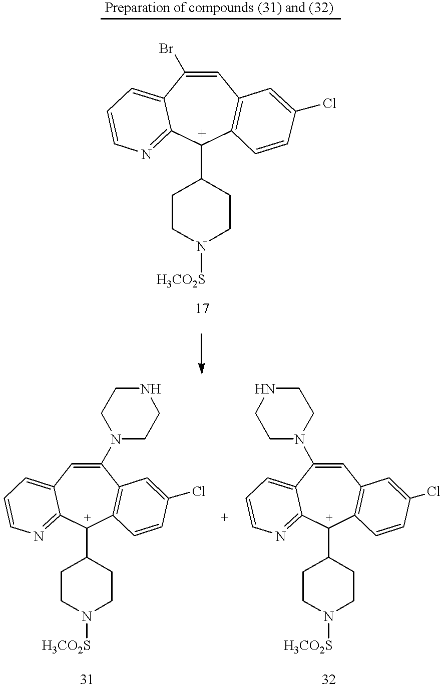

Compound (17) from Preparative Example 2, Step E 0.31 g (0.66 mmol) was treated in the same manner as described in Preparative Example 1, Step E to give a mixture of compounds (31) and (32) that were further separated on a HPLC Chiralpack AD column eluting with 30% isopropanol-70% hexane-0.2% diethylamine to give 0.04 g of target compound (31) and 0.07 g of target compound (32).

Compound 31: mp=174-175; [α]_{D}²²=+96.0° (3.6 mg/2 mL, CH₂Cl₂); MS (FAB) m/z 473 (MH+).

Compound 32: mp=173-174; [α]_{D}²²=+21.7° (8.4 mg/2 mL, CH₂Cl₂); MS (FAB) m/z 473 (MH⁺).

### Example 2

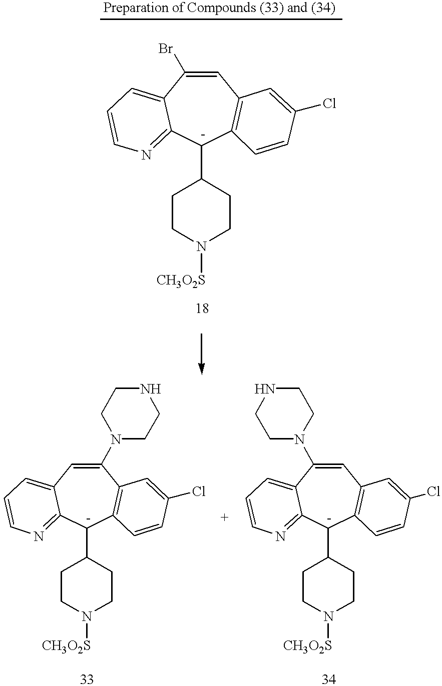

As described for preparation of Example 1 above, 0.31 g of compound (18) from Preparative Example 2 Step E was converted to a mixture of compounds (33) and (34) that were subsequently separated on a Chiralpack AD column HPLC eluting with and 30% isopropanol-70% hexane-0.2% diethylamine as eluent to give 0.12 g of target compound (33) and 0.04 g of target compound (34).

Compound 33: mp=178-179; [α]_{D}²²=−30.5° (9.5 mg/2 mL, CH₂Cl₂); MS (FAB) m/z 473 (MH+).

Compound 34: mp=172-173; [α]_{D}²²=−84° (3.5 mg/2 mL, CH₂Cl₂); MS (FAB) m/z 473 (MH+).

### Example 3

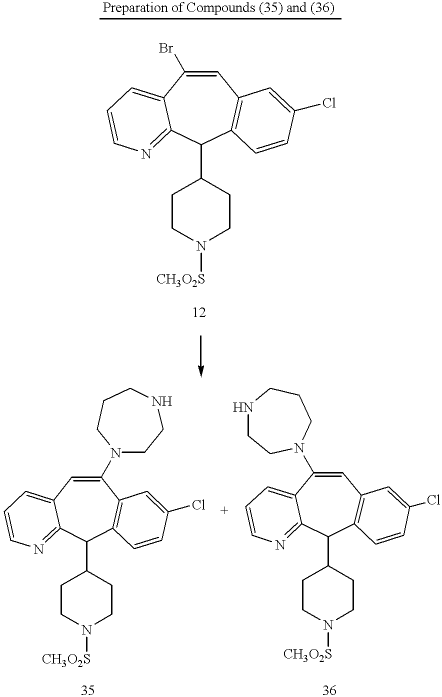

Product from Preparative Example 2, Step B (0.4 g, 0.86 mmol) was treated in the same manner as described in Preparative Example 1 Step E, substituting homopiperazine (Aldrich), to give of a mixture of compounds 35 and 36 that were further separated by flash chromatography, eluting with 10% CH30H:saturated with NH₃/CH₂Cl₂ as eluent to give 0.13 g of target compound (35) and 0.17 g of target compound (36).

Compound (35): mp=116-117; MS (FAB) m/z 487 (MH+).

Compound (36): mp=111-112; MS (FAB) m/z 487 (MH+).

### Example 4

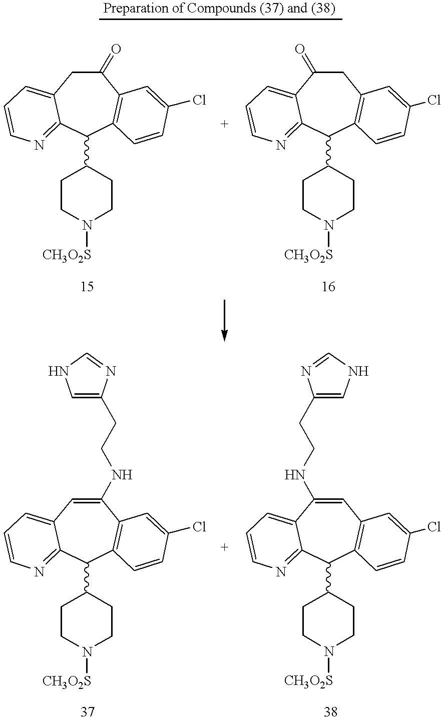

The ketones of Preparative Example 2, Step D (0.50 g, 1.23 mmol), Histamine® (0.21 g, 1.8 mmol) and p-toluene sulfonic acid (monohydrate) were dissolved in anhydrous toluene (40 mL) and refluxed in a Dean Stark trap apparatus for 24 h. The reaction mixture was then cooled, diluted with ethyl acetate and extracted with NaHCO₃. The organic layer was then dried over MgSO₄ and concentrated to dryness. Purification by flash chromatography on silica gel, eluting with 3% CH3OH(saturated with NH₃)-CH₂Cl₂, afforded 0.17 g (28% yield) 5-substituted histamine adduct (38) as the first eluting product and 0.08 g (13% yield) of the 6-substituted histamine adduct (37) as the second eluting product.

Compound (37): mp=124-125; MS (FAB) m/z 498 (MH+).

Compound (38): mp=119-120; MS (FAB) m/z 498 (MH+).

### Examples (5) and (6).

By using the same procedure as above and substituting the appropriate amines, the following mixtures of compounds were prepared:

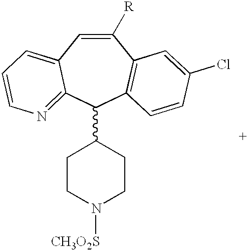

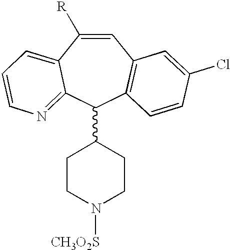

| Ex | R = | Compound #: |
|---|---|---|
| 5 | 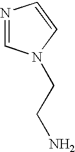 | (39) AND (40). |
| 6 | 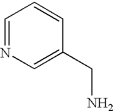 | (41) AND (42). |

### Example 7

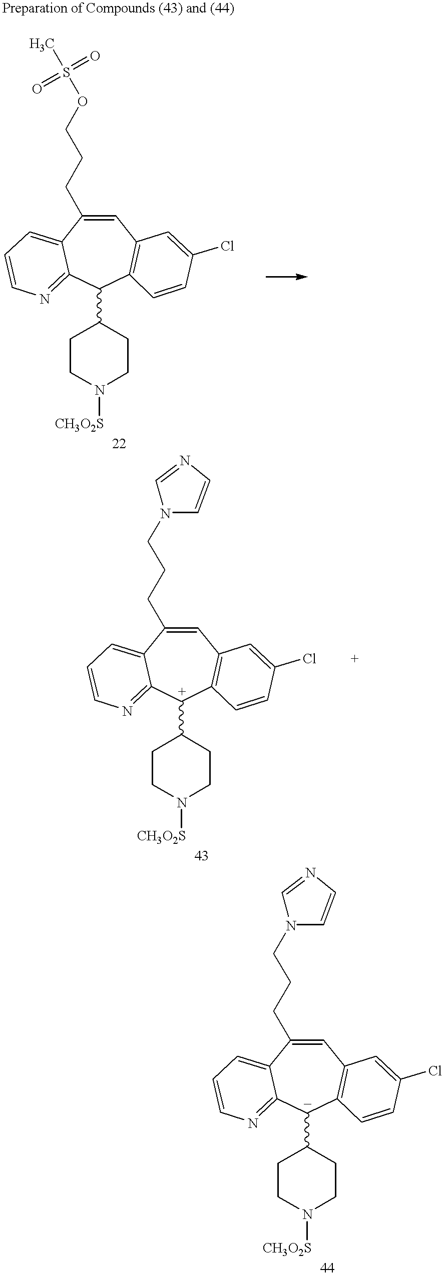

To a solution of the title compound (22) from Preparative Example 3, Step C (1.0 g, 2.03 mmole) in DMF (20 ml) was added imidazolylsodium (0.257 g, 2.85 mmole). The reaction mixture was heated to 90° C. for 2 h. Cooled the reaction and removed DMF. Added saturated sodium bicarbonate and extracted with CH₂Cl₂. Dried organic layer over magnesium sulfate, filtered and concentrated to dryness. Crude product was purified by Biotage column chromatography eluting with 3% CH₃OH: (saturated with ammonia)-CH₂Cl₂, to afford the title compound as an enantiomeric mixture. The mixture was separated into pure enantiomers on Prep HPLC Chiral AD column eluting with 35-40% Isopropanol-Hexane: 0.2% Diethyl amine, to give the title compounds (43) and (44). MS 497 (MH⁺)

### Example 8

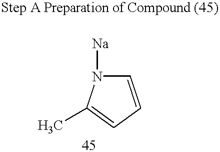

2-methylimidazole was dissolved in DMF (10 ml). To this was added one equivalent of NaH and the reaction was allowed to stir at room temperature for 1 h.

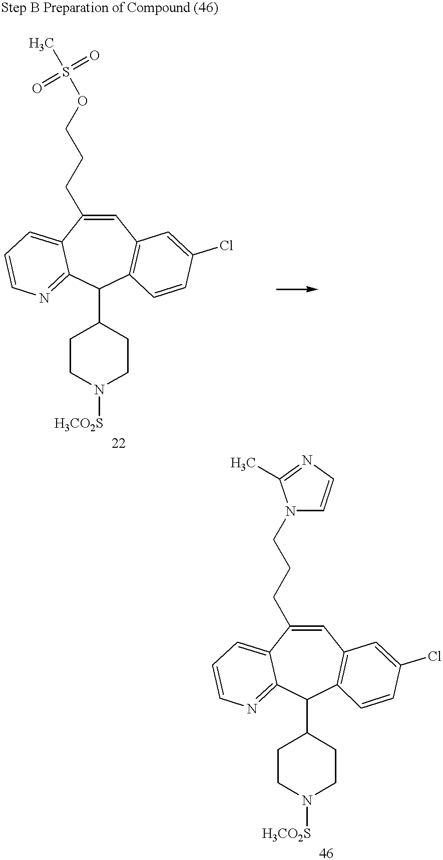

Following a similar procedure as described in Example 7, substituting 2-methyl imidazoyl sodium (45) for imidazoyl sodium, the racemic mixture of the title compound (46) was prepared. MS 511 (MH⁺).

### Example 9

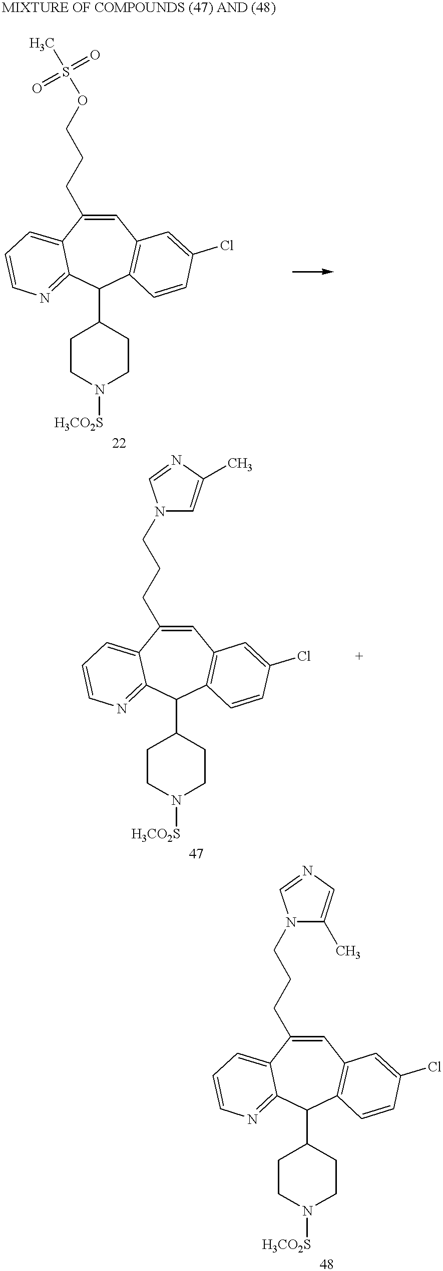

Compound (22) was reacted in the same the same manner as Example 8, substituting 4-methyl imidazole in Step A, affording a mixture of 4 and 5-methyl substituted imidazole derivatives (47) and (48).

### Example 10

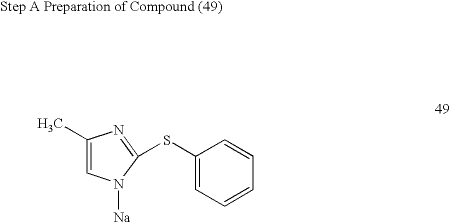

To SEM protected methyl imidazole (30 g, 0.141 mole) prepared according to literature procedure, Whiften, J. P., J. Org. Chem. 1986, 51, 1891-1894., in THF (250 ml) at −78° C. was added 2.5 M n-butyl lithium (74 ml, 0.184 mole) over 1 h. The solution was stirred for 1 h at −78° C., then a solution of diphenyl disulfide (34.27 g, 0.155 mole) in THF (125 ml) was added over ½ h. The mixture was stirred and warmed to room temperature over night. The solvents were removed and then the residue was diluted with ethyl acetate (250 ml) and washed with 1.0 M NaOH (5×50 ml) and then brine (50 ml). The organic layer was dried over Na₂SO₄, filtered and concentrated. The crude product (45.28 g, 0.141 mole) was dissoved in ethanol (100 ml) and 5 M aqueous HCl (100 ml) and stirred for 12 h. at 60° C. The solvent was removed and the residue was dissolved in distilled H₂O. 5M aqueous NaOH was added until pH=8, then the mixture was extracted with ethyl acetate. Combined organic layers and washed with brine, dried over Na₂SO₄, filtered and concentrated. Purified by flash chromatography eluting with 70% Hexanes:Acetone to afford the product as a white solid. The amine was further reacted with NaH (1 equivalent) in DMF for 1 h. affording the title compound (49).

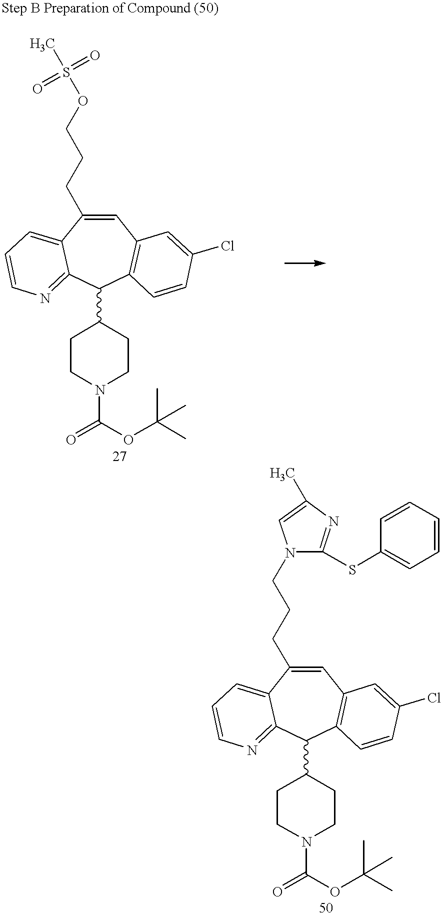

Compound (27) from PREPARATIVE EXAMPLE 4, STEP E was reacted in the same manner as EXAMPLE 8, substituting 4-methyl-2-phenylsulfanyl-1H-imidazole sodium (49), affording the title compound (50) as a light yellow solid. MS 643 (MH⁺).

### Example 11

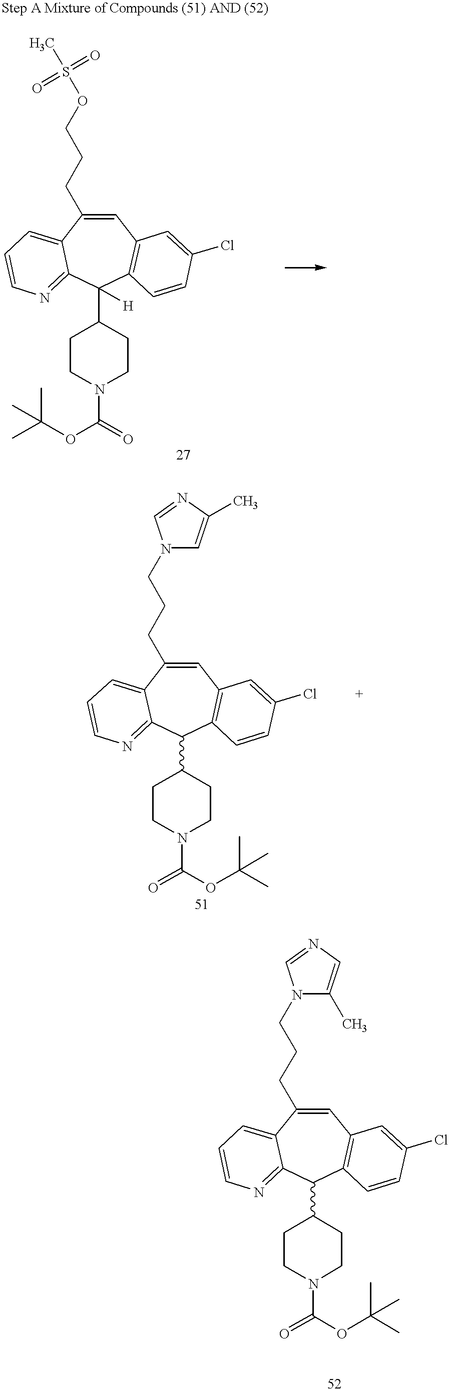

Compound (27) from PREPARATIVE EXAMPLE 4, STEP E, was treated in the same manner as in Example 9 above to afford a mixture of the 4 and 5-substituted imidazol title compounds (51) and (52).

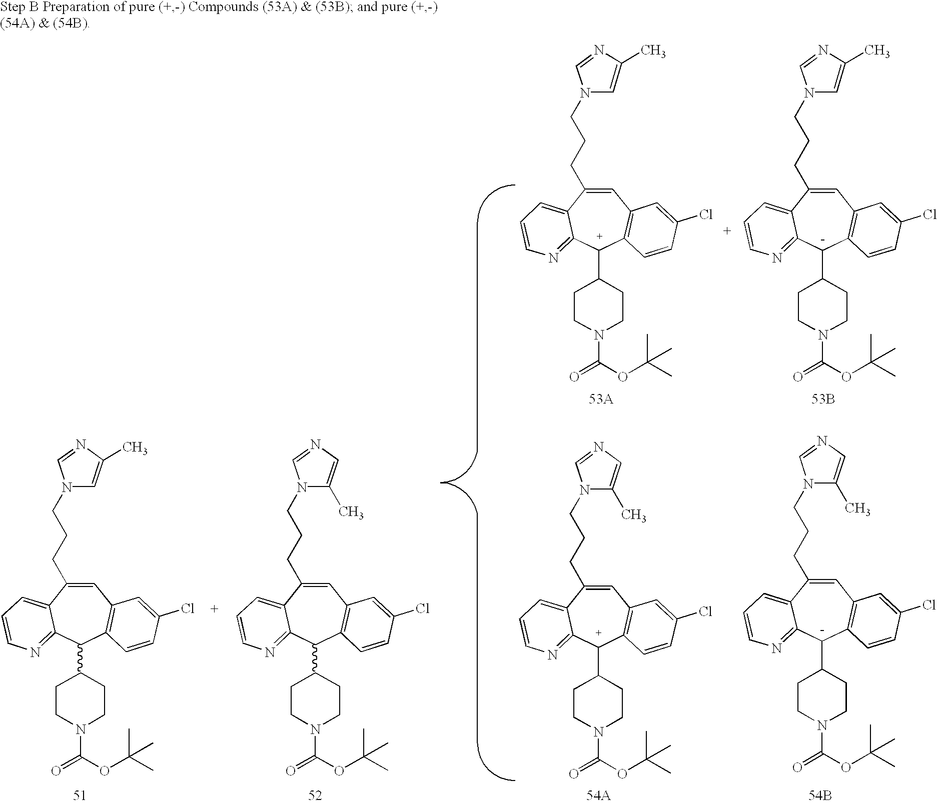

The compounds from Step A above were further separated into a mixture of (4 and 5) (+) enantiomers and (4 and 5) (−) enantiomers using preparatory HPLC Chiral AD column, eluting with 20% Isopropanol-Hexane: 0.2% Diethyl amine. MS 532 (MH⁺). The pure (+) and (−) enantiomeric pairs were then reacted with triphenyl methyl chloride (Aldrich) in CH₂Cl₂ starting at 0° C. and warming to room temperature over 3 h. The crude product was purified by column chromatography eluting with 50% ethyl acetate-acetone, affording the pure (+) and (−) 4-methyl substituted enantiomers (53A) and (53B); MS 533 (MH⁺). The column was then flushed with 100% methanol, the fraction was concentrated and the residue was treated with methanol saturated with ammonia, overnight at reflux temperature. The product was purified by column chromatography eluting with 50% ethyl acetate-acetone, affording the pure (+) and (−) 5-methyl substituted enantiomers (54A) and (54B); MS 533 (MH⁺).

### Example 12

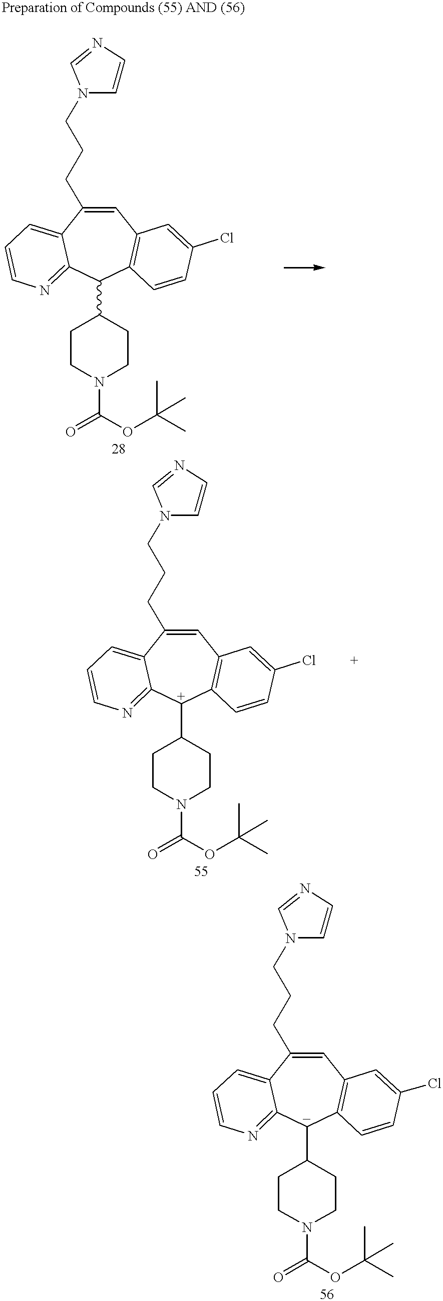

Compound (28) from PREPARATIVE EXAMPLE 4, STEP F, was separated into pure enatiomers by preparatory HPLC using a chiral AD column eluting with 20% Isopropanol:Hexane: 0.2% Diethyl amine to give pure title compounds (55) and (56). MS 519 (MH⁺).

### Example 13

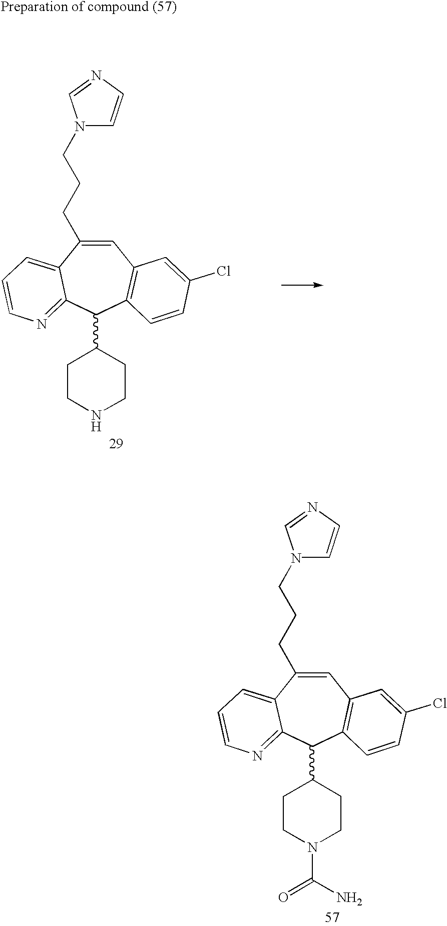

Compound (29) from PREPARATIVE EXAMPLE 4, STEP G (0.20 g, 0.48 mmole) was dissolved in CH₂Cl₂ (10 ml). Added triethyl amine (0.30 ml, 1.92 mmole) followed by trimethylsilyl isocyanate (Aldrich) (1.3 ml, 9.6 mmole) and stirred at room temperature over night. Quenched reaction with 1.0 N NaOH and extracted with CH₂Cl₂. Dried organic layer over MgSO₄, filtered and concentrated. Purified by column chromatography eluting with 3-5% Methanol saturated with Ammonia-CH₂Cl₂, affording the title compound (57) as a white solid. MS 464 (MH⁺).

### Examples 14 and 15

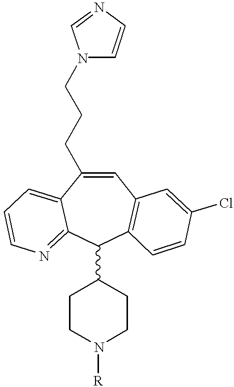

By substituting the appropriate isocyanates, and following the procedure described in EXAMPLE 13 above, the following compounds were prepared:

| Ex | R = | Compound #: |
|---|---|---|
| 14 | 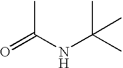 | (58). MS 518 (MH⁺). |
| 15 | 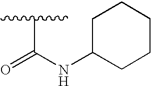 | (59). MS 544 (MH⁺). |

### Example 16

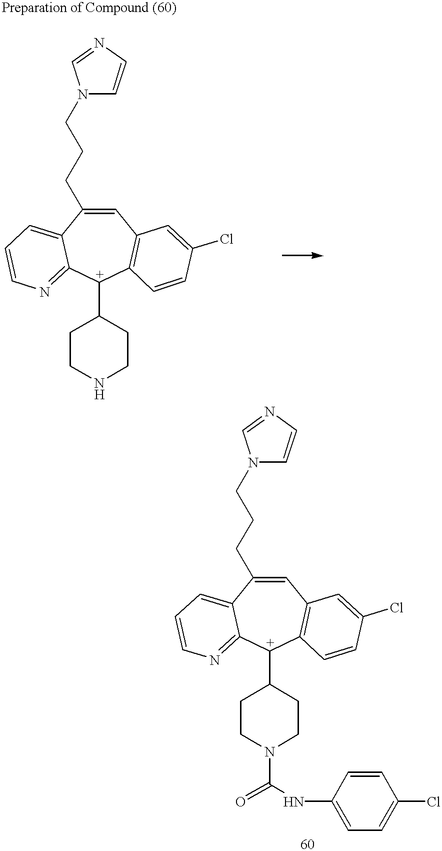

Compound (55) was deprotected following the procedure described in PREPARATIVE EXAMPLE 4, STEP G, to give the (+) enantiomer of the starting amine which was then reacted with 4-Chlorophenyl isocyanate (Aldrich) (0.05 g, 0.34 mmole) in the same manner as Example 13 above, affording the title compound (60) as a white solid. MS 572 (MH⁺).

### Example 17

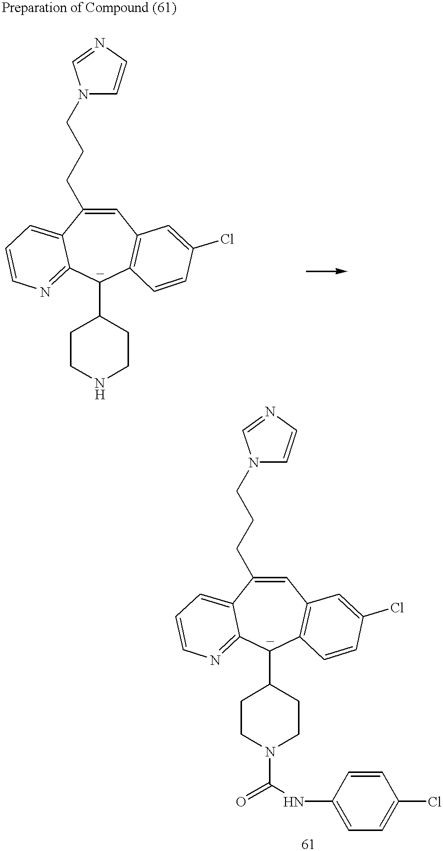

Compound (56) was deprotected following the procedure described in PREPARATIVE EXAMPLE 4, STEP G to give the (−) enantiomer of the starting amine. Reacting in the same fashion as Example 16 above, afforded the title compound (61) as a white solid. MS 572 (MH⁺).

### Example 18

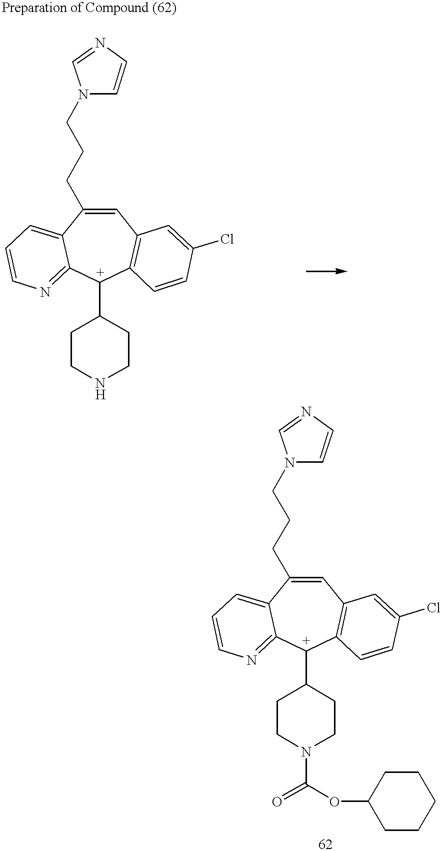

Following the procedure described in EXAMPLE 16, substituting cyclohexyl chloroformate (BASF) in place of the isocyanate, afforded the title compound (62) as a white solid. MS 545 (MH⁺).

### Example 19

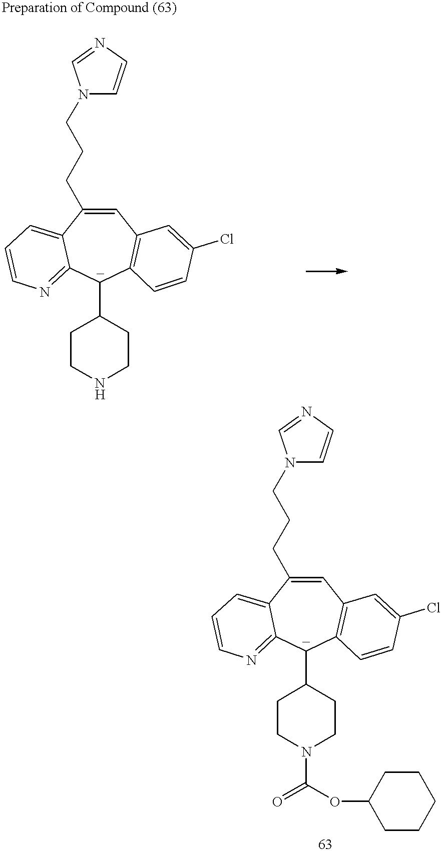

Following the same procedure as described in EXAMPLE 18 above, substituting the (−) enatiomer of the starting amine from EXAMPLE 17, afforded the title compound (63) as a white solid. MS 545 (MH⁺).

### Preparative Example 6

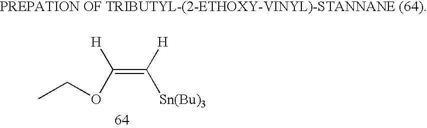

In a sealed tube, was added ethoxy ethyne (Fluka) followed by tributyltin hydride (Aldrich) and heated to 55° C. for two days. The reaction mixture was then concentrated to a brown red liquid. Purification via distillation afforded the title compound (64) as an off-white liquid. BP range 98°-115° C., (0.35 to 0.2 mmHg).

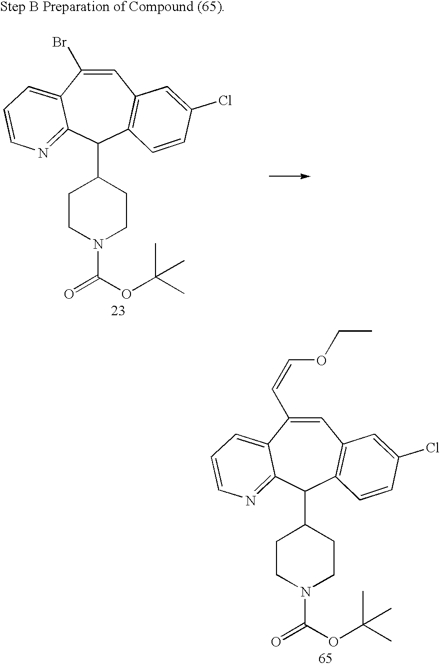

To a solution of compound (23) from Preparative Example 4, Step A (6.51 g, 13.29 mM), dichlorobis(triphenylphosphine) palladium(II) (Alrich) (0.373 g, 0.53 mM), and tetrabutylammonium chloride (Aldrich) (3.69 g, 13.29 mM) in DMF (50 ml) was added compound (64) from PREPARATIVE EXAMPLE 6, STEP A. The reaction stirred over night at 75-80° C. under nitrogen atmosphere. The reaction was cooled to room temperature, then a solution of KF (0.93 g, 15.94 mM) in H20 (70 ml) was added. A precipitate formed upon addition. The reaction mixture was stirred for fifteen minutes then added CH₂Cl₂ and stirred an additional fifteen minutes. The reaction mixture was extracted with CH₂Cl₂, the organic layer was dried over magnesium sulfate, filtered and concentrated. Purified by silica gel column chromatography eluting with 1:3% -1:1% ethyl acetate-hexanes affording the title compound (65) as a yellow solid, mp 86-90° C.

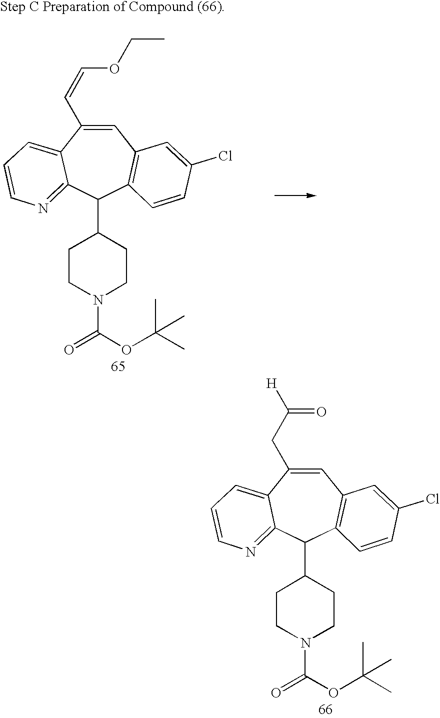

To a solution of compound (65) from Preparative Example 6, Step B (3.25 g, 6.76 mM) in THF/H20 (33.7 ml/7.3 ml), was added mercury (II) acetate. The reaction stirred at room temperature for fifteen minutes during which time a precipitate formed. To the mixture was then added saturated KI solution (70-80 ml) and was stirred for five minutes. Added CH₂Cl₂ and stirred for 1 h. The reaction was extracted with CH₂Cl₂ (2×100 ml). The organic layer was dried over magnesium sulfate, filtered and concentrated to afford the title compound (66) as a light brown solid. MS 453 (MH⁺).

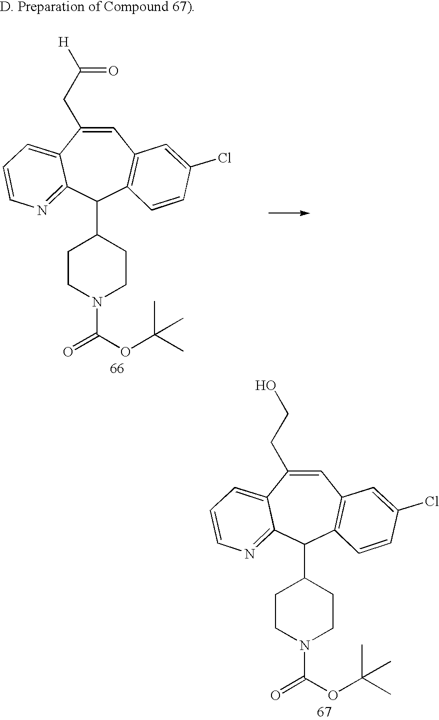

To a solution of compound (66) from Preparative Example 6, Step C (3.06 g, 6.8 mM) in ethanol (40 ml) was added sodium borohydride (0.31 g, 8.1 mM) in two portions over seven minutes. The reaction stirred for 45 minutes was then concentrated, taken up in ethyl acetate and washed with brine. Re-extracted brine layer with additional ethyl acetate and then combined organic layers, dried over magnesium sulfate, filtered and concentrated to a solid. Further purification by silica gel column chromatography eluting with 1:1-5:1 ethyl acetate-hexane afforded the title compound (67) as a white solid. MP range 120-130° C.; MS 455 (MH⁺).

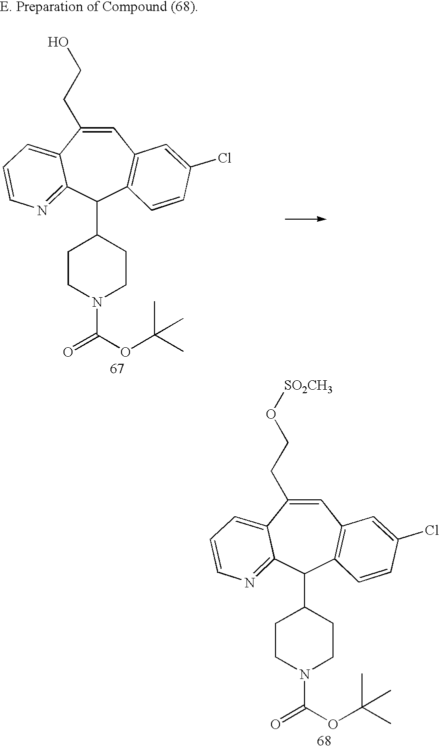

Compound (67) from Preparative Example 6, Step D was reacted in the same manner as described in Preparative Example 3, Step C, to afford the title compound (68) as a peach solid.

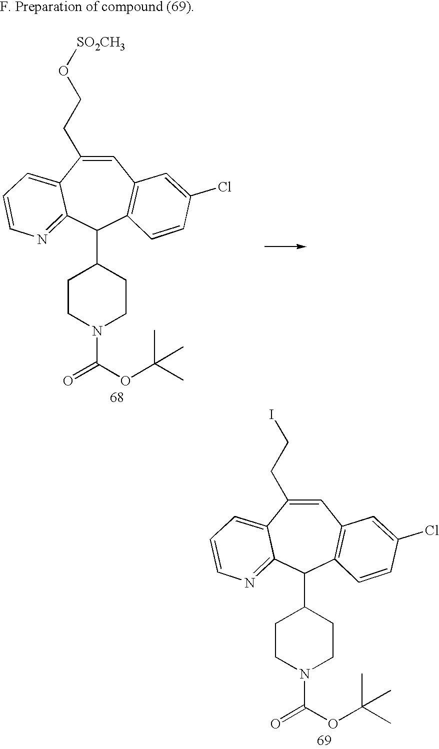

Compound (68) from Preparative Example 6, Step D (0.1 g, 0.19 mM) was dissolved in THF (2.5 ml). To the mixture was added Lil (Aldrich) (0.064 g, 0.48 mM) and stirred over night at room temperature. The reaction mixture was concentrated, taken up in CH₂Cl₂ and washed with brine (25 ml). The organic layer was dried over magnesium sulfate, filtered and concentrated to afford the title compound (69) as a yellow-brown solid.

### Example 20

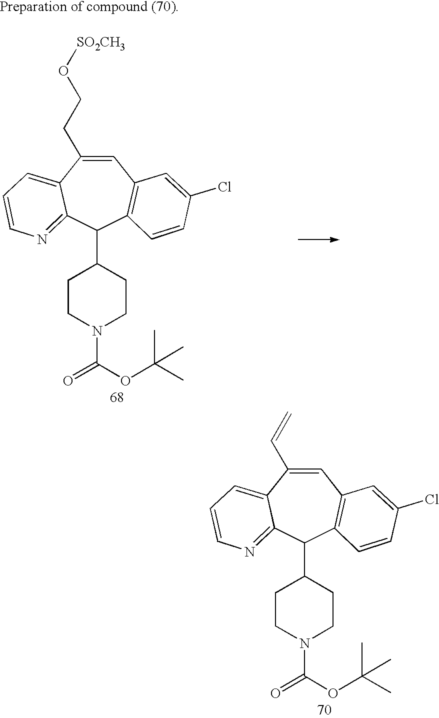

Compound (68) from Preparative Example 6, Step E, was reacted in the same manner as described in Example 8, Step B, resulting in the title compound (70) as a white solid, mp 94-101° C.

### Example 21

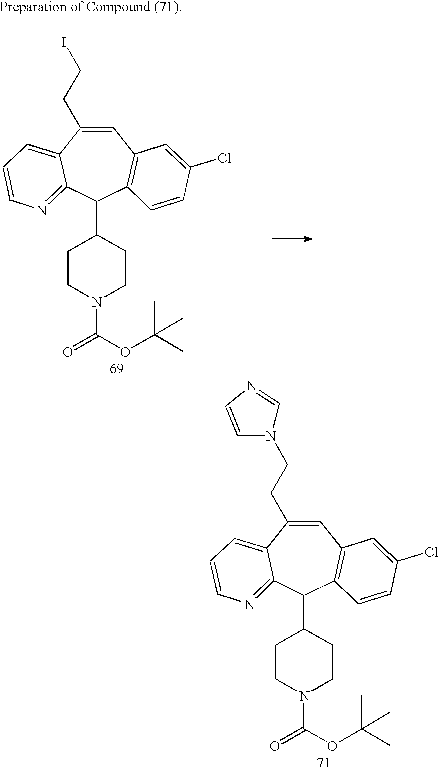

To compound (69) from Preparative Example 6, Step F (0.3 g, 0.05 mM) in CH₃CN (1 ml) was added imidazole (Aldrich) (0.014 g, 0.2 mM). The reaction was heated to 52° C. and stirred over night. The reaction was cooled, concentrated, then diluted with ethyl acetate and washed with brine. The organic layer was dried over magnesium sulfate, filtered and concentrated. The product was purified by silica gel column chromatography eluting with 0-5% methanol/ saturated with ammonia:CH₂Cl₂ to afford the title compound (71)as a white solid. mp 95-104° C.; MS 505 (MH⁺).

### Example 22

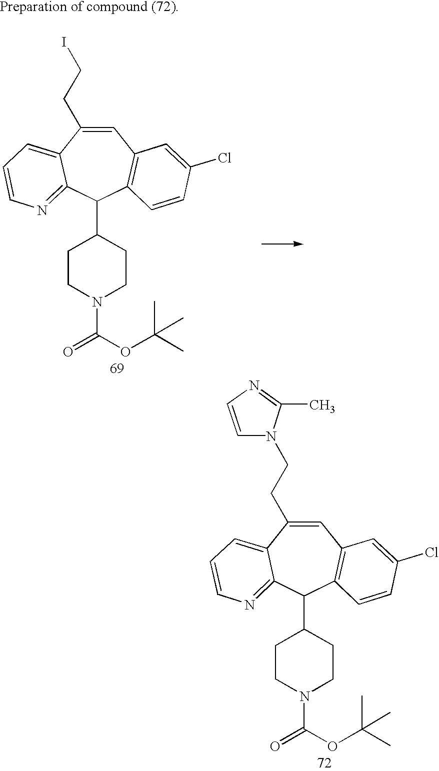

Substituting 2-methyl imidazole for imidazole and reacting in essentially the same manner as Example 21, the title compound (72) was afforded as a light tan solid. mp 93-104° C.

### Example 23

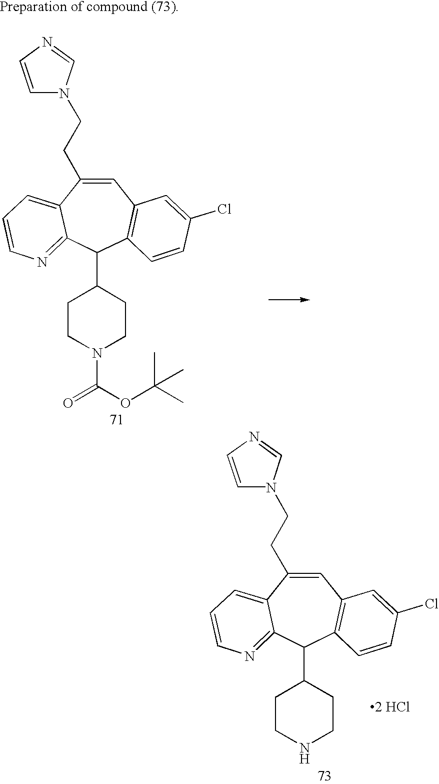

Compound (71) (0.31 g, 0.06 mM) from Example 21 was dissolved in 4M HCl/Dioxane (0.5 ml) and stirred for 1 h. Concentration of the reaction mixture afforded the title compound (73) as a light yellow solid. mp 195-205° C.

### Example 24

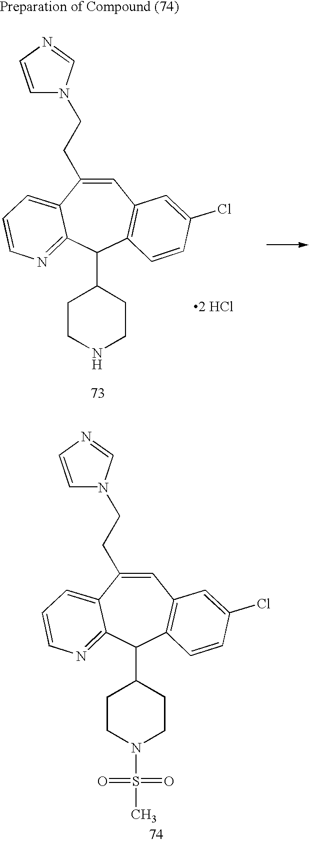

To a solution of compound (73) from Example 23 (0.026 g, 0.05 mM) in CH₂Cl₂, was added, triethyl amine (Aldrich) (0.046 ml, 0.33 mM) followed by methane sulfonyl chloride (Aldrich) (0.01 ml, 0.1 mM). The reaction stirred at room temperature for 36 h. The reaction was quenched with saturated sodium bicarbonate (50 ml) and extracted with ethyl acetate (2×75 ml). The organic layer was dried over magnesium sulfate, filtered and concentrated. The product was purified by preparatory thin layer chromatography eluting with 90:10 CH₂Cl₂: methanol saturated with ammonia to afford the title compound (74), mp 105-116° C.

### Example 25

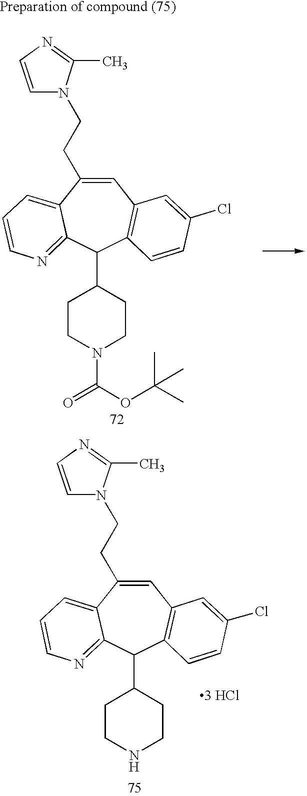

Compound (72) from Example 22 was stirred with 4M HCl/Dioxane over 2 h Concentration of reaction mixture afforded the title compound (75) as an off-white solid, mp 185-203° C.

### Example 26-29

Reacting compound (75) from Example 25, in the same manner as described in Example 13, and substituting the appropriate isocyanate, the following compounds were prepared:

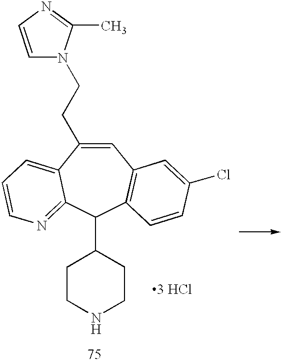

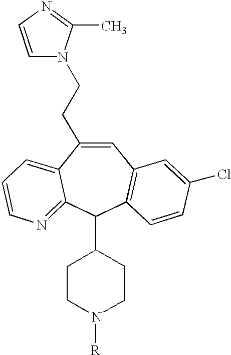

| Ex | R = | Compound #: |
|---|---|---|
| 26 | 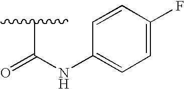 | (76). mp 133-144° C. |
| 27 | 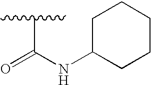 | (77). mp 131-140° C. |
| 28 | 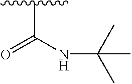 | (78). mp 125-132° C. |
| 29 | 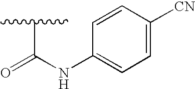 | (79). mp 160-172° C. |

### Example 30

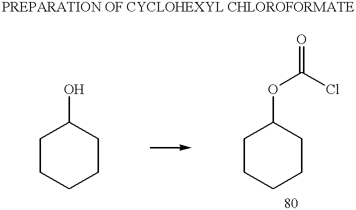

A solution of cyclohexanol (Aldrich) (25 ml, 0.2 mol) in CH₂Cl₂ (50 ml) was added dropwise over 1 h to a solution of phosgene in toluene (262 ml of a 1.93 M solution, 0.5 mol) at 0° C. The reaction was warmed to room temperature over 3 h. and stirred over night. The volatiles were removed to afford the title compound (80) as a colorless liquid.

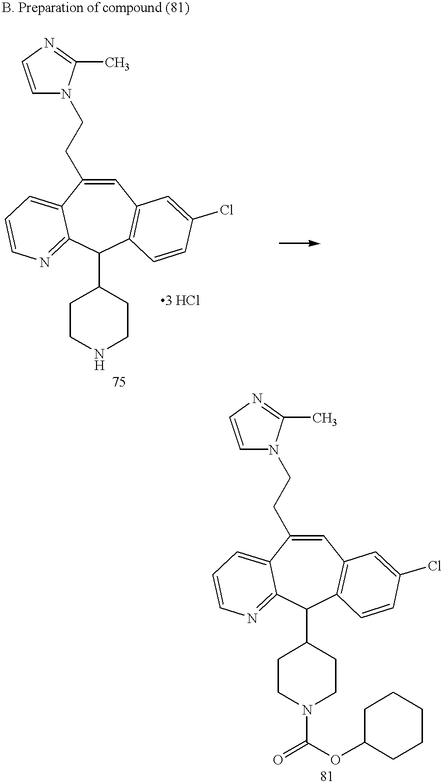

Reacting compound (75) from Example 25 in the same manner as described in Example 13, substituting the acid chloride (80) from Example 30, Step A in place of the isocyanate, afforded the title compound (81) as an off-white semi-solid. mp 89-98° C.

### Example 31

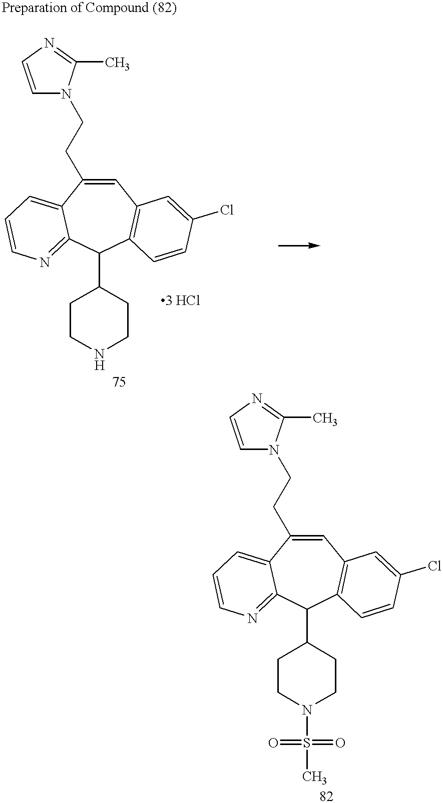

Reacting compound (75) from Example 25 in the same manner as described in Example 13 but substituting methanesulfonyl chloride in place of the isocyanate, afforded the title compound (82) as a tan semi-solid mp 120-129° C.

### Example 32

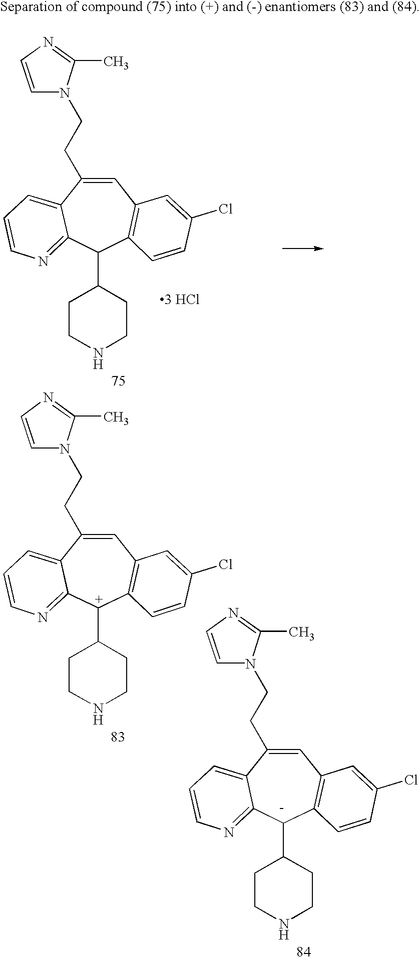

Compound (75) was seperated into pure (+) and (−) enantiomers using preparatory chiralpak-AD column chromatography, eluting with 85:15:0.2% 2-propanol:hexane/ diethyl amine affording the title compounds (83) and (84) respectively.

### Example 33

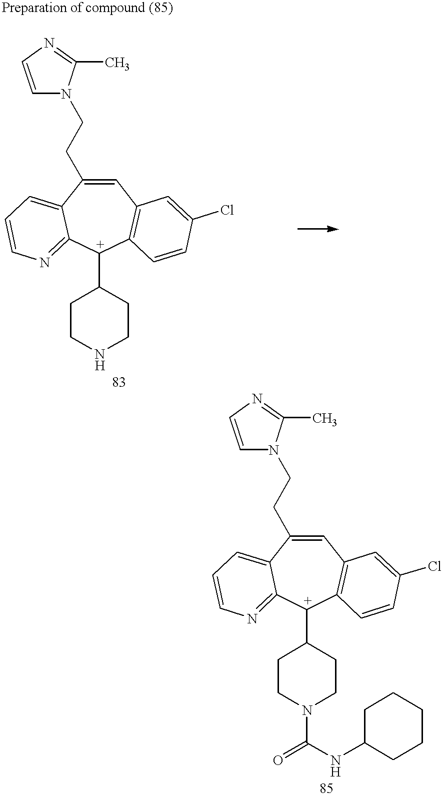

Compound (83) was reacted in the same manner as in Example 27 affording the title compound (85) as a white solid. mp 122-129° C.

### Example 34

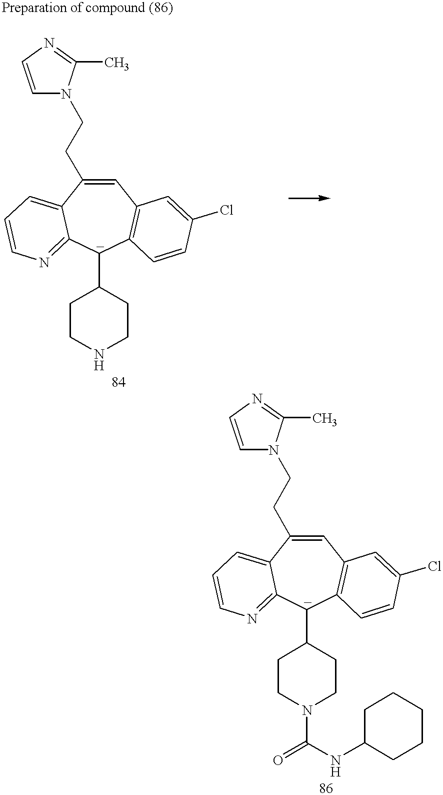

Compound (84) was reacted in the same manner as in Example 27 affording the title compound (86) as a white solid mp 118-133° C.

### Example 35

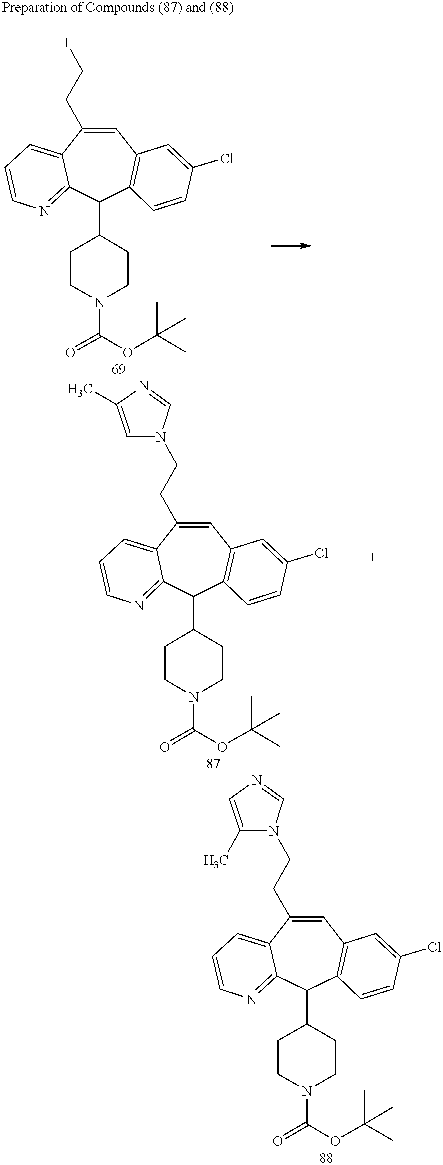

Compound (69) from Example 19 was reacted in the same manner as described in Example 21 substituting 4-methyl imidazole for imidazole, to afford a mixture of the 4 and 5 substituted imidazole derivatives. The mixture (0.234 g, 0.45 mM) was subsequently treated with trityl chloride (Aldrich) (0.047 g, 0.17 mM) and separated by preparatory thin layer chromatography, eluting with 1:6% ethyl acetate-acetone affording the pure isomers (87) and (88) mp (87) 97-107° C. (white solid).

### Example 36

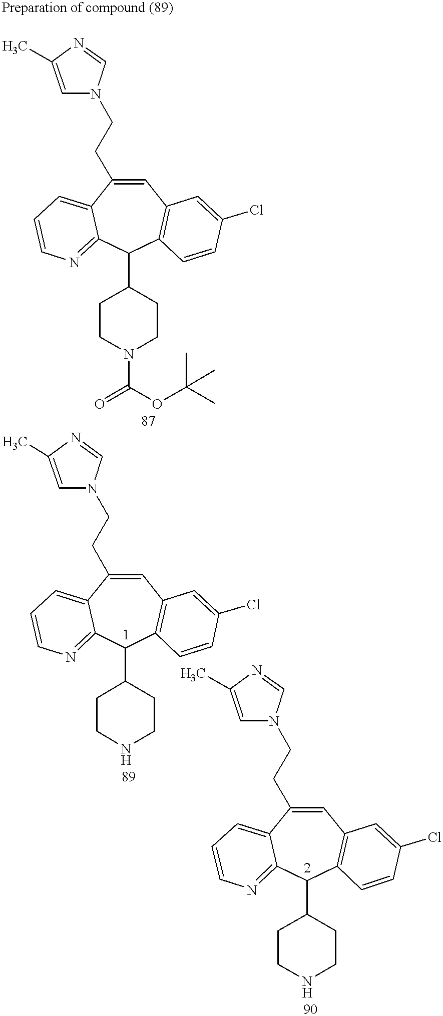

Compound (87) from Example 35 (0.085 g, 0.16 mM) was reacted in the same manner as described in Example 25. The resulting enantiomeric mixture was then separated by Preparatory Chiralpak-AD column chromatography eluting with 15-85% Isopropanol-Hexane, 0.2% diethylamine, affording enantiomers 1 and 2 as off-white solids.

### Example 37

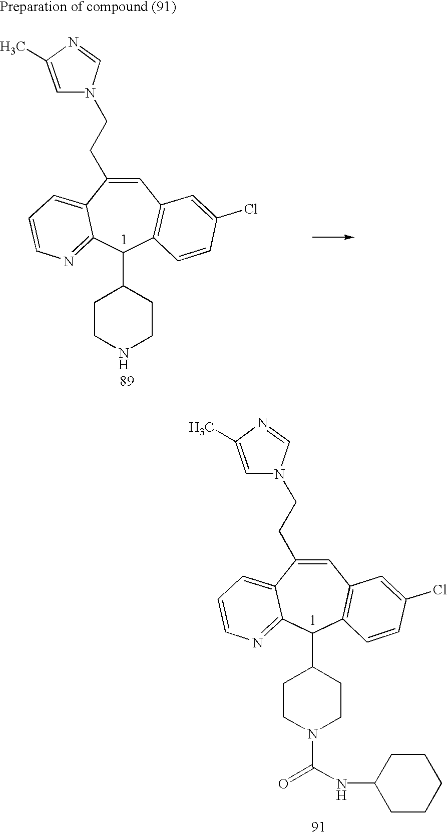

Enantiomerically pure compound (89) from Example 36 (0.02 g, 0.049 mM) was reacted in a similar manner as in Example 27 to afford the title compound (91) as a white solid. mp 130-142° C.

### Example 38

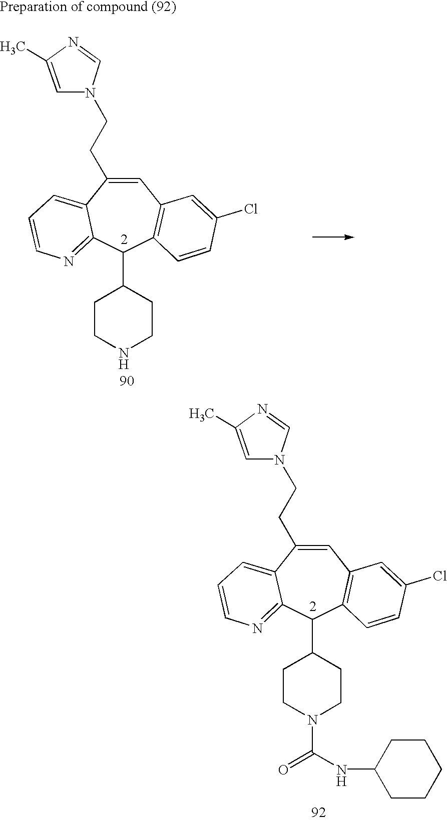

Enantiomerically pure compound (90) from Example 36 (0.023 g, 0.054 mM) was reacted in a similar manner as in Example 27 to afford the title compound (92). mp 125-135° C.

### Preparative Example 7

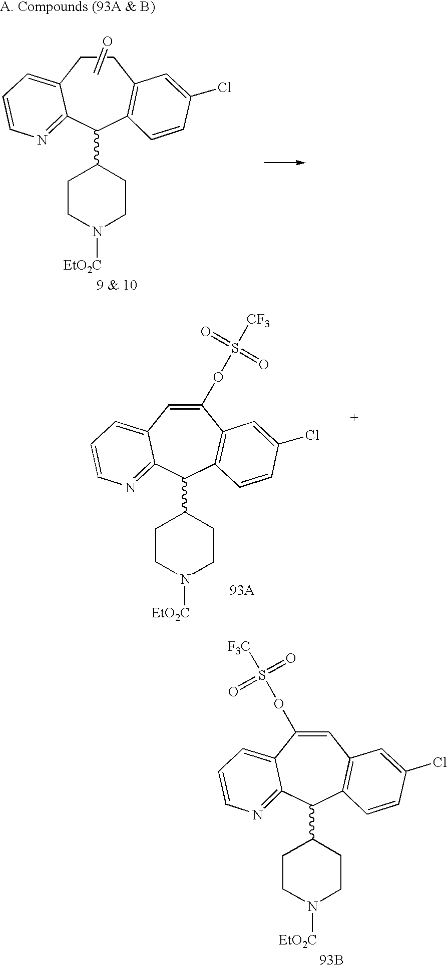

A mixture of piperizinyl compounds (9) and (10) from PREPARATIVE EXAMPLE 1, STEP F in THF at −78° C. was reacted with LDA (1.1 eq.) and stirred for 1.5 h. The mixture was warmed to −20° C. and then N-phenyl trifluoromethane sulfonimide (1.1 eq.) was added. Stirred over night at room temperature then extracted mixture with EtOAc and washed with H₂O. Dried over Na₂SO₄ and concentrated. Purification and separation by flash silica gel column chromatography afforded pure Compounds (93A & 93B).

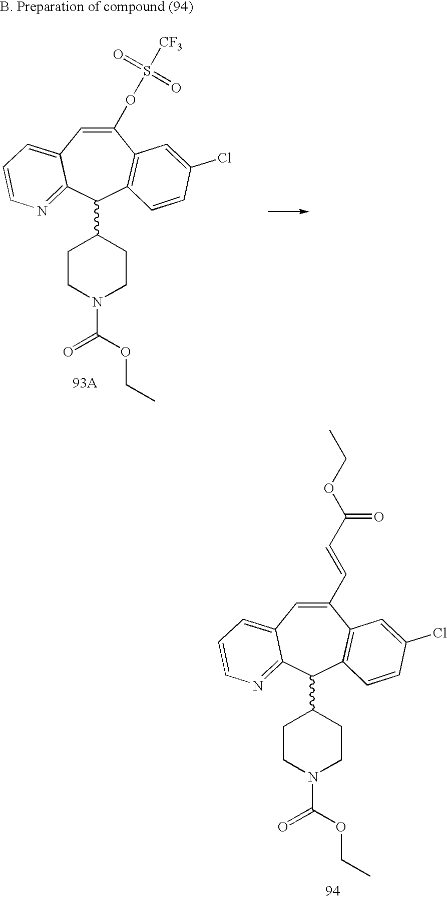

Compound (93A) from above was dissolved in DMF. Successively added, Et₃N (29 eq.), Ethyl acrylate (5.4 eq.), K₂CO₃ (5 eq.), BU₄NBr (2 eq.) and Palladuim (II) acetate (0.13 eq.). The mixture stirred and heated to 100° C. for 4 h. After cooling, the mixture was concentrated and the residue was taken up in CH₂Cl₂ and extracted with CH₂Cl₂/H₂O. The organic layer was dried over Na₂SO₄ then concentrated and the residue purfied by flash silica column chromatography to afford the title compound (94).

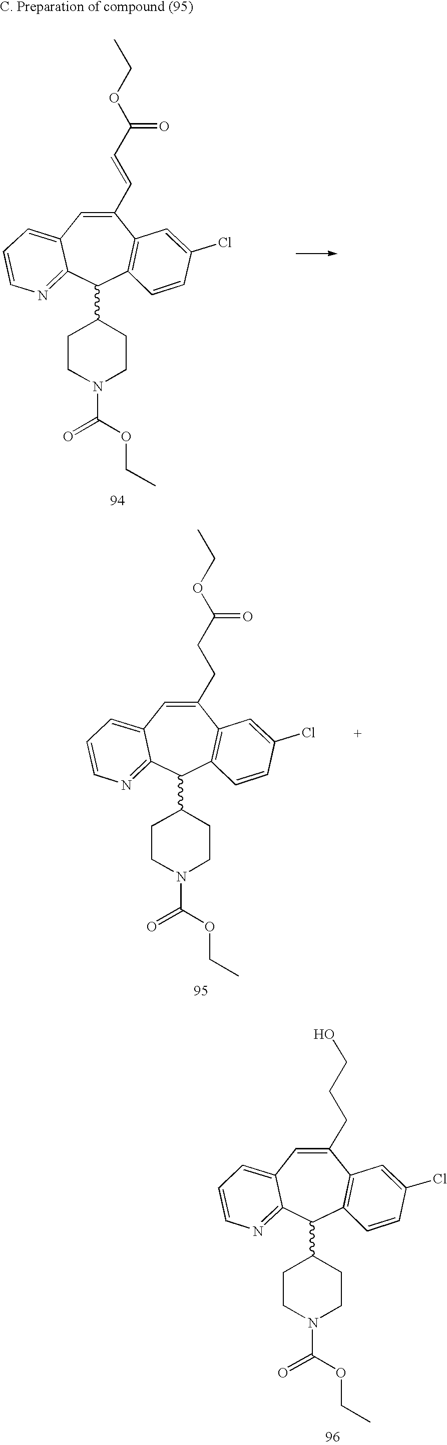

Compound (94) was dissolved in ETOH cooled in an ice bath and reacted with NaBH₄ (15 eq.) for 3 min. Then added CuCl (2 eq) and stirred for 2 h. at room temperature. The mixture was filtered, concentrated and extracted with CH₂Cl₂. Washed with water then brine, dried over Na₂SO₄ and concentrated to a mixture of the title compound (95) and the hydroxy compound (96).

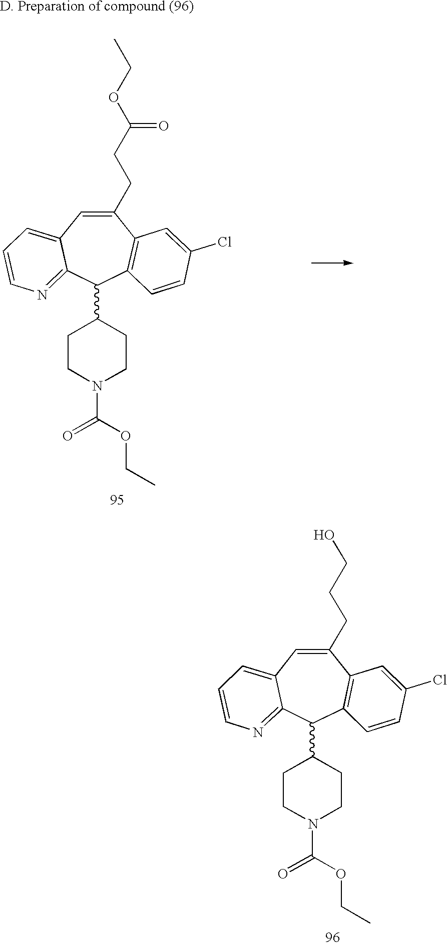

Compound (95), was then further reacted with LiBH₄(3 eq.) in THF at reflux temperature for 4 h. EtOAc was added and the mixture was washed with Na₂CO₃ then dried over Na₂SO₄ and concentrated to afford the title compound (96).

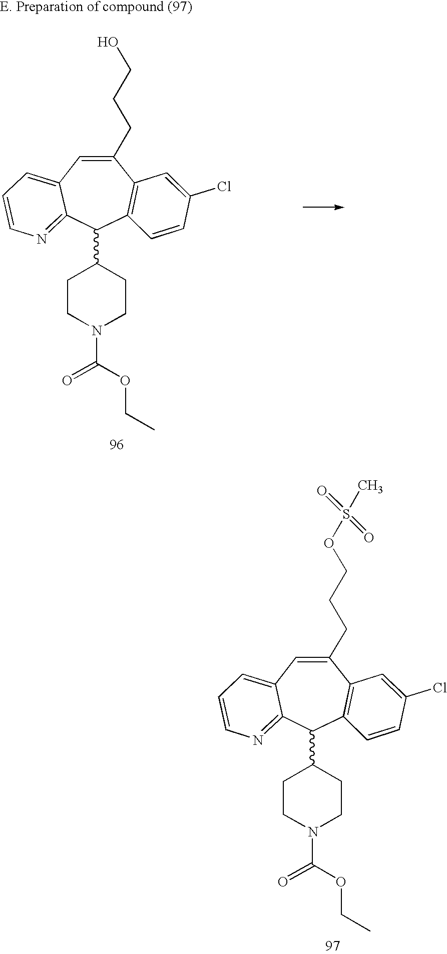

Dissolved compound (96) in CH₂Cl₂, added Et₃N (3 eq.) followed by methane sulfonylchloride (1.5 eq.). The mixture stirred at room temperature over night then diluted with CH₂Cl₂ and washed with Na₂CO₃. Dried over NaSO₄ and concentrated to afford the title compound (97).

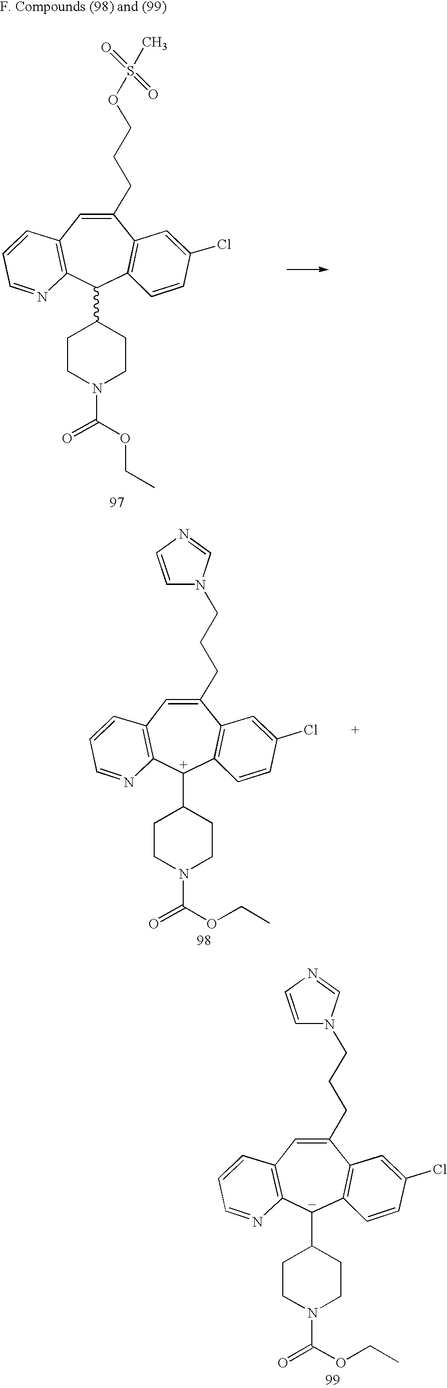

To a solution of sodium imidazole (Aldrich) in DMF was added, NaH (2 eq.). Stirred for 15 min. then added compound (97) (from above) (1 eq.) and stirred over night at room temperature. The reaction mixture was concentrated and then extracted with ethyl acetate. Washed with Na₂CO₃, dried over NaSO₄, filtered then concentrated. Crude product was purified by flash silica column chromatography. Further seperation of pure (+) enantiomers and pure (−) enantiomers was accomplished on a chiracel AD column affording the title compounds (98) and (99).

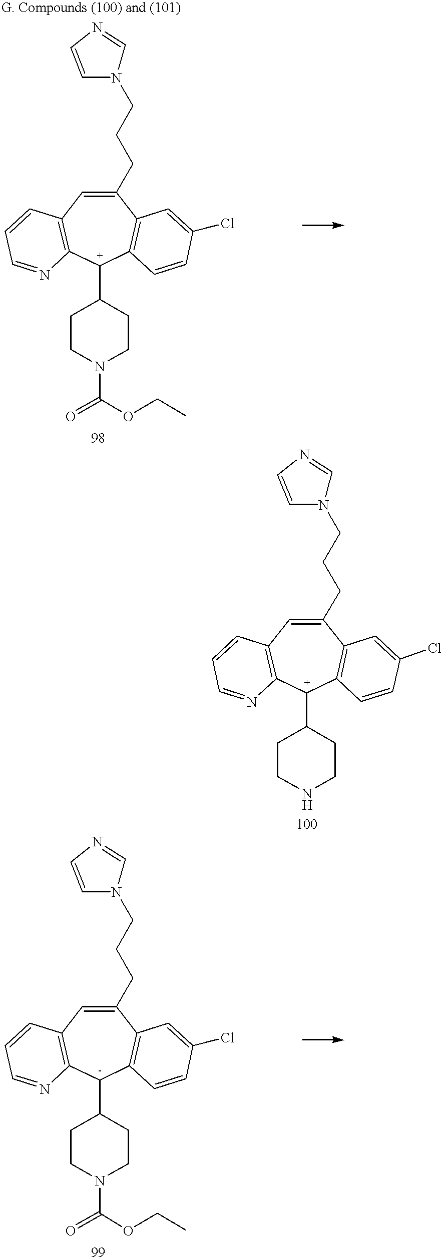

Compounds (98) and (99) were individually hydrolyzed to their free amines by refluxing in conc. HCl for 5 h. The reaction mixtures were seperately poured into ice and basified with NH₄OH. The solutions were then extracted with CH₂Cl₂, dried over Na₂SO₄, filtered and concentrated to afford the title compounds (100) and (101).

### Preparative Example 8

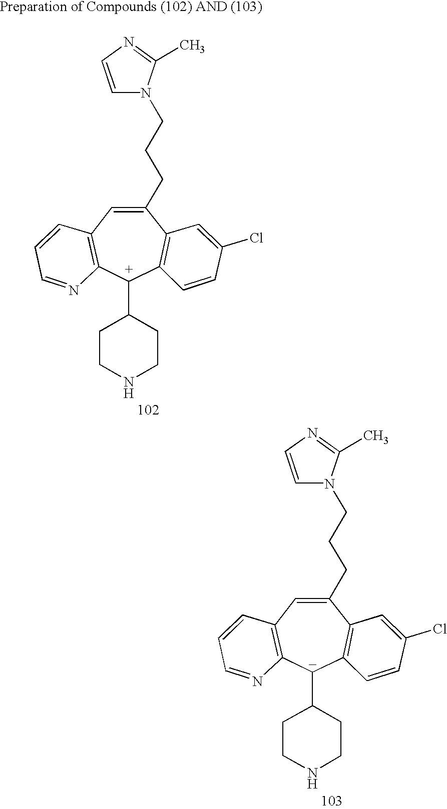

In a similar manner as described in Preparative Example 7, Steps A-G, substituting 2-methyl imidazole for sodium imidazole, in Step F, the title compounds (102) and (103) were prepared.

### Preparative Example 9

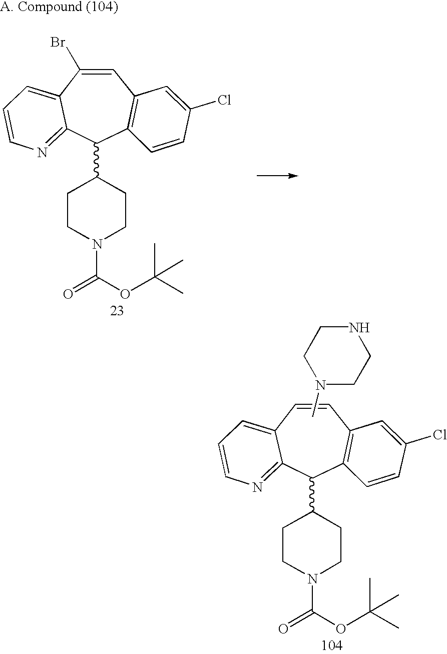

Compound (23) from Preparative Example 4 was reacted with piperazine in the same manner as described in Preparative Example 1, Step E, affording the title compound (104).

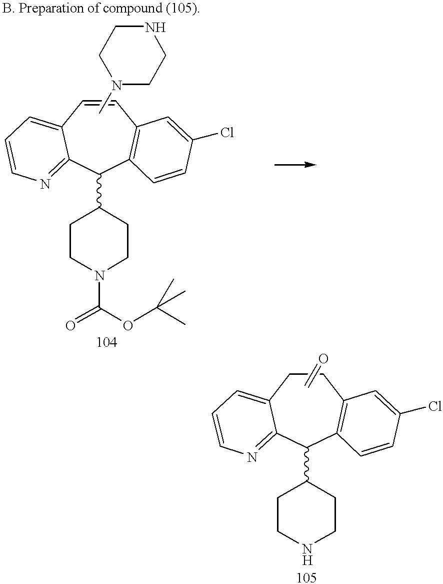

Compound (104) from above was hydrolyzed with 6N HCl over night at reflux temperature. The cooled reaction mixture was basified with 50% w/w NaOH and then extracted with 80% THF-EtOAc. The organic layer was dried over MgSO4, filtered and concentrated to dryness, affording the title compound (105).

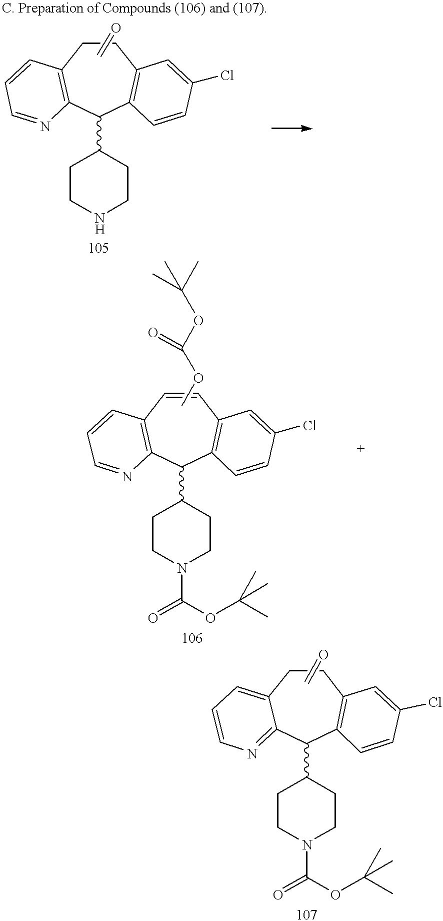

Compound (105) was dissolved in 50:1 MeOH:H₂O then added di-tert-butyl dicarbonate (2 eq.). Adjusted pH to 9 and stirred for 4 h at room temperature. The reaction mixture was concentrated and extracted with CH₂Cl₂. The organic layer was washed with Na₂CO₃, dried, filtered and concentrated to dryness affording a mixture of title compounds (106) and (107).

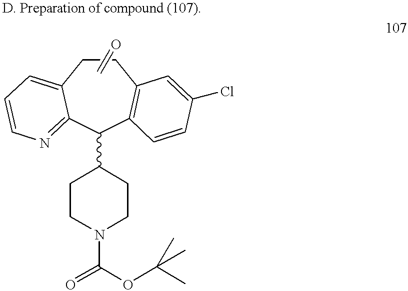

To the mixture of compounds (106) and (107) from Step C above, in 80% MeOH/H₂O at room temperature was added, cesium carbonate (2 eq.). The reaction stirred overnight. The mixture was then concentrated, extracted with CH₂Cl₂, washed with H₂O, dried over MgSO₄, filtered and concentrated to dryness affording the title compound (107).

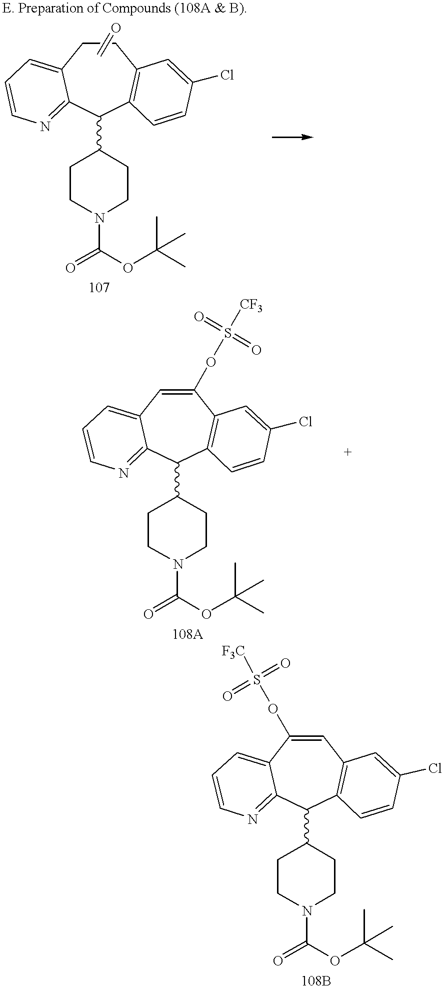

Compound (107) was reacted with N-phenyl trifluoromethane sulfonimide in a similar manner as described in Preparative Example 7, Step A, affording the title compound (108A & 108B).

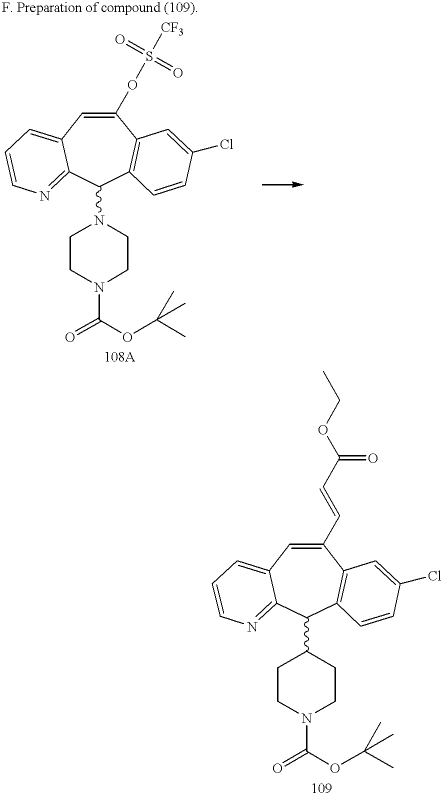

Compound (108A) was reacted with ethyl acrylate in a similar manner as described in Preparative Example 7, Step B affording the title compound (109).

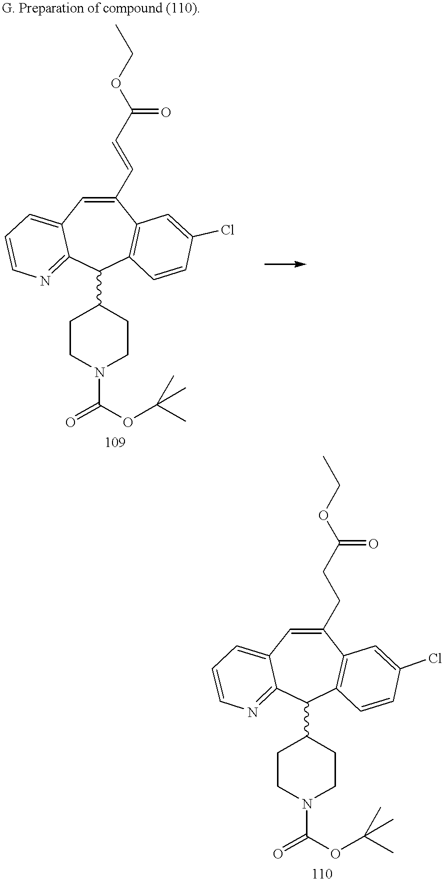

Compound (109) was reacted with NaBH₄ and CuCl in a similar manner as described in Preparative Example 7, Step C affording the title compound (110).

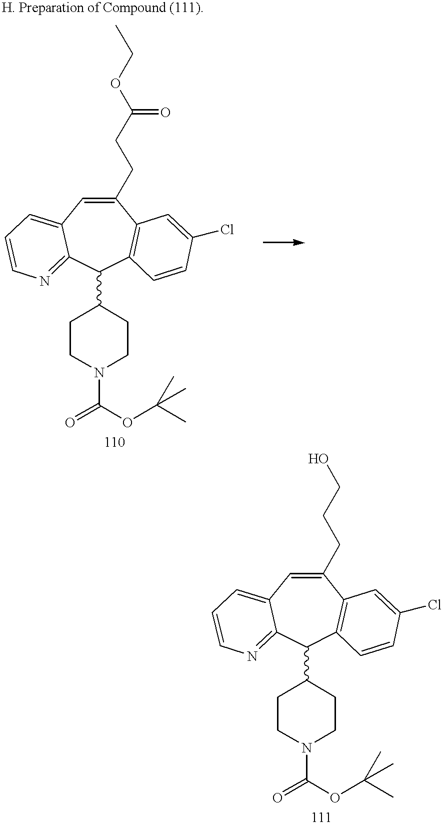

Dissolved compound (110) in THF and then added 1 M LiAlH₄/THF (1 eq.) and stirred for 1.5 h at room temperature. To the mixture was added H₂O and 15% NaOH then extracted with EtOAc. The reaction was washed with brine, dried over MgSO₄, filtered and concentrated. Purification by flash silica column chromatography eluting with 20% EtOAc/CH₂Cl₂ afforded the hydroxy title compound (111).

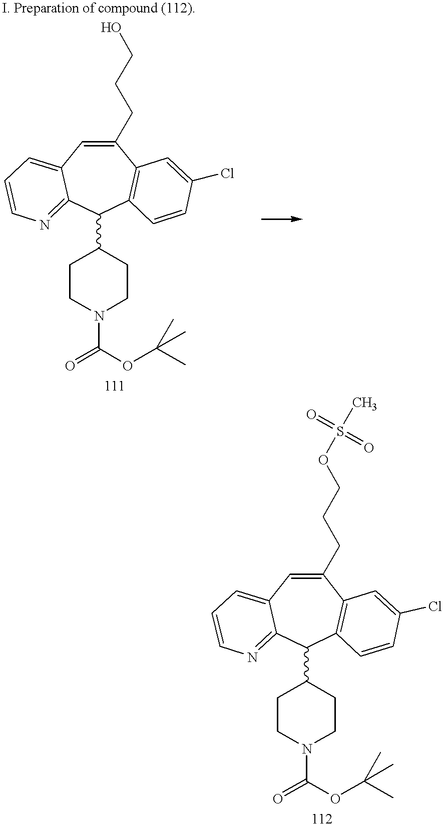

Compound (111) was reacted with methane sulfonyl chloride in a similar manner as described in Preparative Example 7, Step E affording the title compound (112).

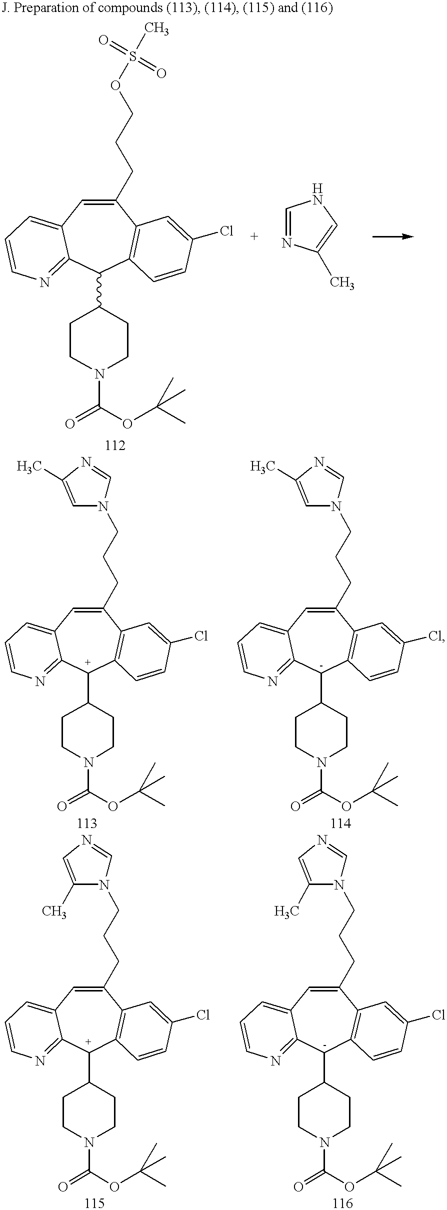

Compound (112) was reacted in a similar manner as Preparative Example 7, Step F substituting 4-methylimidazole for sodium imidazole. A mixture of (+,−)4 and (+,−)5-methyl imidazoles resulted. The mixture was treated in the same manner as described in Example 11 affording pure stereoisomers (113), (114), (115) and (116).

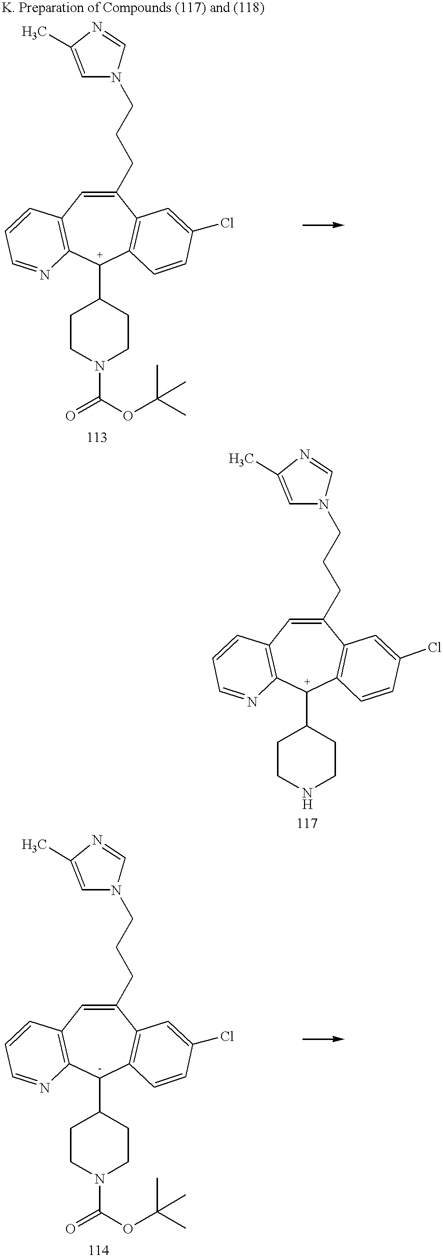

Compounds (113) and (114) were hydrolyzed to their free amines by stirring in HCl/Dioxane for 4 h. The mixtures were then concentrated to dryness affording the title compounds (117) and (118).

### Preparative Example 10

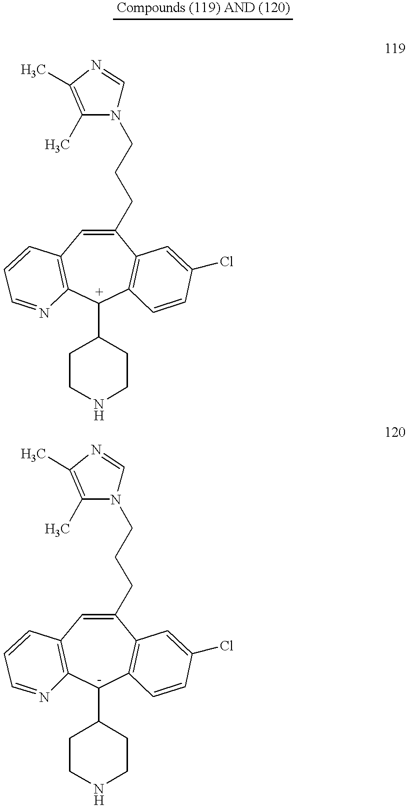

In a similar manner as described in Preparative Example 9, Steps A-K, substituting 4,5-dimethyl imidazole in Step J, the title compounds (119) and (120) were prepared.

### Example 39-45

Reacting compounds (100) or (101) from Preparative Example 7, in the same manner as described in Example 13, substituting the appropriate isocyanate or chloroformate, the following compounds were prepared:

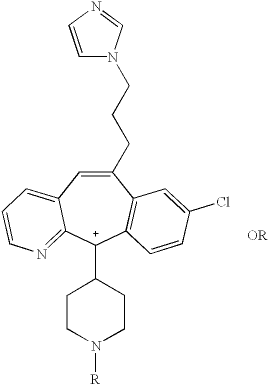

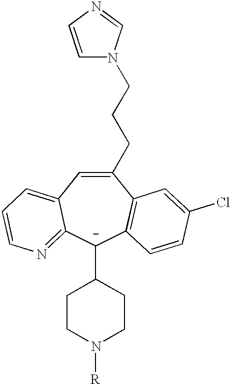

| Ex | R = | Compound #: |
|---|---|---|
| 39 | 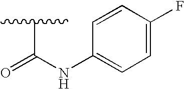 | (121) AND (122) |
| 40 | 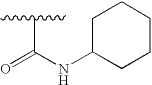 | (123) AND (124) |
| 41 | 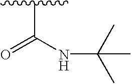 | (125) AND (126). |
| 42 | 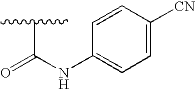 | (127) AND (128). |
| 43 | 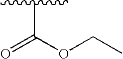 | (129) AND (130). |
| 44 | 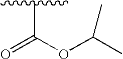 | (131) AND (132). |
| 45 | 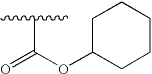 | (133) AND (134). |

### Example 46-51

Reacting compounds (102) or (103) from Preparative Example 8, in the same manner as described in Example 13, substituting the appropriate isocyanate or chloroformate, the following compounds were prepared:

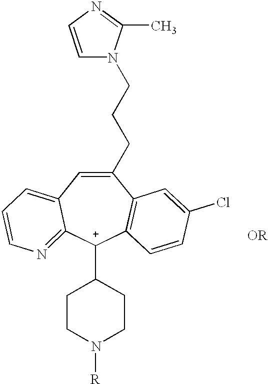

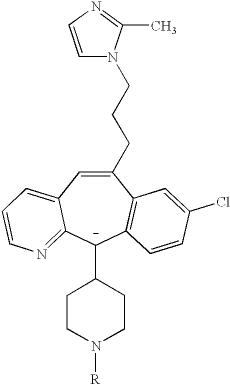

| Ex | R = | Compound #: |
|---|---|---|
| 46 | 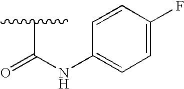 | (135) AND (136). |
| 47 | 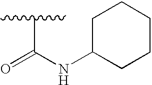 | (137) AND (138). |
| 48 | 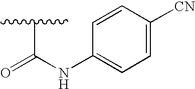 | (139) AND (140). |
| 49 | 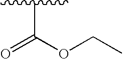 | (141) AND (142). |
| 50 | 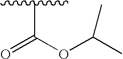 | (143) AND (144). |
| 51 | 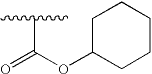 | (145) AND (146). |

### Example 52-59

Reacting compounds (117) or (118) from Preparative Example 9, in the same manner as described in Example 13, substituting the appropriate isocyanate, chloroformate or sulfonyl chloride, the following compounds were prepared:

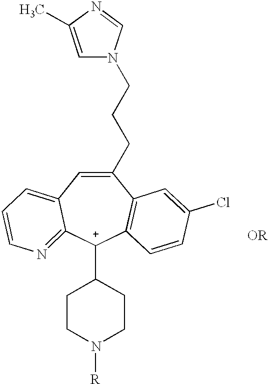

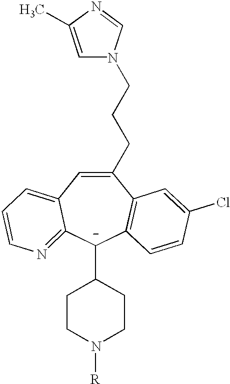

| Ex | R = | Compound #: |
|---|---|---|
| 52 | 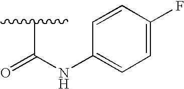 | (147) AND (148) |
| 53 | 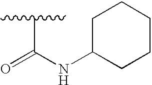 | (149) AND (150) |
| 54 | 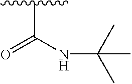 | (151) AND (152). |
| 55 | 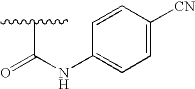 | (153) AND (154). |
| 56 | 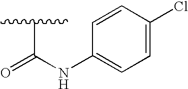 | (155) AND (156) |
| 57 | 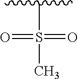 | (157) AND (158). |
| 58 | 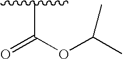 | (159) AND (160). |
| 59 | 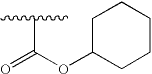 | (161) AND (162). |

### Example 60-69

Reacting compounds (119) or (120) from Preparative Example 10, in the same manner as described in Example 13, substituting the appropriate isocyanate, chloroformate or sulfonyl chloride, the following compounds were prepared:

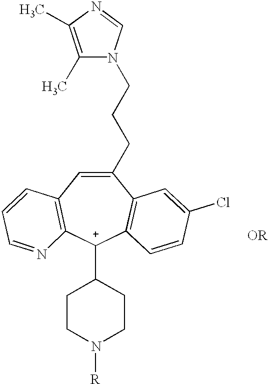

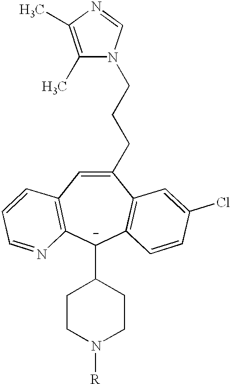

| Ex | R = | Compound #: |
|---|---|---|
| 60 | 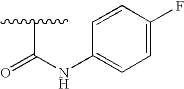 | (163) AND (164) |
| 61 | 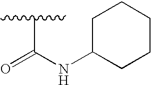 | (165) AND (166) |
| 62 | 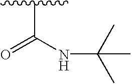 | (167) AND (168). |
| 63 | 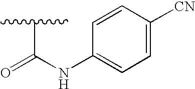 | (169) AND (170). |
| 64 | 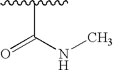 | (171) |
| 65 | 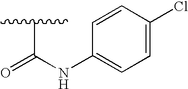 | (172) AND (173) |
| 66 | 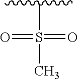 | (174) AND (175). |
| 67 | 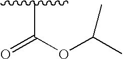 | (176) AND (177). |
| 68 | 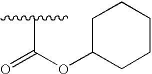 | (178) AND (179). |
| 69 | 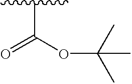 | (180) AND (181). |

### Preparative Example 11

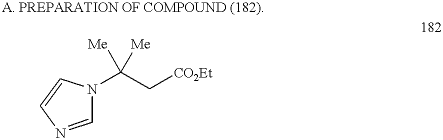

Ethyl 2,2-dimethyl acrylate (50.0 g, 2.0 eq.) was stirred with imidazole (13.28 g, 200 mmol) at 900 for 48 hours. The resulting solution was cooled, diluted with 300 mL H₂O —CH₂Cl₂ (1:1) and separated. The aqueous layer was extracted with CH₂Cl₂ (2×75 mL) and the combined organic layer was dried over Na₂SO₄ and concentrated in vacuo. The crude mixture was purified by flash chromatography using a 10% MeOH in CH₂Cl₂ solution as eluent to give pure product as a clear oil. CIMS: MH⁺=197.

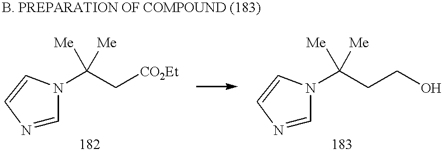

A solution of the title compound from Preparative Example 11, Step A, (10.0 g, 50.96 mmol) was treated with LiAIH₄ (51 mL, 1 M solution in ether, 1.0 eq.). The reaction mixture was stirred one hour before quenching by the dropwise additon of saturated Na₂SO₄ (˜3.0 mL). The resulting slurry was dried with Na₂SO₄ (solid), diluted with EtOAc (100 mL) and filtered through a plug of Celite. The filtrate was concentrated to give crude product which was used without further purification. CIMS: MH⁺=155.

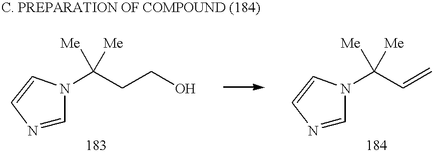

Iodine (3.83 g, 1.2 eq.) was added to a solution of Ph₃P (3.95 g, 1.2 eq.) and imidazole (1.02 g, 1.2 eq.) in CH₂Cl₂ (30 mL) portionwise over 15 minutes followed by a solution of the title compound from Preparative Example 11, Step B, (3.83 g, 12.56 mmol) in CH₂Cl₂ (10 mL). The resulting solution was stirred one hour before concentrating in vacuo. The residue was dissolved in THF (100 mL), treated with KOt-Bu (4.51 g, 3.2 eq.) and stirred at room temperature over night. The reaction mixture was diluted with water (100 mL) and CH₂Cl₂ (100 mL), separated, and the aqueous layer extracted with CH₂Cl₂ (2×50 mL). The combined organics were dried over Na₂SO₄, filtered , and concentrated under reduced pressure. The product was purified by flash chromatography using neat EtOAc then 5% MeOH in EtOAc as eluent to give a pale yellow oil (184). CIMS: MH+=137.

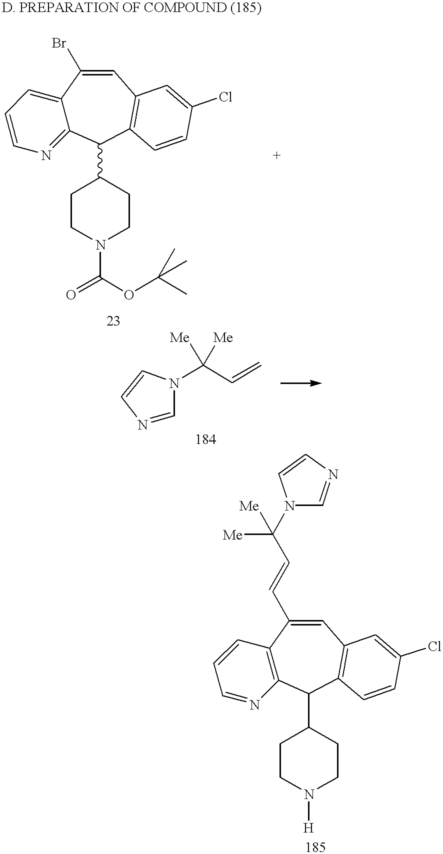

Pd(OAc)₂ (0.023 g, 10 mol %) was added to a solution of the title compound (184) from Preparative Example 11, Step C, (0.30 g, 2.0 eq.), compound (23)(0.50 g, 1.02 mmol), Bu₄NBr (0.66 g, 2.0 eq.), TEA (2.84 mL, 20.eq.) and K₂CO₃ (0.70 g, 5.0 eq) in DMF (10 mL). The resulting solution was heated to 100° C. for 48 hours, cooled to room temperature, and concentrated under reduced pressure. The residue was diluted with water (50 mL) and CH₂Cl₂ (50 mL), separated, and the aqueous layer extracted with CH₂Cl₂ (2×25 mL). The combined organic layer was dried over Na₂SO₄, filtered, and concentrated in vacuo. The crude product was purified by flash column chromatography using an 8% MeOH in CH₂Cl₂ solution as eluent to yield a 4:1 mixture of the compound (184) and coupled product (185). This mixture (0.27 g) was stirred in CH₂Cl₂: TFA (7.0 mL, 5:2) for 1.5 hours. The crude product was concentrated under reduced pressure, neutralized with NaOH (1N), and extracted with CH₂Cl₂ (3×20 mL). The combined organics were dried over Na₂SO₄, filtered, and concentrated in vacuo. The crude residue was purified by flash chromatography using a 15% (10% NH₄OH in MeOH) solution in CH₂Cl₂ as eluent to give the title compound (185) as a tan solid. LCMS: MH⁺=445.

### Example 70

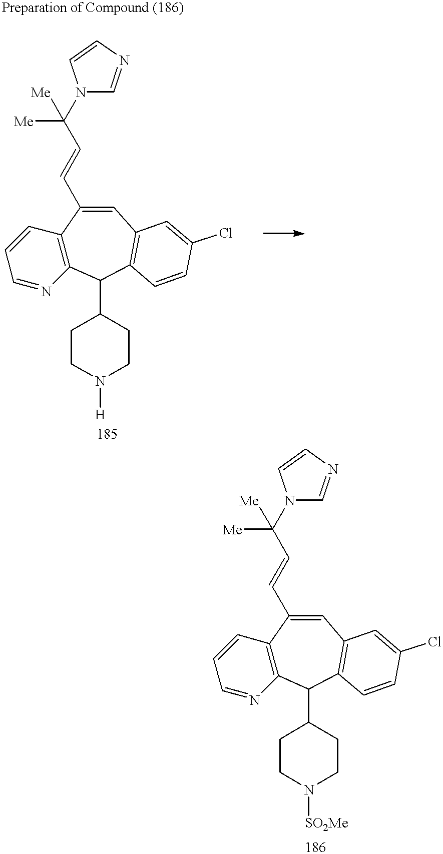

Methanesulfonyl chloride (0.005 mL, 1.3 eq) was added to a solution of Compound (185) from Preparative Example 11, Step D (0.02 g, 0.045 mmol) and TEA (0.010 mL, 1.5 eq.) in CH₂Cl₂ (1 mL). The resulting solution was stirred 12 hours at room temperature and diluted with saturated NaHCO₃ (5 mL), separated, and the aqueous layer extracted with CH₂Cl₂ (3×10 mL). The combined organic layer was dried over Na₂SO₄ and concentrated in vacuo. The crude product was purified by flash chromatography using an 8% (10% NH₄OH in MeOH) solution in CH₂Cl₂ as eluent to give the title compound (186) as a tan solid mp 124-129° C.; LCMS: MH⁺=523.

### Example 71

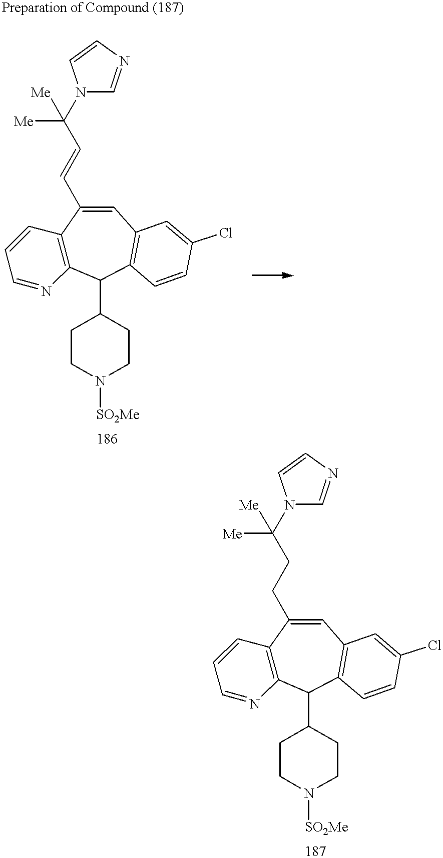

pTosNHNH₂ (0.085 g, 3 eq) was added to a solution of compound (186) from Example 70 (0.08 g, 0.0153 mmol) and DBU (0.11 mL, 5.0 eq.) in toluene (5 mL) and the resulting solution was heated to reflux. Subsequently, every 2 hours over 6 hours the solution was cooled and additional pTosNHNH₂ (3.0 eq) added and the solution heated to reflux. After heating at reflux 2 hours following the final addition the solution was cooled, diluted with CH₂Cl₂ (25 mL) and washed with saturated NaHCO₃ (3×20 mL). The organic layer was dried over Na₂SO₄, filtered, and concentrated under reduced pressure. The crude reaction mixture was purified by flash column chromatography using a 5% (10% NH₄OH in MeOH) solution in CH₂Cl₂ as eluent to give the title compound (187) as a tan solid. mp 112-116° C.; LCMS: MH+=525.

### Preparative Example 12

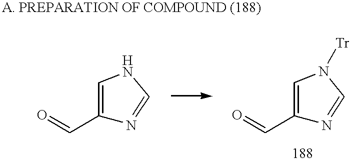

Literature compound 1H-imidazole-4-carbaldehyde was tritylated according to the literature procedure Kelley, et al.; J. Med. Chem 20(5), (1977), 721 affording the title compound (188).

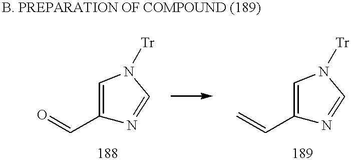

nBuLi (2.00 mL, 2.2 eq; 1.7M in hexanes) was added dropwise to Ph₃PCH₃Br (1.4 g, 2.3 eq) in THF (10 mL). The resulting orange solution was stirred 30 minutes at room temperature before cooling to −78° C. and adding the trityl protected 1 (3)H-imidazole-4-carbaldehyde (0.50 g, 1.48 mmol) in THF (7.0 mL). The resulting solution was warmed slowly to room temperature and stirred overnight. The reaction was quenched by the addition of water (20 mL) and extracted with CH₂Cl₂ (3×20 mL). The combined organics were dried over Na₂SO₄ and concentrated in vacuo. The crude product was purified by flash chromatography using a 45% hexanes in EtOAc solution as eluent to yield the title compound (189) as a white solid.

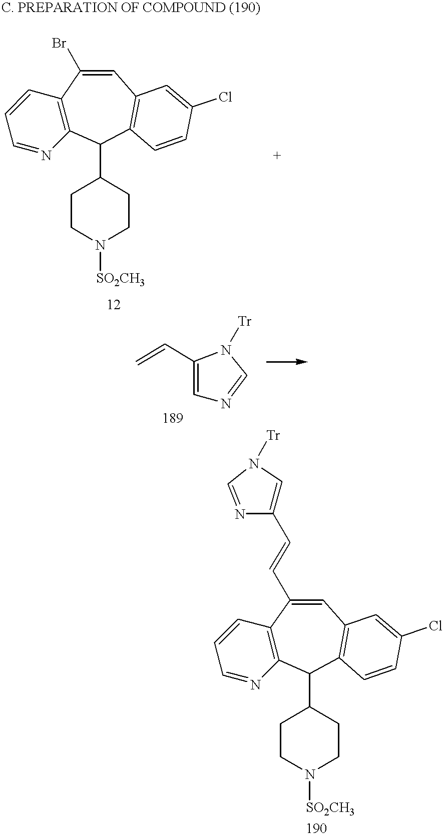

Pd(OAc)₂ (0.021 g, 0.10 eq.) was added to a solution of compound (12) from Preparative Example 2, Step B (0.44 g, 0.95 mmol), compound (189) from Preparative Example 12, Step B (0.32 g, 1.0 eq.), Bu₄NBr (0.61 g, 2.0 eq.), and K₂CO₃ (0.66 g, 5.0 eq.) in DMF (8.0 mL). The resulting solution was heated to 100° C. over night, cooled, and concentrated under reduced pressure. The residue was diluted with water (50 mL) and CH₂Cl₂ (50 mL), serparated, and the aqueous layer extracted with CH₂Cl₂ (2×50 mL). The combined organics were dried over Na₂SO₄ and concentrated in vacuo. The crude product was purified by flash chromatography using 100% EtOAc as eluent. LCMS: 723 (MH⁺).

### Example 72

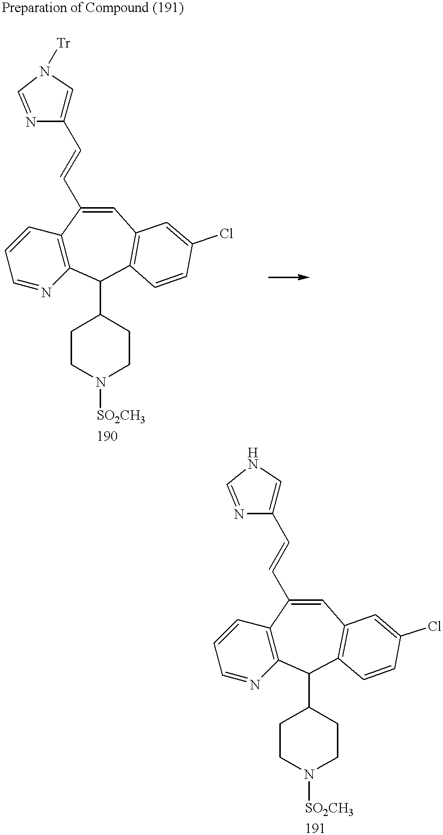

To a solution of the title compound from Preparative Example 12, Step C (1.43g, 1.97 mmol) in water (70 mL) was added AcOH (70 mL). The resulting solution was heated at reflux two hours, cooled to room temperature and neutralized by the dropwise addition of 50% (w/w) NaOH. The solution was then extracted with CH₂Cl₂ (3×200 mL) and the combine organics were dried over Na₂SO₄ and concentrated under reduced pressure. The crude product was purified by flash chromatography using a 10% (10% NH₄OH in MeOH) solution in CH₂Cl₂ as eluent. mp=190° C. (dec.); LCMS: MH⁺=483.

### Example 73

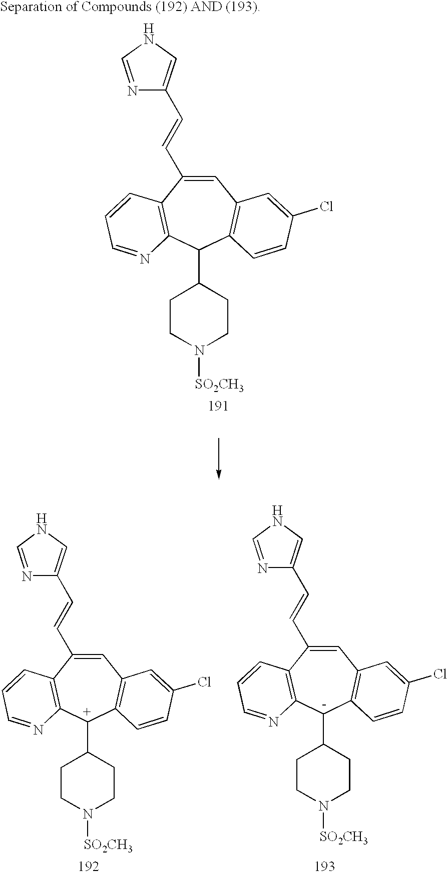

The title compound (191) from Example 72 was separated into individual (+)- and (−)- enantiomers by preparative HPLC using a ChiralPak AD column eluting with 70:30 hexanes: iPrOH containing 0.2% diethylamine as eluent.

Compound (192): FABMS: MH⁺=481; mp=109-112° C.; [α]²⁰_{D}=+398° (2.0 mg in 2.0 mL MeOH).

Compound (193): FABMS: MH⁺=481; mp=126-129° C.; [α]²⁰_{D}=−367° (2.0 mg in 2.0 mL MeOH).

### Example 74

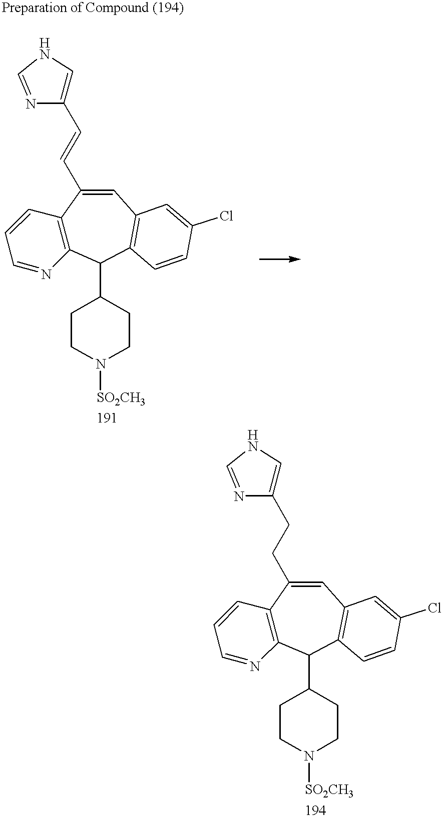

The title compound (191) from Example 72 was dissolved in toluene (50 mL) and DBU (0.26 mL, 5.0 eq.) and pTosNHNH₂ (0.33 g, 3.3 eq.) were added. The resulting solution was heated to reflux 2.5 hours before cooling to room temperature and adding additional pTosNHNH₂ (0.33 g, 3.3 eq.). The reaction mixture was heated at reflux for an additional 2 hours and cooling to room temperature. The resulting solution was diluted with saturated NaHCO₃ (100 mL) and extracted with CH₂Cl₂ (3×100 mL). The combined organics were washed with brine, dried over Na₂SO₄, filtered, and concentrated in vacuo. The crude product was purified by flash chromatography using a 5% (10% NH₄OH in MeOH) solution in CH₂Cl₂ as eluent to give pure product (194). mp=158-162; LCMS: MH⁺=483.

### Example 75

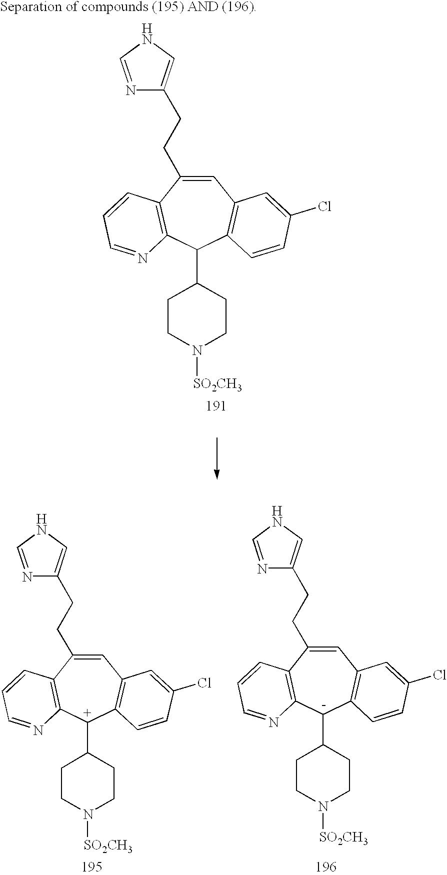

In a similar manner as described in Example 73 above, the following enantiomers were separated:

Compound (195): LCMS: MH⁺=483; mp=129-131° C.; [α]²⁰_{D}=+134° (2.0 mg in 2.0 mL MeOH).

Compound (196): LCMS: MH⁺=483; mp=125-126° C.; [α]²⁰_{D}=−105° (2.0 mg in 2.0 mL MeOH).

### Preparative Example 13

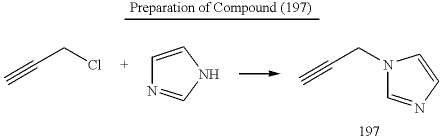

Imidazole (2.50 g, 36.72 mmol) and basic alumina (15 g) were combined and shaken 15 minutes before adding propargyl chloride (2.66 mL, 1.0 eq.). The resulting mixture was stirred 84 hours and suspended in EtOAc. The slurry was filtered and the filtrate was washed with H₂O and brine and dried over Na₂SO₄. The solution was filtered and concentrated under reduced pressure to give a clear oil.

### Example 76

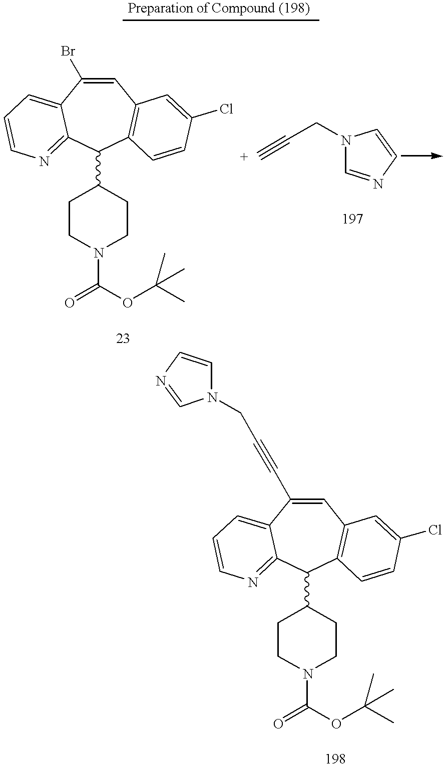

A solution of compound (23) (0.50 g, 1.02 mmol) and compound (197) from Preparative Example 13 (0.22 g, 2.0 eq.) in TEA (3.0 mL) and pyridine (0.5 mL) was deoxygenated 15 minutes before adding PdCl₂(PPh₃)₂ (0.018 g, 2.5 mol %) and Cul (0.002 g, 1.0 mol %). The resulting solution was heated for 48 hours. The reaction mixture was cooled to room temperature, diluted with H₂O, and extracted with CH₂Cl₂. The combined organic layer was dried over Na₂SO₄, filtered, and concentrated. The s crude product was purified by flash chromatography using an 8% MeOH in CH₂Cl₂ solution as eluent. mp 109-112° C.; LCMS: 515 (MH+).

### Preparative Example 14

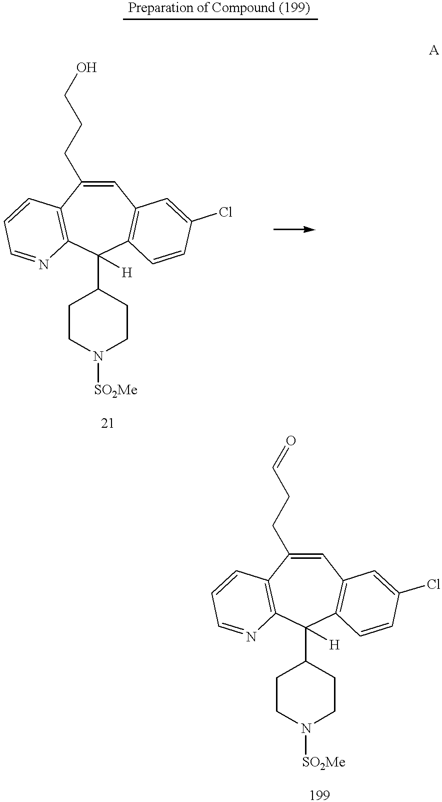

Compound (21) from Preparative Example 3, Step C, (2.83 g, 6.37 mmol) was dissolved in 120 ml of dichloromethane and 0.16 ml of de-ionized water. Dess-Martin periodinane (3.85 g, 9 mmol) was added as a solid at ambient temperature and the reaction mixture stirred for 4 hours. Then added a 20% Na₂S₂O₃ solution (50 ml) and stirred for 15 minutes. The layers were separated and the dichloromethane layer washed with saturated NaHCO₃, dried over magnesium sulfate, filtered and evaporated to obtain the title product (199). FABMS: 445 (MH⁺).

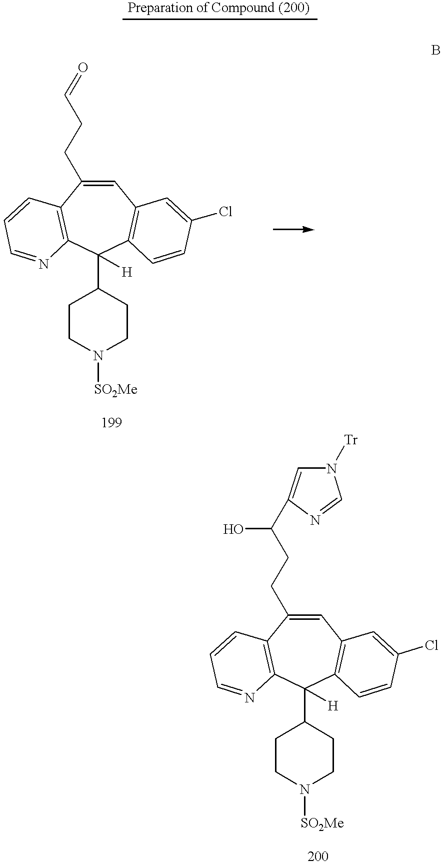

4-Iodo-1-trityl-imidazole (prepared according to the literature procedure Kirk, Kenneth L.;J. Heterocycl. Chem.; EN; 22; 1985; 57-59) (0.48 g, 1.1 mmol) was dissolved in 5 ml of dichloromethane under a dry nitrogen atmosphere. Ethylmagnesium bromide (0.36 ml) was added and the reaction mixture stirred. After 30 minutes compound (199) (0.44 g, 1 mmol) was dissolved in 5 ml of dichloromethane and added to the reaction mixture while stirring. After stirring 4 hours at ambient temperature, the mixture was washed with saturated ammonium chloride solution, dried over magnesium sulfate, filtered, and evaporated to give a solid residue. The product was chromatographed on a flash silica gel column using ethyl acetate as the eluent to obtain the title compound (200). FABMS: 756 (MH⁺).

### Example 77

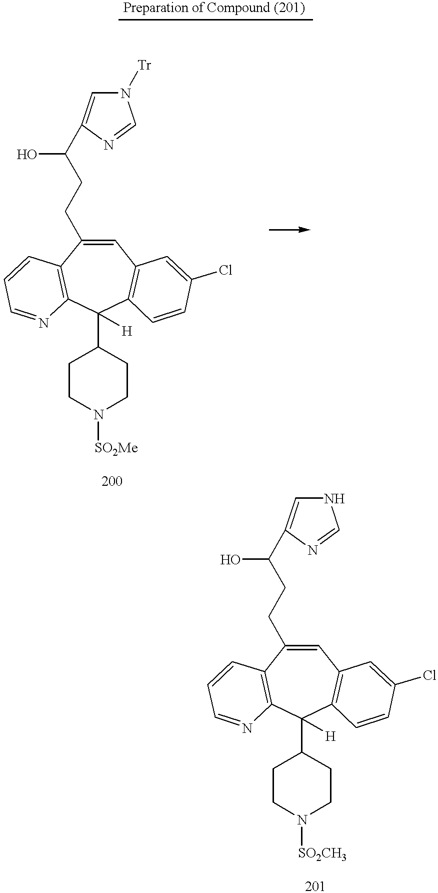

Compound (200) (0.6 gm) was dissolved in 10 ml of trifluoroacetic acid and stirred at ambient temperature. After 7 hours the reaction mixture was evaporated to dryness under vacuum and chromatographed on silica gel using 5% 2N methanol:ammonia/dichloromethane to obtain title compound (201). FABMS: 514 (MH⁺).

### Preparative Example 15

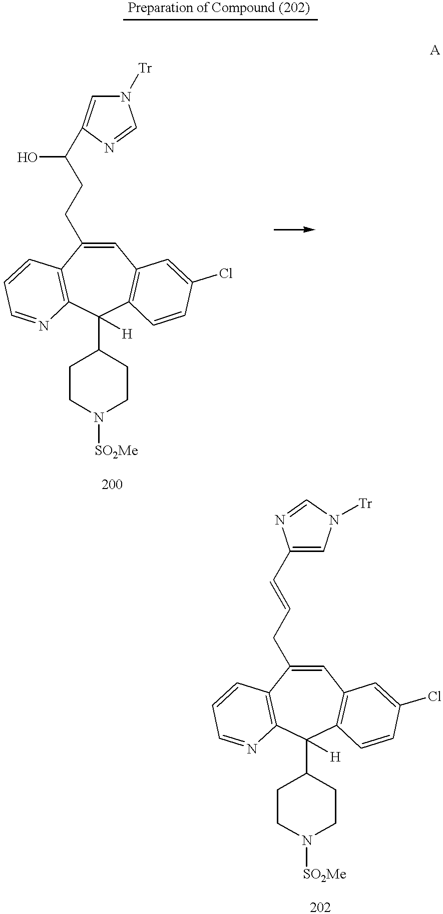

Compound (200) (0.5 g, 0.66 mmol) was dissolved in 5 ml of dichloromethane. Triethylamine (0.14 ml, 0.99 mmol) and methanesulfonyl chloride (0.062 ml, 0.79 mmol) were added and the reaction mixture stirred for 18 hours. The reaction mixture was added to brine and extracted with dichloromethane three times. Dried over magnesium sulfate, filtered and concentrated to dryness under vacuum to give a residue which was chromatographed on silica gel using ethyl acetate as the eluent to obtain the title compound (202). FABMS: 537 (MH⁺).

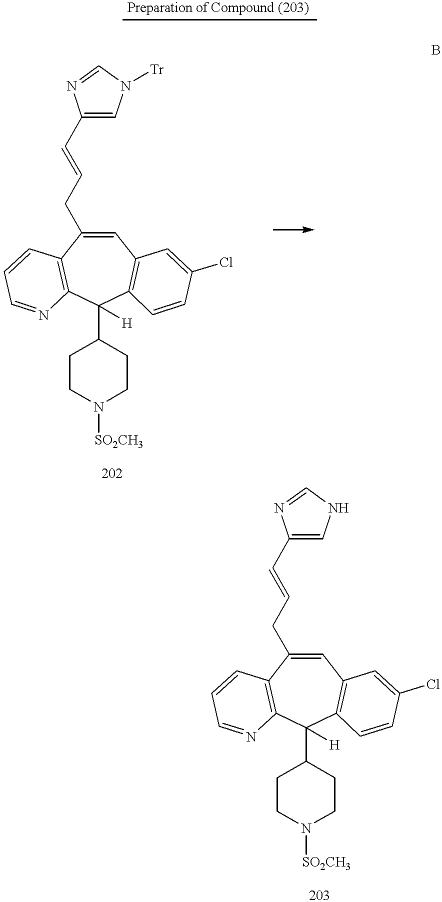

Compound (202) was detritylated in the same manner as EXAMPLE 77 affording the title compound (203). FABMS: 495 (MH⁺).

### Example 78

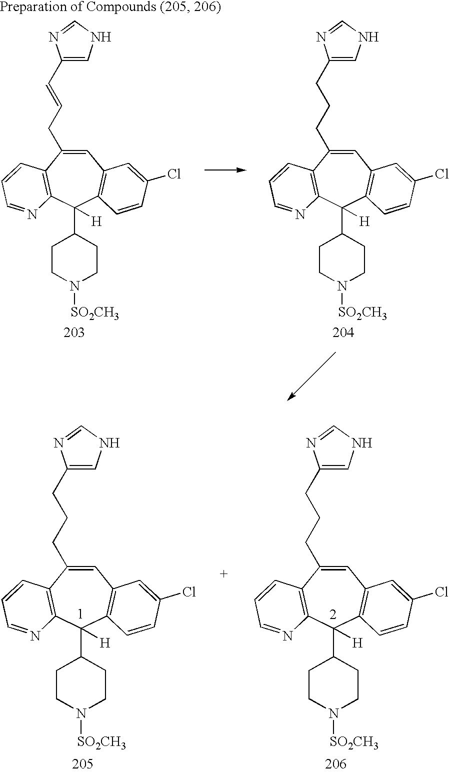

Compound (203) (77 mg) was hydrogenated over PtO₂ in ethanol at atmospheric hydrogen for 24 hours. After filtration of the catalyst followed by evaporation of the ethanol and chromatography on a Chiral Technologies® AD HPLC column the title product was obtained as two pure enantiomers (205) and (206). FABMS: 497 (MH⁺).

### Preparative Example 16

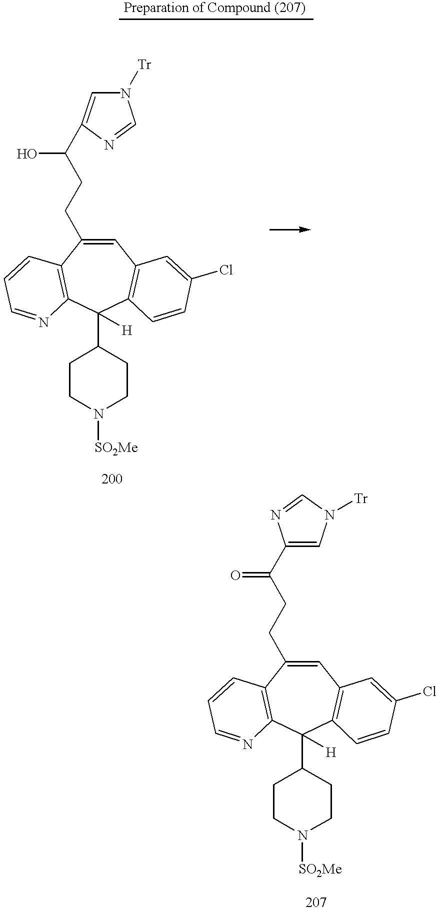

Compound (200) (0.15 g, 0.198 mmol) was dissolved in 4 ml of dichloromethane and 5 uL of de-ionized water. Dess-Martin periodinane (0.12 g, 0.3 mmol) was added and the reaction mixture stirred for 4 h. 5 ml of a 20% Na₂S₂O₃ solution was added and the reaction mixture stirred for another 15 minutes. The layers were separated and the dichloromethane layer was washed with saturated NaHCO₃, dried over magnesium sulfate, filtered and evaporated to obtain the title compound (207). FABMS: 753 (MH⁺).

### Example 79

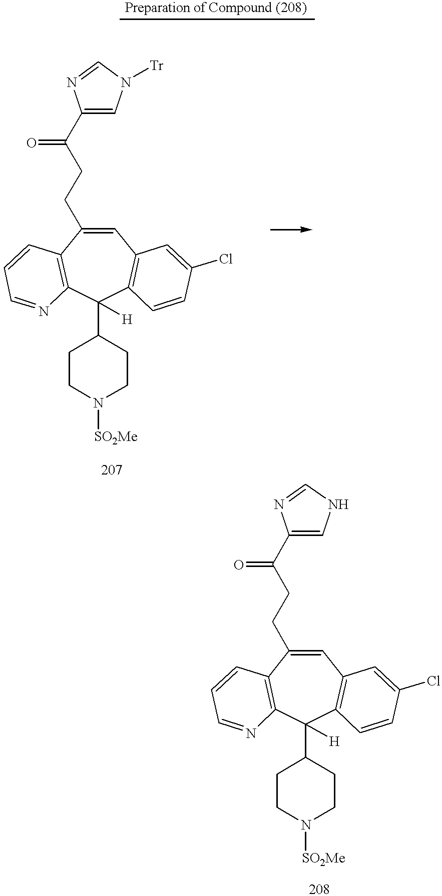

Compound (207) was detritylated in the same manner as Example 77 affording the title compound (208). FABMS: 511 (MH⁺).

### Preparative Example 17

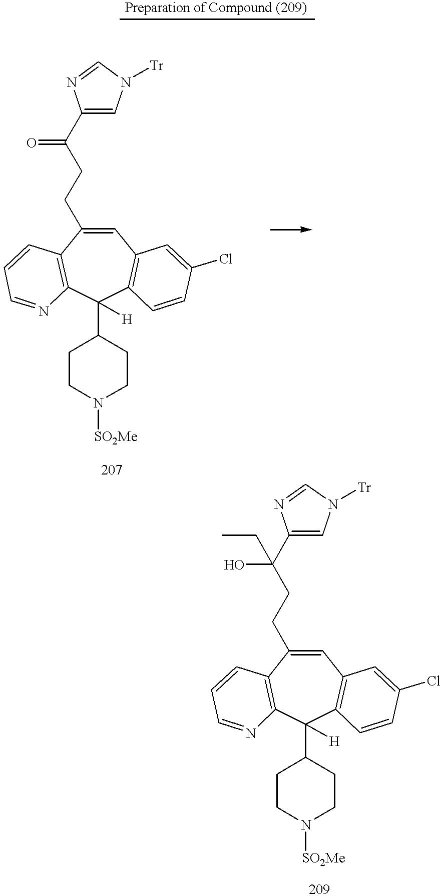

Compound (207) (0.15 g, 0.2 mmol) was dissolved in 5 ml of tetrahydrofuran. Ethylmagnesium bromide (0.1 ml, 3 M in ether) was added at ambient temperature and stirred under a dry nitrogen atmosphere. After 2 hours, added another portion of ethylmagnesium bromide (0.1 ml, 3 M in ether). After 4 hours the reaction mixture was washed with saturated ammonium chloride, dried over magnesium sulfate, filtered and evaporated to obtain the title compound (209). The product was further purified by flash silica column chromatography eluting with 50% ethylacetate/hexanes. FABMS: 783 (MH⁺).

### Example 80

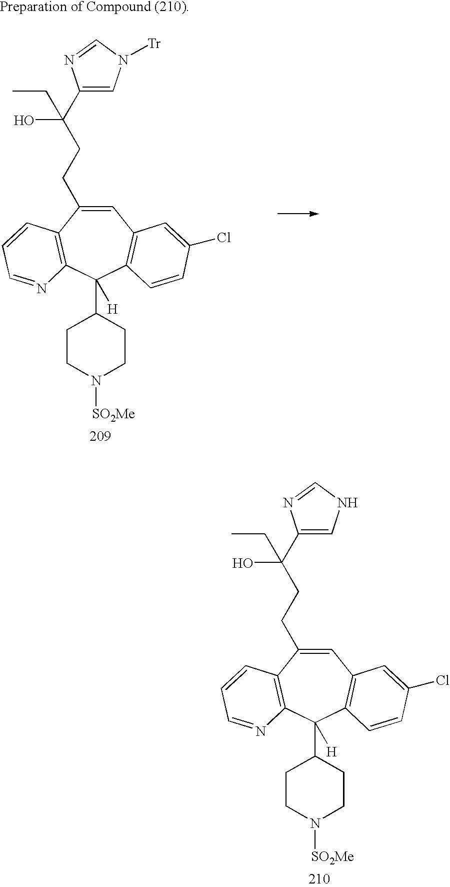

Compound (209) was detritylated in the same manner as Example 77 affording the title compound (210). FABMS: 541 (MH⁺).

### Preparative Example 18

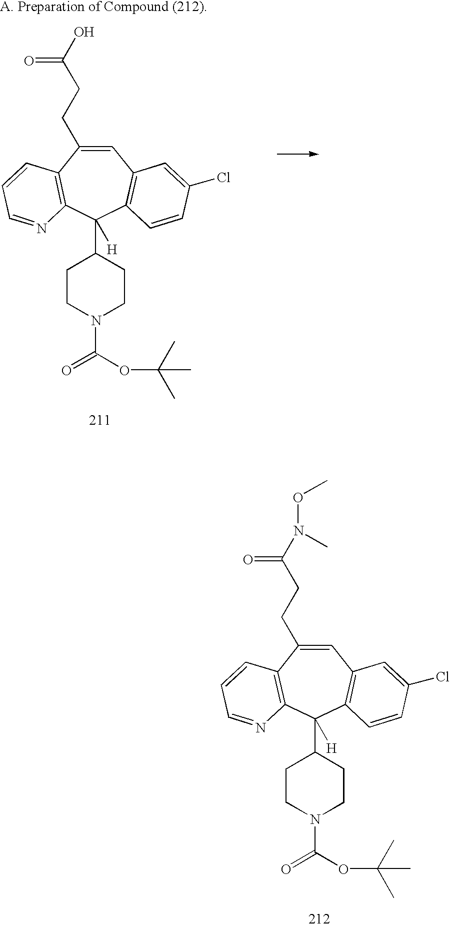

Compound (211) (14 g, 29 mmol) prepared by NaOH hydrolysis of Compound (20) from Preparative Example 3, Step B, was dissolved in 400 ml of DMF. 1-(3-dimethylamino propyl)-3-ethylcarbodiimide hydrochloride (8.3 g, 43 mmol), 1-hydroxybenzotriazole (5.9 g, 43 mmol), triethylamine (40 ml), and N,O-dimethylhydroxylamine hydrochloride(3.8 g, 40 mmol) were added and the reaction mixture stirred at room temperature under a dry nitrogen atmosphere. After 24 hours the reaction mixture was poured into brine and the product extracted with ethylacetate two times. After drying over magnesium sulfate, filtration, and chromatography on silica gel using 10% ethyl acetate/hexanes the title compound (212) was obtained.

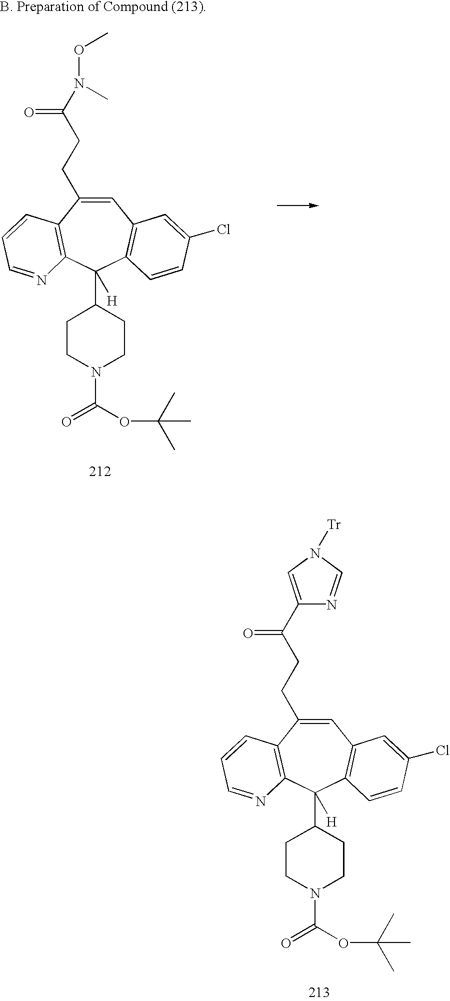

Compound (212) (0.53 g, 1.01 mmol) was treated as in PREPARATIVE Example 14, Step B to obtain the title compound (213) after silica gel chromatography.

### Example 81

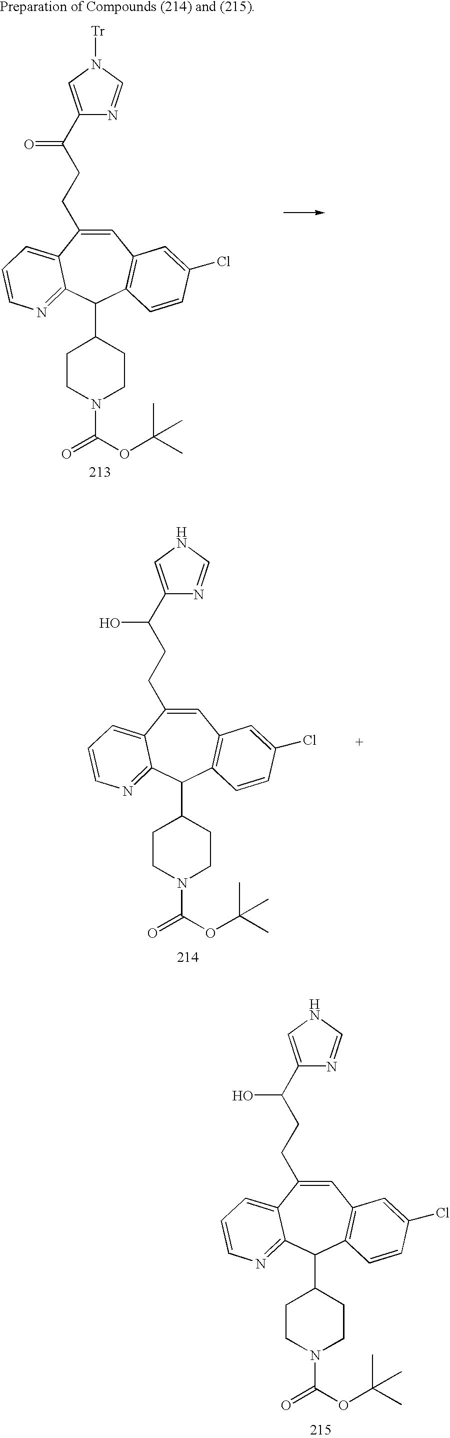

Compound (213) (300 mg, 0.387 mmol) was dissolved in methanol and sodium borohydride (50 mg) was added portionwise while stirring. After 1 hour the mixture was added to 1N HCl followed by the addition of 1N NaOH and extracted with ethylacetate to obtain a crude product which was treated with neat trifluoroacetic acid for 5 hrs, and evaporated to dryness. The mixture was dissolved in methanol and reacted with di-tert.butyldicarbonate (0.2 gm) while maintaining the pH at 10 with 1N NaOH for 1 hour. The mixture was then treated with 2N Methanolic ammonia for 15 minutes followed by evaporation of the solvents and chromatography on silica gel. Further separation of isomers was accomplished on a Chiral Technologies® AD HPLC column obtaining the pure isomers. (214) and (215). FABMS M+1=535

### Example 82

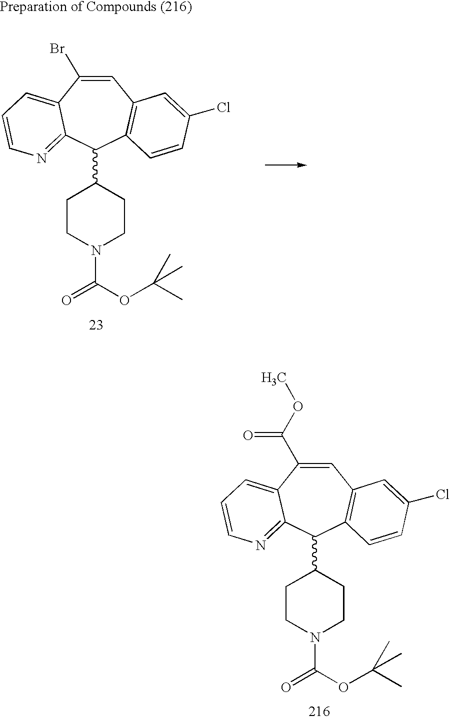

Compound (23) from Preparative Example 4, Step A (25.47 gm, 52 mmol) was dissolved in 300 ml of dry toluene and 39.5 ml of methanol. Palladium chloride (0.92 gm), triphenylphosphine (6.887 gm) and DBU (10.5 ml) were added and the reaction mixture transferred to a pressure reaction vessel. The reaction vessel was purged with carbon monoxide and then pressurized to 100 psi with carbon monoxide and the mixture stirred at 80° C. for 5 hours. The reaction was cooled in an ice bath and purged with nitrogen 3-4 times. The reaction mixture was transferred to a separatory funnel and 500 ml of ethylacetate was added. The mixture was washed with water three times, dried over magnesium sulfate, filtered and evaporated to dryness under vacuum to give a dark brown gum. The gum was purified by column chromatography on silica gel using 12.5%-25% ethylacetate/hexanes to obtain 12.58 gm of pure title product (216) FABMS: 469 (MH⁺) and 9.16 gm of a mixture of two compounds.

### Preparative Example 19

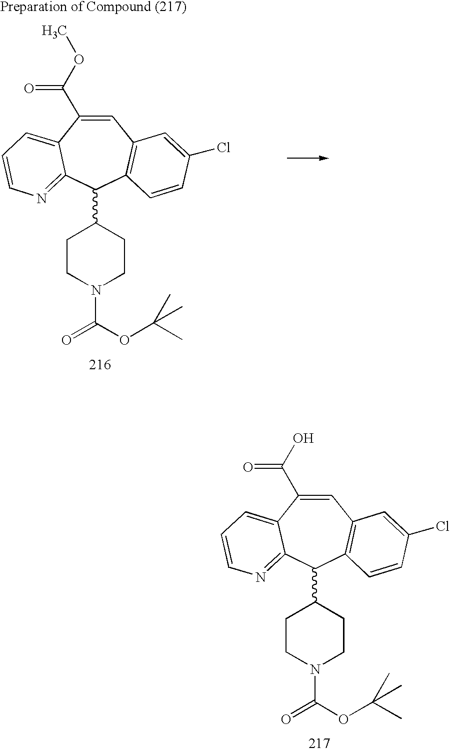

Compound (216) from Example 82 (5.16 gm, 11 mmol) was dissolved in methanol (150 ml). 10% lithium hydroxide (2.9 ml) was added along with dioxane (50 ml) and the reaction stirred for 4 hours. Added an additional portion of 10% lithium hydroxide (5.7 ml) and the reaction stirred for 18 hours. The reaction mixture was concentrated to s small volume and diluted with 50 ml of water. The mixture was acidified to pH=3 with 10% citric acid and the product extracted with dichloromethane to obtain the title compound (217). FABMS: 455 (MH⁺)

### Preparative Example 20

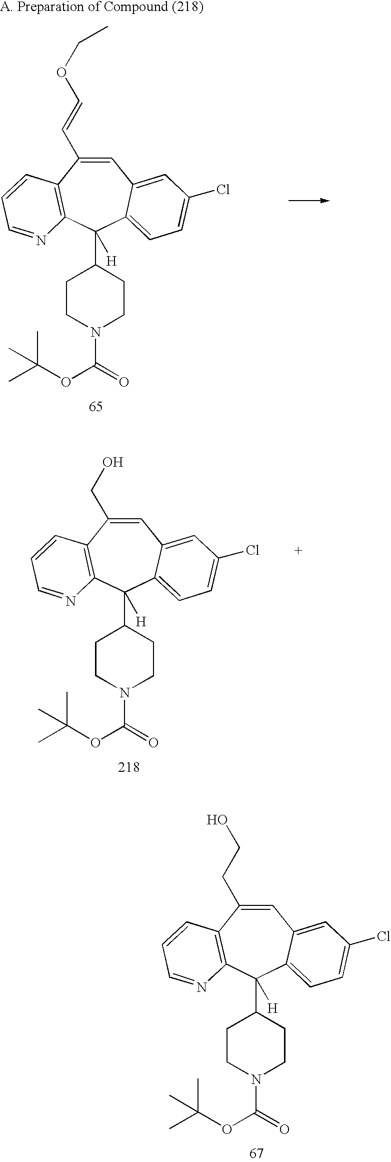

Compound (65) from Preparative Example (6), Step B, was let stand for approximately two weeks at room temperature, after which time the pressence of some aldehyde was observed by NMR of the crude material. This material was then treated as in Preparative Example 6, Steps C and D to afford a mixture of Compounds (218) and (67). The crude mixture was separated on flash silica column chromatography eluting with 1:1-3:1 ethyl acetate:hexanes to afford pure Compound (218).

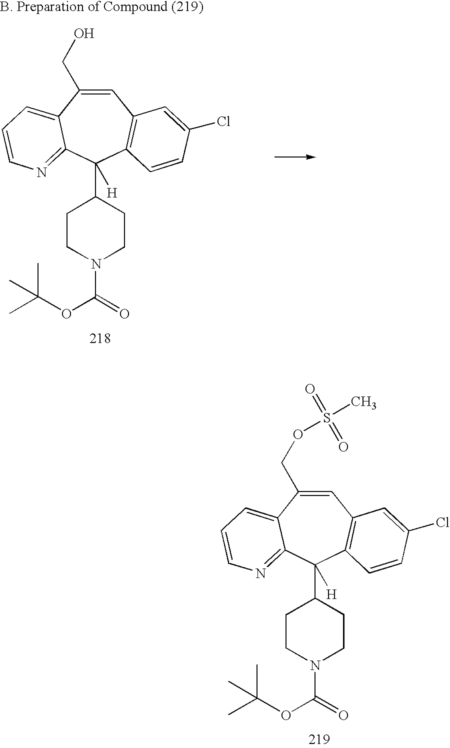

Compound (218) from Step A above, was combined with triethylamine (64.4 ml; 0.462 mmol) in CH₂Cl₂ (4 ml) treated with methyl sulfonyl chloride (17.93 ml; 0.231 mmol) and let stir over night at room temperature. The reaction mixture was diluted with CH₂Cl₂ (70 ml), quenched with brine (25 ml) and extracted. The organic layer was dried over MgSO₄, filtered and concentrated to give an off-white solid (219) (93 mg; 100%).

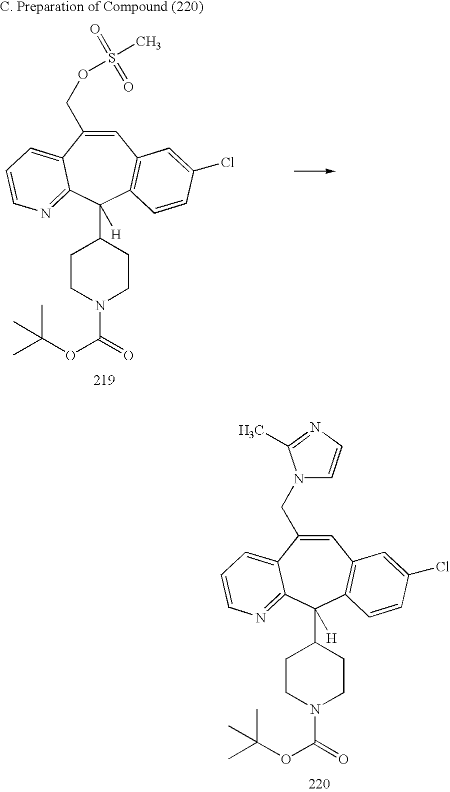

Compound (219) from Step B above, was taken up in DMF. To this solution was added a previously reacted solution of 2-methyl imidazole (145.27 mg; 1.734 mmol) and NaH (60%) (69.4 mg; 1.734 mmol) in DMF. The reaction mixture was allowed to stir at room temperature for two hours. The DMF was removed and the residue taken up in CH₂Cl₂ quenched with sat. aqueous NaHCO₃ and extracted with 2×100 ml CH₂Cl₂. The organic layers were combined and purified by preparative TLC plates to give an off-white solid. (220)

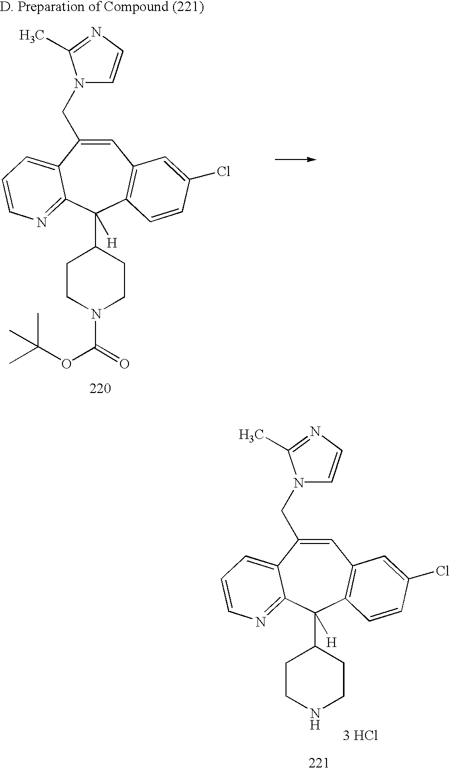

Compound (220) from Step C above, was dissolved in 1,4-Dioxane (3 ml). To this solution was then added 4M HCl in Dioxane (5 ml) and the reaction stirred for 3 hours at room temperature. The mixture was then concentrated and dried over night under high vacuum to afford the hydrochloride salt as an off-white solid. (221)

### Example 83

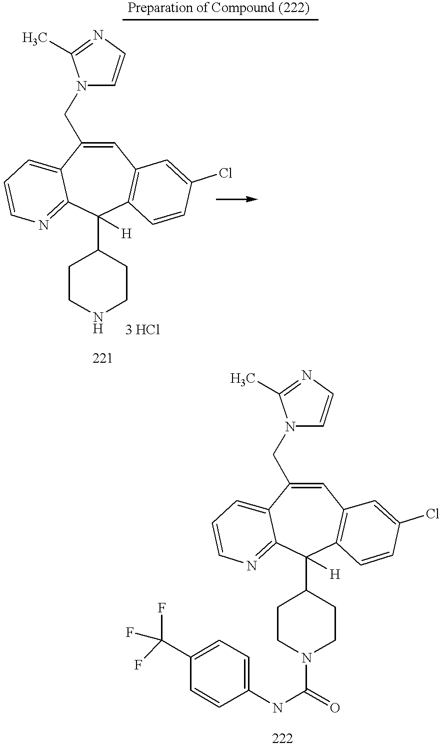

To a solution of compound (221) from Preparative Example 20, Step D (51 mg; 0.126 mmol) and triethylamine (61.47 ml; 0.441 mmol) in CH₂Cl₂ (2 ml) was added 4-trifluoromethylphenyl isocyanate (20.26 ml; 0.139 mmol) at 0° C. The reaction stirred for 2-3 hours under N₂ atmosphere. The CH₂Cl₂ and excess triethylamine were removed under vacuo and the resultant product was purified by preparatory thin layer chromatography eluting with 98:2 CH₂Cl₂/(sat.)MeOH/NH₃) affording the title compound as a white solid (222).

### Preparative Example 21

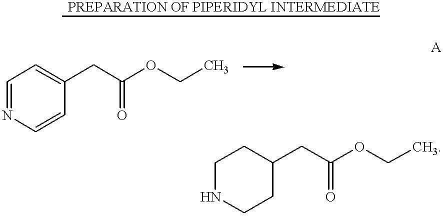

Commercially available Ethyl 4-Pyridyl Acetate (4.5 g; 27.2 mmol), EtOH (70 ml) and 10% Palladium on Charcoal (catalytic) was shaken under 55 psi hydrogen at room temperature for 94 hrs. The mixture was filtered through Celite and the cake was washed with (4×40 ml) of EtOH. The filtrate was concentrated and purified by flash silica column chromatography eluting with 3% (10% NH₄OH:MeOH)/CH₂Cl₂.

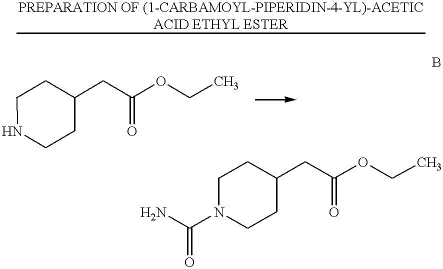

4-Pyridyl Acetic Acid (2.362 g) from Step A above, was taken up in CH₂Cl₂ (118 ml). To this was added trimethylsilyl isocyanate (27.87 ml). The reaction stirred for 67 hr then was diluted with CH₂Cl₂ (700 ml) and washed with saturated aqueous NaHCO₃ (150 ml). The aqueous layer was extracted with 2×200 ml CH₂Cl₂. The organic layers were combined, dried over MgSO₄, filtered and concentrated. The crude product was purified by flash silica column chromatography eluting with 2% (10% NH₄OH:MeOH)/CH₂Cl₂.

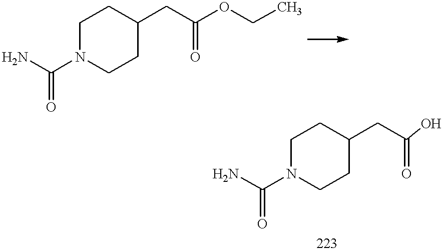

C. Product from Step B above (40.63 mg; 0.1896 mmol) was taken up in EtOH (2 ml) and CH₂Cl₂ (2 ml) and treated with 1 M LiOH (.5 ml; 0.455 mmol). The reaction mixture was heated to 50° C. and stirred for 5 hr. The reaction was cooled to room temperature treated with 1N HCl (.57 ml; 0.531 mmol) and stirred for 5 minutes. The resultant mixture was concentrated and dried under high vacuum for 4 days affording the title compound as a white solid. (223)

### Example 84

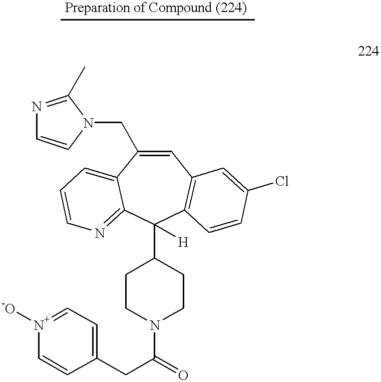

To a solution of Compound (221) from Preparative Example 20, Step D (51 mg; 0.126 mmol), 4-methylmorpholine (69.3 ml; 0.630 mmol), DEC (31.44 mg; 0.164 mmol), and HOBT (22.2 mg; 0.164 mmol) in DMF (2 ml) was added, 4-Pyridylacetic Acid 1-N-Oxide (disclosed in U.S. Pat. No. 5,719,148; Feb. 17, 1998). The reaction stirred for 3 hours at room temperature. The reaction was diluted with CH₂Cl₂ and washed two times with saturated aqueous NaHCO₃. The organic layers were combined, concentrated and purified by preparative thin layer chromatography eluting with 95:5 CH₂Cl₂: sat. MeOH/NH₃ affording the title compound as a white solid (224).

### Example 85

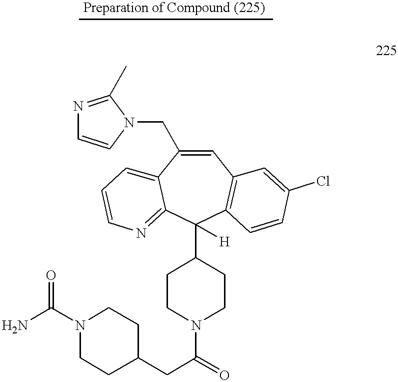

Compound (221) from Preparative Example 20, Step D (51 mg; 0.126 mmol) was combined with compound (223) from Preparative Example 21, Step C and reacted in the same manner as Example 84 to afford the title compound as a white solid. (145-155° C. dec.) MH⁺573.(225)

### Example 86

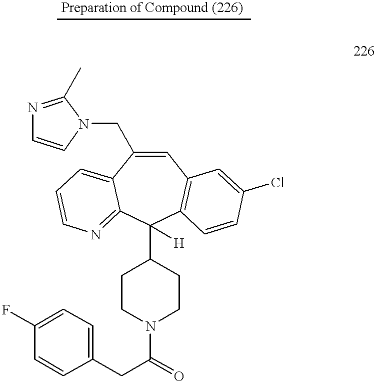

Compound (221) from Preparative Example 20, Step D (51 mg; 0.126 mmol) was combined with 4-Fluorophenylacetic acid (Acros) (29.29 mg; 0.190 mmol) and reacted in the same manner as Example 84 to afford the title compound as an off-white solid. (108-125° C. dec.) MH⁺541.(226)

### Preparative Example 22

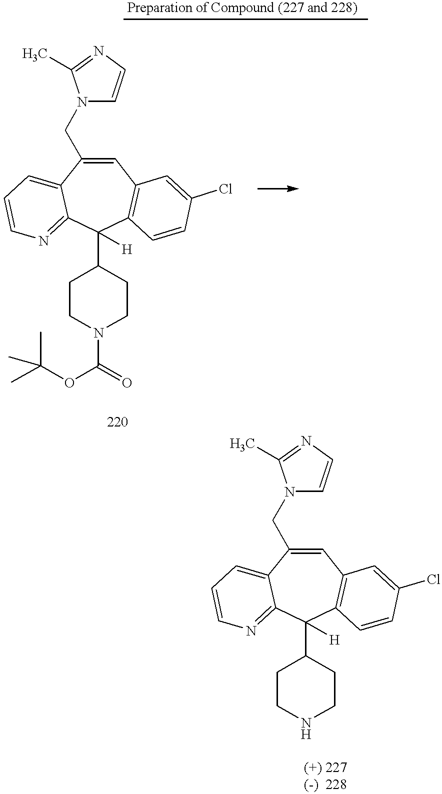

Compound (220) from Preparative Example 20, Step C, (150 mg; 0.289 mmol) was treated with 4M HCl in Dioxane and allowed to stir for 2-3 hr at room temperature under a N₂ atmosphere. The crude mixture was separated into pure (+) isomer (227) and (−) isomer (228) by preparative chiral HPLC using an AD column, eluting with 85:15:2Hexanes:IPA:DEA.

### Examples 87-90

The appropriate (+) compound (227) or (−) compound (228) isomer from Preparative Example 22 above, was taken up in CH₂Cl₂ treated with the corresponding isocyanate and stirred at room temperature over night. Crude product was purified directly by preparative thin layer chromatography to afford the following compounds (229-232):

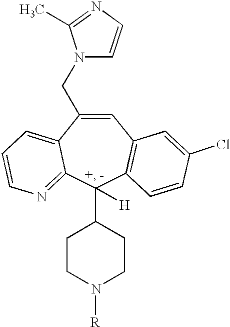

| | | Com- | |
|---|---|---|---|
| Ex. | R | pound # | |
| 87 | 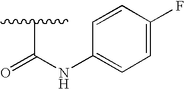 | (229) | (+)(148-156° C. dec.) MH⁺ 556. |
| 88 | 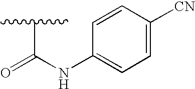 | (230) | (+)(155-166° C. dec.) MH⁺ 563. |
| 89 | 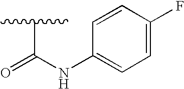 | (231) | (−)(145-153° C. dec.) MH⁺ 556. |
| 90 | 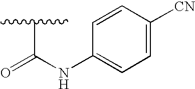 | (232) | (−)(159-168° C. dec.) MH⁺ 563. |

### Preparative Example 23

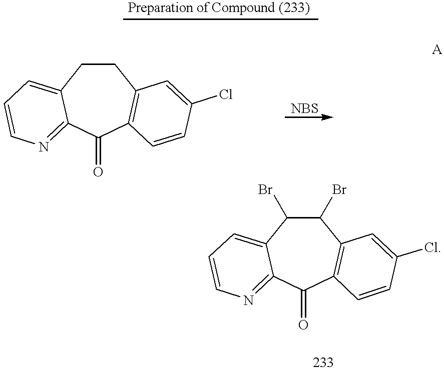

The tricyclic keto-compound (disclosed in U.S. Pat. No. 5,151,423) (30.0 g; 123.2 mmol) was combined with NBS (48.2 g; 271.0 mmol) and benzoyl peroxide (0.42 g) in CCl₄ (210 ml). The reaction was heated to 80° C. for 10 hr. The mixture was cooled and let stand for 8 hr. The resulting precipitate was filtered. Added MeOH (200 ml) and stirred the mixture over 2 days. The solid was filtered and dried under vacuum to a constant weight.

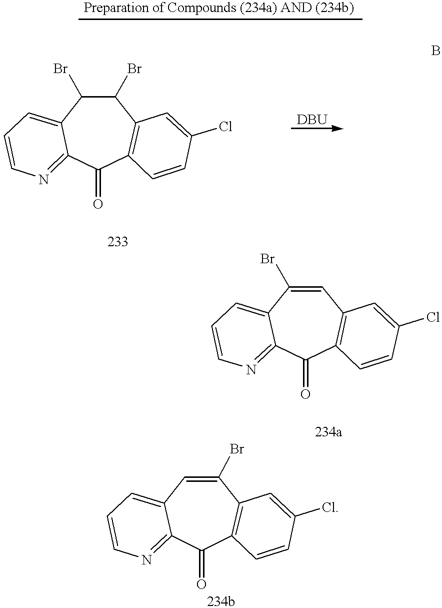

The dibromo compound (233) from Step A (35.72 g; 88.97 mmol) above was dissolved in CH₂Cl₂ (1.5 L) and cooled to 0° C. Dropwise, DBU (15.96 ml) was added and the suspension stirred for 3 hr. The reaction mixture was concentrated redissolved in CH₂Cl₂ (1.5 L) filtered through a bed of silica gel and rinsed with 5% EtOAc/CH₂Cl₂ (4 L). The combined rinses were concentrated and purified by flash silica gel column chromatography into pure 5 and 6 mono-bromo substituted compounds eluting with 10-30% EtOAc/Hex then 3%EtOAc/CH₂Cl₂.

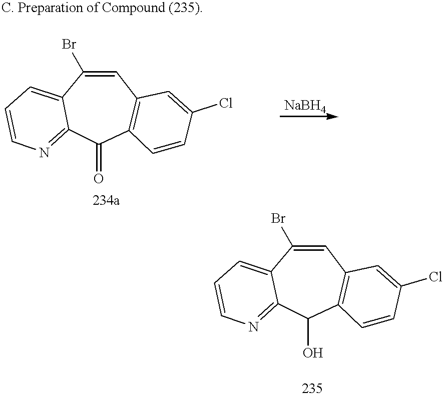

The 5-bromo substituted compound (234a) from Step B above (4.0 g; 12.45 mmol) was taken up in MeOH and cooled to 0° C. NaBH₄ (916.4 mg; 24.2 mmol) was added and the reaction mixture stirred for 5.5 hr. The solvent was removed and the resulting residue was used directly.

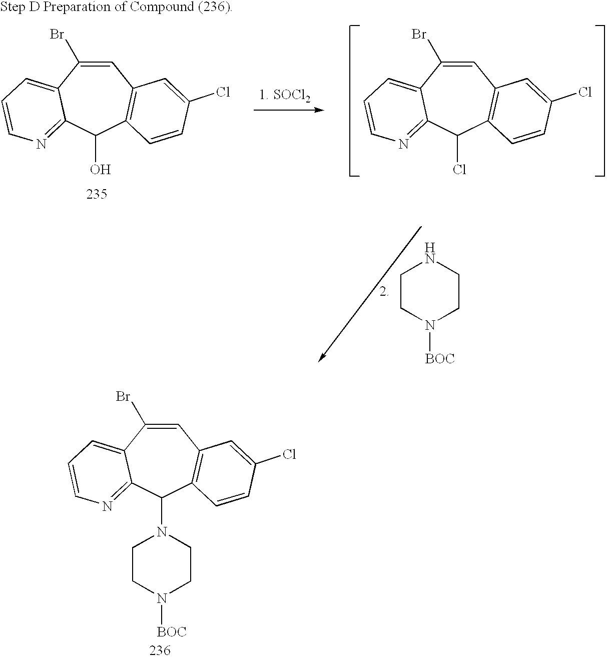

The alcohol compound (235) from Step C above (3.98 g; 12 mmol) was dissolved in CH₂Cl₂ cooled to 0° C. and treated with 2,6-Lutidine (5.73 ml; 49 mmol). SOCl₂ (1.8 ml; 24.6 mmol) was added and the reaction was allowed to stir and come to room temperature over 3 hr. The reaction mixture was poured into 0.5 N NaOH (80 ml) extracted and concentrated in vacuo. The crude product was taken up in CH₃CN and treated with 1,2,2,6,6-Pentamethylpiperidine (4.45 ml; 24.6 mmol) (Aldrich). The reaction was heated to 60-65° C. treated with tert-butyl 1-piperazinecarboxylate (2.32 g; 12 mmol) (Aldrich) and stirred over night under N₂ atmosphere. The reaction mixture was concentrated to dryness, redissolved in CH₂Cl₂ and washed with sat. aqueous NaCO₃. The organic layer was dried over Na₂SO₄, filtered and purified by flash silica gel column chromatography eluting with 1:4-1:2 EtOAc/Hexanes to afford the product as a white solid.

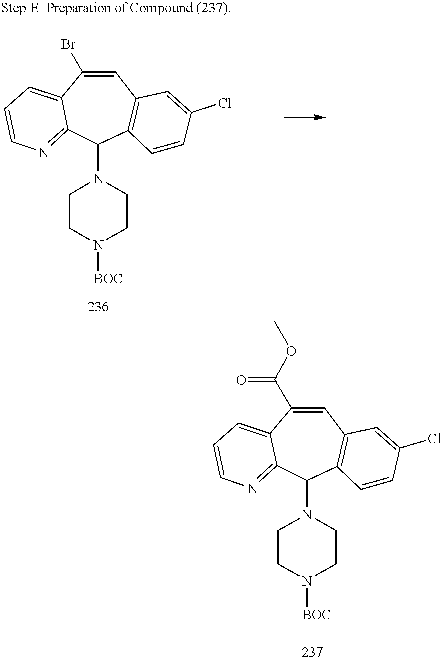

The BOC-protected bromo-compound (236) from Step D above (2 g; 4 mmol), triphenyl phosphine (.54 g; 2 mmol), and palladium chloride (.0723 g; 0.4 mmol) were combined in MeOH (10 ml) and toluene (30 ml). To this mixture was added DBU (0.835 ml; 5.5 mmol) and the mixture was sealed in a Parr bomb. The reaction mixture was stirred and subjected to 90 psi of CO at 80° C. for 5 hr. The reaction was diluted with EtOAc (200 ml) and washed with 2×80 ml H₂O. The organic layer was dried over MgSO₄, filtered and purified by flash silica column chromatography eluting with 1:3 EtOAc/Hexanes.

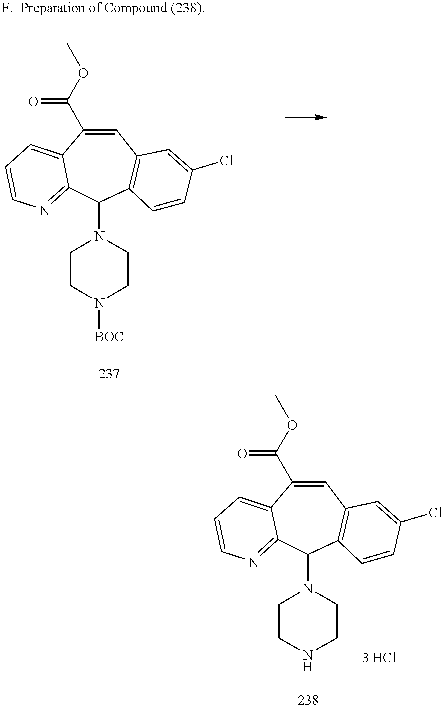

Compound (237) from Step E above (1.73 g; 3.681 mmol) was treated with 4 M HCl in Dioxane (35 ml) and allowed to stir at room temperature for 3 hr. The reaction mixture was concentrated in vacuo and the resulting tan solid was further dried under high vaccuum.

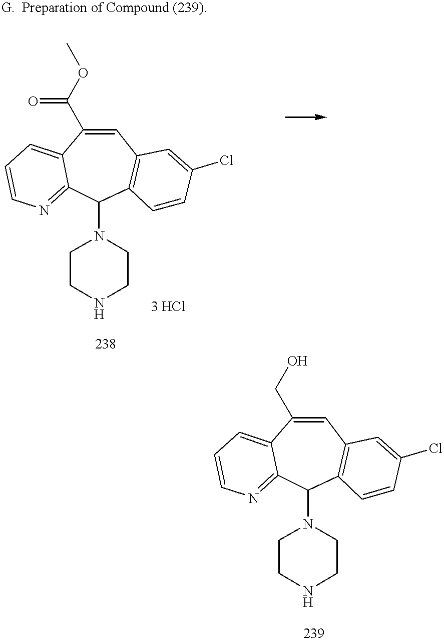

The HCl salt (238) from Step F above (1.36 g; 3.68 mmol) was dissolved in THF, cooled to 0° C., treated with 1 M DIBAL in cyclohexane (18.41 ml; 18 mmol) and stirred over night at room temperature. The mixture was concentrated to dryness and used directly in the next step.

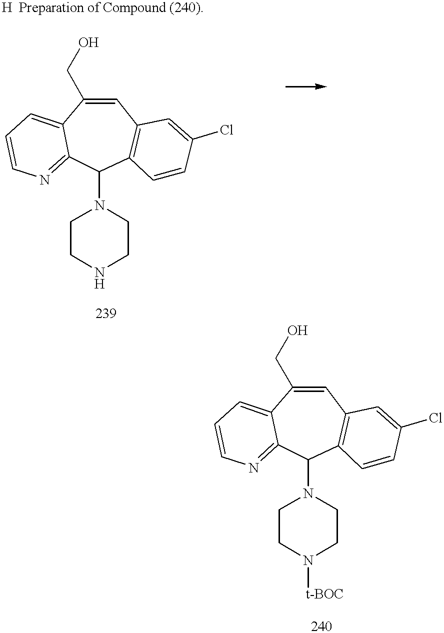

The alcohol (239) from Step G above was taken up in MeOH (50 ml) and H₂O (5 ml) and treated with Boc anhydride (1.56 g; 7.14 mmol). The pH was adjusted to approximately 10 with 1N NaOH. The reaction mixture was concentrated, taken up in CH₂Cl₂ and washed with H₂O (2×) The organic layer was dried over MgSO₄, filtered and concentrated to a tan solid containing both product and an impurity.

Alternatively, compound (237) was converted to compound (240) by first preparing the acyl imidazole followed by NaBH₄ reduction using the following procedure:

Compound (237) from Step E above (7.0 mmol) was dissolved in a mixture of 15 mL methanol, 60 mL dioxane and 6 mL water containing 25 mL of 10% aqueous LiOH. The mixture was heated at 60° C. for 4 hr, then it was concentrated under vacuum and the pH adjusted to 5.2 with 10% aqueous citric acid. The residue was dissolved in CH₂Cl₂, washed with brine, dried over MgSO₄ and concentrated under vacuum to give the carboxylic acid. The acid was then dissolved in 20 mL THF containing 14 mmol of 1,1′-carbonyl diimidazole and heated at 38° C. for 18 hr. The mixture was then concentrated under vacuum to give the acyl imidazole. The residue was dissolved in a mixture of 21.2 mL of THF and 5.3 mL water and cooled to 0° C. To the solution was added 35 mmol of NaBH₄ and it was stirred for 1.5 hr. 5 mL brine and 25 mL CH₂Cl₂ was then added The organic layer was dried over MgSO₄ and concentrated under vacuum to give compound (240) in essentially a quantitative yield.

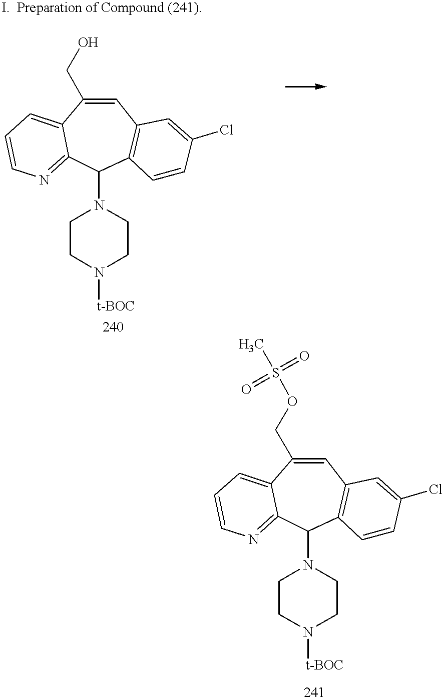

The crude product (240) from Step H above (200 mg; 0.45 mmol) was taken up in CH₂Cl₂ (2 ml) and treated with triethyl amine (126 ml; 0.91 mmol) followed by methanesulfonyl chloride (35 ml; 0.45 mmol). The reaction stirred over night at room temperature. The mixture was diluted with CH₂Cl₂ and quenched with sat. aqueous NaCl. The organic layer was dried over MgSO₄, filtered and concentrated to afford compound (241).

### Example 91

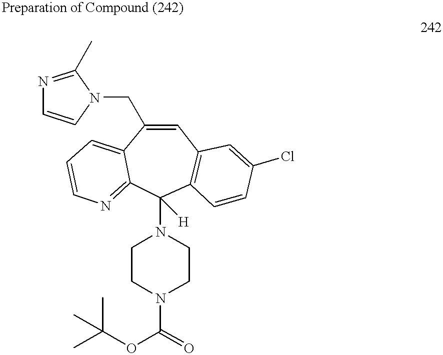

The mesylate compound (241) from Preparative Example 23, Step I above (230 mg; 0.442 mmol) was reacted in the same manner as Preparative Example 20, Step C. Purification of the crude product was accomplished by preparative TLC plates eluting with 95:5 CH₂Cl₂/MeOH(NH₃) followed by 1:1 EtOAc:Hexanes to afford the title compound as a light tan solid (242) 105-116° C. (dec) MH⁺506.

### Preparative Example 24

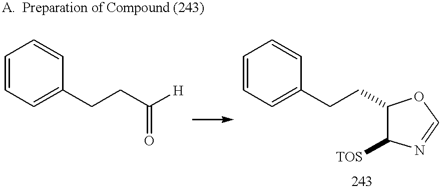

NaCN and 3-Phenylpropionaldehyde (ACROS) were dried overnight under vacuum. The aldehyde was then passed through activated Al₂O₃. Tosylmethyl isocyanide (5 g, 25.6 mmol) (ACROS) and dry 3-Phenylpropionaldehyde (3.36 g; 25.1 mmol) were combined in EtOH (42 ml) and stirred for 5 minutes. To the turbid mixture was added the dry NaCN (1.23 g; 25.1 mmol). An exothermic reaction was observed and after 5 minutes TLC showed consumption of starting material. The reaction was transferred to a sealed tube and used directly in the next experiment.

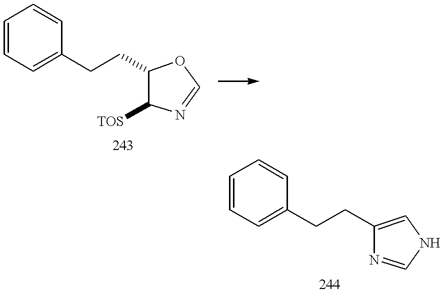

B. The crude product (243) from Step A above (25 mmol), was diluted up to 65 ml total volume with EtOH. To this mixture was added 7N NH₃ in MeOH (100 ml) and the reaction was heated to 90° C. over night (20 hr). The reaction was allowed to cool to room temperature and stirred for 2 hr then concentrated to dryness. The crude product was purified by flash silica column chromatoghraphy eluting with a gradient of 1-5% MeOH(sat. NH₃)/CH₂Cl₂ (244).

### Preparative Example 25

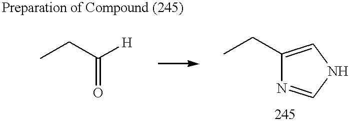

Propionaldehyde (1.5 g; 25.11 mmol) (AC ROS) and tosyl methyl isocyanide (5 g; 25.6 mmol) were reacted in the same manner as Preparative Example 24 above to afford the title compound (245).

### Preparative Example 26

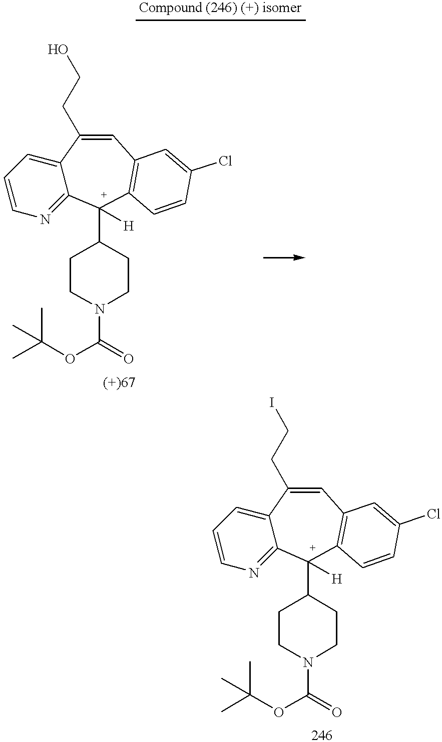

The (+) isomer of compound (67) from Preparative Example 6 isolated by chiral AD column chromatography was further reacted as in Preparative Example 6 to obtain compound (246).

### Example 92 AND 93

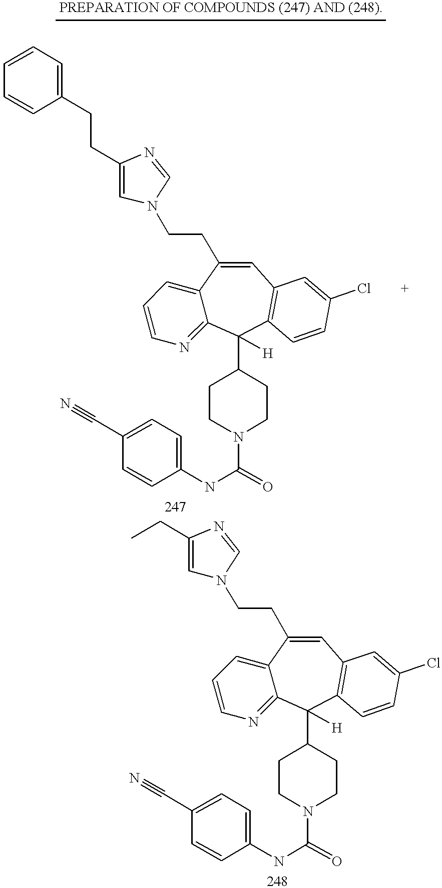

Compound (246) from Preparative Example 26 above was reacted in the same manner as Examples (22), (25) and (29) using the appropriate imidazole or isocyanate respectively to afford the title compounds (247) and (248).

### Examples 94-96

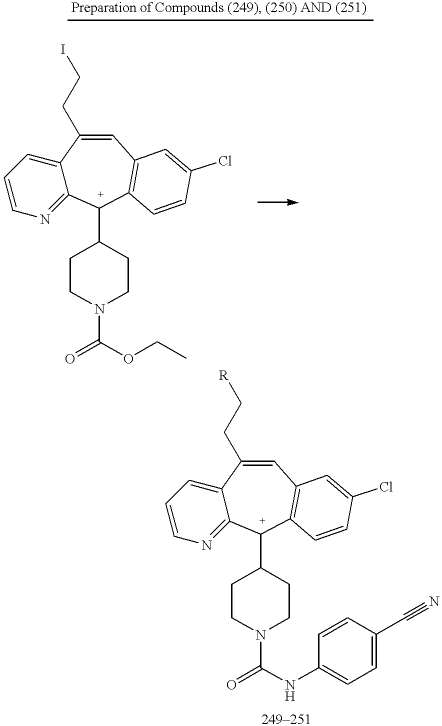

In a similar manner as Preparative Example 26 above, the (+) isomer of the carbamate was obtained and reacted in essentially the same manner as Examples 92 and 93 substituting with the appropriate imidazoles, to provide compounds (249)-(251) shown in the table below.

| Ex. # | R = | Cmp. # | Phys. Data |
|---|---|---|---|
| 94 | 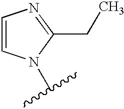 | 249 | mp 133.2-144.3° C. dec. MH(+) 577.14 |
| 95 | 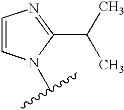 | 250 | mp 132.1-143.8° C. dec. MH(+) 591.16 |
| 96 | 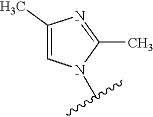 | 251 | mp 134.1-144.9° C. dec. MH(+) 563.10 |

### Examples 97-101

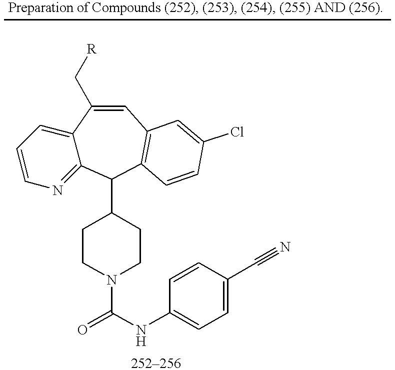

In essentially the same manner as in Preparative Example (20) and Example the following compounds were prepared:

| EX. | R = | # | PHYS. DATA |
|---|---|---|---|
| 97 | 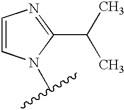 | 252 | mp 148-159° C. dec. MH(+) 577. |
| 98 | 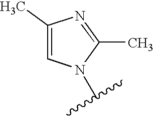 | 253 | mp 134-142° C. dec. MH(+) 563. |
| 99 | 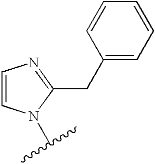 | 254 | mp 90-102° C. dec. MH(+) 625. |
| 100 | 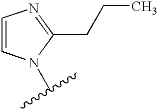 | 255 | mp 126-139° C. dec. MH(+) 577. |
| 101 | 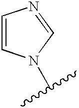 | 256 | mp 151-164° C. dec. MH(+) 535. |

### Example 102

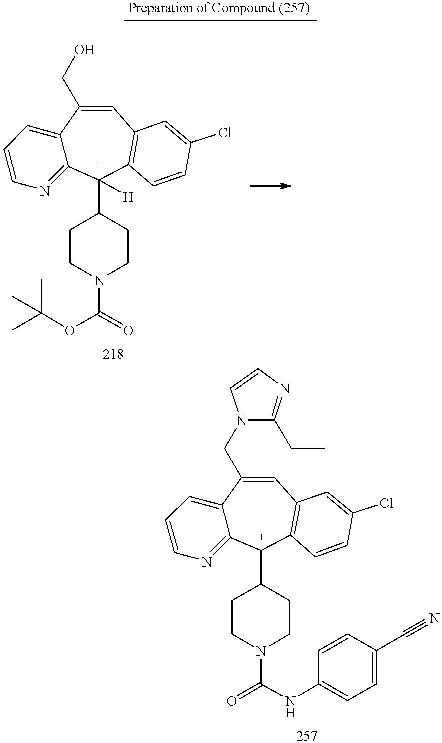

The (+) isomer of compound (218) obtained in essentially the same manner as Preparative Example (22), was further reacted in the same manner as in Preparative Example (6), Steps E and F, Examples (21), (23) and (29) sustituting with 2-Ethyl imidazole in Ex. (21) to afford the title compound (257). (146-157° C. dec.), MH+564

### Preparative Example 27

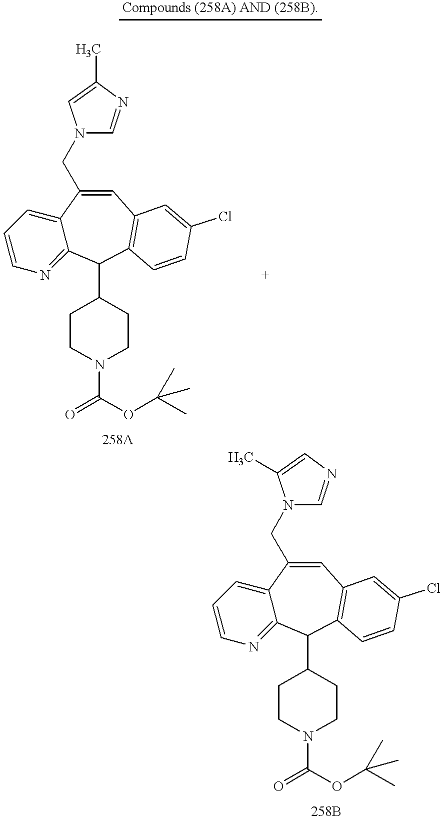

In essentially the same manner as Preparative Example (20), substituting 4-methylimidazole, compound (258) was prepared as a mixture of 4 and 5 substituted imidazole derivatives. This mixture was then reacted in a similar manner as Example 35 and the isomers separated (258A) and (258B).

### Example 103

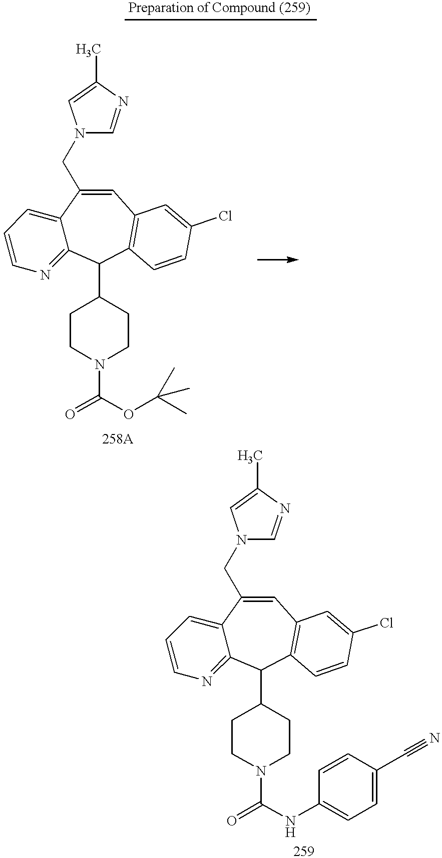

The pure 4-methyl imidazole isomer (258A) was reacted as in Preparative Example 20, Step D, and Example (29) to afford the title compound as a white solid (259). (128-138° C. dec.) MH⁺549

### Example 104

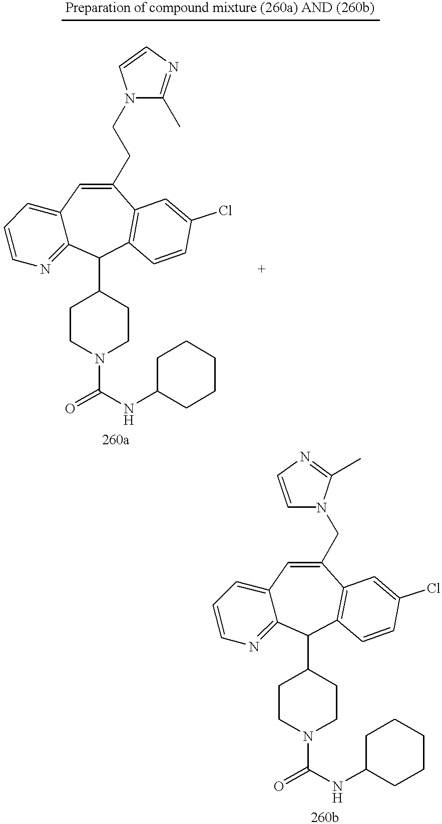

Step A Compound (108) from Preparative Example 9, Step E, was reacted with compound (64) from Preparative Example 6, Step A in essentially the same manner as in Preparative Example 6, Steps B-F, to afford a mixture of one and two methylene spaced iodo intermediates.

Step B The mixture of intermediates from Step A above was reacted in essentially the same manner as in Example 22 to afford a mixture of one and two methylene spaced imidazole derivatives.

Step C The mixture from Step B above was reacted in the same manner as Preparative Example 20, Step D, followed by a reaction with phenyl isocyante in the same manner as Example 15 to afford the title compound as a 1:1 mixture (260a) and (260b) (133-145° C. dec.); MH⁺544.

### Preparative Example 28

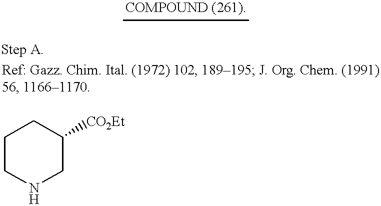

Ethyl nipecotate (70.16 g, 0.446 mmol) and D-tartaric acid (67 g, 1.0 eq) were dissolved in hot 95% EtOH (350 mL). The resulting solution was cooled to room temperature and filtered and the crystals washed with ice-cold 95% EtOH. The crystals were then recrystallized from 95% EtOH (550 mL) to give the tartrate salt (38.5 g, 56% yield). The salt (38.5 g) was dissolved in water (300 mL) and cooled to 0° C. before neutralizing with 3M NaOH. The solution was extracted with CH₂Cl₂ (5×100 mL) and the combined organics dried over Na₂SO₄ and concentrated under reduced pressure to give a clear oil (19.0 g, 89% yield).

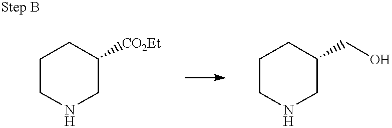

LAH (118 mL, 1.0 M in Et₂O, 1.0 eq.) was added to a solution of the product from Step A (18.5 g, 0.125 mmol) in THF (250 mL) at 0° C. over 20 minutes. The resulting solution was warmed slowly to room temperature and then heated at reflux 2 hours. The reaction was cooled to room temperature and quenched by the slow addition of saturated Na₂SO₄. The resulting slurry was dried by the addition of Na₂SO₄, filtered through Celite and concentrated to give a colorless oil (13.7 g, 98% crude yield). CIMS: MH⁺=116; [α]²⁰_{D}=−8.4° (5.0 mg in 2 mL MeOH).

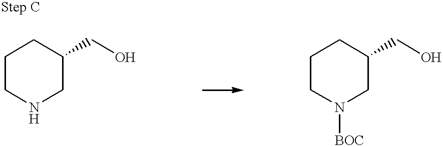

The product of Step B (13.6 g, 0.104 mmol) was dissolved in MeOH (100 mL) and H₂O (100 mL) di-tert-butyl dicarbonate (27.24, 1.2 eq.) was then added portionwise keeping the pH>10.5 by the addition of 50% NaOH. The reaction mixture was stirred at room temperature an additional 2.5 hours and concentrated in vacuo. The residue was diluted with H₂O (350 mL) and extracted with CH₂Cl₂ (3×150 mL). The combined organics were dried over Na₂SO₄, filtered, and concentrated under reduced pressure. The crude product was purified by flash chromatography using a 50% EtOAc in hexanes solution as eluent to give a white solid (12.13 g, 48% yield). FABMS: MH⁺=216; [α]²⁰_{D}=+15.2 (5.0 mg in MeOH).

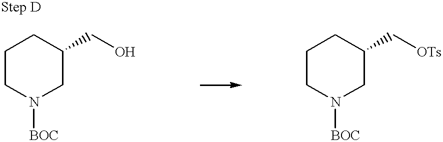

p-Toluenesulfonyl chloride (12.75 g, 1.2 eq.) was added portionwise to a solution of the product from Step C (12.00 g, 55.74 mmol) in pyridine (120 mL) at 0° C. The resulting solution was stirred at 0° C. overnight. The reaction mixture was diluted with EtOAc (300 mL) and washed with cold 1N HCl (5×300 mL), saturated NaHCO₃ (2×150 mL), H₂O (1×100 mL), and brine (1×100 mL), dried over Na₂SO₄ and concentrated in vacuo to give a pale yellow solid (21.0 g, 100% crude yield). FABMS: MH⁺=370.

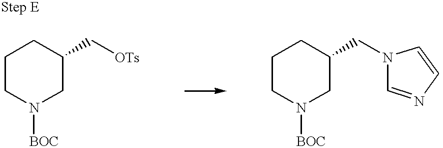

The product of Step D (21.0 g, 55.74 mmol) in DMF (300 mL) was treated with sodium imidazole (8.37 g, 1.5 eq.) and the resulting solution heated at 60° C. for 2 hours. The reaction mixture was cooled to room temperature and concentrated in vacuo. The residue was diluted with H₂O (300 mL) and extracted with CH₂Cl₂ (3×150 mL). The combined organic layer was dried over Na₂SO₄, filtered, and concentrated. The crude product was purified by flash chromatography using a 7% MeOH in CH₂Cl₂ solution as eluent to give a pale yellow solid (7.25 g, 49% yield). FABMS: MH⁺=266; [α]²⁰_{D}=+8.0 (5.0 mg in MeOH).

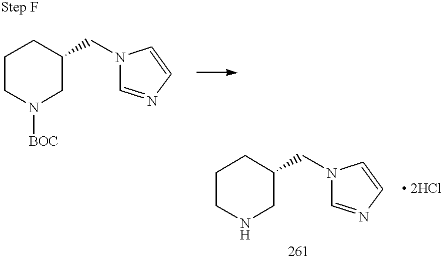

The product of Step E (5.50 g, 20.73 mmol) was stirred at room temperature in 4M HCl in dioxane (50 mL) overnight. The resulting solution was concentrated and the residue triturated with Et₂O to give Compound (261) as a yellow solid (4.90 g, 99% yield). CIMS: MH⁺=166.

### Preparative Example 29

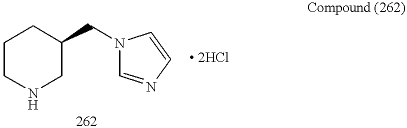

By essentially the same procedure set forth in Preparative Example 28 above, using L-tartaric acid instead of D-tartaric acid in Step A, Compound (262) was prepared.

### Preparative Example 30

### Preparation of Compounds (263) and (264)

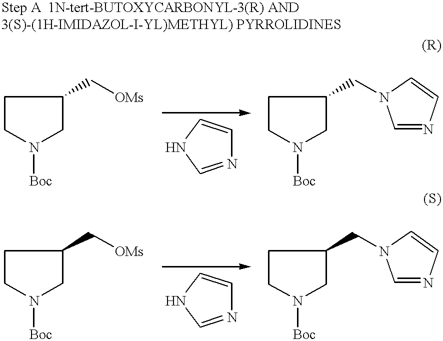

3(R)-(3-Methanesulfonyloxymethyl)pyrrolidine (J. Med. Chem. 1990, 33, 77-77) (0.993 g, 3.56 mmoles) was dissolved in anhydrous DMF (25 mL) and sodium imidazole (0.6 g, 10 mmoles) was added. The mixture was heated at 60° C. for 2 h and then evaporated to dryness. The product was extracted with CH₂Cl₂ and washed with brine. The CH₂Cl₂ extract was evaporated to dryness to give the titled compound (263) (1.1409 g, 100%), ESMS: FABMS (M+1)=252; ¹HNMR (CDCl₃) 1.45 (s, 9H), 1.5-1.7 (m, 1H), 1.9-2.1 (m, 1H), 2.5-2.7 (m, 1H), 3.0-3.2 (m, 1H), 3.3-3.6 (m, 2H), 3.9 (dd, 2H), 6.9 (s,1H), 7.1 (s,1H), 7.45 (s,1H).

In a similar manner, the (S) isomer was prepared from 3(S)-(3-Methanesulfonyloxymethyl)pyrrolidine (0.993 g, 3.56 mmol) to give the title compound (1.14 g, 100%).

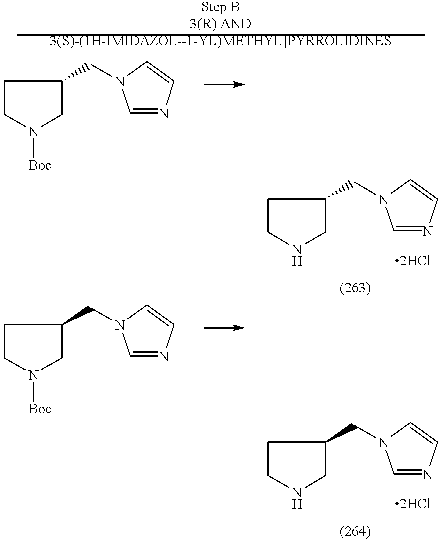

The (R) product (0.48 g, 1.91 mmoles) from Step A was stirred in 4N HCl in dioxane (10 mL) for 2h and then evaporated to dryness to give the title compound (263) as the HCl salt.

In a similar manner the (S) isomer was prepared to give compound (264) as the HCl salt.

### Preparative Example 31

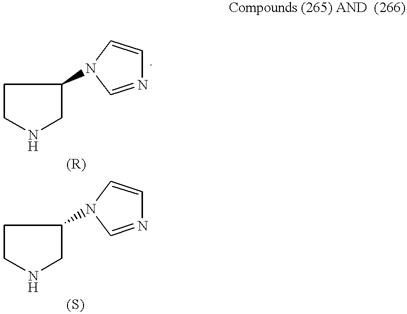

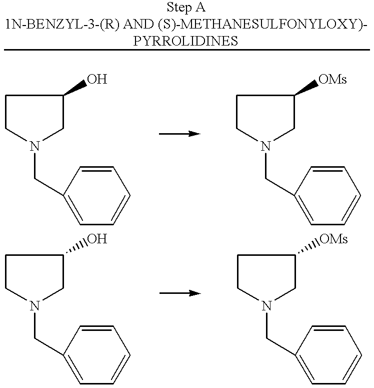

1N-Benzyl-3(R)-hydroxy -pyrrolidines (5 g, 28.21 mmol) and triethylamine (7.86 mL, 56.35 mmol) were dissolved in CH₂Cl₂ (50 mL) and the mixture was stirred under nitrogen at 0° C. Methanesulfonylchloride (2.62 mL, 33.87 mmol) was added and the solution was stirred at room temperature for 2 h. The solution was diluted with CH₂Cl₂ and washed with saturated aqueous sodium bicarbonate, water and dried (MgSO₄), filtered and evaporated to dryness to give the (R) title compound (7.2 g, 96.4%). FABMS (M+1)=256; ¹HNMR (CDCl₃) 2.2 (m, 1H), 2.3 (m, 1H), 2.52 (m, 1H), 2.7-2.85 (m, 3H), 2.95 (s, 3H), 3.65 (q, 2H), 5.16 (m, 1H), 7.3 (s, 5H).

In a similar way the (S) isomer was prepared from 1N-Benzyl-3(S)-hydroxy-pyrrolidines (5 g, 28.21 mmoles) to give the (S) title compound (7.15 g, 98%).

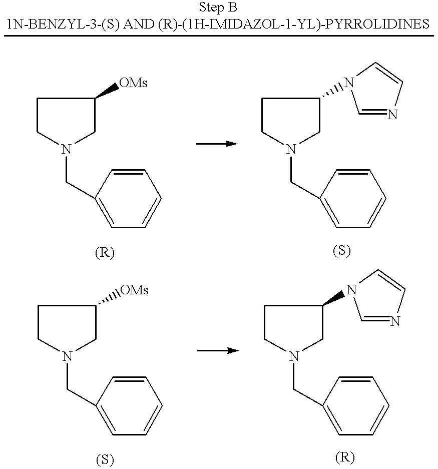

A solution of the (R) product from Step A (2.0 g, 7.84 mmoles) was added to a stirred solution of imidazole (1.1 g, 16.17 mmoles) in DMF (25 mL) under nitrogen atmosphere. The mixture was stirred at 60° C. for 16 h. DMF was evaporated in vacuo. The resulting crude product was extracted with CH₂Cl₂ and the extract was successively washed with water and brine, and the CH₂Cl₂ was evaporated to leave the title residue which was chromatographed on silica gel using 3% (10% conc NH₄OH in methanol)- CH₂Cl₂ as eluant to give the title compound (0.95 g, 50.56%). FABMS (M+1)=228.

In a similar fashion the other isomer was prepared.

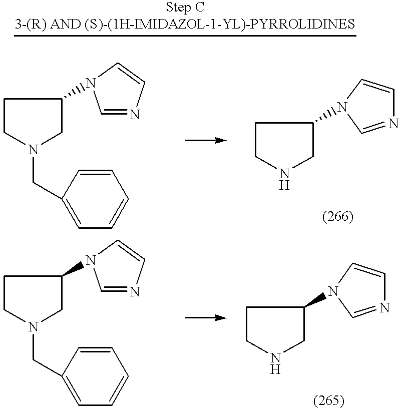

A mixture of the (S) product (0.95 g) from Step B and 10% Pd on carbon (0.5 g) in EtOH (20 mL) was shaken at 50 psi under an atmosphere of hydrogen for 24 h. The catalyst was filtered and the solvent removed to give the title compound (266) (0.522 g, 99.9%).

In a similar manner the (R) isomer was prepared from 1.0 g of the starting (R) product from Step B and 10% Pd on carbon (0.6 g) to give compound (265) in 99% yield.

### Preparative Example 32

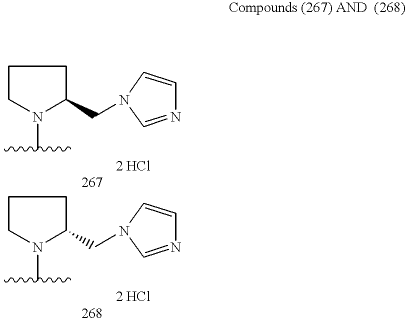

By essentially the same procedure set forth in Preparative Example 31 above, beginning with L or D-prolinol, the title compounds (267) and (268) were prepared.

### Example 105

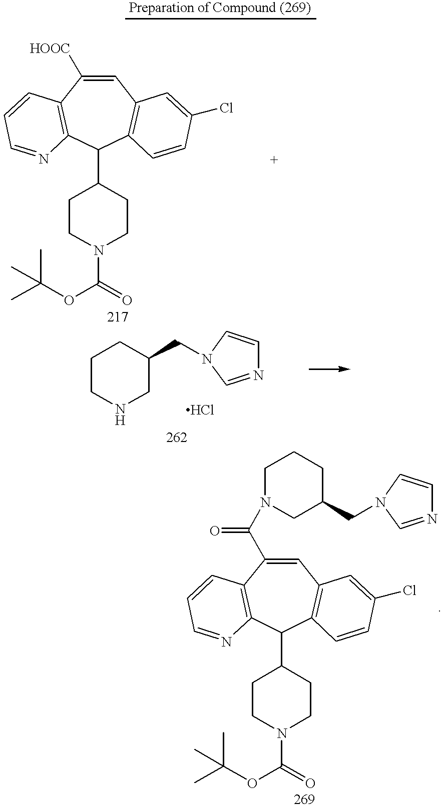

Compound (217) from Preparative Example 19 (0.227 g, 0.499 mmol) was added to a solution of Compound (262) from Preparative Example 29 (0.131 g, 0.,649 mmol), DEC (0.249 g, 1.3 mmol), HOBT (0.175 g, 1.3 mmol) and NMM (0.5 mL) in DMF (25 mL). The resulting solution was stirred at room temperature for 24 hours. The reaction mixture was diluted with H₂O until precipitation ceased and the slurry was filtered. The precipitate was diluted with CH₂Cl₂, washed with brine, dried over Na₂SO₄ and concentrated. The crude product was purified by chromatography using a 5% (10% NH₄OH in MeOH) solution in CH₂Cl₂ as eluent to give the title compound (269) (0.184 g, 62% yield).

### Examples 106-111

Preparation of Compounds (270)-(275). Using the appropriate amine from the Preparative Examples 28-32, and following essentially the same procedure as in Example 105 above, the following compounds were prepared:

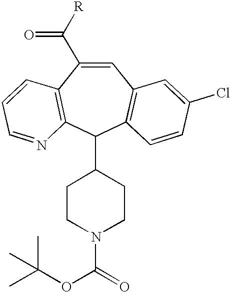

| EX. | R = | Compound # | PHYS. DATA |
|---|---|---|---|
| 106 | 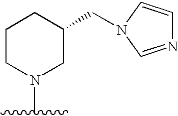 | 270 | MH⁺ = 603 |
| 107 | 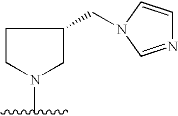 | 271 | MH⁺ = 589 |
| 108 | 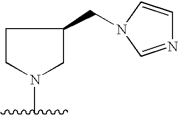 | 272 | MH⁺ = 589 |
| 109 | 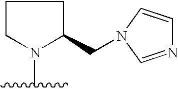 | 273 | MH⁺ = 589 |
| 110 | 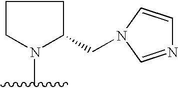 | 274 | MH⁺ = 603 |
| 111 | 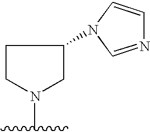 | 275 | MH⁺ = 603 |

### Example 112

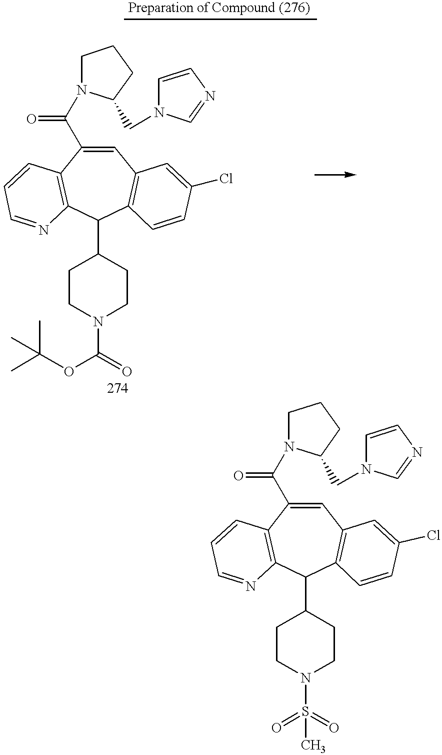

Compound (274) from Example 110 above (0.125 g, 0.213 mmoles) in CH₂Cl₂ (50 mL) was stirred with TFA (10 mL) at room temperature overnight. The reaction mixture was evaporated to give the TFA salt (0.28 g) which was redissolved in CH₂Cl₂ (50 mL) and cooled (ice water bath). Triethyl amine (0.1 mL) followed by methane sulfonyl chloride (0.038 g, 0.319 mmoles) were added and the reaction mixture was stirred at room temperature overnight. The reaction mixture was washed with sodium bicarbonate and water. The organic layer was dried over MgSO₄ and evaporated to dryness to give the title compound (276) (0.05 g, MH₊=567)

### Example 113

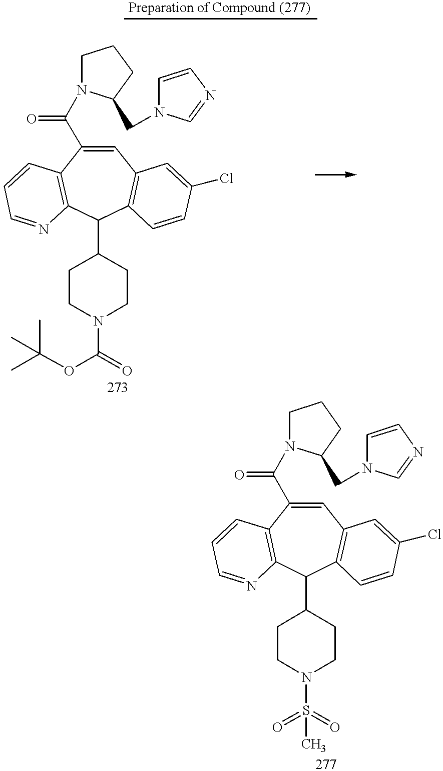

Starting with Compound (273) from Example 109 above and following essentially the same procedure as in Example 112 above, Compound (277) was prepared (MH₊=567).

### Preparative Example 33

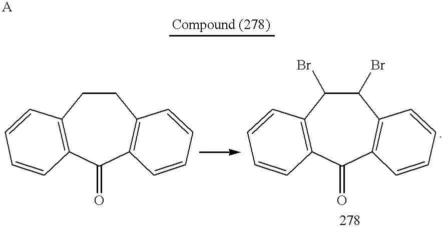

To a stirred solution of bromine (33.0 g, 210 mmol) in CCl₄ (100 ml) was added a solution of dibenzosuberenone (37.0 g, 179 mmol) in CCl₄ (200 ml) at room temperature. The resulting solution was stirred at room temperature for 1.5 hours. The white crystals were collected by filtration to give the product (278) (60.12 g, 92% yield, M+H=367).

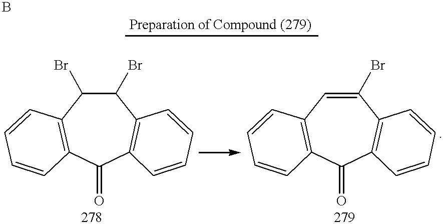

A solution of the di-bromo compound (278) from step A (60.0 g, 163 mmol) and NaOH (20.0 g, 491 mmol) in MeOH (500 ml) was stirred and heated to reflux for 1.5 hours. The reaction mixture was then cooled to room temperature and stirred overnight. The mixture was evaporated to dryness then extracted with CH₂Cl₂—H₂O.

The combined organic layer was dried over MgSO₄, filtered and evaporated to dryness to give a yellow solid (279) (46.34 g, 100% yield, M=285)

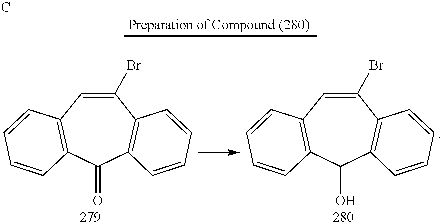

To a stirred solution of the mono-bromo compound (279) from step B (10.0 g, 35.07 mmol) in MeOH (200 ml) under nitrogen at 0° C. was added NaBH₄ (1.94 g, 51.2 mmol). The resulting solution was stirred at 0° C. for 1.5 hours, then evaporated, followed by extraction with CH₂Cl₂—H₂O. The combined organic layer was dried over MgSO₄, filtered, and evaporated to dryness to give a white solid (280) (10.3 g, 100%, M=287).

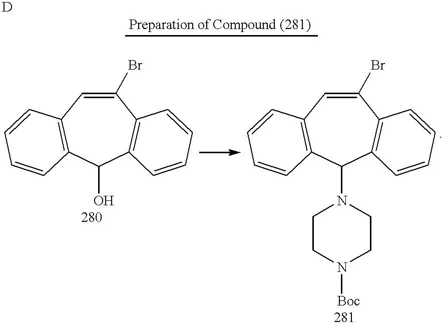

To a stirred solution of the alcohol (280) from Step C (10.0 g, 34.8 mmol) in CH₂Cl₂ (200 ml) at 0° C. was added 2,6-lutidine (14.9 g, 139.3 mmol) and thionyl chloride (8.28 g, 69.66 mmol). The resulting solution was warmed to room temperature and stirred overnight. The solution was then poured onto 0.5N NaOH solution, followed by extraction with CH₂Cl₂. The combined aqueous layer was dried over Na₂SO₄, filtered, and concentrated to dryness to give a crude brown oil (15.5 g). To a solution of this crude oil (15.5 g) in acetonitrile (200 ml) was added 2,6-Bis (dimethyl)-1-methyl piperidine (10.81 g, 69.66 mmol) and N-Boc piperidine (6.49 g, 34.83 mmol). The resulting mixture was warmed to 65° C. overnight. The mixture was evaporated to dryness, followed by extraction with CH₂Cl₂/saturated NaHCO₃. The combined organic layer was dried over Na₂SO₄, concentrated and purified by column chromatography on silica gel, eluting with 5% EtOAc/95% Hexane to give the protected N-Boc compound (281) (5.68 g, 36% yield, MH+=455).

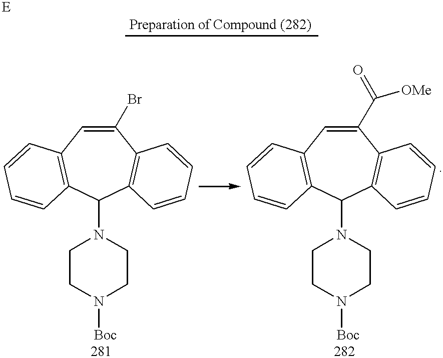

To a solution of the N-Boc compound (281) from Step D (4.0 g, 8.78 mmol) in anhydrous toluene (100 ml) and methanol (20 ml) was added triphenylphosphine (1.15 g, 4.39 mmol), DBU (1.81 g, 11.9 mmol) and palladium (II) chloride (0.15 g, 0.88 mmol). The resulting mixture was purged with carbon oxide at 80 psi to 100 psi and heated to 78° C.-82° C. for 5 hours, followed by stirring at room temperature for overnight. The solution was then extracted with EtOAc. The combined organic layer was washed with water, brine, dried over Na₂SO4, filtered, evaporated and the crude product was purified by column chromatography on silica gel, eluting with 10% EtOAc/90% Hexane to give the ester compound (282) (2.1 g, 55% yield, MH⁺=435).

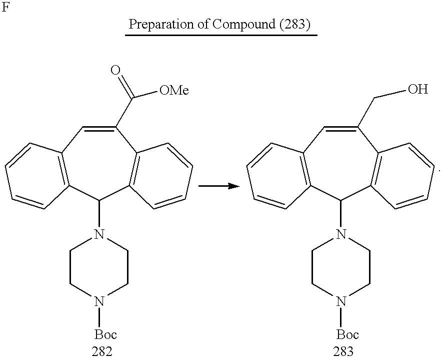

To a stirred solution of the ester compound (282) from Step E (1.2 g, 2.77 mmol) in THF (15 ml) at 0° C. was added a 1 M solution of DIBAL (16.62 ml, 16.62 mmol). The resulting solution was stirred at room temperature for 4 hours. To the solution was then added 10% potassium sodium tartarate, followed by extraction with EtOAc. The combined organic layer was dried over Na₂SO4, filtered, and evaporated to give a solid (283) (1.1 g, 100% yield, MH⁺=406).

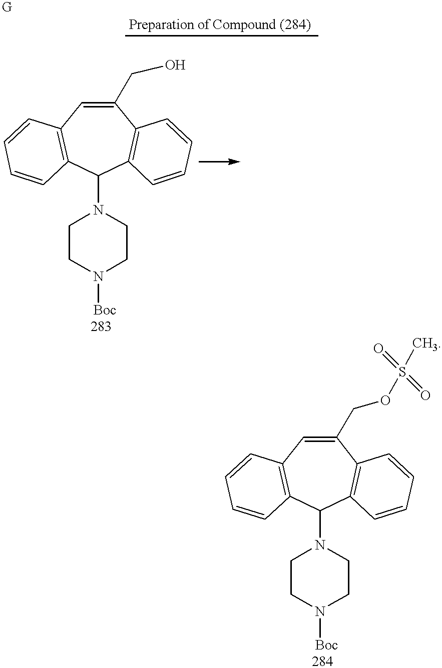

To a solution of the alcohol (283) from Step F (0.62 g, 1.52 mmol) in CH₂Cl₂ (15 ml) under nitrogen was added triethyl amine (0.64 ml, 4.56 mmol) and methane sulfonyl chloride (0.26 g, 2.29 mmol). The resulting solution was stirred at room temperature overnight. The mixture was washed with NaHCO₃ solution, dried over Na₂SO4, filtered and concentrated to dryness to give the mesylate compound (284) (0.53 g, 76% yield, M-CH₃SO₃H=389.1).

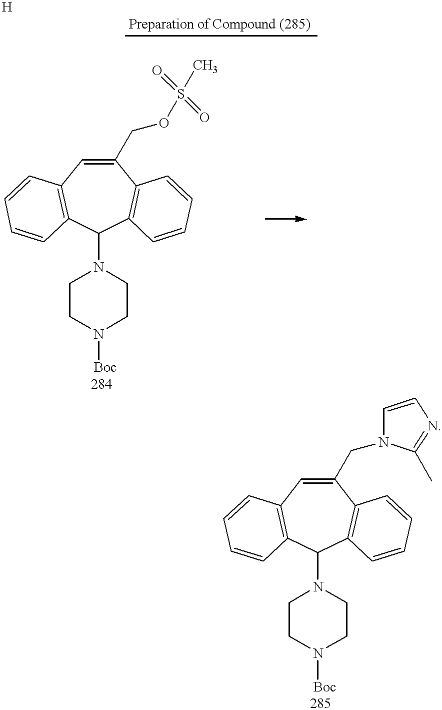

To a stirred solution of 1-methyl-imidazole (1.04 g, 12.7 mmol) in DMF (10 ml) under nitrogen, was added NaH (0.305 g, 12.7 mmol). The resulting solution was stirred at room temperature for 15 minutes, followed by the addition of the mesylate compound (284) from step G (2.05 g, 4.23 mmol). The reaction mixture was stirred at room temperature overnight, then evaporated to dryness, and extracted with an EtOAc-NaHCO₃ solution. The combined organic layer was dried over Na₂SO4, concentrated and the crude product purified by silica gel column chromatography eluting with 2% MeOH/98% NH₃—CH₂Cl₂ to give the product (285) (0.775 g, 39% yield, MH⁺=471).

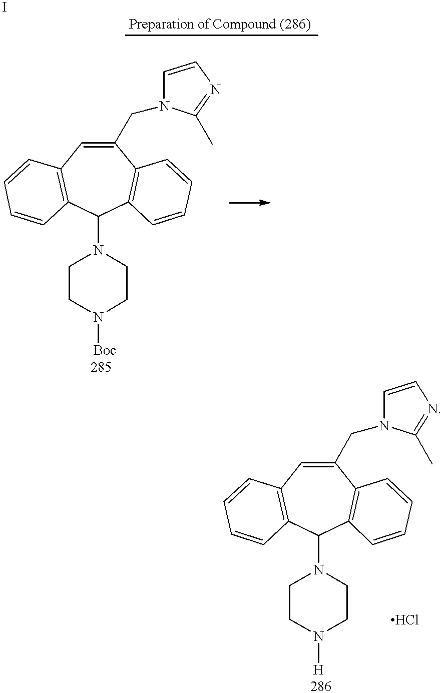

A solution of the product (285) from step H (0.3 g, 0.64 mmol) in 4M HCl in dioxane (40 ml) was stirred at room temperature for 3 hours and then concentrated to dryness to give the hydrochloride salt of the title product (286) (0.42 g, 100% yield, MH⁺=371).

Examples 114 and 115

Compounds (287) AND (288).

The racemic mixture of Preparative Example 33, Step H above was separated into its pure isomers by HPLC, using a Chiral AD column eluting with 15% IPA/75% Hexane/0.2% DEA to afford the compounds in the table below:

| EX. # | PROCEDURE | R = | CMPD # | PHYS. DATA |
|---|---|---|---|---|
| 114 | Prep. Ex. 33, | BOC | 287 | MS M⁺ = 471 |
| | Steps A-H | | isomer 1 | |
| 115 | Prep. Ex. 33, | BOC | 288 | MS M⁺ = 471 |
| | Steps A-H | | isomer 2 | |

### Examples 116-119

Starting with the piperazine compound (286) from Preparative Example 33 Step I, and reacting it with the appropriate isocyanate or sulfonyl chloride, following essentially the same procedure as indicated in the table below, the following compounds were prepared:

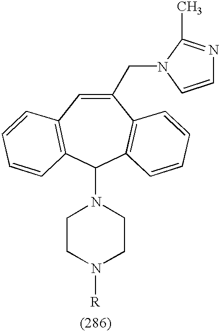

| EX. # | PROCEDURE | R = | CMPD # | PHYS. DATA |
|---|---|---|---|---|
| 116 | Example 13 | 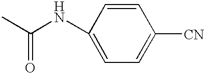 | 289 isomer 1 | MS M⁺ = 515 |
| 117 | Example 13 | 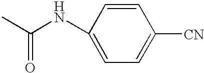 | 290 isomer 2 | MS M⁺ = 515 |
| 118 | Example 24 | 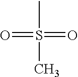 | 291a isomer 1 | MS M⁺ = 449 |
| 119 | Example 24 | 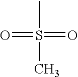 | 291b isomer 2 | MS M⁺ = 449 |

### Preparative Example 34

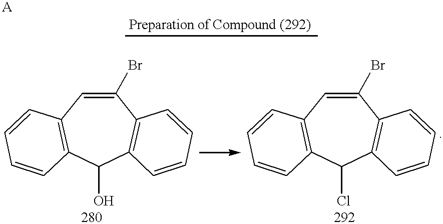

To a stirred solution of alcohol (280) from Preparative Example 33, Step C (30.0 g, 104.5 mmol) in CH₂Cl₂ (500 mL) under nitrogen at −20° C. was added thionyl chloride (106.7 mL, 1,46 mmol). The resulting solution was stirred at room temperature overnight and then evaporated to dryness. The crude mixtue was diluted with toluene (50 mL), followed by the addition of more SOCl₂ (106.7 mL) at room temperature. The resulting solution was heated to reflux for 2 hours until the reaction went to completion. The reaction mixture was then cooled to room temperature and concentrated to dryness to give a light brown solid (292) (35.67 g, 100% yield, MBrCl=191).

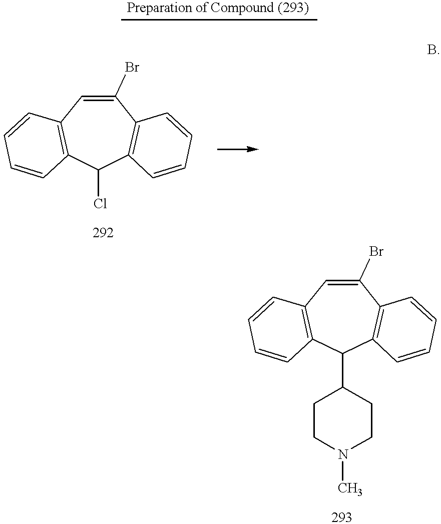

To a suspension of Mg (3.63 g) in anhydrous THF (95 mL) under nitrogen at room temperature was added 4-chloro-1-methyl piperidine (3 mL, 10% of the total amount) and one small crystal of iodine. The resulting solution was heated to reflux, followed by the addition of iodomethane (0.5 mL) and the remainder of the 4-chloro-1-methyl piperidine (27 mL). The reaction was stirred for one hour and then concentrated to dryness to give the crude Grignard reagent (0.8M).

To a stirred solution of the chloro compound (292) from Preparative Example 34, Step A (35.67 g, 116.7 mmol) in anhydrous THF (200 mL) under nitrogen at room temperature , was added dropwise the Grignard reagent (as obtained above) (0.8M, 146 mL, 116.7 mmol).The resulting solution was stirred at room temperature for 3 hours, followed by the extraction with EtOAc-H₂O. The combined organic layer was dried over MgSO₄, filtered and evaporated to dryness to give the product (293) (49.25 g, 100% yield, MH⁺=368).

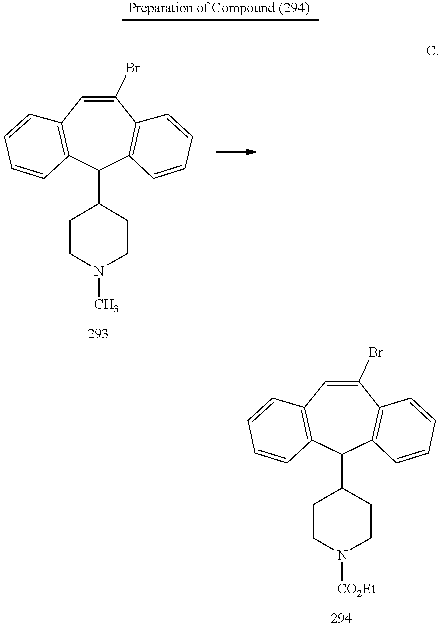

To a stirred solution of Compound (293) from Step B above (42.9 g, 116.5 mmol) in toluene (400 mL) under nitrogen was added triethylamine (49 mL, 349.5 mmol). The resulting solution was heated to refux, followed by the dropwise addition of ethyl chloroformate (126 g, 1165 mmol). Continued to heat the solution at the reflux temperature for 2 hours. The reaction was then stirred at room temperature overnight, followed by extraction with an EtOAc-1N NaOH solution. The combined organic layer was dried over MgSO₄, filtered, concentrated to dryness and the crude product purified by column chromatography on normal phase silica gel, eluting with 30% EtOAc/70% Hexane to give a light yellow solid (294) (2.99 g, 12% yield, MH⁺=426.3).

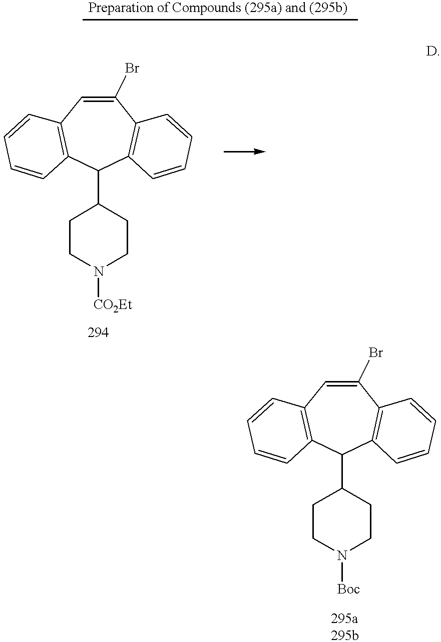

A solution of the ester (294) from step C above (3.34 g, 7.83 mmol) in 6N HCl (20 mL) was heated to reflux overnight. The reaction was cooled to room temperature and basified with NH₄OH solution, followed by extraction with CH₂Cl₂. The combined organic layer was dried over MgSO₄, filtered, and evaporated to dryness to give a crude free piperidine (2.80 g, 100% yield, MH+=534) To the crude material (as obtained above) (2.77 g, 7.82 mmol) in 50% MeOH/1% H₂O (200 mL) was added Di-tert-butyl dicarbonate (3.41 g, 15.64 mmol). The reaction mixture was adjusted to pH=9 and stirred at room temperature for 4 hours, evaporated to dryness then extracted with CH₂Cl₂—H₂O. The combined organic layer was dried over MgSO₄, filtered, concentrated to dryness and purified by HPLC, using chiral AD column, eluting with 15% IPA/75% Hexane/0.2% DEA to give the pure isomers of the N-Boc compounds (295a) and (295b) (3.42 g, 96% yield, MH⁺=454).

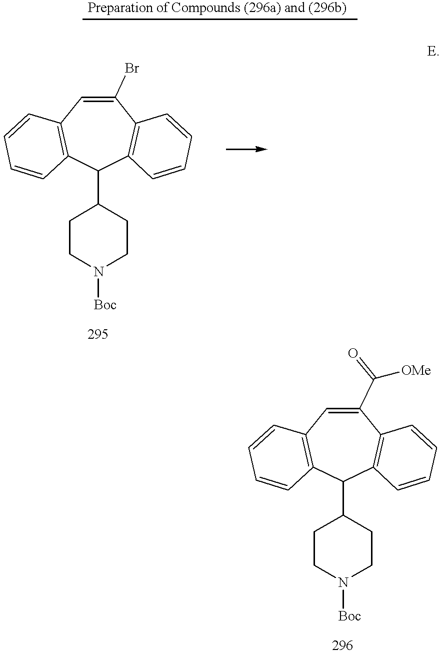

To a stirred solution of the pure (+) or (−) isomer of the N-Boc compound from Step D above (4.0 g, 8.78 mmol) in anhydrous toluene (100 mL) and methanol (20 mL) was added triphenyl phosphine (1.15 g, 4.39 mmol), DBU (1.81 g, 11.9 mmol) and palladium (II) chloride (0.15 g, 0.88 mmol). The resulting mixture was purged with carbon monooxide at 80 psi to 100 psi and heated to 78° C.-82° C. for 5 hours, followed by stirring at room temperature overnight. The solution was then extracted with EtOAc. The combined organic layer was washed with water, brine, dried over Na₂SO4, filtered, evaporated and purified by column chromatography on silica gel, eluting with 10% EtOAc/90% Hexane to give the ester (296a) or (296b) (2.1 g, 55% yield, MH⁺=435).

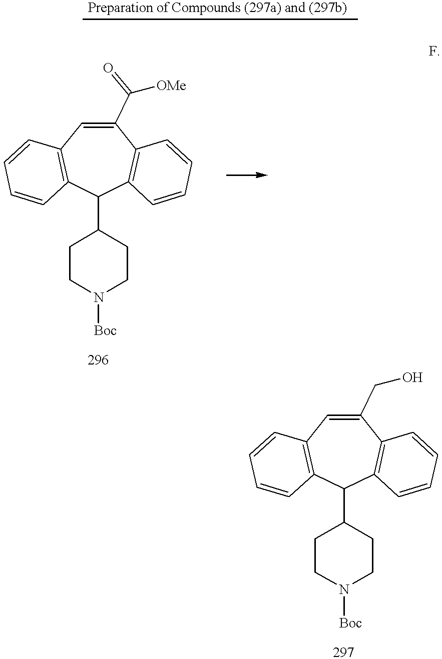

To a stirred solution of the (+) or (−) isomer of the ester from Step E above, (1.2 g, 2.77 mmol) in THF (15 mL) at 0° C. was added 1M solution of DIBAL (16.62 mL, 16.62 mmol). The resulting solution was stirred at room temperature for 4 hours. To the solution was then added 10% potential sodium tartarate, followed by extraction with EtOAc. The combined organic layer was dried over Na₂SO₄, filtered, and evaporated to give a solid (297a) or (297b) (1.1 g, 100% yield, MH⁺=406).

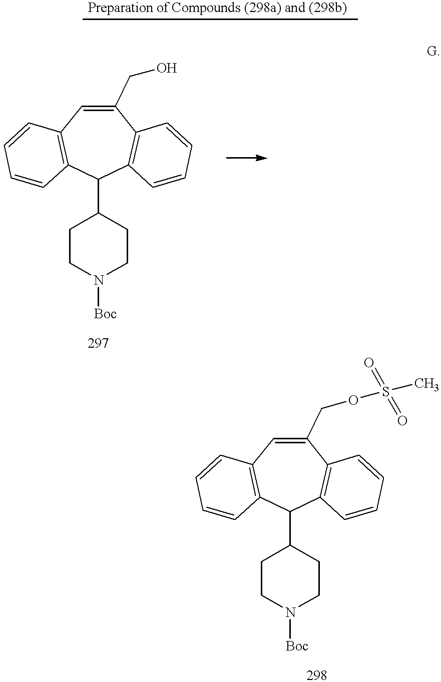

To a stirred solution of the (+) or (−) isomer of the alcohol from Step F, above (0.62 g, 1.52 mmol) in CH₂Cl₂ (15 mL) under nitrogen was added triethyl amine (0.64 mL, 4.56 mmol) and methane sulfonyl chloride (0.26 g, 2.29 mmol). The resulting solution was stirred at room temperature for overnight. The mixture was washed with NaHCO₃ solution, dried over Na₂SO4, filtered and concentrated to dryness to give the mesylate compound (298a) or (298b) (0.53 g, 76% yield, M-CH₃SO₃H=389.1).

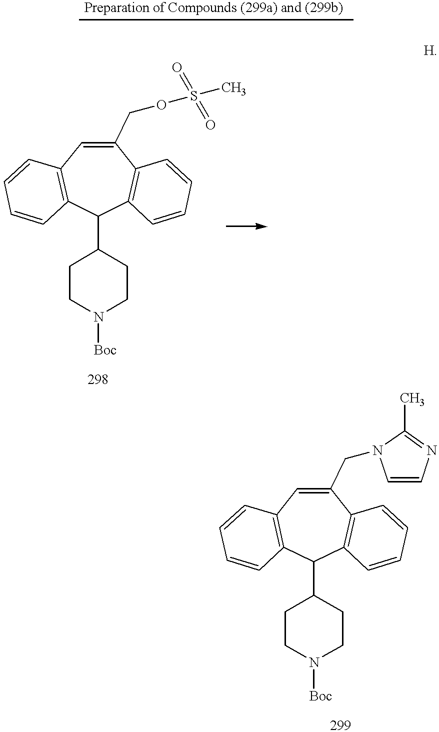

To a stirred solution of 1-methyl-imidazole (1.04 g, 12.7 mmol) in DMF (10 mL) under nitrogen, was added NaH (0.305 g, 12.7 mmol). The resulting solution was stirred at room temperature for 15 minutes, followed by the addition of the (+) or (−) isomer of the mesylate compound (299) from Step G above (2.05 g, 4.23 mmol). The reaction mixture was stirred at room temperature overnight then evaporated to dryness, followed by extraction with an EtOAc-NaHCO₃ solution. The combined organic layer was dried over Na₂SO4, concentrated and the crude product was purified by silica gel column chromatography, eluting with 2% MeOH/98% NH₃—CH₂Cl₂ to give the product (299a) or (299b) (0.775 g, 39% yield, MH+=471).

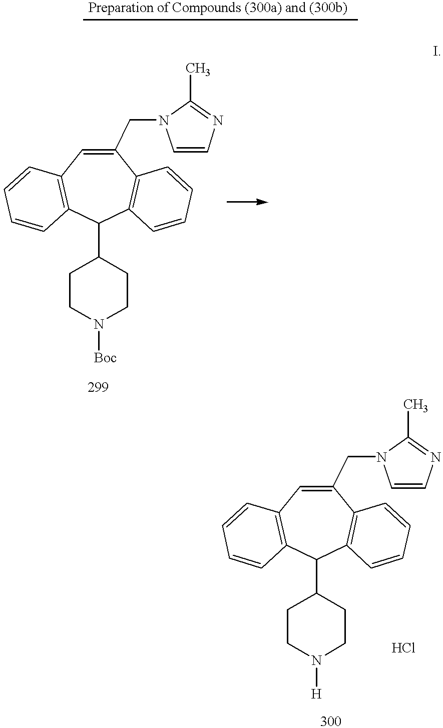

A solution of the (+) or (−) isomer of the product from Step I above (0.3 g, 0.64 mmol) in 4M HCl in dioxane (40 mL) was stirred at room temperature for 3 hours and then concentrated to dryness to give the HCl salt of the product (300a) or (300b) (0.42 g, 100% yield, MH⁺=371).

### Examples 120 and 121

Starting with the appropriate (+) or (−) isomer of Compound (300) and reacting in a similiar manner as in Example 13 using the appropriate isocyanate, the following compounds were prepared:

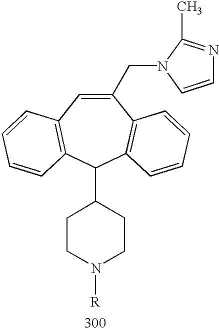

| EX. # | PROCEDURE | R = | CMPD # | PHYS. DATA |
|---|---|---|---|---|
| 120 | Example 13 | 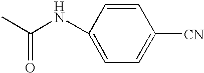 | 301 isomer 1 | MS MH⁺ = 514 |
| 121 | Example 13 | 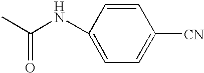 | 302 isomer 1 | MS MH+ = 514 |

### Preparative Example 35

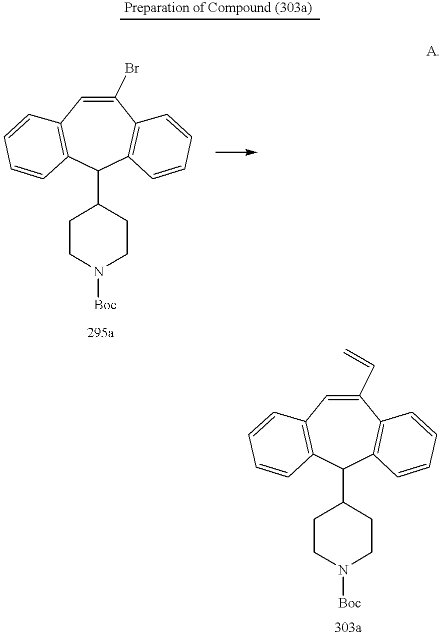

To a stirred solution of isomer 1 of the bomo-compound (295a) from Preparative Example 34, Step D,(0.5 g, 1.10 mmol) in 1-methyl-2-pyrrolidinone (4.3 mL) under nitrogen, was added lithium chloride (0.14 g, 3.3 mmol), tri-2-furylphosphine (0.013 g, 0.04 mmol) and tris(dibenzylideneacetone)-dipalladium(0) (0.02 g, 0.02 mmol). The resulting solution was stirred at room temperature for 5 minutes, followed by the addition of tributyl (vinyl) tin (0.39 g, 1.24 mmol). The reaction was then heated to 85° C. for 2 hours, followed by extraction with EtOAc-H₂O. The combined organic layer was dried over MgSO₄, filtered, concentrated to dryness and purified by column chromatography on normal phase silica gel, eluted with 10% EtOAc/90% CH₂Cl₂ to give a light yellow liquid (303a) (0.06 g, 15% yield, MH⁺=390).

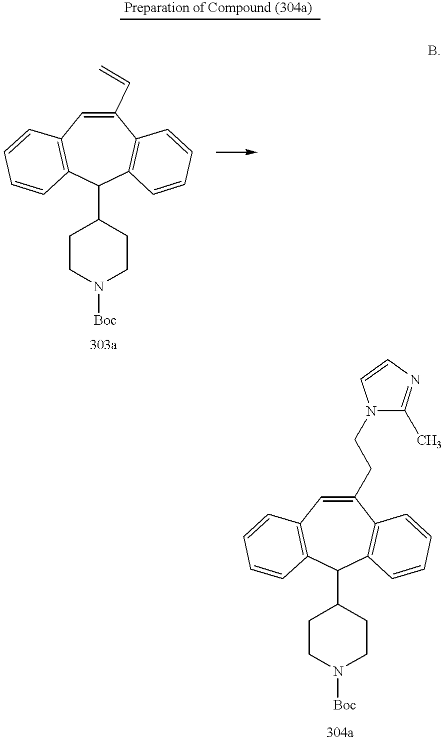

To a stirred solution of 1- methyl imidazole (0.377 g, 4.6 mmol) in anhydrous THF (4 mL) under nitrogen at −78° C., was added 2.5M n-BuLi/Hexane (0.33 mL). The resulting solution was stirred at −78° C. for 30 minutes and then allowed to warm at room temperature. To this stirred solution was added the alkene compound (303a) from step A above,(0.78 g, 2.1 mmol) in THF. The resulting solution was then heated to 120° C. overnight then cooled to room temperature, and extracted with EtOAc-H₂O. The combined organic layer was dried over MgSO₄, filtered, evaporated and purified by column chromatography on normal phase silica gel, eluted with 3% MeOH/97% NH₃—CH₂Cl₂ to give a light yellow solid (304a) (0.09 g, 10% yield, MH⁺=456.1).

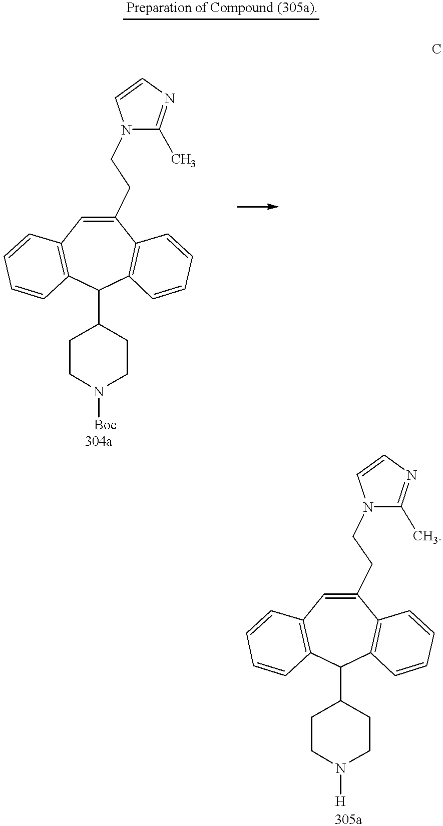

A solution of the product (304a) from Step B above (0.18 g, 3.72 mmol) in 4M HCl/dioxane (5 mL) was stirred at room temperature for 2 hours, then concentrated to dryness to give a crude off white solid (305a) (0.22 g, 100% yield, MH+=384.2).

Using the same procedure as defined in Preparative Example 35 above starting with Isomer 2 of the Boc-protected Bromo compound (295b), Isomer 2 (305b) was prepared (MH+=384.2).

### Examples 122-125

Starting with the appropriate (+) or (−) isomer of Compound (305) and reacting in a similiar manner as in Example 13 using the appropriate isocyanate, the following compounds were prepared:

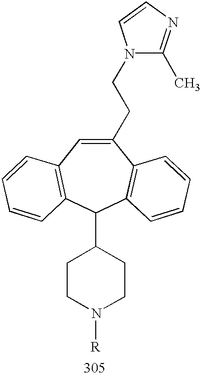

| EX. # | PROCEDURE | R = | CMPD # | PHYS. DATA |
|---|---|---|---|---|
| 122 | Example 13 | 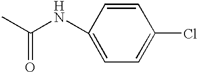 | 306 isomer 1 | MS MH⁺ = 537.1 m.p. = 118.1-119.0° C. |
| 123 | Example 13 | 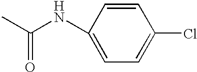 | 307 isomer 2 | MS MH⁺ = 537.1 m.p. = 107.8-108.4° C. |
| 124 | Example 13 | 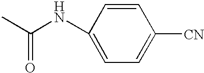 | 308 isomer 1 | MS MH⁺ = 528.2 m.p. = 119.6-120.2° C. |
| 125 | Example 13 | 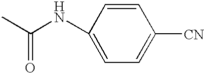 | 309 isomer 2 | MS MH⁺ = 528.2 m.p. = 120.5-121.3° C. |

### Preparative Example 36

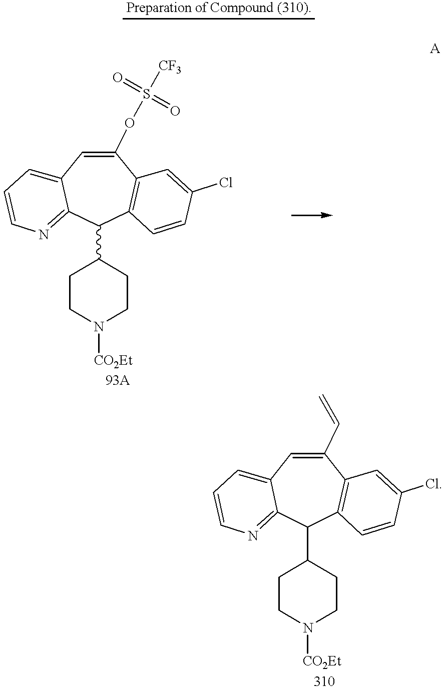

To a solution of Compound (93A) from Example 7, Step A (5.0 g, 10.02 mmol) in 1-methyl-2-pyrrolidinone (40 mL) under nitrogen at room temperature, was added LiCl (1.27 g, 30.06 mmol), Tri-2-furrylphosphine (0.093 g, 0.4 mmol) and tris(dibenzylidene acetone)dipalladium(0) (0.18 g, 0.2 mmol).The resulting solution was stirred at room temperature for 5 minutes, followed by the addition of tributyl(vinyl) tin (3.3 mL, 11.3 mmol) and stirred overnight at 80° C.-85° C. The solution was cooled to room temperature, followed by extraction with EtOAc-H₂O. The organic layer was dried over MgSO4, filtered, concentrated to dryness and purified by column chromatography on silica gel, eluted with 20% EtOAc/80% CH₂Cl₂ to give the product (310) (3.88 g, 95% yield, MH+=409.1)

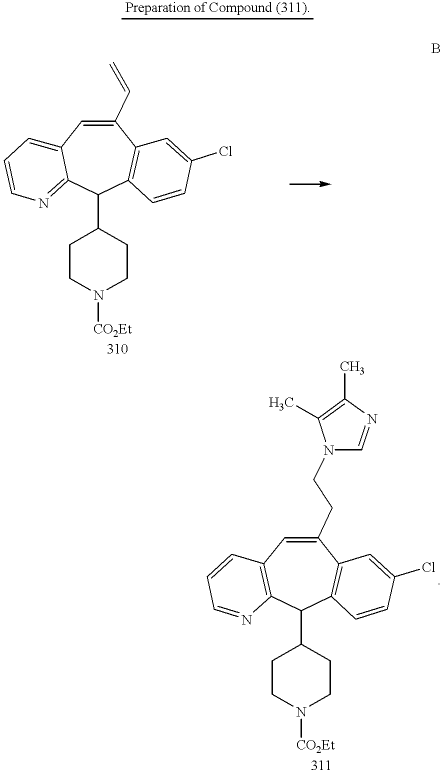

To a stirred solution of 4,5-dimethylimidazole (25.8 mg, 0.268 mmol) in anhydrous THF (0.2 mL) at −78° C. under Argon, was added 2.5M n-BuLi (0.032 mL, 0.08 mmol). The resulting solution was warmed to room temperature, followed by the addition of the alkene compound (310) from Step A above (0.1 g, 0.24 mmol) in anhydrous THF (0.2 mL). The solution was then heated in an oil bath to 120° C. for 25 hours, followed by extraction with CH₂Cl₂—H₂O. The combined organic layer was then washed with brine, dried over Na₂SO₄, filtered and purified by column chromatography on silica gel, eluting with 5% MeOH/95% CH₂Cl₂ to give the product (311) (0.046 g, 100% yield, MH+=505).

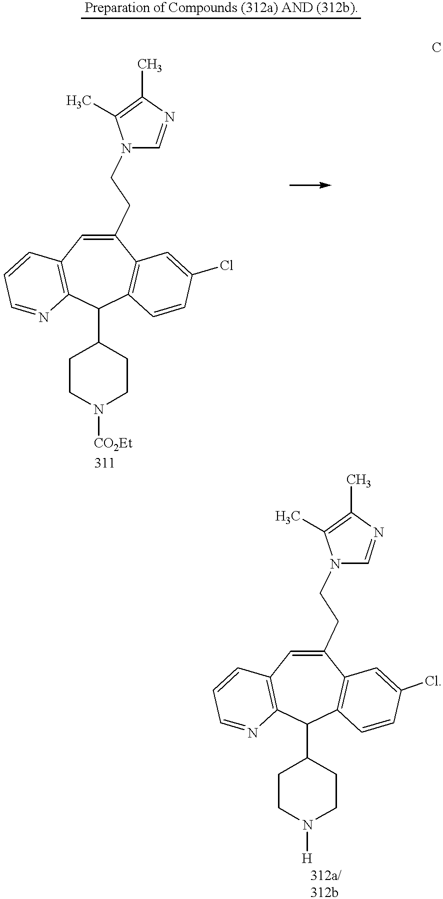

A solution of Compound (311) from Step B above (0.57 g, 1.28 mmol) in 6N HCl (20 mL) was heated to reflux for 24 hours then concentrated to dryness. To the residue was then added saturated NaHCO₃ and NaCl. The solution was extracted twice with CH₂Cl₂. The combined organic layer was dried over Na₂SO4 and concentrated to dryness to give the crude product (0.52 g, 93% yield). The crude material was then dissolved in 20% EtOH/80% Hexane/0.2% DEA and purified by HPLC on a preparative AD column, eluting with 20%-50% IPA/Hexane/0.2% DEA (UV=254 nm, Attn=1024, ABS=2) to give pure isomers of the product (312a) and (312b) (0.225 g, MH⁺=433).

### Examples 126-133

Starting with the appropriate (+) or (−) isomer of Compound (312) and reacting in a similiar manner as in Example 13 using the appropriate isocyanate or sulfonyl chloride, the following compounds were prepared:

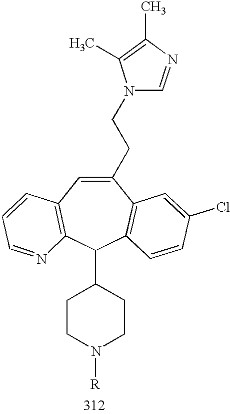

| EX. # | PROCEDURE | R = | CMPD # | PHYS. DATA |
|---|---|---|---|---|
| 126 | Example 13 | 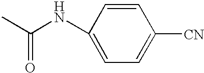 | 313 | Mass spec. M⁺ = 577 |
| 127 | Example 13 | 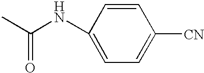 | 314 | Mass spec. M⁺ = 577 |
| 128 | Example 13 | 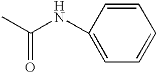 | 315 | Mass spec. M⁺ = 558 |
| 129 | Example 13 | 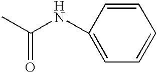 | 316 | Mass spec. M⁺ = 558 |
| 130 | Example 13 | 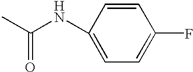 | 317 | Mass spec. M⁺ = 570 |
| 131 | Example 13 | 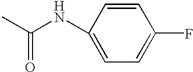 | 318 | Mass spec. M⁺ = 570 |
| 132 | Example 13 | 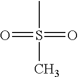 | 319 | Mass spec. M⁺ = 511 |
| 133 | Example 13 | 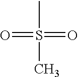 | 320 | Mass spec. M⁺ = 511 |

### Preparative Example 37

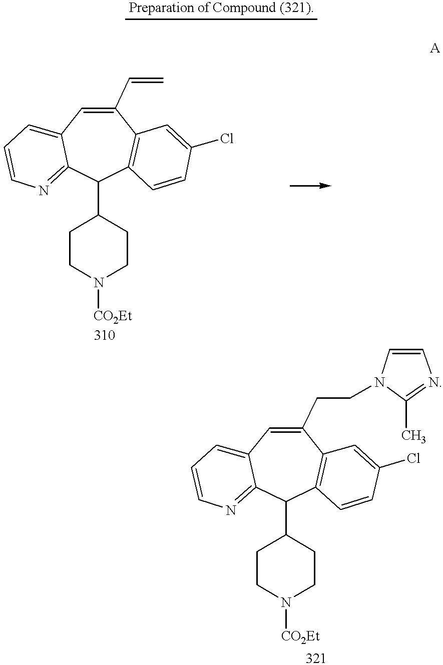

To a solution of Compound (310) from Preparative Example 36, Step A (0.66 g, 8.1 mmol) in THF (4.0 mL) under nitrogen at −78° C., was added dropwise 2.5M n-BuLi/Hexane (1.5 mL). The resulting solution was stirred at −78° C. for 30 minutes, then allowed to warm to room temperature, followed by the addition of 1-methylimidazole (3.0 g, 7.3 mmol) in THF (3.0 mL). The solution was then heated to 120° C. over the weekend and then cooled down to room temperature and concentrated to dryness.

The mixture was extracted with EtOAc-H₂O, dried over MgSO₄, filtered and purified by column chr omatography on silica gel, eluting with 3% MeOH/97% NH₃—CH₂Cl₂ to give the product (321)(1.64 g, 46% yield, MH⁺=491.1).

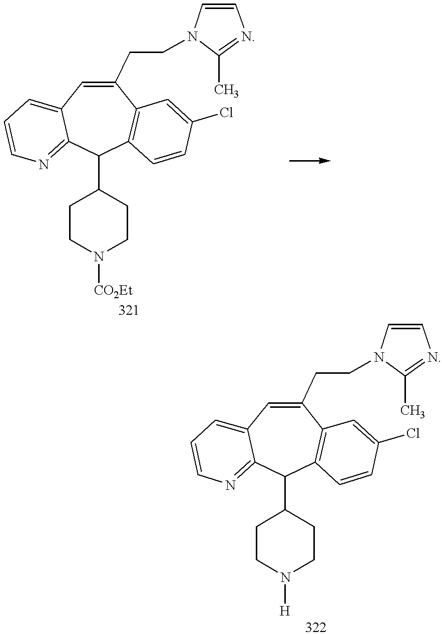

A solution of Compound (321) from Preparative Example 37, Step A above (0.6 g, 1.22 mmol) in 12N HCl (10 mL) was heated to reflux overnight then concentrated to dryness to give the residue as a gum. This residue was dissolved in saturated NaHCO₃, stirred for 10 minutes, saturated with NaCl and then stirred with CH₂Cl₂ for 10 minutes. The solid was filtered and the aqueous layer was extracted twice with CH₂Cl₂, and the organic layer was dried over Na₂SO₄, filtered and concentrated to dryness to give the Compound (322) as a light brown solid (566 mg, MH⁺=419.1).

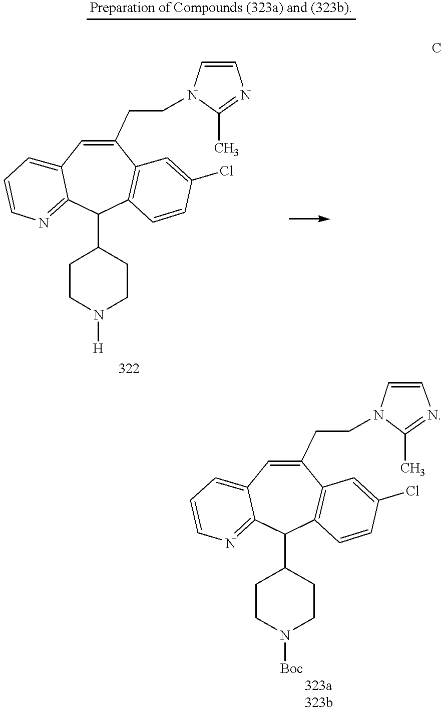

To a solution of Compound (322) from Step B above (0.566 g, 1.35 mmol) in MeOH (20 mL) and H₂O (1 mL) at 0° C., was added Boc anhydride (0.44 g, 2.02 mmol). The solution was basified with 1N NaOH solution to maintain pH=8.5-9.5 and concentrated to dryness, followed by extraction with CH₂Cl₂-H₂O. The combined organic layer was washed twice with H₂O then brine, dried over Na₂SO₄, filtered and concentrated to dryness to give a mixture of isomers 1 and 2 (0.63 g, 100% yield). The isomers were separated by HPLC on a prep AD column, eluting with 15%1PA/85%hexane/0.2%DEA (wave length=254 nm, Attn=64, ABS=1) to give isomer 1 (323a) (0.28 g, MH⁺=519.2) and isomer 2 (323b) (0.28 g, MH⁺=519.2)

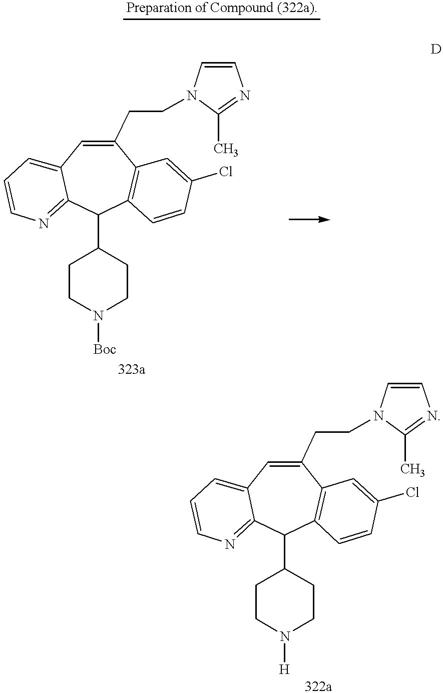

A solution of Compound (323a) isomer 1 from Step C above (0.24 g, 0.46 mmol) in 4N HCl/Dioxane (20 mL) was stirred at room temperature for 1 hr. CH₂Cl₂ (7 mL) was added to the solution and the reaction continued to stir for 2 hrs before being concentrated to dryness. The solution was stirred for 5 minutes with saturated NaHCO₃, then saturated with NaCl and extracted three times with CH₂Cl₂. The combined organic layer was dried over Na₂SO₄, filtered and evaporated to dryness to give Compound (322a) isomer 1(0.163 g, 84% yield, MH⁺=419.2).

Compound (322b) was prepared in a similar manner as in Step D above, starting with Compound (323b) to give the other isomer (0.193 g, 84% yield, MH⁺=419.2)

### Examples 134-147

Starting with compound 322a (isomer 1) or 322b (isomer 2) and reacting in a similiar manner as in Example 13 using the appropriate chloroformate, isocyanate, or sulfonyl chloride (or in the case of carboxylic acid, using DEC mediated coupling) the following compounds were prepared:

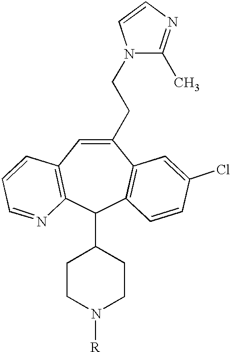

| EX. # | PROCEDURE | R = | CMPD # | PHYS. DATA |
|---|---|---|---|---|
| 134 | Example 13 | 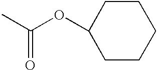 | 324 Isomer 1 | MS M⁺ = 545.2 |
| 135 | Example 13 | 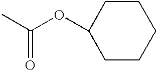 | 325 Isomer 2 | MS M⁺ = 545.2 |
| 136 | Example 13 | 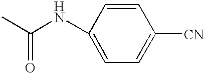 | 326 Isomer 1 | MS M⁺ = 563.2 |
| 137 | Example 13 | 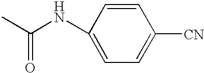 | 327 Isomer 2 | MS M⁺ = 563.2 |
| 138 | Example 13 | 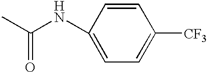 | 328 Isomer 1 | MS M⁺ = 606.1 m.p. = 62.7-63.0° C. |
| 139 | Example 13 | 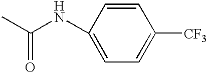 | 329 Isomer 2 | MS M⁺ = 606.1 m.p. = 70.1-71.0° C. |
| 140 | Example 13 | 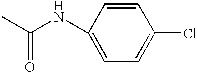 | 330 Isomer 1 | MS M⁺ = 572.1 m.p. = 120.1-121.4° C. |
| 141 | Example 13 | 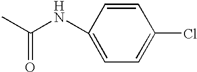 | 331 Isomer 2 | MS M⁺ = 572.1 m.p. = 128.0-129.0° |
| 142 | Example 13 | 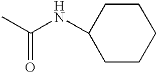 | 332 Isomer 1 | MS M⁺ = 544.2 |
| 143 | Example 13 | 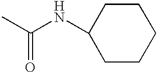 | 333 Isomer 2 | MS M⁺ = 544.2 |
| 144 | Example 13 | 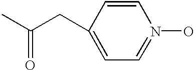 | 334 Isomer 1 | MS M⁺ = 544.1 m.p. = 111.9-112.0° C. |
| 145 | Example 13 | 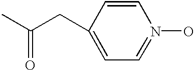 | 335 Isomer 2 | MS M⁺ = 554.1 m.p. = 114.3-115° |
| 146 | Example 13 | 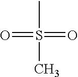 | 336 Isomer 1 | MS M⁺ = 497.1 m.p. = 52.4-53.3° C. |
| 147 | Example 13 | 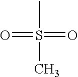 | 337 Isomer 2 | MS M⁺ = 497.1 m.p. = 47.1-48.0° |

### Preparative Example 38

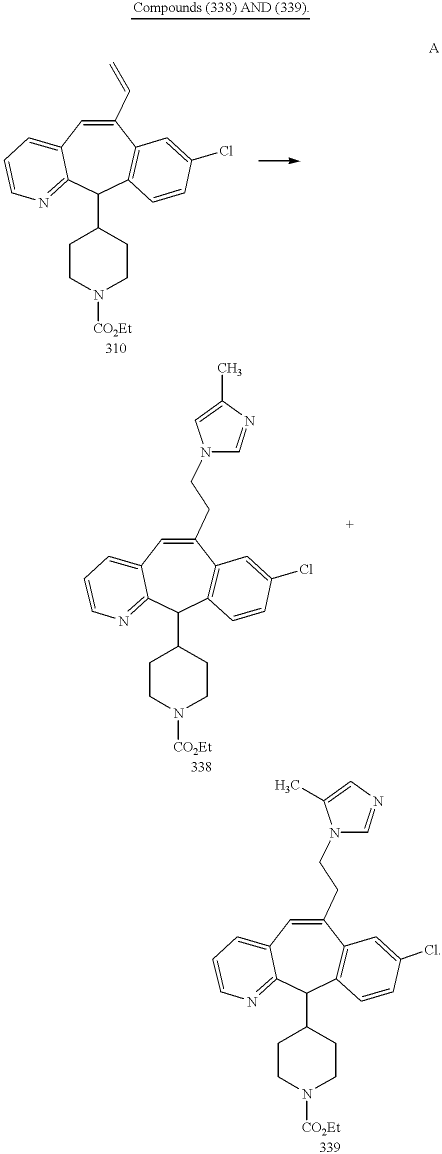

To a solution of Compound (310) from Preparative Example 36 Step A (3.0 g, 7.34 mmol) in THF (8 mL) under nitrogen at −78° C., was added dropwise 2.5M n-BuLi/Hexane (0.65 mL, 8.07 mmol). The resulting solution was stirred at −78° C. for 30 minutes, then allowed to warm to room temperature, followed by the addition of 4-methylimidazole (0.66 g, 8.07 mmol) in THF. The solution was heated to 120° C. over night cooled down to room temperature and concentrated to dryness The reaction mixture was extracted with EtOAc-H₂O, and the organic layer was dried over MgSO₄, filtered and concentrated to give a mixture of 4-methyl substituted (338) and 5-methyl substituted (339) products (2.76 g, 76% yield, M⁺=491.1).

B. Separation of compounds (338a/b) and (339a/b).

In a similar manner as described in Example 11, the mixture of products from Step A, above were first seperated into a mixture of pure 4 and 5-substitured (+) enantiomers and pure 4 and 5-substituted (−) enantiomers using chiral HPLC column chromatography, then upon treatment with triphenyl methyl chloride following the procedure in Example 11, the compounds were further separated into the pure isomers of the 4-substituted compound (338a) (MS M₊=491; mp=72.1-73.0° C.) and (338b) (MS M₊=491; mp=68.9-69.0° C.) and the 5-substituted compound (339a) and (339b).

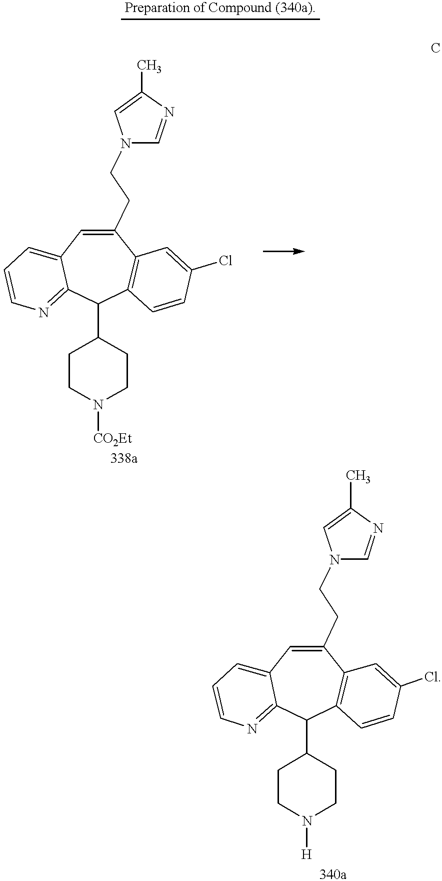

A solution of Compound (338a) from step B above (0.035 g, 0.071 mmol) in 6N HCl (2.0 mL) was heated to reflux overnight. The solution was cooled to room temperature, basified with NH₄OH solution and extracted with CH₂Cl₂. The combined organic layer was dried over MgSO₄, filtered and concentrated to give pure isomer 1, Compound (340a) (0.0334 g, 100% yield, MH⁺=419.1; mp=60.3-61.0° C.).

In a similar manner as above, starting with Compound (338b) (isomer 2), Compound (340b) (MH⁺=419.1) was prepared.

### Examples 148-156

Starting with the appropriate (+) or (−) isomer of Compound (340) and reacting in a similiar manner using the procedure shown in the table below, with the appropriate chloroformate, isocyanate or sulfonyl chloide, the following compounds were prepared:

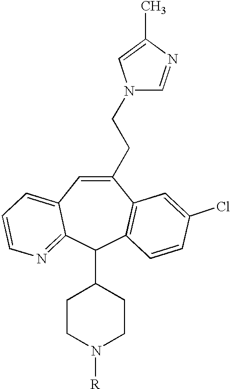

| EX. # | PROCEDURE | R = | CMPD # | PHYS. DATA |
|---|---|---|---|---|
| 148 | Preparative Ex. 4; | BOC | 341 | MS MH⁺ = 519 |
| | Step A | | | m.p. = 90.2-91.0° C. |
| 149 | Example 13 | 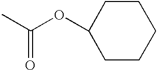 | 342 isomer 1 | MS MH⁺ = 545 m.p. = 58.8-59.6° C. |
| 150 | Example 13 | 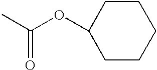 | 343 isomer 2 | MS MH⁺ = 545 m.p. = 60.8-61.2° C. |
| 151 | Example 13 | 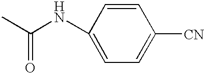 | 344 isomer 1 | MS MH⁺ = 545 m.p. = 98.7-99.5° C. |
| 152 | Example 13 | 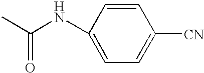 | 345 isomer 2 | MS MH⁺ = 545 m.p. = 111.3-112.0° C. |
| 153 | Example 13 | 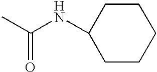 | 346 isomer 1 | MS MH⁺ = 544 m.p. = 77.1-77.8° C. |
| 154 | Example 13 | 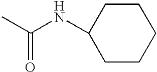 | 347 isomer 2 | MS MH⁺ = 544 m.p. = 78.9-79.0° C. |
| 155 | Example 13 | 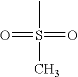 | 348 isomer 1 | MS MH⁺ = 497 m.p. = 87.4-88.0° C. |
| 156 | Example 13 | 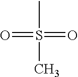 | 349 isomer 2 | MS MH⁺ = 497 m.p. = 88.8-89.0° C. |

### Preparative Example 39

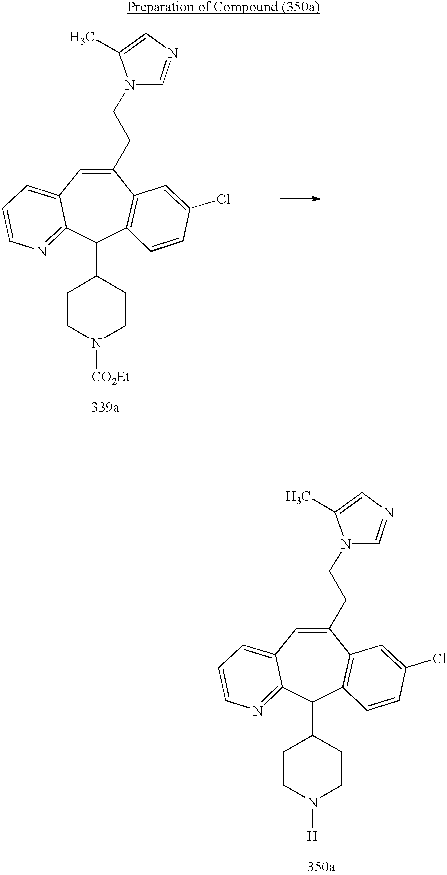

Compound (339a) was reacted in a similar manner as in Preparative Example 38, Step C to give Compound (350a) (isomer 1) (0.13 g, 76% yield, MH⁺=419.3).

Compound (350b) (isomer 2) was prepared in the same manner as above.

### Examples 157-160

Starting with the appropriate (+) or (−) isomer of Compound (350) and reacting in a similiar manner using the procedure indicated in the table below and the appropriate Boc or isocyanate reagent, the following compounds were prepared:

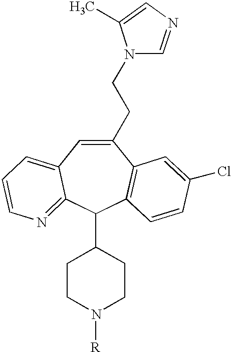

| EX. # | PROCEDURE | R = | CMPD # | PHYS. DATA |
|---|---|---|---|---|
| 157 | Preparative Ex. 4; | BOC | 351 isomer 1 | MS MH⁺ = 519 |
| | Step A | | | m.p. = 87.8-88.2° C. |
| 158 | Preparative Ex. 4; | BOC | 352 isomer 2 | MS MH⁺ = 519 |
| | Step A | | | m.p. = 89.0-89.9° C. |
| 159 | Example 13 | 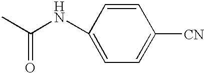 | 353 isomer 1 | MS MH⁺ = 563 |
| 160 | Example 13 | 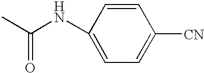 | 354 isomer 2 | MS MH⁺ = 563 m.p. = 130.1-131.0° C. |

### Preparative Example 40

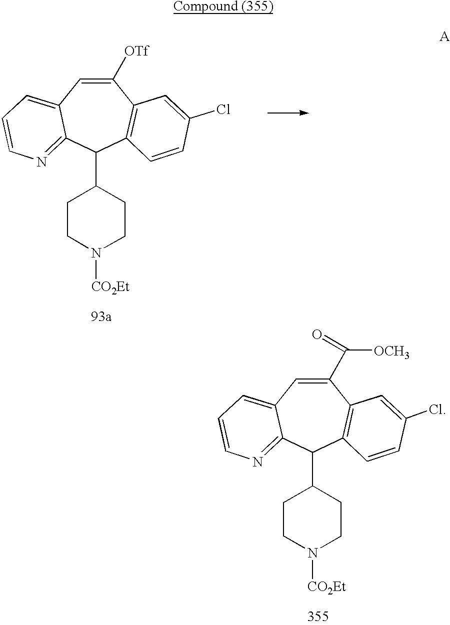

To a solution of Compound (93A) from Preparative Example 7, Step A (2.92 g, 5.5 mmol) in anhydrous toluene (70 mL) and MeOH (10 mL) was added triphenyl phosphine (0.729, 2.75 mmol), DBU (1.11 mL, 7.42 mmol) and PdCl₂(0.097 g, 0.55 mmol). The resulting solution was purged with CO (100 psi), then heated to 80° C. for five hours. The solution was cooled to room temperature, purged with nitrogen and evaporated to dryness to give a brown oil. The product was purified by silica gel column chromatography eluting with 1% MeOH/99% CH₂Cl₂ to 4% MeOH/96%CH₂Cl₂ to give Compound (355) (2.22 g, 92.5% yield, MH+=441.1).

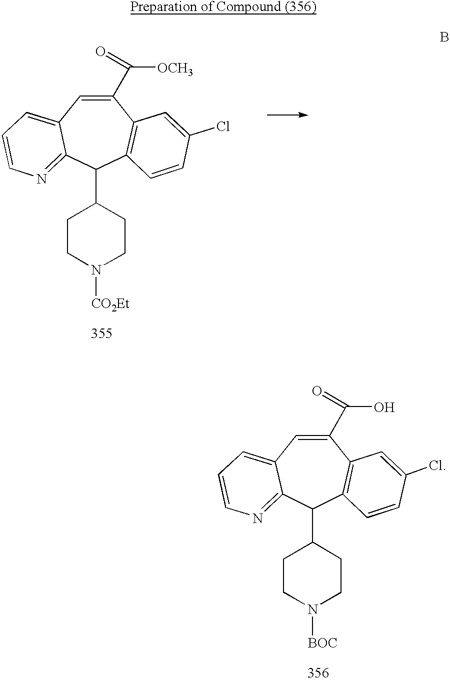

A solution of Compound (355) from Preparative Example 40, Step A (2.2 g, 4.99 mmol) in 6N HCl (50 mL) was heated to 100° C.-110° C. overnight. The solution was cooled to room temperature and evaporated to dryness to give the crude product. To a solution of the crude material in MeOH (50 mL) and H₂O (1 mL) at 0° C., was added Boc anhydride (1.63 g, 7.48 mmol). The resulting solution was basified with 1N NaOH to pH=8.5-9.5 and stirred for two hours at 0° C., then evaporated to dryness and extracted with EtOAc-5% Citric acid solution. The organic layer was washed with H₂O, then brine, dried over Na₂SO₄, filtered and concentrated to dryness to give Compound (356) as a yellow solid (2.29 g, 100% yield, MH⁺=455.1).

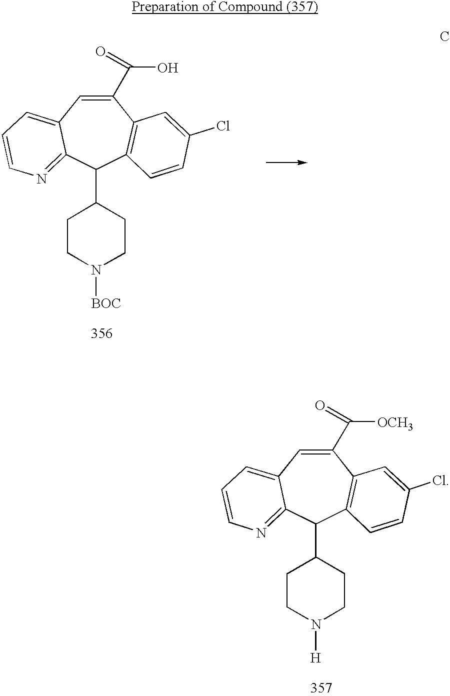

To a solution of Compound (356) from Preparative Example 40, Step B above(2.26 g, 4.97 mmol) in anhydrous benzene (18.0 mL) and MeOH (2 mL), was added, over five minutes, (trimethylsilyl)diazomethane (3 mL, 5.99 mmol) in 2M 1N Hexane. The resulting solution was stirred at room temperature for one hour then evaporated to dryness to give 2.33 g of crude material (MH+=369).

A solution of the crude material (obtained above) in 4N HCl in Dioxane (25 mL) was stirred at room temperature for one hour. The reaction was then evaporated to dryness and purified by flash silica gel column chromatography, eluting with 2% MeOH/98% CH₂Cl₂ to 6% MeOH/94% CH₂Cl₂ and then with 50% (10% NH₄OH/CH₃OH/50% CH₂Cl₂). The collected fractions were evaporated to dryness and diluted with CH₂Cl₂. The organic solution was then washed with saturated NaHCO₃ and brine, dried with Na₂SO₄, filtered and evaporated to dryness to afford Compound (357) (1.26 g, 68.3% yield, MH⁺=369).

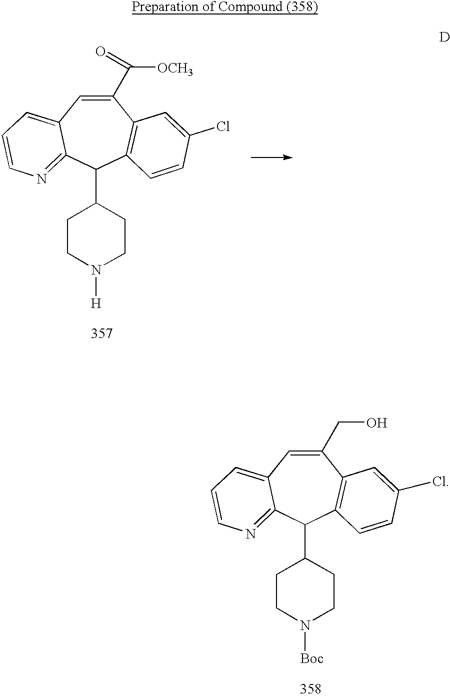

To a solution of Compound (357) from Preparative Example 40, Step C (0.6 g, 1.62 mmol) in anhydrous THF (6 mL) at 0° C. was added DIBAL (1 M solution in toluene) (9.78 mL, 9.78 mmol). The resulting solution was warmed to room temperature and stirred overnight. The solution was then quenched with MeOH and evaporated to dryness to give a crude product.

To the crude material (obtained above) in MeOH at 0° C. was added Boc anhydride (1.06 g, 4.9 mmol). The resulting solution was basified with 1N NaOH to pH=8.5-9.5, stirred for 1 hour and evaporated to dryness. The crude material was diluted with CH₂Cl₂ to give a slurry. The precipitate was then filtered through celite and the CH₂Cl₂ was washed with H₂O, brine, filtered over Na₂SO4 and concentrated to dryness. The crude alcohol product (358) (1.27 g, 100% yield) was used in the next step without further purification.

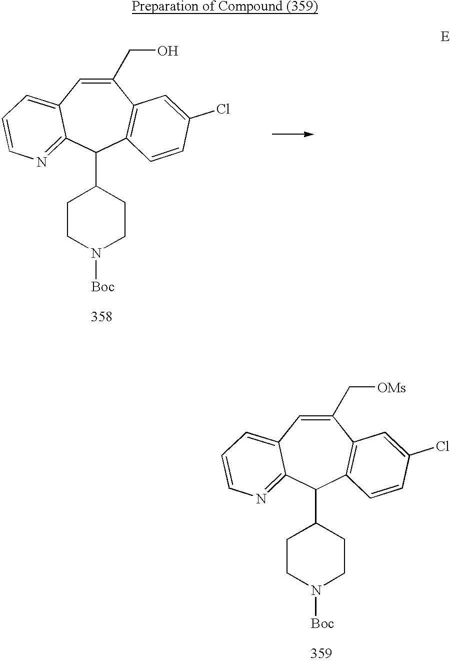

To a cooled solution of the alcohol (358) from Step D above (1.2 g, 2.73 mmol) in anhydrous CH₂Cl₂ (12 mL) at 0° C. was added triethyl amine (1.14 mL, 8.18 mmol) and methanesulfonyl chloride (0.3 mL, 4.1 mmol). The resulting solution was warmed to room temperature stirred overnight, then quenched with H₂O and stirred for 10 minutes. The reaction was washed with water, then brine, dried over Na₂SO₄, filtered and evaporated to dryness to give Compound (359) (1.22 g, 86% yield).

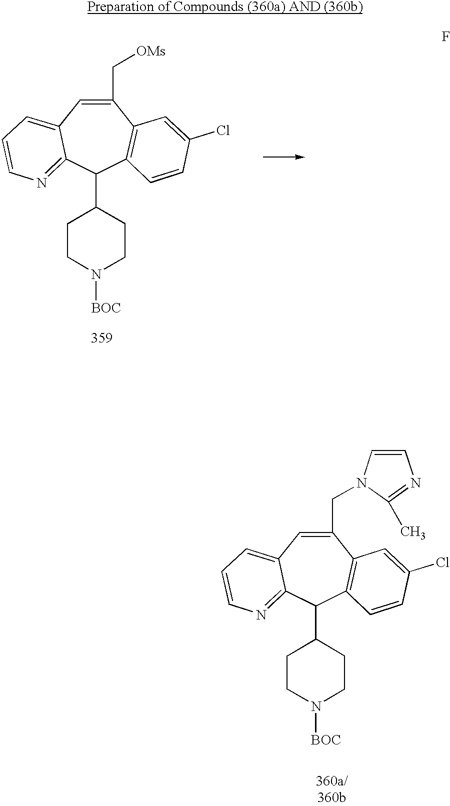

To a solution of anhydrous DMF (5 mL) at 0° C. was added, NaH (0.19 g, 8.18 mmol) and 2-methylimidazole (0.67 g, 8.18 mmol). The resulting solution was warmed to room temperature and stirred for 20 minutes. To the reaction was added a solution of Compound (359) from Step E above (1.22 g, 2.3 mmol) in anhydrous DMF (5 mL). The resulting of solution was stirred at room temperature overnight, then diluted with EtOAc and washed with water then brine. The organic layer was dried over Na₂SO₄, concentrated to dryness and purified by silica gel column chromatography eluting with 1% MeOH/99% CH₂Cl₂ to 5%MeOH/CH₂Cl₂ to give the product as a mixture of isomers (1.18 g, 100% yield, MH+=505.2). Separation of the product mixture by HPLC using a prep AD column, eluting with 25%1PA/75%hexane/0.2%DEA (isocratic 60 m/min.) afforded pure isomer 1 (360a) (0.251 g, MH+=505.1) and isomer 2 (360b) (0.251 g, MH⁺=505.1) as light pink solids.

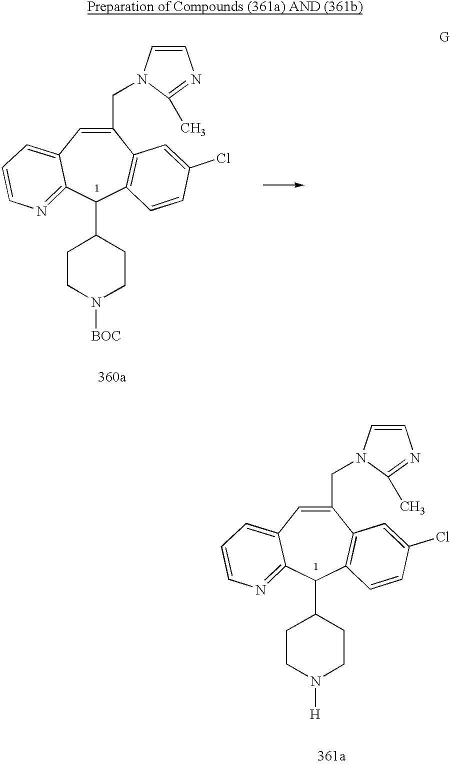

A solution of Compound (360a) (isomer 1) from Step F above (0.2 g, 0.4 mmol) in 4N HCl in Dioxane (10 mL) was stirred at room temperature for 2 hours and then evaporated to dryness to afford Compound (361 a) (0.292 g, 100% yield).

Compound (361b) (isomer 2) was prepared in a similar manner as above beginning with Compound (360b) from Preparative Example 40, Step F.

### Examples 161-166

Starting with the appropriate (+) or (−) isomer of Compound (361) and reacting in a similiar manner as in Example 13 using the appropriate isocyanate shown in the table below, the following compounds were prepared:

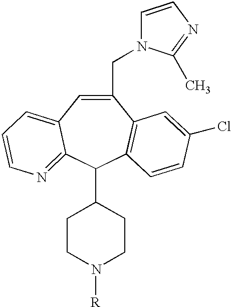

| EX. # | PROCEDURE | R = | CMPD # | PHYS. DATA |
|---|---|---|---|---|
| 161 | Example 13 | 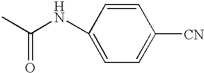 | 362a isomer 1 | MS MH+ = 548 |
| 162 | Example 13 | 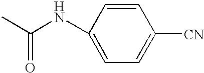 | 362b isomer 2 | MS MH+ = 548 |
| 163 | Example 13 | 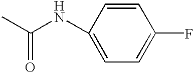 | 363a isomer 1 | MS MH+ = 541 |
| 164 | Example 13 | 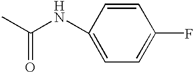 | 363b isomer 2 | MS MH+ = 541 |
| 165 | Example 13 | 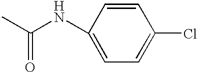 | 364a isomer 1 | MS MH+ = 558 |
| 166 | Example 13 | 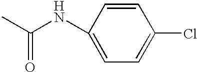 | 364b isomer 2 | MS MH° = 558 |
| 166.1 | Example 13 | 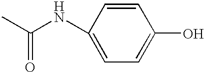 | 364c | Mp 201.5-208.3+ C. |

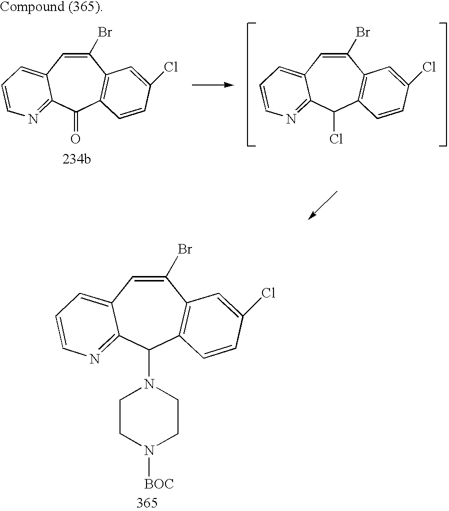

In essentially the same manner as in Preparative Example 23, Steps A-D, using the 6-Bromo substituted product from Step B, Compound (234b), the product Compound (365) was prepared (76.6 g, 100% yield).

### Preparative Example 42

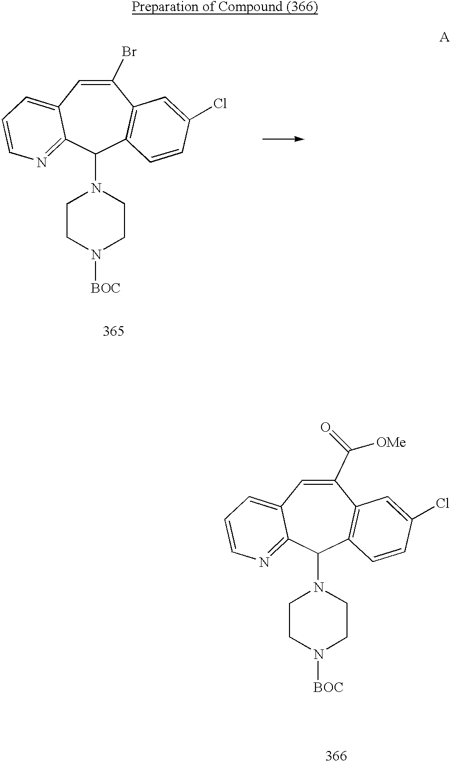

To a solution of Compound (365) from Preparative Example 41 (4.0 g, 8.16 mmol) in toluene (75 mL) and MeOH (20 mL), was added triphenyl phosphine (1.099 g, 4.08 mmol), DBU (1.7 g, 11.02 mmol) and palladium chloride (0.145 g, 0.82 mmol). The resulting solution was evacuated with CO at 100 psi and heated at 78° C.-82° C. for 5 hours, followed by the extraction with EtOAc-H₂O. The combined organic layer was then washed with brine, dried over Na₂SO4, concentrated to dryness and purified by column chromatography, eluting with 30% EtOAc/70% Hexane to give a Compound (366) (3.12 g, 100% yield, MH+-470.1).

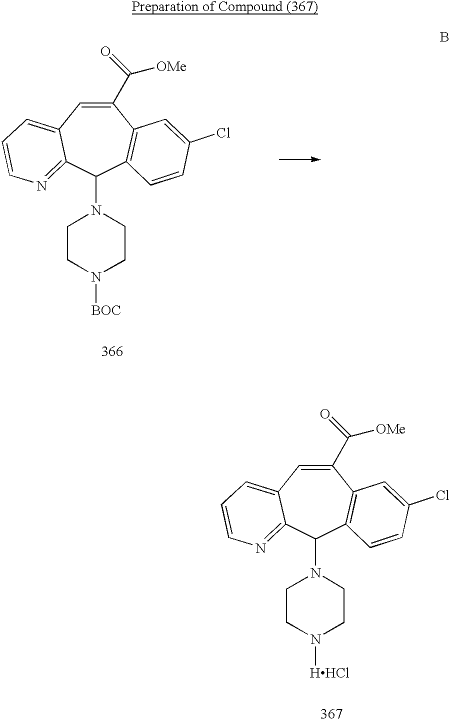

A solution of Compound (366) from Step A above (3.1 g, 6.6 mmol) in 4M HCl/Dioxane (120 mL) was stirred for 3 hours and then concentrated to dryness to give the crude salt of Compound (367) (3.89 g, 100% yield, MH⁺=370.2)

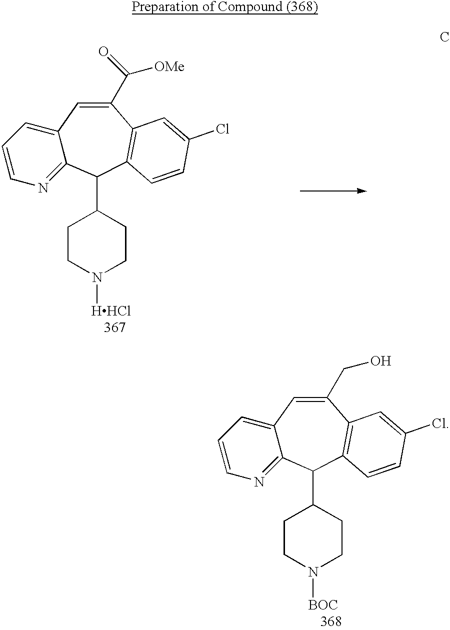

To a solution of Compound (367) from Step B above (3.43 g, 8.45 mmol) in THF (60 mL) at 0° C., was added DIBAL (7.21 g, 50.7 mmol). The resulting solution was warmed to room temperature, stirred overnight and then concentrated to dryness, followed by the addition of Boc anhydride (3.69 g, 16.9 mmol). The reaction was then extracted with CH₂Cl₂-H₂O, filtered over Na₂SO₄ and concentrated to dryness to afford Compound (368) (3.75 g, 100% yield, MH+=442.4).

C.1 Alternate Preparation of Compound (368).

A solution of compound 366 from step A above (23.46 g, 50.98 mmol) in CH₂Cl₂—MeOH—H₂O (120 mL, 600 mL, 60 mL respectively) combined with LiOH (12.0 g, 350.88 mmol) was refluxed at 40° C. overnight. Solvent was removed from the reaction mixture and the residue diluted with CH₂Cl₂, was acidified to pH 6 with 1N HCl. The organic layer was separated and washed with water, dried over Na₂SO₄ and concentrated. The product was dissolved in THF (285 mL) at 0° C. Triethyl amine (6 mL, 42.97 mmol) and ethyl chloroformate (4.1 mL, 42.97 mmol) were added and stirred at 0° C. for 1 h. The reaction mixture was filtered and the filtrate was cooled to −70° C. To this filtrate was added NaBH₄ (3.97 g, 104.94 mmol) and stirred for 1 h at −70° C. after which time 40 mL of MeOH was added dropwise. The solvents were removed and the residue taken up in methylene chloride, washed with sat. (aq) NaHCO₃, then brine, dried over Na₂SO₄ and concentrated to give Compound (368) as a solid.

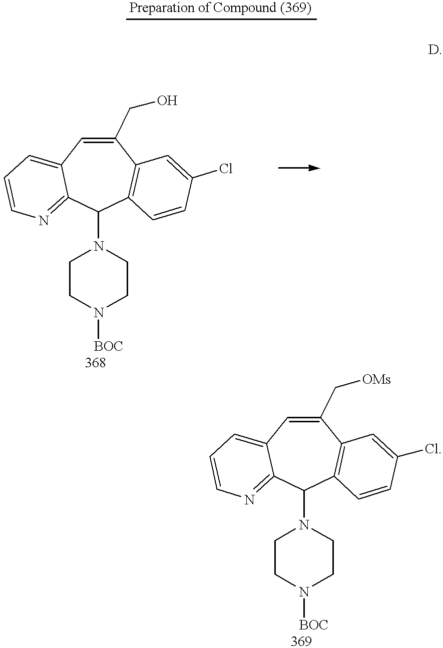

To a solution of Compound (368) from Step C above (3.74 g, 8.46 mmol) in CH₂Cl₂ (100 mL) was added triethyl amine (3.5 mL, 25.38 mmol) and methanesulfonyl chloride (1.45 g, 2.7 mmol). The resulting solution was stirred under nitrogen at room temperature for overnight and then washed with saturated NaHCO₃, then brine, and dried over Na₂SO₄ to give the mesylate compound (369) (3.86 g, 88% yield).

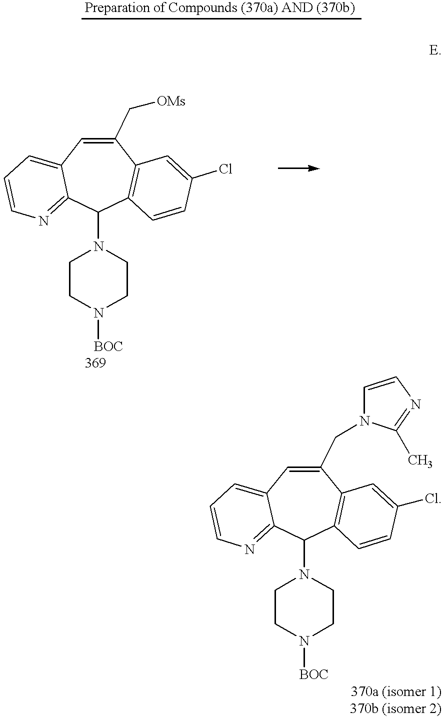

To a solution of 2-methylimidazole (2.43 g, 29.68 mmol) in DMF (30 mL) under N₂ was added NaH (0.53 g, 22.3 mmol) and stirred for 10 min, followed by the addition of Compound (369) from Step D above (3.86 g, 7.42 mmol). The solution was stirred over night. The solution was then concentrated to dryness and extracted with EtOAc—NaHCO₃, dried over Na₂SO₄, and concentrated. The crude product was purified by column chromatography, eluting with 2% MeOH—NH₃/98% CH₂Cl₂ to afford a mixture of isomers. Further separation was accomplished by Preparative HPLC Chiral AD Column chromatography, eluting with 25% IPA/75% hexane/0.2% DEA to give pure Compound (370a) (isomer 1) (0.160 g) and Compound (370b) (isomer 2) (0.140 g) (MH⁺=506.1)

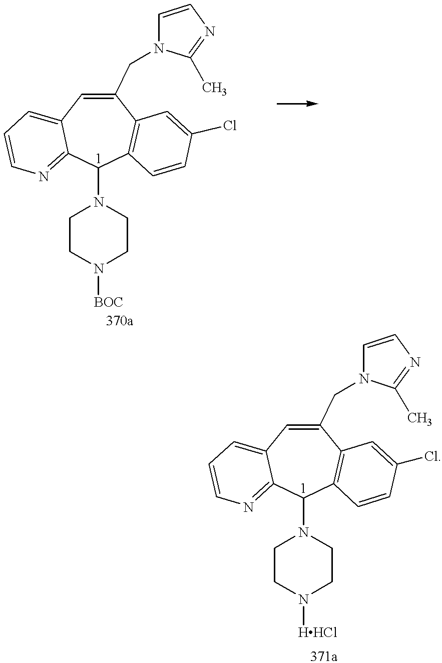

A solution of Compound (370a) (isomer 1) from Step E above (0.105 g, 0.21 mmol) in 4M HCl/Dioxane (10 mL) was stirred at room temperature for 3 hours and concentrated to dryness to afford Compound (371 a) (0.147 g, 100% yield) Compound (370b) (isomer 2) from Step E was treated in the same manner as isomer 1 above, to afford Compound (371 b) (isomer 2).

### Example 167

Preparation of Compound (372)

To a solution of compound 371 a (1.3 g, 2.94 mmol) in CH₂Cl₂ (60 mL) was added triethyl amine (1.3 mL, 9.4 mmol) and p-cyano phenyl isocyanate (0.466 g, 3.24 mmol). The resulting solution was stirred at room temperature overnight, followed by the extraction with CH₂Cl₂ and saturated NaHCO₃. The organic layer was dried over Na₂SO₄, evaporated and the residue purified by column chromatography, eluting with 1% -2% MeOH—NH₃/98% CH₂Cl₂ to afford compound (372) (0.870 g, 48% yield) see table below.

### Example 168

Preparation of Compound (373)

Compound 371 b (isomer 2) was reacted in a similar manner as in Example 13 with p-cyano phenyl isocyanate to afford compound (373) see table below.

### Example 169

Preparation of Compound (374)

Compound 371 a (isomer 1) was reacted in a similar manner as in Example 13 with p-chloro phenyl isocyanate to afford compound (374) see table below.

### Example 170

Preparation of Compound (375)

Compound 371 b (isomer 2) was reacted in a similar manner as in Example 13 with p-chloro phenyl isocyanate to afford compound (375) see table below.

### Examples 167-170

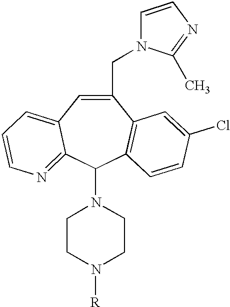

| EX. # | PROCEDURE | R = | CMPD # | PHYS. DATA |
|---|---|---|---|---|
| 167 | Example 13 | 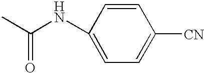 | 372 isomer 1 S-isomer | MS MH+ = 550 |
| 168 | Example 13 | 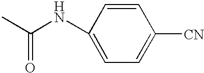 | 373 isomer 2 R-isomer | MS MH+ = 550 |
| 169 | Example 13 | 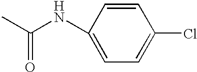 | 374 isomer 1 S-isomer | MS MH+ = 559 |
| 170 | Example 13 | 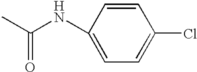 | 375 isomer 2 R-isomer | MS MH+ = 559 |
| 170.1 | Example 13 | 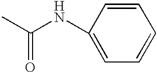 | 375.1 isomer 1 | MS MH+ = 525 |

### Preparative Example 43

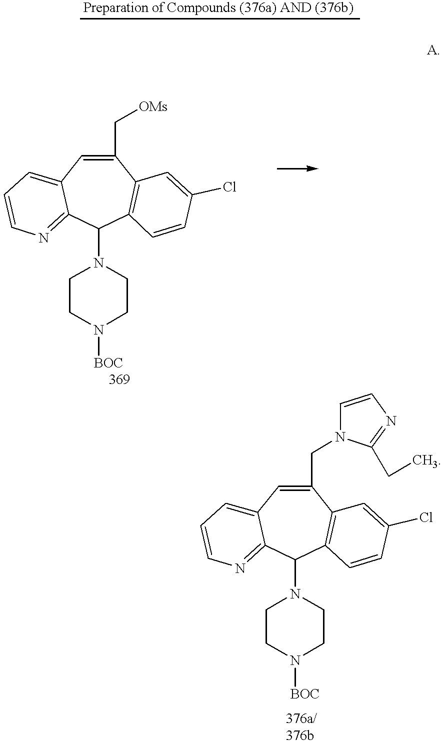

To a solution of 1-ethylimidazole (0.33 g, 3.46 mmol) in DMF (5 mL) under nitrogen was added NaH (0.083 g, 3.46 mmol) and stirred for 10 minutes, followed by the addition of Compound (369) from Preparative Example 42, Step D (0.6 g, 1.15 mmol) and stirred for over night. The solution was then evaporated to dryness, diluted with ethyl acetate, washed with sodium bicarbonate, dried over sodium sulfate and concentrated to dryness. The reaction mixture was purified by column chromatography on silica gel, eluted with 3% MeOH/97% CH₂Cl₂ to give a mixture of isomers. Further separation was accomplished using prep. HPLC with a chiral AD column to afford pure Compound (376a) (isomer 1) and Compound (376b) (isomer 2) (MH₊=520.1).

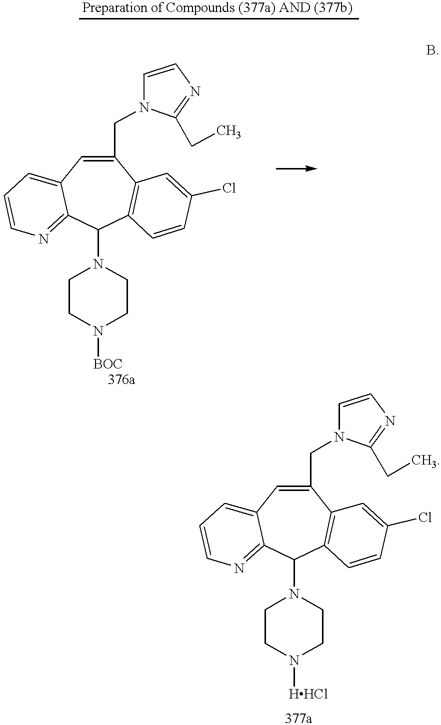

A solution of Compound (376a) from Step A (0.107 g, 0.2 mmol) in 4M HCl in Dioxane (10 mL) was stirred for two hours at room temperature then concentrated to dryness to afford Compound (377a) (isomer 1) (0.13 g, 100% yield, MH⁺=420.1).

Compound (376b) was reacted in a similiar manner as above to afford Compound (377b) (isomer 2) (MH⁺=420.1).

### Examples 171-174

Starting with the appropriate (+) or (−) isomer of Compound (377) and reacting in a similiar manner as in Example 13 using the appropriate isocyanate as shown in the table below, the following compounds were prepared:

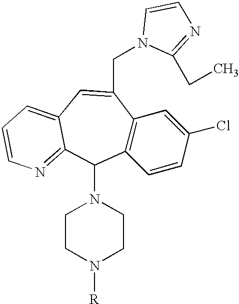

| EX. # | PROCEDURE | R = | CMPD # | PHYS. DATA |
|---|---|---|---|---|
| 171 | Example 13 | 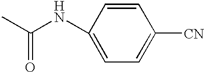 | 378 isomer 1 | MS MH+ = 504 |
| 172 | Example 13 | 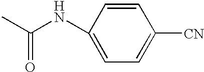 | 379 isomer 2 | MS MH+ = 504 |
| 173 | Example 13 | 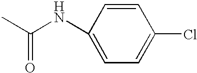 | 380 isomer 1 | MS MH+ = 573 |
| 174 | Example 13 | 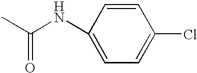 | 381 isomer 2 | MS MH+ = 573 |

### Preparative Example 44

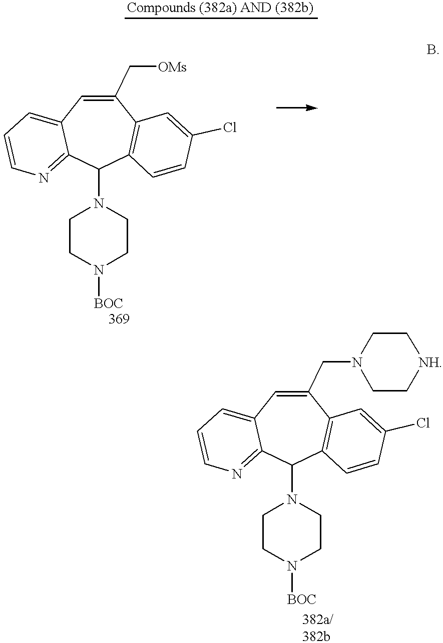

To a solution of Compound (369) from Preparative Example 42, Step D (0.5 g, 0.96 mmol) in CH₃CN (80 mL), was added piperazine (0.25 g, 2.88 mmol) and 2,6-bis (dimethyl)-1-methylpiperidine (0.597 g, 3.84 mmol). The resulting solution was stirred at room temperature for 4hrs, concentrated to dryness and extracted with CH₂Cl₂—NaHCO₃. The combined organic layer was dried over Na₂SO₄ and purified by column chromatography on silica gel, eluting with 3%MeOH/ 97%CH₂Cl₂ to give the product of 2 isomers (0.28 g, 57% yield). These two isomers were separated by HPLC on chiral AD column to give pure Compound (382a) (isomer 1) (0.136 g, MH⁺=510.3) and Compound (382b) (isomer 2) (0.14 g, MH⁺=510.3)

### Preparative Example 45

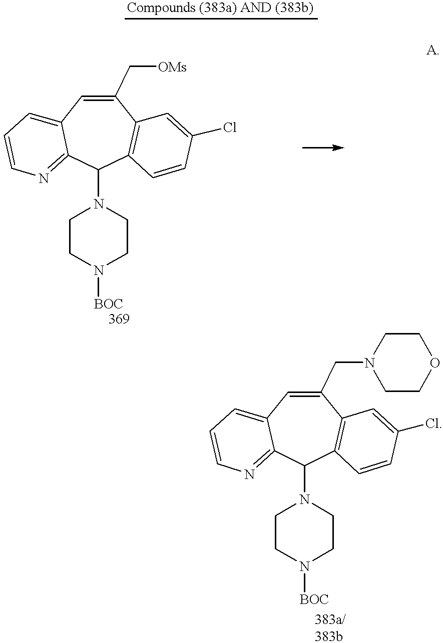

To a solution of Compound (369) from Preparative Example 42, Step D (1.2 g, 2.31 mmol) in CH₃CN (100 mL), was added morpholine (0.8 g, 9.23 mmol) and 2,6-bis (dimethyl)-1-methylpiperidine (1.9 g, 12.24 mmol). The resulting solution was stirred at room temperature overnight and concentrated to dryness, followed by extraction with CH₂Cl₂—NaHCO₃. The combined organic layer was dried over Na₂SO₄ and purified by column chromatography on silica gel, eluting with 1%NH₃—MeOH/99%CH₂Cl₂ to give the product of two isomers (1.1 g, 82% yield). These two isomers were separated by HPLC on chiral AD column to give pure Compound (383a) (isomer 1) (0.24 g, MH⁺+425.1) and Compound (383b) (isomer 2) (0.112 g, MH⁺=425.1).

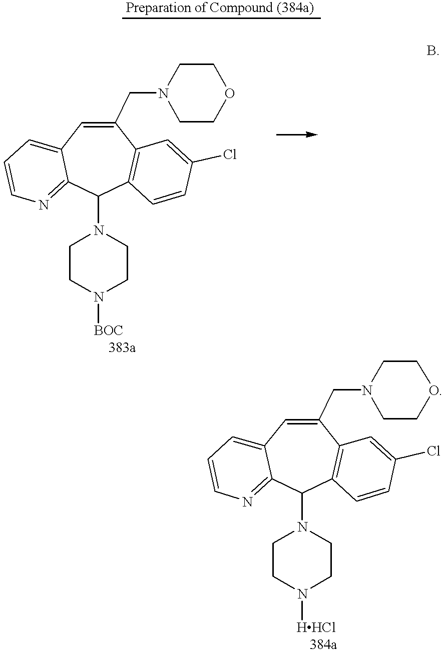

A solution of Compound (383a) from Step A (0.19 g, 0.37 mmol) in 4M HCl/Dioxane (25 mL) was stirred at room temperature for 2.5 hrs and concentrated to dryness to give Compound (384a) (0.194 g, MH⁺=411.1).

Compound (384b) was prepared in a similar manner as above starting with Compound (383b) from Step A.

### Example 175

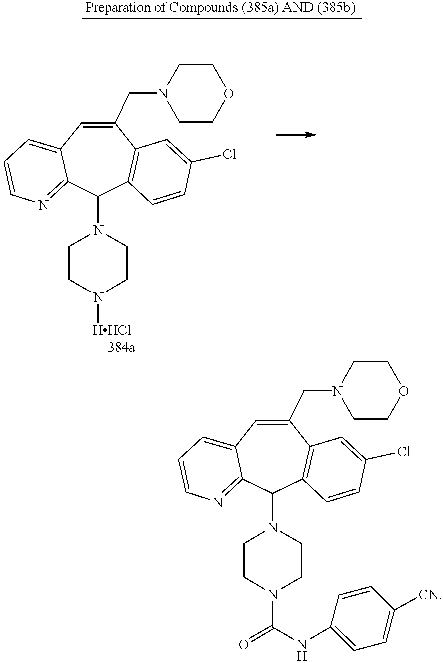

To a solution of Compound (384a) from Preparative Example 45, Step B above (0.05 g, 0.11 mmol) in anhydrous CH₂Cl₂ (5 mL) was added triethyl amine (0.036 g, 0.36 mmol) and 4-cyanophenyl isocyanate (0.018 g, 0.173 mmol). The resulting solution was stirred at room temperature for 4 hrs under nitrogen and concentrated to dryness, followed by extraction with CH₂Cl₂—NaHCO₃. The combined organic layer was dried over Na₂SO₄ and concentrated to dryness to give Compound (385a) (isomer 1) (0.06 g, 100% yield, MH⁺=555.4).

Starting with Compound (384b) from Preparative Example 45, Step B and reacting it in the same manner as above, Compound (385b) (isomer 2) was prepared (MH⁺=555.4).

### Preparative Example 46

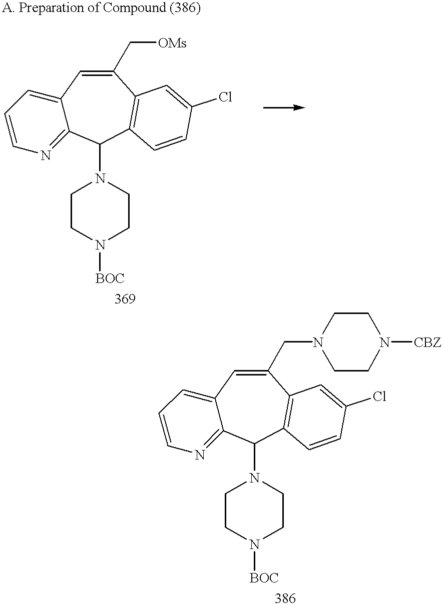

To a solution of Compound (369) from Preparative Example 42 Step D (3.0 g, 5.77 mmol) in CH₃CN (150 mL) was added 2,6-bis (dimethyl)-1 methyl piperidine (7.16 g, 16.16 mmol) and benzyl-1-piperazinecarboxylate (7.61 g, 34.62 mmol). The resulting solution was stirred overnight, concentrated to dryness, followed by s extraction with CH₂Cl₂—NaHCO₃. The combined organic layer was dried over Na₂SO₄, concentrated to dryness and purified by column chromatography on silica gel, eluting with 1% NH₃—MeOH/99% CH₂Cl₂ and then 30%EtOAc/70% hexane to give the title product Compound (386) (1.24 g, 67% yield, MH+=644.2)

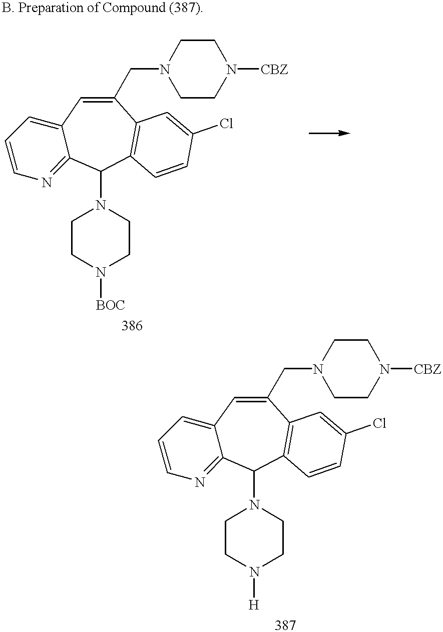

A solution of Compound (386) from Step A above (0.5 g, 0.77 mmol) in 4M HCl/Dioxane (50 mL) was stirred at room temperature for 2 hrs. The solution was then poured onto ice and basified with 1N NaOH solution, followed by extraction with CH₂Cl₂. The combined organic layer was dried over Na₂SO₄ and concentrated to dryness to give Compound (387) (0.43 g, 100% yield, MH⁺=544.5).

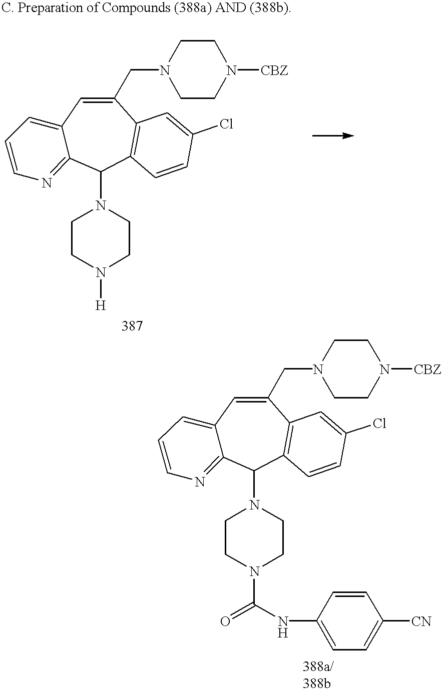

Compound (387) from Step B above was reacted In a similar manner to that described in Example 175 to give a mixture of 2 isomers (0.102 g, 55% yield). Further separation by HPLC, using a chiral AD column afforded pure Compound (388a) (isomer 1) (0.05 g, MH⁺=688.2) and Compound (388b) (isomer 2) (0.048 g, MH⁺=688.2).

### Examples 176 and 177

Reacting Compound (387) from Preparative Example 46, Step B in a similiar manner as in Example 175 using the appropriate isocyanate as shown in the table below, the following compounds were prepared:

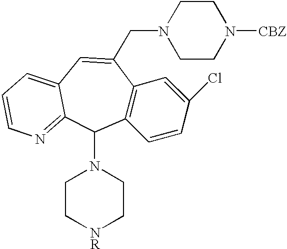

| EX. # | PROCEDURE | R = | CMPD # | PHYS. DATA |
|---|---|---|---|---|
| 176 | Example 175 | 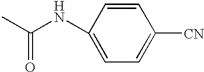 | 389 isomer 1 | MS MH+ = 688 |
| 177 | Example 175 | 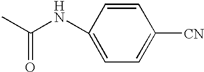 | 390 isomer 2 | MS MH+ = 688 |

### Example 178

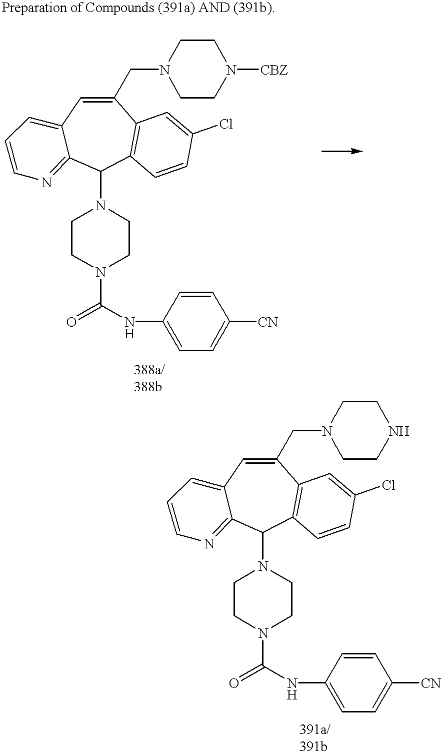

To a solution of Compound (388a) from Preparative Example 46, Step C (0.05 g, 0.086 mmol) in CH₃CN (1 mL) at 0° C. was added iodotrimethylsilane (0.05 mL, 0.343 mmol). The resulting solution was stirred at 0° C. for 1 hr and concentrated to dryness. The residue was then poured onto 1N HCl solution, followed by extraction with ether. The aqueous layer was then basified with 10% NH₄OH solution and then extracted with CH₂Cl₂. The combined organic layer was dried over Na₂SO₄ and concentrated to dryness affording Compound (391 a) (isomer 1) (0.02 g, 42.5% yield, MH⁺=554.1).

Starting with Compound (388b) from Preparative Example 46, Step C, and reacting in the same manner as above, Compound (391b) (isomer 2) was prepared (MH⁺=554.1).

### Preparative Example 47

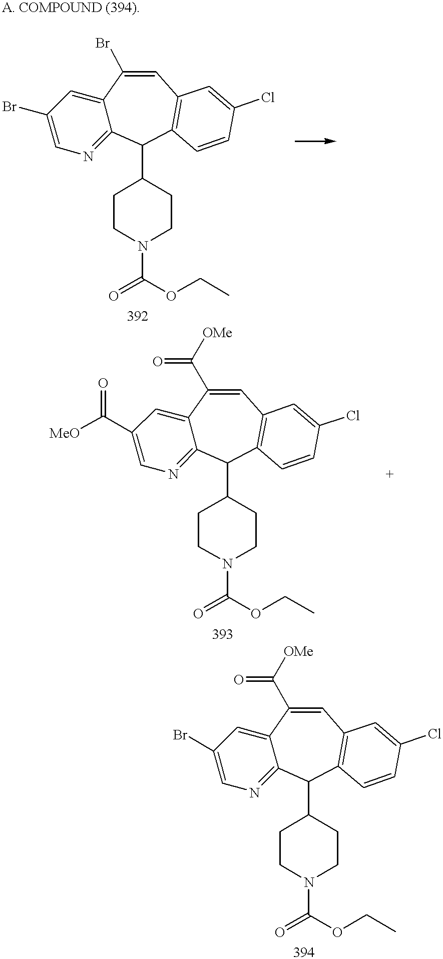

To a solution of Compound (392) prepared according to the procedure in, The Journal of Medicinal Chemistry (1998),41(10),1563 (5.0 g, 9.24 mmol) in MeOH (20 mL) and toluene (50 mL), at room temperature, was added triphenylphosphine (1.21 g, 4.62 mmol), DBU (1.90 g, 12.48 mmol) and palladium chloride (0.16 g, 0.92 mmol). The resulting solution was stirred at 80° C. for 6 hrs, then stirred at room temperature overnight. The solution was then concentrated to dryness to give two products. The desired product was purified by column chromatography on normal phase silica gel, eluting with 30% EtOAc/70%hexane to give a white solid compound (394) (2.24 g, 47% yield, MH₊=521.1)

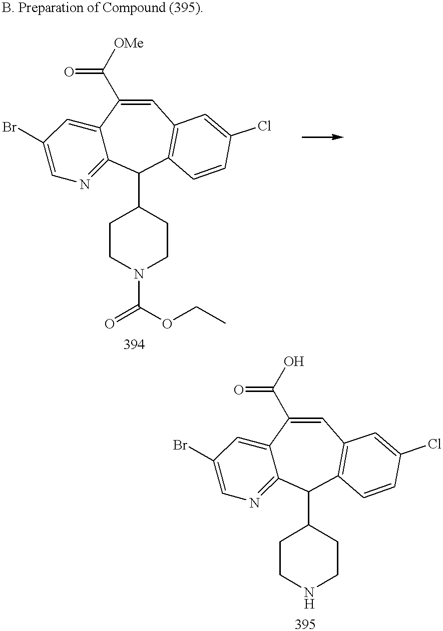

A solution of Compound (394) from Step A above (2.38 g, 4.58 mmol) in concentrated HCL (40 mL) was heated to reflux over night. The solution was then cooled down at room temperature and basified with NH₄OH solution, followed by extraction with CH₂Cl₂. The combined organic layer was dried over MgSO₄, filtered and concentrated to dryness to give a white solid Compound (395) (1.03 g, 52% yield, MH⁺=435.1).

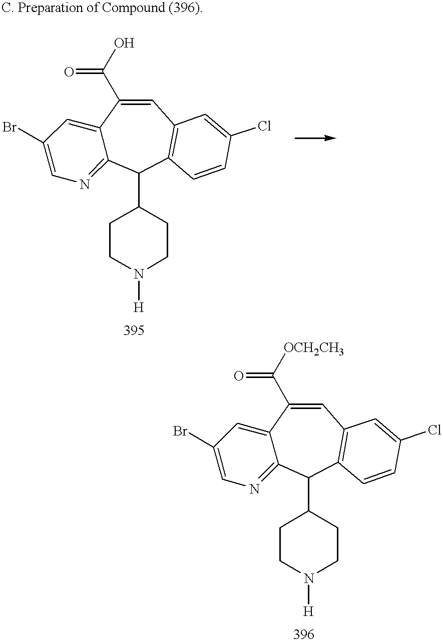

To a solution of Compound (395) from Step B (1.03 g, 2.37 mmol) in EtOH (50 mL, 200 proof) at room temperature, was bubbled in anhydrous CH₂Cl₂ gas for 5 minutes. The solution was then heated at 60° C. for 30 minutes, cooled down to room temperature and concentrated to dryness to afford Compound (396) (1.1 g, 100% yield, MH⁺=463.1)

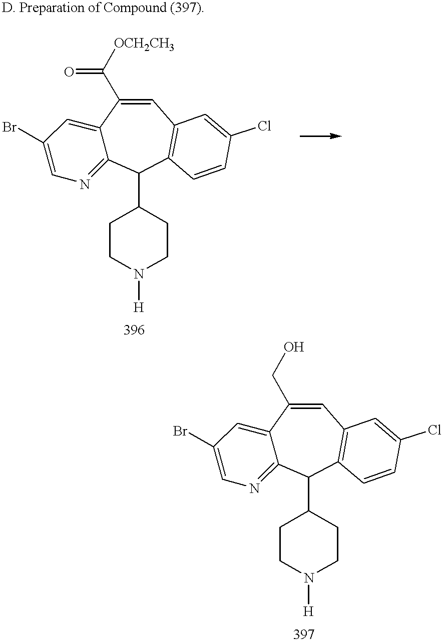

To a solution of Compound (396) from Step C (1.09 g, 2.19 mmol) in THF (10 mL) at 0° C. was added dropwise DIBAUtoluene (11.0 mL, 10.95 mmol). The resulting solution was stirred overnight at room temperature, then quenched with H₂O and concentrated to dryness to give a light brown solid Compound (397) (1.2 g, 100% yield, MH⁺=421.1).

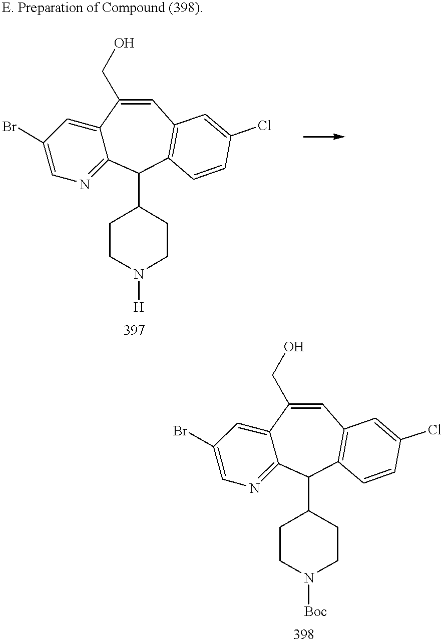

To a solution of Compound (397) from Step D (0.92 g, 2.19 mmol) in 50% MeOH/1% H₂O (50 mL) at room temperature, was added Boc anhydride (0.95 g, 4.38 mmol). The resulting solution was adjusted to pH=9 and stirred at room temperature for 4 hrs and concentrated to dryness, followed by extraction with CH₂Cl₂-H₂O. The combined organic layer was dried over MgSO₄, filtered and concentrated to dryness to give a light brown solid Compound (398) (0.91 g, 80% yield, MH⁺=521.1).

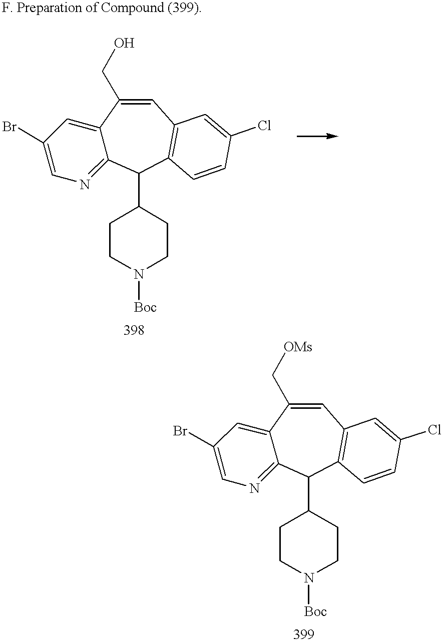

To a solution of Compound (398) from Step E (0.91 g, 1.75 mmol) in CH₂Cl₂ (10 mL) was added triethyl amine (0.73 mL, 5.25 mmol) and methanesulfonyl chloride (0.3 g, 2.62 mmol). The resulting solution was stirred at room temperature overnight and then washed with NaHCO₃ solution, dried over Na₂SO₄, filtered and concentrated to dryness to give the mesylate as a light yellow solid Compound (399) (0.94 g, 90% yield).

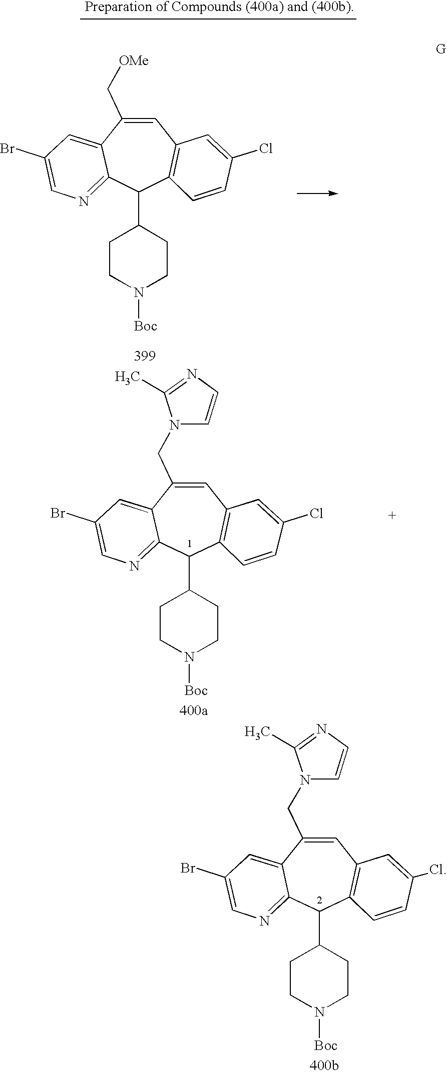

To a solution of Compound (399) from Step F (0.93 g, 1.60 mmol) in DMF (10 mL) under nitrogen, was added 2-methylimidazole (0.19 g, 2.3 mmol) and NaH (0.037 g). The resulting solution was stirred at room temperature for 15 minutes, then at 90° C. for 3hrs. The solution was then cooled down to room temperature and concentrated to dryness, followed by extraction with CH₂Cl₂—NaHCO₃. The combined organic layer was dried over MgSO₄, filtered, concentrated and purified by column chromatography on normal phase silica gel, eluting with 5%MeOH—NH₃/95%CH₂Cl₂ to give mixture of two isomers as a light red solid (0.39 g, 42% yield, MH⁺=585.1). The 2 isomers were separated by prep HPLC, using a chiral AD column, eluting with 15%IPA/85%hexane/0.2%DEA to give Compound (400a) (isomer 1) as a light brown solid (0.10 g, 11% yield) and Compound (400b) (isomer 2) as a white solid (0.10 g, 11% yield)

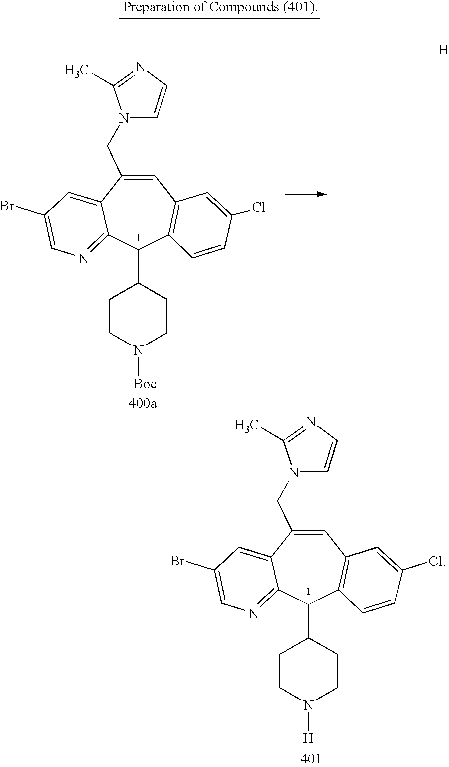

A solution of Compound (400a) (isomer 1) from Step G above (0.07 g, 0.12 mmol) in 4M HCl/Dioxane (3 mL) was stirred at room temperature for 3 hrs then concentrated to dryness to give a white solid Compound (401) (0.06 g, 100% yield)

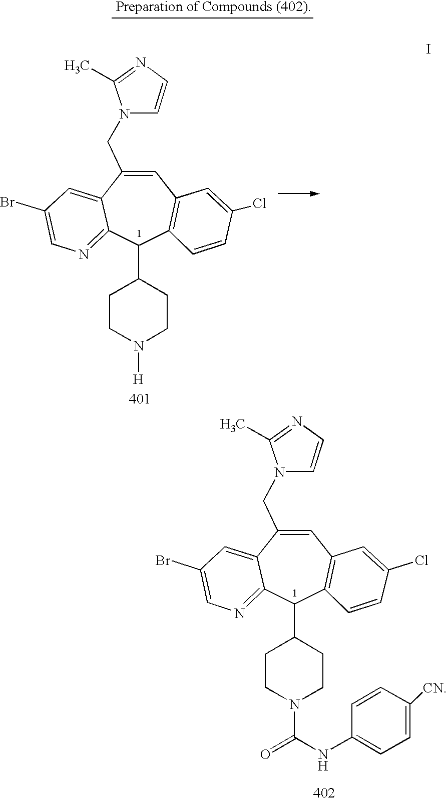

To a solution of Compound (401) from Step H above (0.057 g, 0.12 mmol) in CH₂Cl₂ (5 mL) under nitrogen, was added triethyl amine (0.026 g, 0.20 mmol) and 4-cyanophenyl isocyanate (0.019 g, 0.13 mmol). The resulting solution was stirred at room temperature overnight and then extracted with CH₂Cl₂-NaHCO₃. The combined organic layer was dried over Na₂SO₄, filtered, concentrated to dryness to afford Compound (402) (isomer 1) as a white solid (0.053 g, 70% yield, MH⁺=629.3)

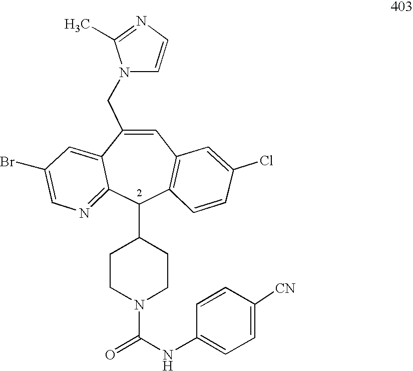

Compound (400b) was reacted in a similar manner as in Steps H and I above to afford Compound (403) (isomer 2) (0.059 g, 79% yield, MH₊=629.3)

### Preparative Example 48

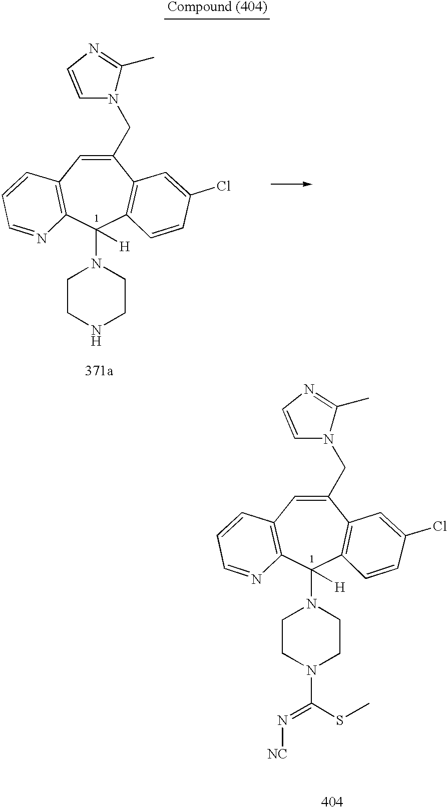

Compound (371 a) (isomer 1) from Preparative Example 42, Step F (70 mg, 0.17 mmol) was dissolved in 1 mL of ethanol and 50 uL of triethylamine. Dimethyl-N-cyanimidothiocarbonate (45 mg, 0.29 mmol) was added and the reaction mixture and stirred at 85° C. for 24 hours. The ethanol was evaporated under reduced pressure and the product chromatographed on silica gel using 5% methanolic-ammonia dichloromethane to obtain 47 mg of title product Compound (404) (FABMS M+1=504).

### Example 179

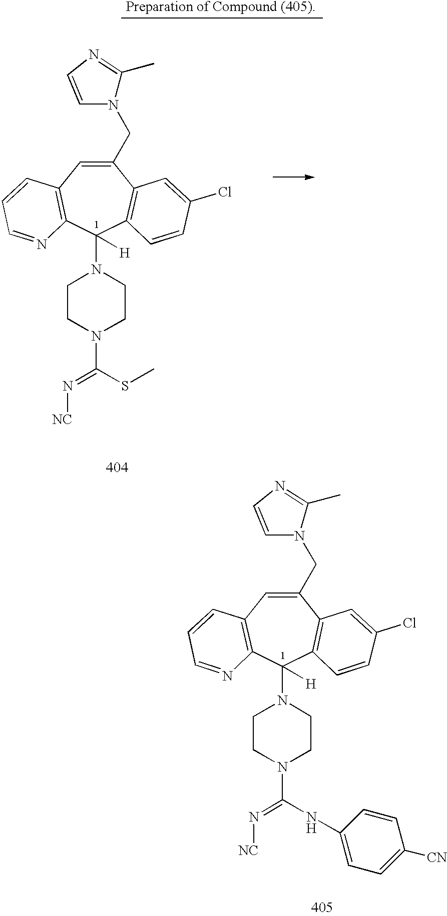

To a solution of para-cyanoanaline (53 mg, 0.45 mmol) in 1 ml N,N-dimethylformamide was added sodium hydride (18 mg, 0.45 mmol). After stirring under a dry nitrogen atmosphere for ½ hour, Compound (404) (isomer 1) from Preparative Example 48 above (40 mg, 0.08 mmol) was added and the reaction mixture stirred at 55° C. for 4 hours. The reaction mixture was cooled to ambient temperature and added to brine. The crude product was extracted with dichloromethane 3 times. The extracts were concentrated and the crude product chromatographed on silica gel using 5% methanolic-ammonia/dichloromethane to obtain 17.6 mg of title product. (405) FABMS M+1=574.1

### Examples 180 and 181

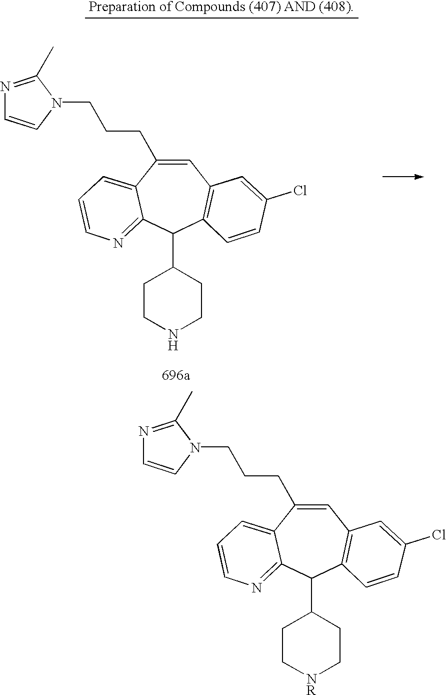

Compound (696a) from Preparative Example 59, Step B, was reacted in the same manner as in Preparative Example 48 and Example 179 substituting the appropriate R reagent to afford the following compounds:

| EX. # | R = | CMPD # | PHYS. DATA |
|---|---|---|---|
| 180 | 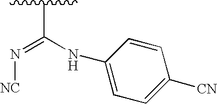 | 407 | FABMS MH+ = 601.1 |
| 181 | 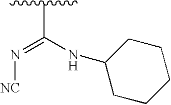 | 408 | FABMS MH+ = 531.1 |

### Preparative Example 49

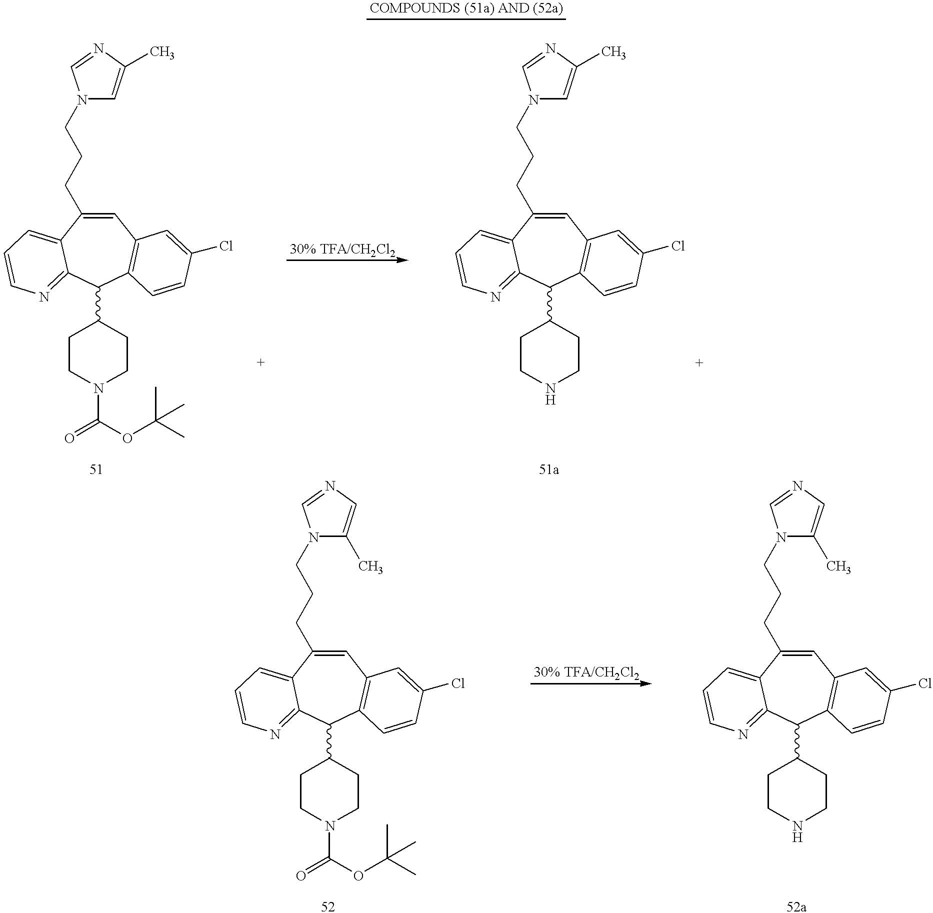

Compounds (51) and (52) from Example 11, Step A, were reacted with TFA in CH₂Cl₂ to afford compounds (51a) and (52a). Library Preparation

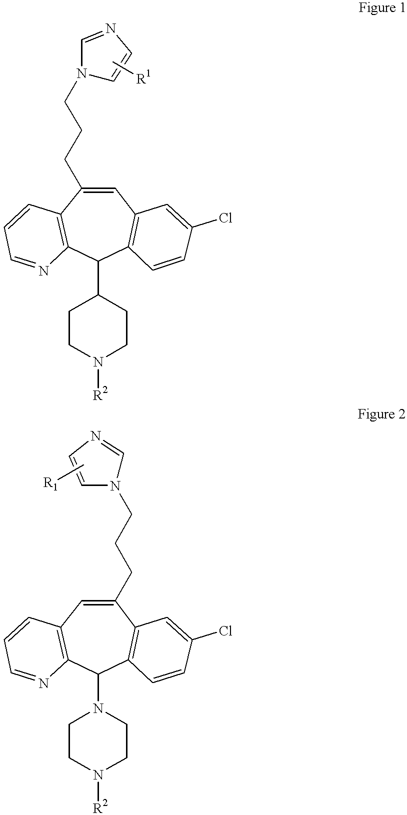

A library of compounds was prepared by solution phase parallel synthesis. A generic structure of these compounds is shown in FIG. 1 above. The R¹ group on the imidazole ring can be H or CH₃, the R² on N-1 of the piperidine is varied in the library.

Library compounds were prepared using compound (29) from Preparative Example 4 or Compounds (51a) or (52a) from Preparative Example 49 above as templates as shown in Scheme A. Synthesis is initiated in test tubes by reacting compound (29), (51a) or (52a) with multiple equivalents of a variety of isocyanates, amines, acids, acid chlorides, sulfonyl chlorides and chloroformates in dichloromethane or chloroform. When urea is the desired product, the reaction can be carried out using isocyanates directly, or alternatively, treating an amine with CDI for several hours, then subject the templates to this solution overnight. When acids are used, the reaction is carried out in the presence of a coupling reagent such as PyBrop and a base such as DIEA overnight. When acid chlorides, sulfonyl chlorides or chloroformates are used, the reaction is typically conducted in the presence of triethylamine. After reaction, an excess amount of polystyrene aminomethyl resin is added to the reaction test tubes, and the reaction allowed to stand overnight. At which time each test tube is filtered through a Bio-Rad Poly-Prep chromatography column into another test tube, and the resin is washed with dichloromethane and MeOH. The combined filtrate solution is concentrated by rotovap evaporation. The residue in each test tube is then dissolved in H₂O /CH₃CN (50/50, containing 1% TFA) and purified by Gilson 215 liquid Handling-HPLC system to give pure product. The product was identified by mass spectroscopy. Library compounds prepared in this fashion are shown in Table 1 and Table 2.

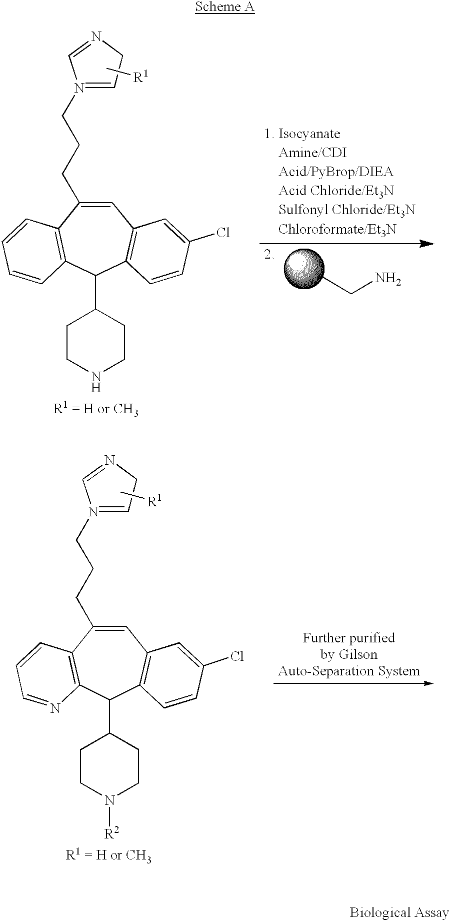

### Examples 182-283

TABLE 1

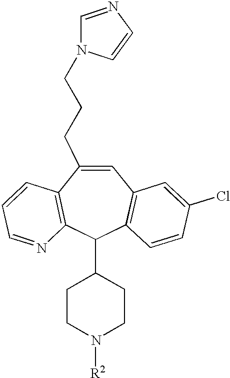

| EXAMPLE #. | R² | COMPOUND # | PHYSICAL DATA |
|---|---|---|---|
| 182 | 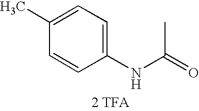 | 409 | Mass spec. MH⁺ = 552 |
| 183 | 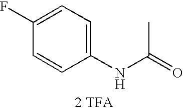 | 410 | Mass spec. MH⁺ = 556 |
| 184 | 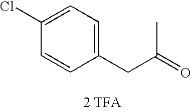 | 411 | Mass spec. MH⁺ = 571 |
| 185 | 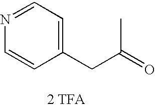 | 412 | Mass spec. MH⁺ = 538 |
| 186 | 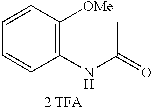 | 413 | Mass spec. MH⁺ = 568 |
| 187 | 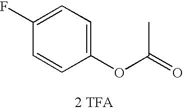 | 414 | Mass spec. MH⁺ = 557 |
| 188 | 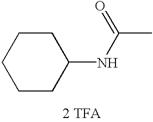 | 415 | Mass spec. MH⁺ = 544 |
| 189 | 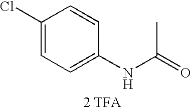 | 416 | Mass spec. MH⁺ = 572 |
| 190 | 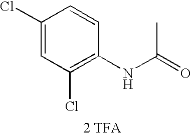 | 417 | Mass spec. MH⁺ = 606 |
| 191 | 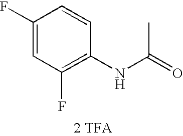 | 418 | Mass spec. MH⁺ = 574 |
| 192 | 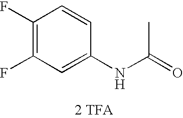 | 419 | Mass spec. MH⁺ = 574 |
| 193 | 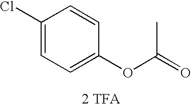 | 420 | Mass spec. MH⁺ = 573 |
| 194 | 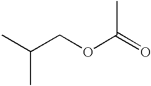 | 421 | Mass spec. MH⁺ = 519 |
| 195 | 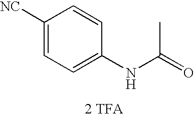 | 422 | Mass spec. MH⁺ = 563 |
| 196 | 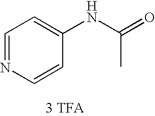 | 423 | Mass spec. MH⁺ = 539 |
| 197 | 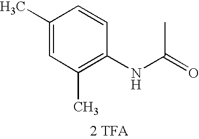 | 424 | Mass spec. MH⁺ = 566 |
| 198 | 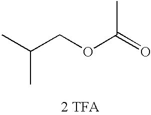 | 425 | Mass spec. MH⁺ = 505 |
| 199 | 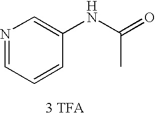 | 426 | Mass spec. MH⁺ = 539 |
| 200 | 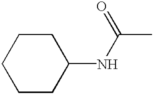 | 427 | Mass spec. MH⁺ = 544 |
| 201 | 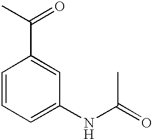 | 428 | Mass spec. MH⁺ = 580 |
| 202 | 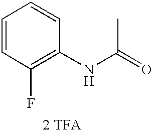 | 429 | Mass spec. MH⁺ = 556 |
| 203 | 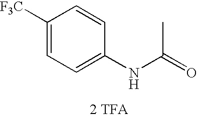 | 430 | Mass spec. MH⁺ = 606 |
| 204 | 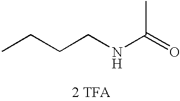 | 431 | Mass spec. MH⁺ = 518 |
| 205 | 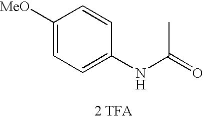 | 432 | Mass spec. MH⁺ = 568 |
| 206 | 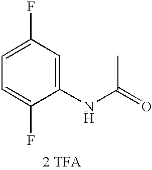 | 433 | Mass spec. MH⁺ = 574 |
| 207 | 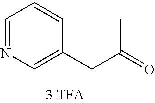 | 434 | Mass spec. MH⁺ = 538 |
| 208 | 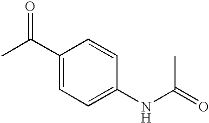 | 435 | Mass spec. MH⁺ = 580 |
| 209 | 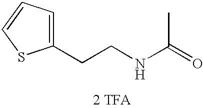 | 436 | Mass spec. MH⁺ = 572 |
| 210 | 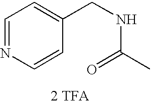 | 437 | Mass spec. MH⁺ = 553 |
| 211 | 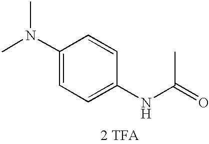 | 438 | Mass spec. MH⁺ = 581 |
| 212 | 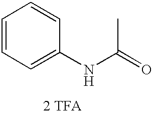 | 439 | Mass spec. MH⁺ = 538 |
| 213 | 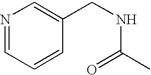 | 440 | Mass spec. MH⁺ = 553 |
| 214 | 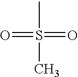 | 441 | Mass spec. MH⁺ = 497 |
| 215 | 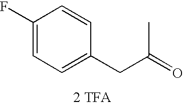 | 442 | Mass spec. MH⁺ = 555 |
| 216 | 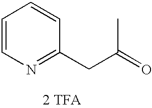 | 443 | Mass spec. MH⁺ = 538 |
| 217 | 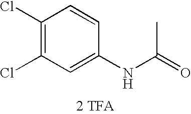 | 444 | Mass spec. MH⁺ = 606 |
| 218 | 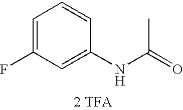 | 445 | Mass spec. MH⁺ = 556 |
| 219 | 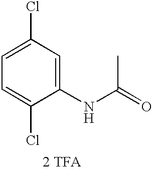 | 446 | Mass spec. MH⁺ = 606 |
| 220 | 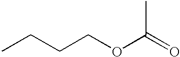 | 447 | Mass spec. MH⁺ = 519 |
| 221 | 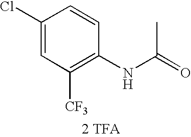 | 448 | Mass spec. MH⁺ = 640 |
| 222 | 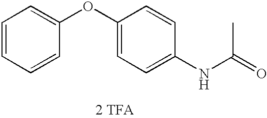 | 449 | Mass spec. MH⁺ = 630 |
| 223 | 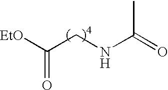 | 450 | Mass spec. MH⁺ = 604 |
| 224 | 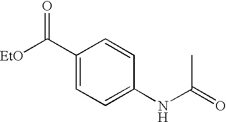 | 451 | Mass spec. MH⁺ = 610 |
| 225 | 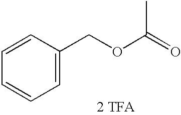 | 452 | Mass spec. MH⁺ = 553 |
| 226 | 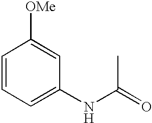 | 453 | Mass spec. MH⁺ = 568 |
| 227 | 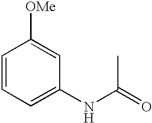 | 454 | Mass spec. M⁺ = 572 |
| 228 | 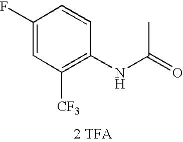 | 455 | Mass spec. MH⁺ = 624 |
| 229 | 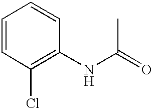 | 456 | Mass spec. MH⁺ = 572 |
| 230 | 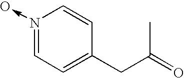 | 457 | Mass spec. MH⁺ = 554 |
| 231 | 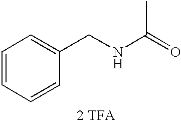 | 458 | Mass spec. MH⁺ = 552 |
| 232 | 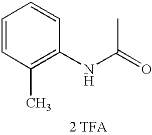 | 459 | Mass spec. MH⁺ = 552 |
| 233 | 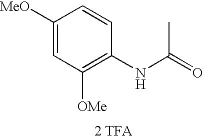 | 460 | Mass spec. MH⁺ = 598 |
| 234 | 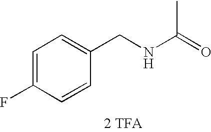 | 461 | Mass spec. MH⁺ = 570 |
| 235 | 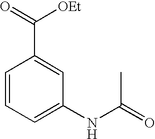 | 462 | Mass spec. MH⁺ = 610 |
| 236 | 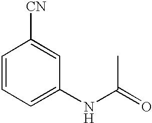 | 463 | Mass spec. MH⁺ = 563 |
| 237 | 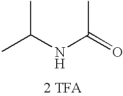 | 464 | Mass spec. MH⁺ = 504 |
| 238 | 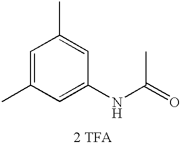 | 465 | Mass spec. MH⁺ = 566 |
| 239 | 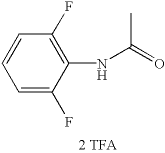 | 466 | Mass spec. MH⁺ = 574 |
| 240 | 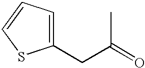 | 467 | Mass spec. MH⁺ = 543 |
| 241 | 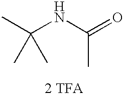 | 468 | Mass spec. MH⁺ = 518 |
| 242 | 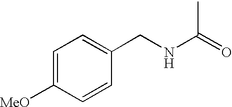 | 469 | Mass spec. MH⁺ = 582 |
| 243 | 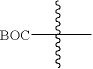 | 470 | Mass spec. MH⁺ = 519 |
| 244 | 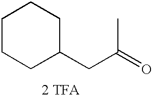 | 471 | Mass spec. MH⁺ = 543 |
| 245 | 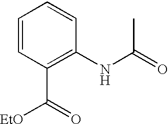 | 472 | Mass spec. MH⁺ = 610 |
| 246 | 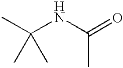 | 473 | Mass spec. MH⁺ = 518 |
| 247 | 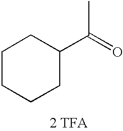 | 474 | Mass spec. MH⁺ = 529 |
| 248 | 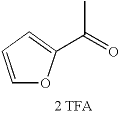 | 475 | Mass spec. MH⁺ = 513 |
| 249 | 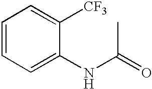 | 476 | Mass spec. MH⁺ = 606 |
| 250 | 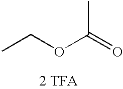 | 477 | Mass spec. MH⁺ = 491 |
| 251 | 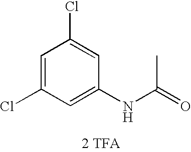 | 478 | Mass spec. MH⁺ = 606 |
| 252 | 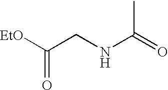 | 479 | Mass spec. MH⁺ = 548 |
| 253 | 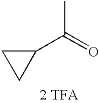 | 480 | Mass spec. MH⁺ = 487 |
| 254 | 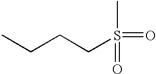 | 481 | Mass spec. MH⁺ = 539 |
| 255 | 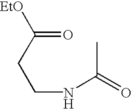 | 482 | Mass spec. MH⁺ = 562 |
| 256 | 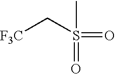 | 483 | Mass spec. MH⁺ = 565 |
| 257 | 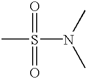 | 484 | Mass spec. MH⁺ = 526 |
| 258 | 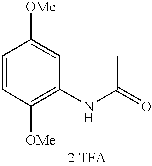 | 485 | Mass spec. MH⁺ = 598 |
| 259 | 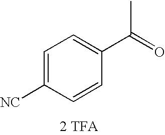 | 486 | Mass spec. MH⁺ = 548 |
| 260 | 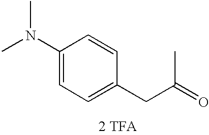 | 487 | Mass spec. MH⁺ = 580 |
| 261 | 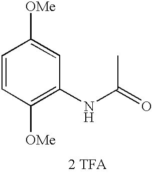 | 488 | Mass spec. MH⁺ = 598 |
| 262 | 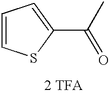 | 489 | Mass spec. MH⁺ = 529 |
| 263 | 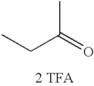 | 490 | Mass spec. MH⁺ = 475 |
| 264 | 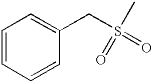 | 491 | Mass spec. MH⁺ = 573 |
| 265 | 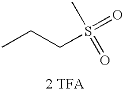 | 492 | Mass spec. MH⁺ = 525 |
| 266 | 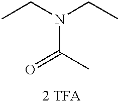 | 493 | Mass spec. MH⁺ = 518 |
| 267 | 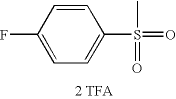 | 494 | Mass spec. MH⁺ = 577 |
| 268 | 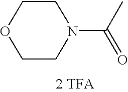 | 495 | Mass spec. MH⁺ = 532 |
| 269 | 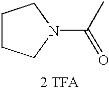 | 496 | Mass spec. MH⁺ = 516 |
| 270 | 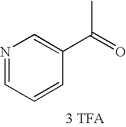 | 497 | Mass spec. MH⁺ = 524 |
| 271 | 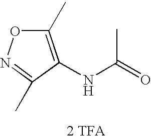 | 498 | Mass spec. MH⁺ = 557 |
| 272 | 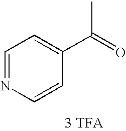 | 499 | Mass spec. MH⁺ = 524 |
| 273 | 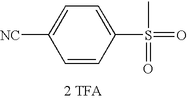 | 500 | Mass spec. MH⁺ = 584 |
| 274 | 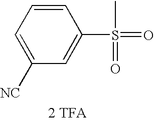 | 501 | Mass spec. MH⁺ = 584 |
| 275 | 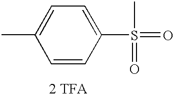 | 502 | Mass spec. MH⁺ = 573 |
| 276 | 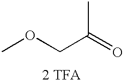 | 503 | Mass spec. MH⁺ = 491 |
| 277 | 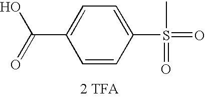 | 504 | Mass spec. MH⁺ = 603 |
| 278 | 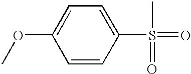 | 505 | Mass spec. MH⁺ = 589 |
| 279 | 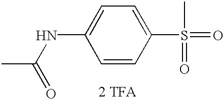 | 506 | Mass spec. MH⁺ = 616 |
| 280 | 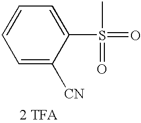 | 507 | Mass spec. MH⁺ = 584 |
| 281 | 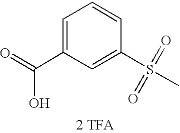 | 508 | Mass spec. MH⁺ = 603 |
| 282 | 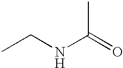 | 509 | Mass spec. MH⁺ = 490 |
| 283 | 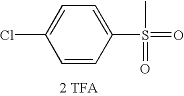 | 510 | Mass spec. MH⁺ = 593 |

### Examples 284-377

TABLE 2

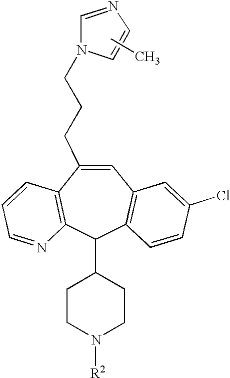

| EXAMPLE # | R² | COMPOUND # | MH⁺ |
|---|---|---|---|
| 284 | 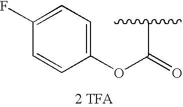 | 511 | 571 |
| 285 | 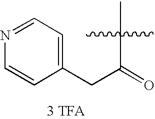 | 512 | 552 |
| 286 | 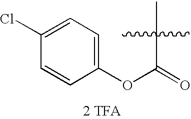 | 513 | 587 |
| 287 | 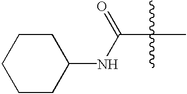 | 514 | 558 |
| 288 | 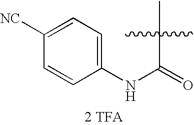 | 515 | 577 |
| 289 | 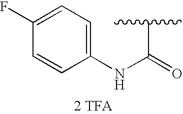 | 516 | 570 |
| 290 | 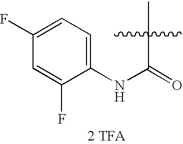 | 517 | 588 |
| 291 | 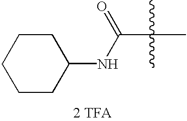 | 518 | 558 |
| 292 | 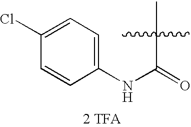 | 519 | 586 |
| 293 | 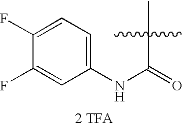 | 520 | 588 |
| 294 | 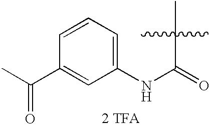 | 521 | 594 |
| 295 | 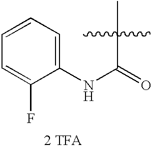 | 522 | 570 |
| 296 | 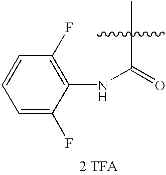 | 523 | 588 |
| 297 | 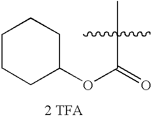 | 524 | 559 |
| 298 | 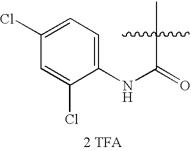 | 525 | 620 |
| 299 | 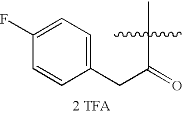 | 526 | 569 |
| 300 | 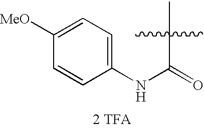 | 527 | 582 |
| 301 | 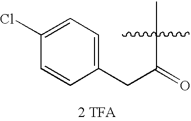 | 528 | 585 |
| 302 | 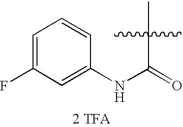 | 529 | 570 |
| 303 | 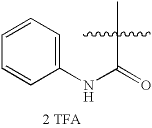 | 530 | 552 |
| 304 | 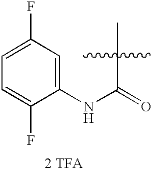 | 531 | 588 |
| 305 | 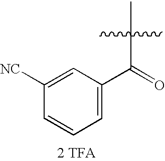 | 532 | 562 |
| 306 | 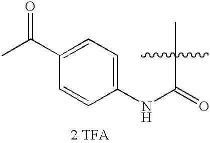 | 533 | 594 |
| 307 | 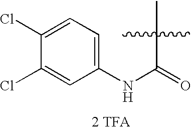 | 534 | 620 |
| 308 | 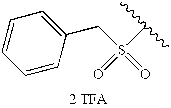 | 535 | 587 |
| 309 | 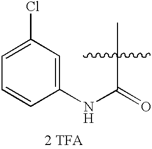 | 536 | 586 |
| 310 | 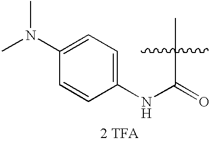 | 537 | 595 |
| 311 | 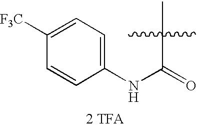 | 538 | 620 |
| 312 | 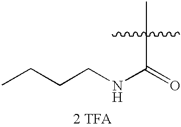 | 539 | 532 |
| 313 | 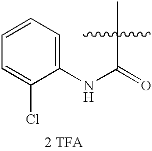 | 540 | 586 |
| 314 | 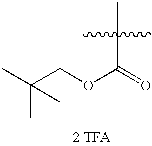 | 541 | 547 |
| 315 | 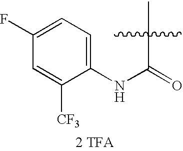 | 542 | 638 |
| 316 | 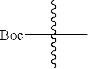 | 543 | 533 |
| 317 | 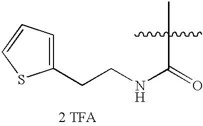 | 544 | 586 |
| 318 | 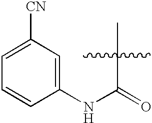 | 545 | 577 |
| 319 | 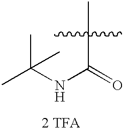 | 546 | 532 |
| 320 | 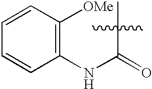 | 547 | 582 |
| 321 | 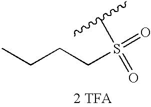 | 548 | 553 |
| 322 | 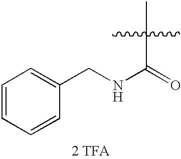 | 549 | 566 |
| 323 | 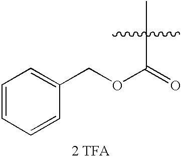 | 550 | 567 |
| 324 | 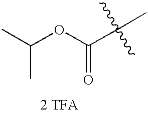 | 551 | 519 |
| 325 | 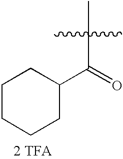 | 552 | 543 |
| 326 | 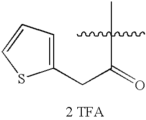 | 553 | 557 |
| 327 | 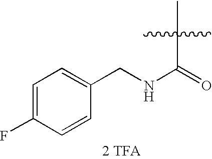 | 554 | 584 |
| 328 | 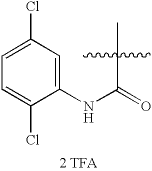 | 555 | 620 |
| 329 | 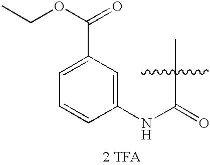 | 556 | 624 |
| 330 | 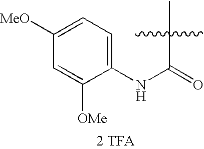 | 557 | 612 |
| 331 | 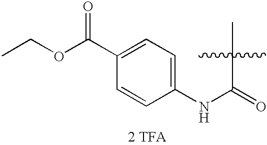 | 558 | 624 |
| 332 | 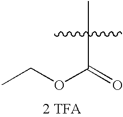 | 559 | 505 |
| 333 | 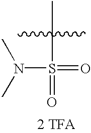 | 560 | 540 |
| 334 | 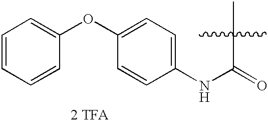 | 561 | 644 |
| 335 | 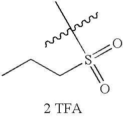 | 562 | 539 |
| 336 | 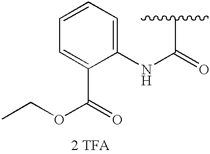 | 563 | 624 |
| 337 | 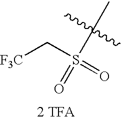 | 564 | 579 |
| 338 | 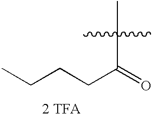 | 565 | 517 |
| 339 | 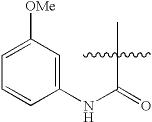 | 566 | 582 |
| 340 | 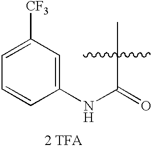 | 567 | 620 |
| 341 | 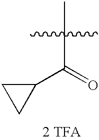 | 568 | 501 |
| 342 | 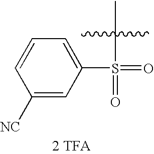 | 569 | 598 |
| 343 | 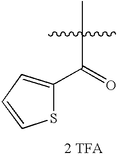 | 570 | 543 |
| 344 | 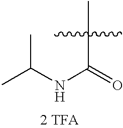 | 571 | 518 |
| 345 | 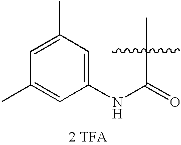 | 572 | 580 |
| 346 | 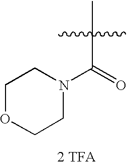 | 573 | 546 |
| 347 | 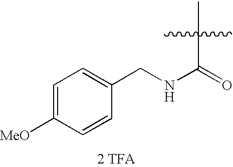 | 574 | 596 |
| 348 | 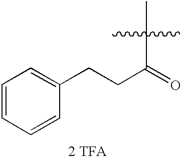 | 575 | 565 |
| 349 | 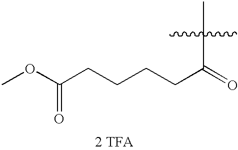 | 576 | 575 |
| 350 | 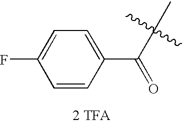 | 577 | 555 |
| 351 | 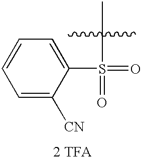 | 578 | 598 |
| 352 | 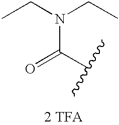 | 579 | 532 |
| 353 | 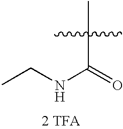 | 580 | 504 |
| 354 | 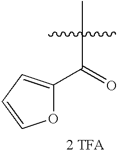 | 581 | 527 |
| 355 | 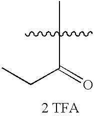 | 582 | 489 |
| 356 | 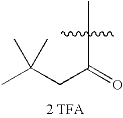 | 583 | 531 |
| 357 | 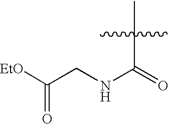 | 584 | 562 |
| 358 | 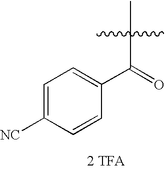 | 585 | 562 |
| 359 | 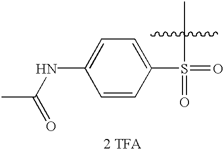 | 586 | 630 |
| 360 | 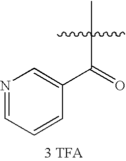 | 587 | 538 |
| 361 | 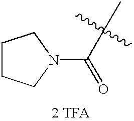 | 588 | 530 |
| 362 | 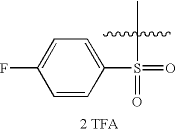 | 589 | 591 |
| 363 | 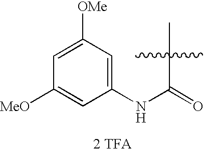 | 590 | 612 |
| 364 | 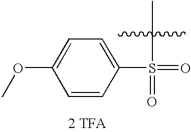 | 591 | 603 |
| 365 | 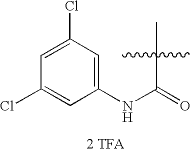 | 592 | 620 |
| 366 | 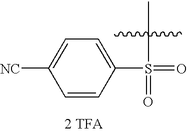 | 593 | 598 |
| 367 | 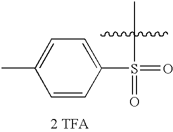 | 594 | 587 |
| 368 | 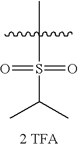 | 595 | 539 |
| 369 | 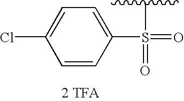 | 596 | 607 |
| 370 | 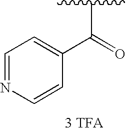 | 597 | 538 |
| 371 | 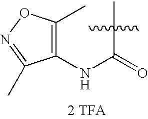 | 598 | 571 |
| 372 | 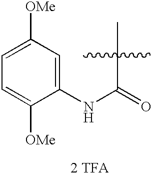 | 599 | 612 |
| 373 | 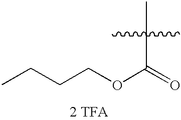 | 600 | 533 |
| 374 | 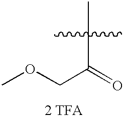 | 601 | 505 |
| 375 | 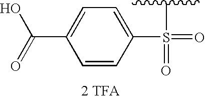 | 602 | 617 |
| 376 | 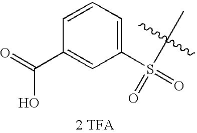 | 603 | 617 |
| 377 | 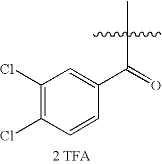 | 604 | 605 |

### Preparative Example 50

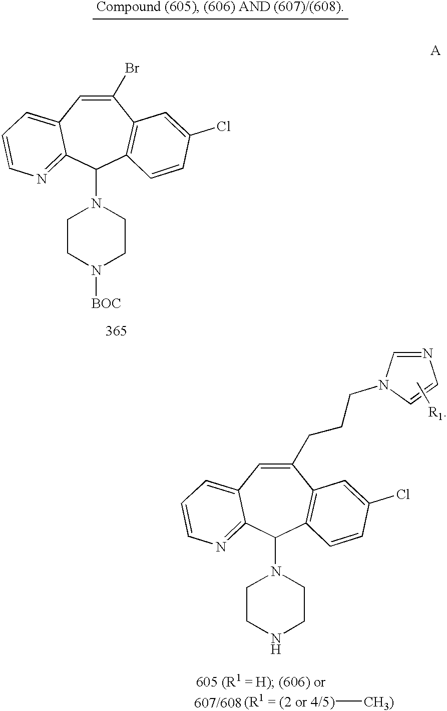

Compound (365) from Preparative Example 41 was reacted in essentially the same manner as in Preparative Example 4, substituting the appropriate imidazole to obtain Compound (605) wherein R¹=H or Compounds (606) and (607)/(608) wherein R¹=(2 or 4/5)CH₃.

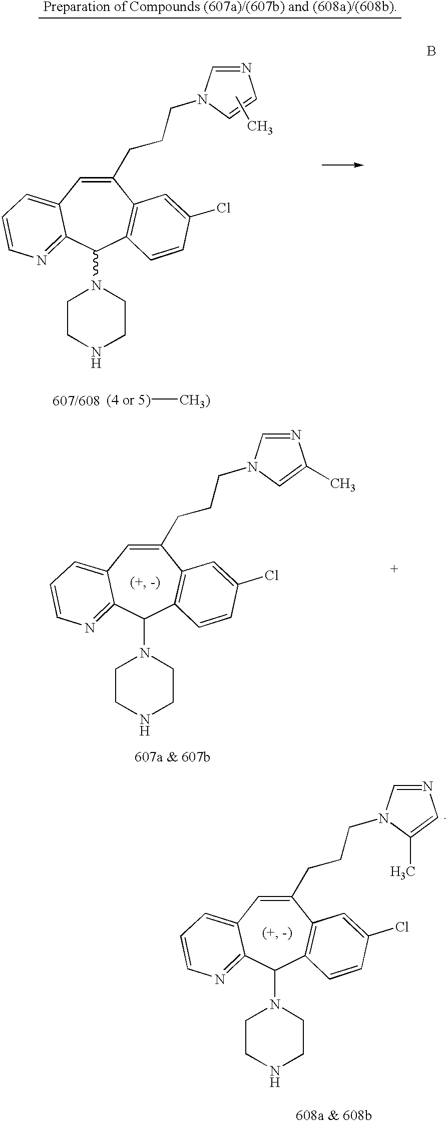

Compounds (607) and (608) from Step A above were treated in the same manner as described in Example 11 to afford pure (+,−) 4-methyl imidazole, and pure (+,−) 5-methyl imidazole enantiomers; Compound (607a),(607b) and Compound (608a), (608b) respectively.

A library of compounds was prepared by the method described above starting with Compound (605), Compound (606), Compounds (607)/(608), (607a), (607b) or Compounds (608a) or (608b) used as the templates in Scheme 2. A generic structure of these compounds is shown in FIG. 2 above. The R¹ group on the imidazole ring can be H or CH₃, the R² on N-1 of the piperazine is varied in the library. Library compounds prepared in this fashion are shown in Table 3, Table 4 and Table 5.

### Examples (378)-(396)

TABLE 3

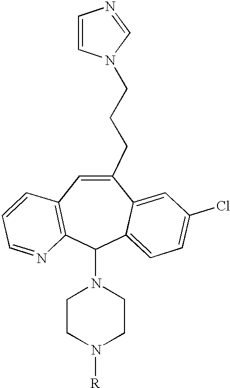

| EXAMPLE # | R² | COMPOUND # | PHYSICAL DATA |
|---|---|---|---|
| 378 | 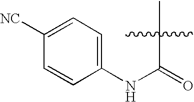 | 607 | 564 |
| 379 | 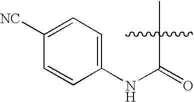 | 608 1^{st} Enantiomer | 564 |
| 380 | 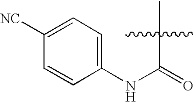 | 609 2^{nd} Enantiomer | 564 |
| 381 | 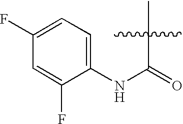 | 610 | 575 |
| 382 | 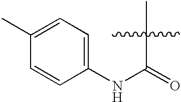 | 611 | 553 |
| 383 | 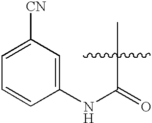 | 612 | 564 |
| 384 | 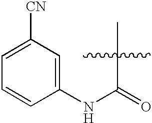 | 613 | 564 |
| 385 | 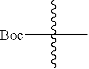 | 614 | 520 |
| 386 | 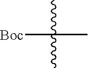 | 615 1^{st} Isomer | 520 |
| 387 | 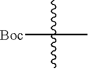 | 616 2^{nd} Isomer | 520 |
| 388 | 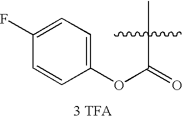 | 617 | 558 |
| 389 | 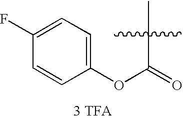 | 618 | 557 |
| 390 | 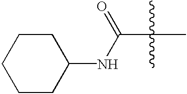 | 619 | 545 |
| 391 | 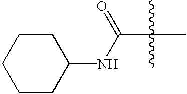 | 620 1^{st} Isomer | 545 |
| 392 | 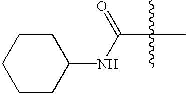 | 621 2^{nd} Isomer | 545 |
| 393 | 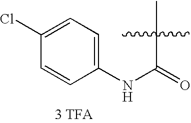 | 622 | 573 |
| 394 | 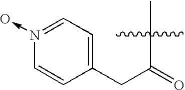 | 623 | 555 |
| 395 | 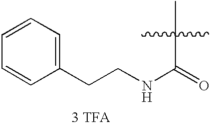 | 624 | 567 |
| 396 | H 4 TFA | 625 | 420 |

### Examples 397-401

TABLE 4

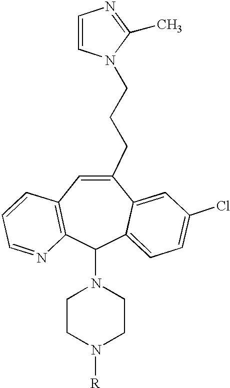

| EXAMPLE # | R² | COMPOUND # | PHYSICAL DATA |
|---|---|---|---|
| 397 | 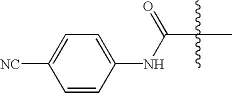 | 626 2 Isomers | Mass spec. MH+ = 578 |
| 398 | 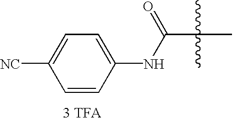 | 627 2^{nd} Enantiomer | Mass spec. MH+ = 578 |
| 399 | 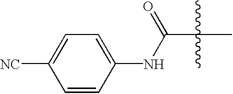 | 628 2^{nd} Enantiomer | Mass spec. MH+ = 578 |
| 400 | 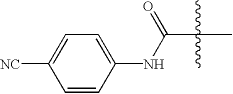 | 629 1^{st} Enantiomer | Mass spec. MH+ = 578 |
| 401 | 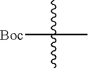 | 630 2 Isomers | Mass spec. MH+ = 534 |

### Examples 402-406

TABLE 5

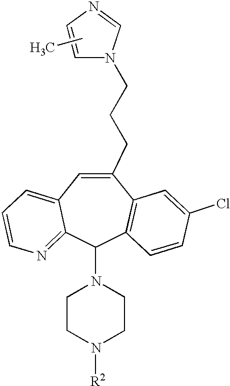

| EXAMPLE # | R² | COMPOUND # | PHYSICAL DATA |
|---|---|---|---|
| 402 | 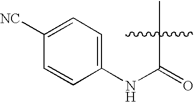 | 631 Mixture of 4-Me and 5-Me | Mass spec. MH+ = 578 |
| 403 | 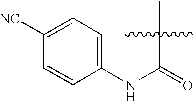 | 632 2^{nd} enantiomer of 4-Me | Mass spec. MH+ = 578 |
| 404 | 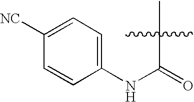 | 633 2^{nd} enantiomer of 5-Me 1^{st} enantiomer of 4-Me | Mass spec. MH+ = 578 |
| 405 | 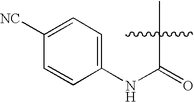 | 634 1^{st} enantiomer of 5-Me | Mass spec. MH+ = 578 |
| 406 | 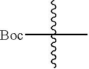 | 635 Mixture of 4-Me and 5-Me | Mass spec. MH+ = 534 |

### Preparative Example 51

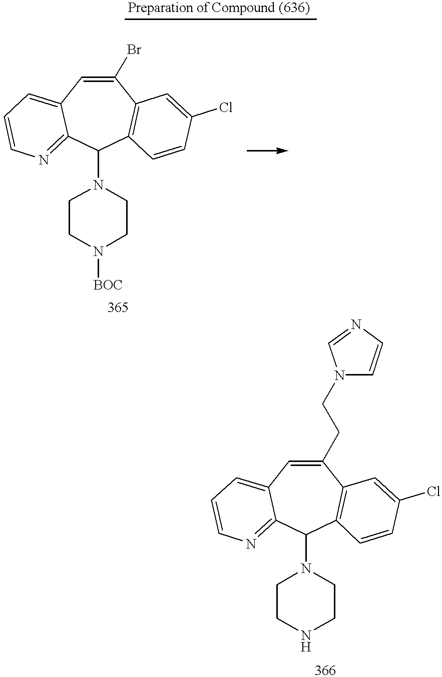

Compound (365) from Preparative Example 41, was reacted in essentially the same manner as Preparative Example 35 substituting Imidazole for 1-Methyl Imidazole in Step B to afford Compound (636) (MH₊=406). Compound (636) was then reacted in the library fashion as described above following the procedure of Scheme 2 to afford the compounds in Table 6 below:

TABLE 6

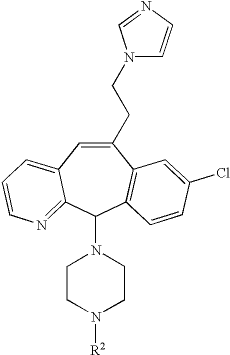

| EXAMPLE # | R² | COMPOUND # | PHYSICAL DATA |
|---|---|---|---|
| 407 | 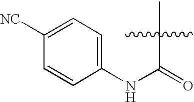 | 637 | Mass spec. MH+ = 550 |
| 408 | 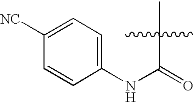 | 638 2^{nd} Enantiomer | Mass spec. MH+ = 550 |
| 409 | 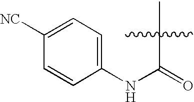 | 639 1^{ST} Enantiomer | Mass spec. MH+ = 550 |
| 410 | 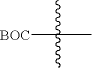 | 640 | Mass spec. MH+ = 506 |

### Preparative Example 52

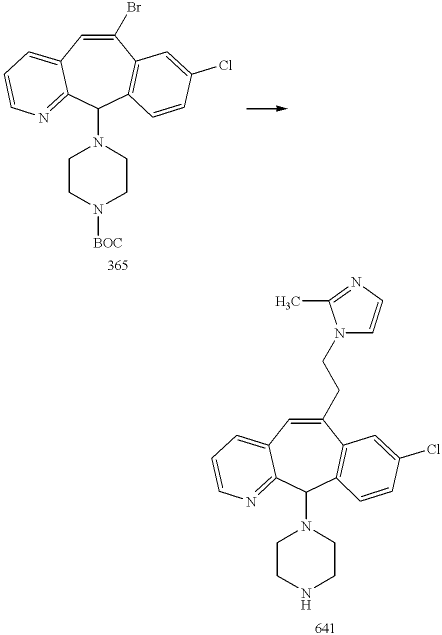

Compound (365) was reacted as above in Preparative Example 51, substituting 1-Methyl Imidazole for Imidazole to afford Compound (641) (MH₊=420). Compound (641) was then further reacted in the Library fashion described above following the procedure in Scheme 2 to afford the compounds in Table 7 below:

TABLE 7

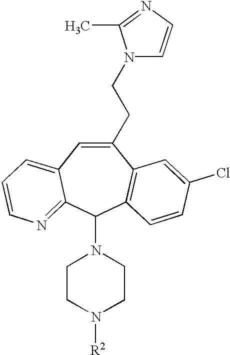

| EXAMPLE # | R² | COMPOUND # | PHYSICAL DATA |
|---|---|---|---|
| 411 | 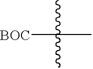 | 642 | Mass spec. MH⁺ = 520 |
| 412 | 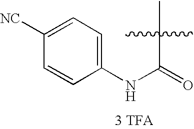 | 643 | Mass spec. MH⁺ = 564 |
| 413 | 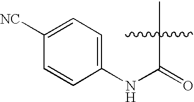 | 644 1^{st} Enantiomer | Mass spec. MH⁺ = 564 |
| 414 | 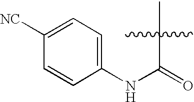 | 645 2^{nd} Enantiomer | Mass spec. MH⁺ = 564 |

### Example 415

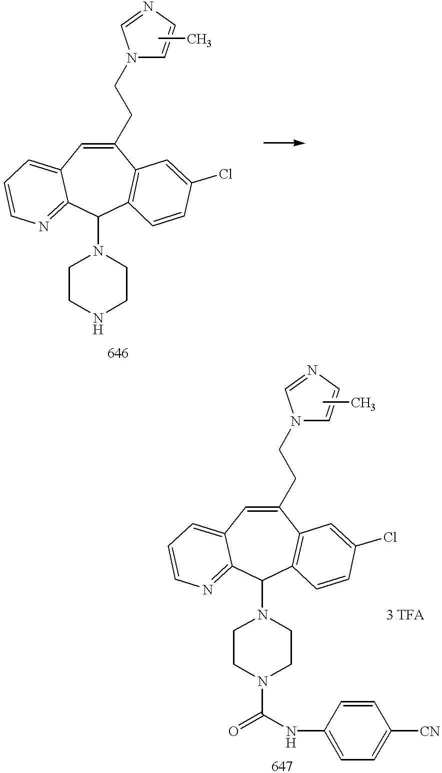

In the essentially the same manner as in Preparative Example 52 above, g 4-methylimidazole, the intermediate amine template was prepared (646). This was then reacted in essentially the same manner as in 411-414 above to afford the product Compound (647) as a mixture of 4 and idazole isomers (Mass spec. MH⁺=564).

### Preparative Example 53

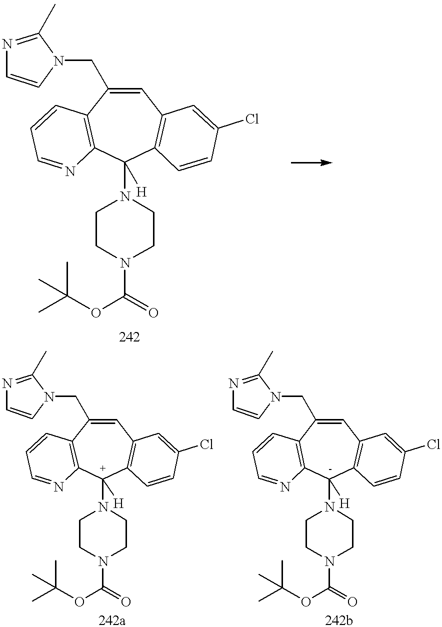

The racemic Compound (242) from Example 91 was separated by preparative chiral chromatography (Chiralpack AD, 5 cm×50 cm column, flow rate 100 mL/min., 20% 2-propanol/hexane+0.2% diethylamine) to afford the two enantiomers (242a) and (242b).

Compound (242a), [α]_{D}²⁵=+144.8° (3.16 mg/2 mL MeOH)

Compound (242b), [α]_{D}²⁵=−144.8° (2.93 mg/2 mL MeOH)

### Preparative Example 54

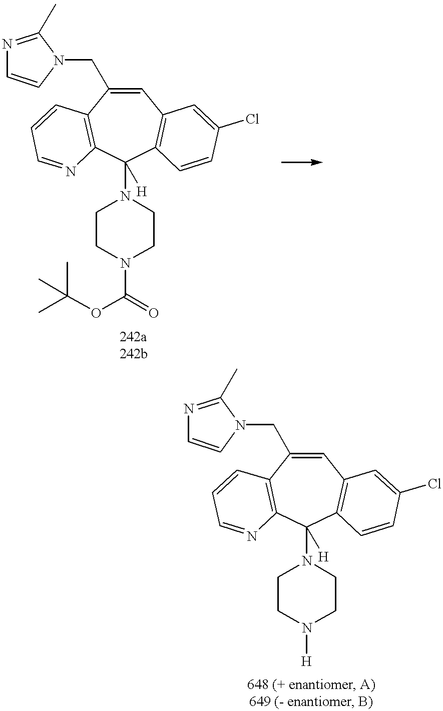

Compounds (242a) and (242b) from Preparative Example 53 above were reacted separately in essentially the same manner as Preparative Example 19, Step D to obtain the hydrochloride salt of compounds Compound (648) and Compound (649).

(648) (+ enantiomer, isomer A), MH+=406.1793

(649) (− enantiomer, isomer B), MH+=406.1789

### Preparative Example 55

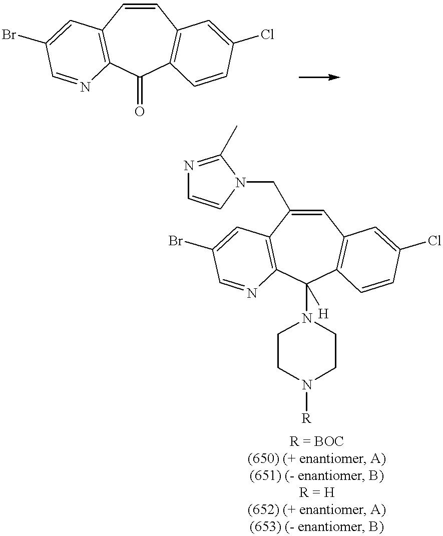

3-bromo-8-chloroazaketone (U.S. Pat. No. 5,977,128, Preparative Example 11, step A, (1999)) was reacted in essentially the same manner as in Preparative Example 23, and Example 91 to obtain the N-BOC derivatives (650) and (651). Compounds (650) and (651) were then reacted separately in essentially the same manner as in Preparative Example 19, Step D to obtain the enantiomers (652) (+ enantiomer, isomer A) and (653) (− enantiomer, isomer B).

Compound (650), BOC derivative, [α]_{D}²⁵=+69.6° (2.5 mg/2 mL MeOH)

Compound (651), BOC derivative, [α]_{D}²⁵=−90.0° (3.3 mg/2 mL MeOH)

Compound (652) (+ enantiomer, isomer A), MH₊=485

Compound (653) (− enantiomer, isomer B), MH₊=485

### Preparative Example 56

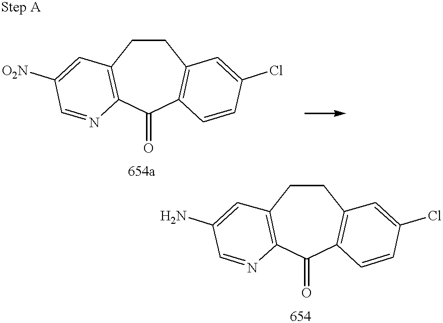

Compound (654a) (202 g; 0.7 mole) (J. Org. Chem. 1998, 63, 445) was dissolved in ethanol (5 L). To this mixture was added 12 N HCl (80 ml) and iron powder (180 g) and the reaction was refluxed over night. Additional HCl and iron was added to complete the reaction. The reaction mixture was filtered and the precipitate washed with hot methanol (1 L). The filtrate was concentrated under vacuum to approximately 600 ml then partitioned between 4 L CH₂CL₂ and 1.3 L of 1.3 N NaOH. The organic layer was dried over MgSO₄ and filtered hot. The filtrate was concentrated under vacuum to give the aminoketone Compound (654) (184 g).

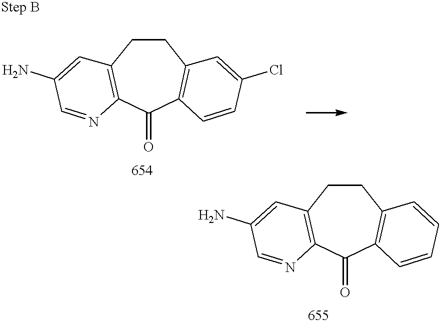

Compound (654) from Step A above (15 g; 57.98 mmol), was dissolved in 750 mL of ethanol containing 3.75 g of 5% Pd/C (50% in water) and 37.69 g (579.82 m mol) of ammonium formate. The mixture was brought to reflux for 2.5 hr then stirred at room temperature overnight. The reaction was filtered concentrated under vacuum and chromatographed on silica gel using 95:5 methylene chloride (saturated with ammonia) and methanol to give 6.15 g of the pure product Compound (655) as a yellow solid.

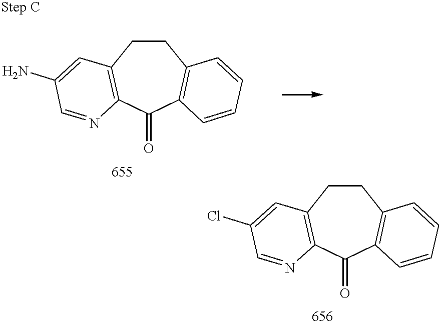

To a slurry of Compound (655) (4.79 g; 21.37 mmol) from Step A above, in 75 mL of acetonitrile cooled to 0° C. and under nitrogen, was added t-butylnitrite (10.31 g; 32.05 mmol) and CuCl₂ (3.45 g; 24.64 mmol). The mixture was warmed to room temp stirrd over night and then concentrated under vacuum. The residue was slurried in 30 mL of 1N HCl, then neutralized with aqueous NH₄OH and extracted with 3×100 mL of ethyl acetate. The organic layer was dried over Na₂SO₄. concentrated under vacuum, and chromatographed on silica gel using hexane:ethyl acetate (70:30) to obtain the pure product Compound (656).

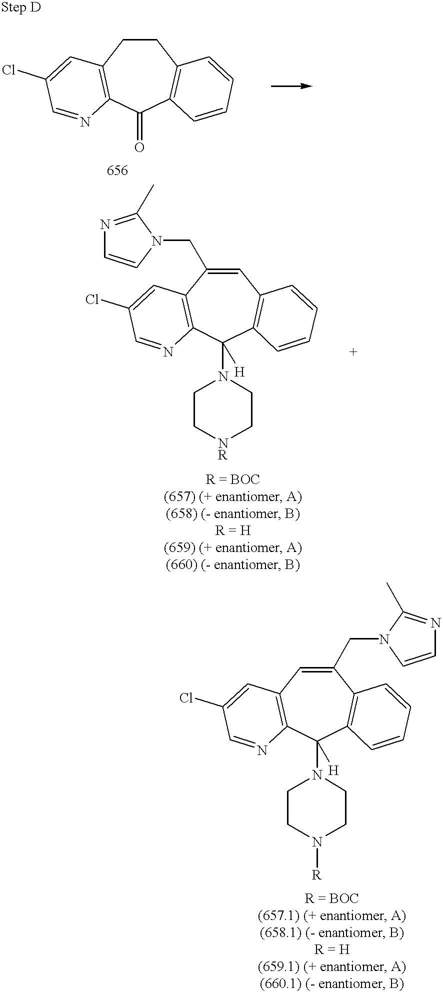

Compound (656) from Step B above was reacted in essentially the same manner as in Preparative Example 23, and then Example 91 to obtain the N-BOC derivatives (657), (658), (657.1) and (658.1). Compounds (657), (658), (657.1) and (658.1) were then reacted separately in essentially the same manner as in Preparative Example 19, Step D to obtain the enantiomers (659) (+enantiomer, isomer A), (659.1) (+ enantiomer, isomer A), (660) (− enantiomer, isomer B) and (660.1) (− enantiomer, isomer B).

Compound (657), BOC derivative, [α]_{D}²⁵=+59,9° (3.3 mg/ 2 mL MeOH)

Compound (658), BOC derivative, [α]_{D}²⁵=−57.1° (3.3 mg/ 2 mL MeOH)

Compound (659), (+ enantiomer, isomer A), MH₊=406

Compound (660), (− enantiomer, isomer B), MH₊=406

Compound (659.1), (+ enantiomer, isomer A), MH₊=406

Compound (660.1), (− enantiomer, isomer B), MH₊=406

### Preparative Example 57

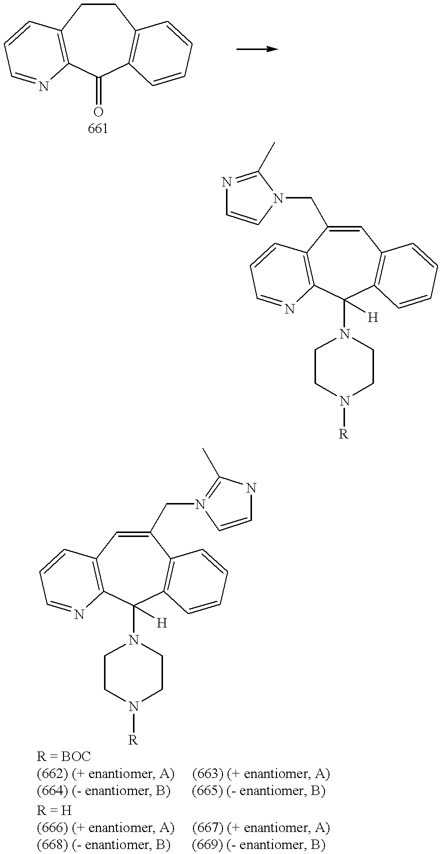

Compound (661) was reacted in essentially the same manner as in Preparative Example 23, and then Example 91 to obtain the N-BOC derivatives (662), (663), (664) and (665). Compounds (662), (663), (664) and (665) were then reacted separately in essentially the same manner as in Preparative Example 19, Step D to obtain the enantiomers (666) and (667) (+ enantiomer, isomer A) and (668) and (669)(− enantiomer, isomer B). The C5 and C-6 vinyl bromide intermediates were separated by silica gel chromatography using hexane:ethyl acetate (80:20) in essentially the same manner as was described in Preparative Example 23, Step B.

Compound (662), BOC derivative

Compound (663), BOC derivative

Compound (664), BOC derivative

Compound (665), BOC derivative

Compound (666) (+ enantiomer, isomer A), MH+=372

Compound (667) (+ enantiomer, isomer A), MH+=372

Compound (668) (− enantiomer, isomer B), MH+=372

Compound (669) (− enantiomer, isomer B), MH+=372

### Preparative Example 58

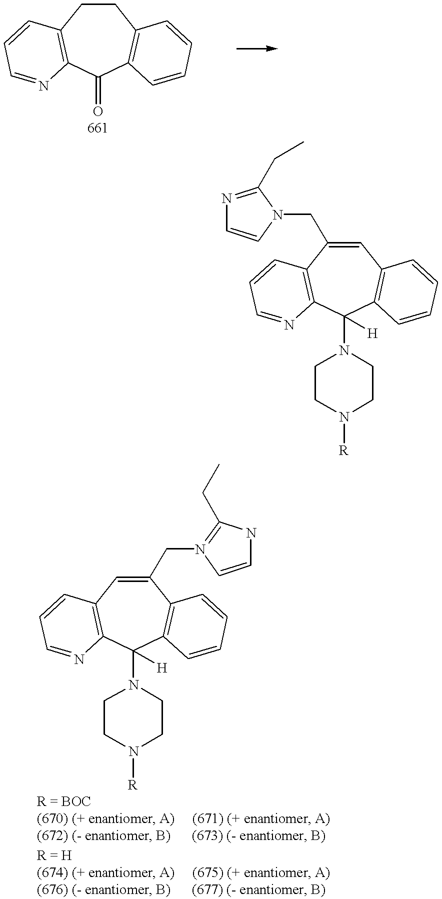

Compound (661) was reacted in essentially the same manner as in Preparative Example 23, and Example 91 substituting 2-ethylimidazole for 2-methylimidazole, to obtain the N-BOC derivatives (670), (671), (672) and (673). Compounds (670), (671), (672) and (673) were then reacted separately in essentially the same manner as in Preparative Example 19, Step D, to obtain the enantiomers (674) and (675) (+ enantiomer, isomer A) and (676) and (677) (− enantiomer, isomer B). The C5 and C-6 vinyl bromide intermediates were separated by silica gel chromatography using hexane:ethyl acetate (80:20) as described in Preparative Example 23, Step B.

Compound (670), BOC derivative, (+ enantiomer, A)

Compound (671), BOC derivative, (+ enantiomer, A)

Compound (672), BOC derivative, (− enantiomer, B)

Compound (673), BOC derivative, (− enantiomer, B)

Compound (674), (+ enantiomer, isomer A), MH+=386

Compound (675), (+ enantiomer, isomer A), MH+=386

Compound (676), (− enantiomer, isomer B), MH+=386

Compound (677), (− enantiomer, isomer B), MH+=386

### Examples 416-419

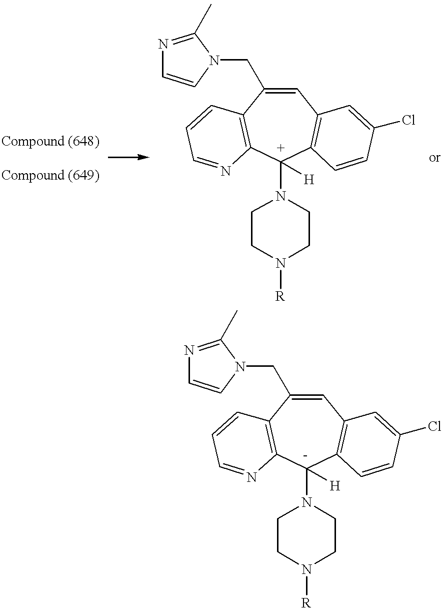

The appropriate (+) enantiomer (648) or (−) enantiomer (649) from Preparative Example 54 above, was taken up in CH₂Cl₂ treated with the corresponding isocyanate and stirred at room temperature over night. The crude product was purified directly by silica gel preparative thin layer chromatography or silica gel column chromatography to afford the following compounds in Table 8 below:

TABLE 8

| Example # | R | Enantiomer | Comp # | Phys. Data. |
|---|---|---|---|---|
| 416 | 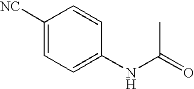 | + | 678 | Mp = 162.2-165.6° C. [α]_{D}²⁵ = +98.2°(3 mg/2 mL MeOH) |
| 417 | 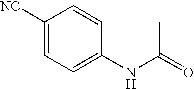 | − | 679 | Mp = 158.1-164.5° C. [α]_{D}²⁵ = −81.2°(2.6 mg/2 mL MeOH) |
| 418 | 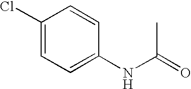 | + | 680 | Mp = 161.5-164.8° C. MH+ = 559.1787 |
| 419 | 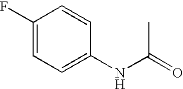 | + | 681 | Mp = 157.7-161.7° C. MH+ = 543.2069 |

### Examples 420 and 421

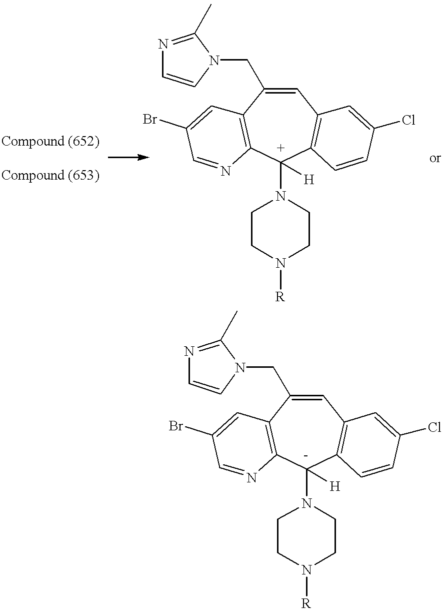

The appropriate (+) enantiomer (652) or (−) enantiomer (653) from Preparative Example 55 above, was taken up in CH₂Cl₂ treated with the corresponding isocyanate and stirred at room temperature over night. The crude product was purified directly by silica gel preparative thin layer chromatography or silica gel column chromatography to afford the following compounds in Table 9 below:

TABLE 9

| Example # | R | Enantiomer | Comp # | Phys. Data. |
|---|---|---|---|---|
| 420 | 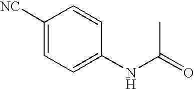 | + | 682 | Mp = 168.8-172.3° C. |
| 421 | 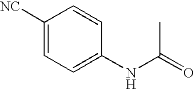 | − | 683 | Mp = 172.5-177.7° C. |
| 421.1 | 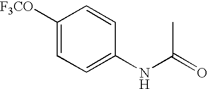 | + | 683.1 | Mp = 157.1-160.5° C. (dec) |
| 421.2 | 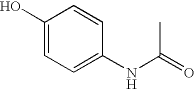 | + | 683.2 | Mp = 223.6-229.1° C. (dec) |

### Examples 422 and 423

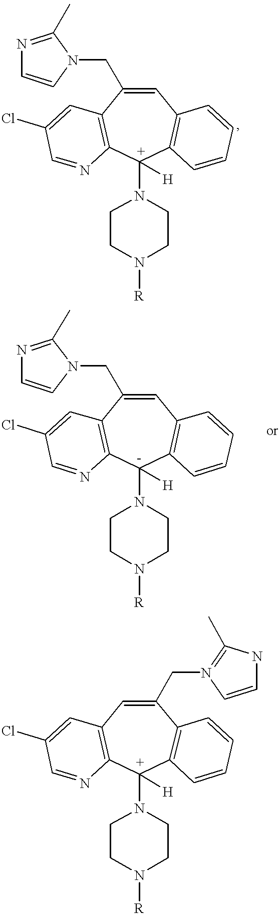

The appropriate compound (659) (+) enantiomer, (660) (−) enantiomer or (659A) (+) enantiomer from Preparative Example 56 above, was taken up in CH₂Cl₂ treated with the corresponding isocyanate and stirred at room temperature over night. The Crude product was purified directly by silica gel preparative thin layer chromatography or silica gel column chromatography to afford the following compounds in Table 10 below:

TABLE 10

| Example # | R | Enantiomer | Comp # | Phys. Data. |
|---|---|---|---|---|
| 422 | 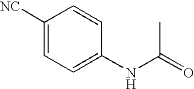 | + | 684 | Mp = 155.9-165.1° C. |
| 423 | 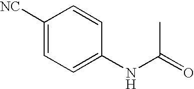 | − | 685 | Mp = 154.2-164.8° C. |
| 492 | 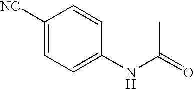 | + | 806 | Mp = 157.1-160.5° C. MH⁺ 32 689 |

### Examples 424 and 425

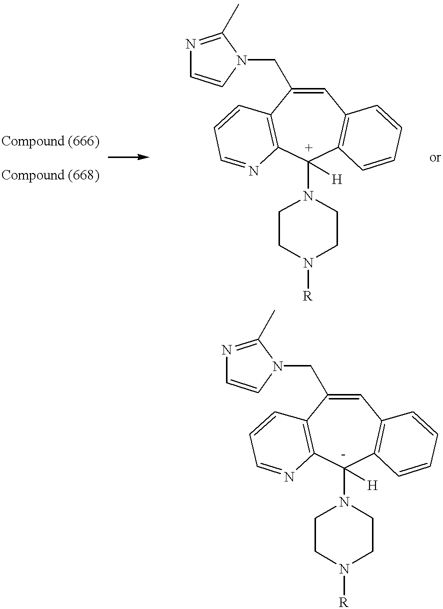

The appropriate (+)enantiomer (666) or (−)enantiomer (668) from Preparative Example 57 above, was taken up in CH₂Cl₂, treated with the corresponding isocyanate and stirred at room temperature over night. The crude product was purified directly by silica gel preparative thin layer chromatography or silica gel column chromatography to afford the following compounds in Table 11 below:

TABLE 11

| Example # | R | Enantiomer | Comp # | Phys. Data. |
|---|---|---|---|---|
| 424 | 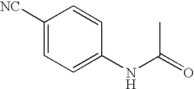 | + | 686 | Mp = 166-170° C. [α]_{D}²⁵ = +106.8°(1.45 mg/2 mL MeOH) |
| 425 | 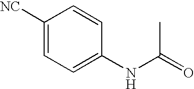 | − | 687 | Mp = 170-176° C. [α]_{D}²⁵ = −91°(2.78 mg/2 mL MeOH) |

### Examples 426 and 427

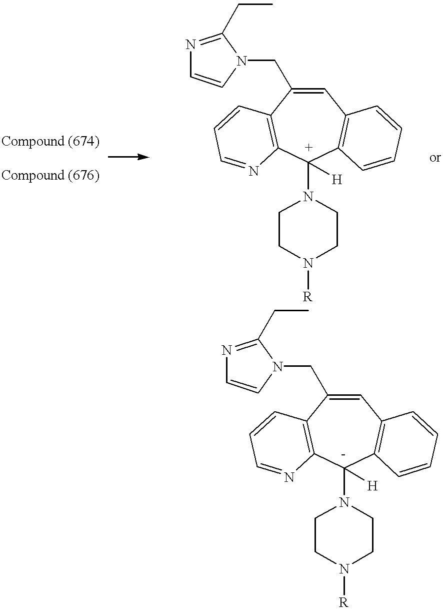

The appropriate (+) enantiomer (674) or (−) enantiomer (676) from Preparative Example 58 above, was taken up in CH₂Cl₂, treated with the corresponding isocyanate and stirred at room temperature over night. The crude product was purified directly by silica gel preparative thin layer chromatography or silica gel column chromatography to afford the following compounds in Table 12 below:

TABLE 12

| Example # | R | Enantiomer | Comp # | Phys. Data. |
|---|---|---|---|---|
| 426 | 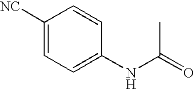 | + | 688 | Mp = 150-153° C. |
| 427 | 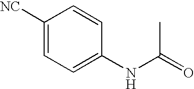 | − | 689 | Mp = 154-158° C. |

### Examples 428 and 429

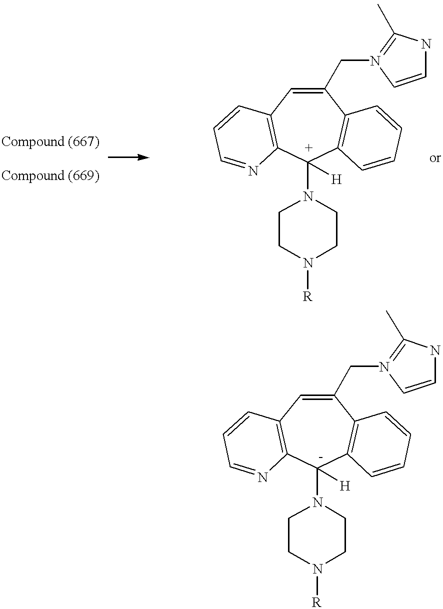

The appropriate (+) enantiomer (667) or (−) enantiomer (669) from Preparative Example 57 above, was taken up in CH₂Cl₂, treated with the corresponding isocyanate and stirred at room temperature over night. The crude product was purified directly by silica gel preparative thin layer chromatography or silica gel column chromatography to afford the following compounds in the table below:

| | | Enan- | | Phys. |
|---|---|---|---|---|
| Example # | R | tiomer | Comp # | Data. |
| 428 | 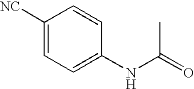 | Isomer 1 | 690 | MH⁺ =516 |
| 429 | 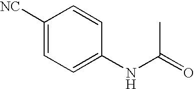 | Isomer 2 | 691 | MH⁺ =516 |

### Examples 430 and 431

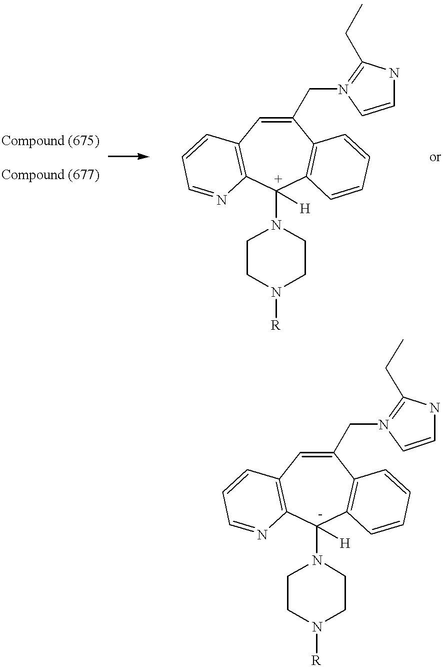

The appropriate (+) enantiomer (675) or (−) enantiomer (677) from Preparative Example 58 above, was taken up in CH₂Cl₂, treated with the corresponding isocyanate and stirred at room temperature over night. The crude product was purified directly by silica gel preparative thin layer chromatography or silica gel column chromatography to afford the following compounds in the table below:

| | | Enan- | | Phys. |
|---|---|---|---|---|
| Example # | R | tiomer | Comp # | Data. |
| 430 | 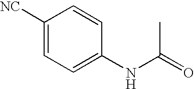 | Isomer 1 | 692 | MH⁺ =530 |
| 431 | 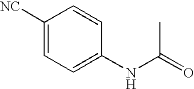 | Isomer 2 | 693 | MH⁺ =530 |

### Preparative Example 59

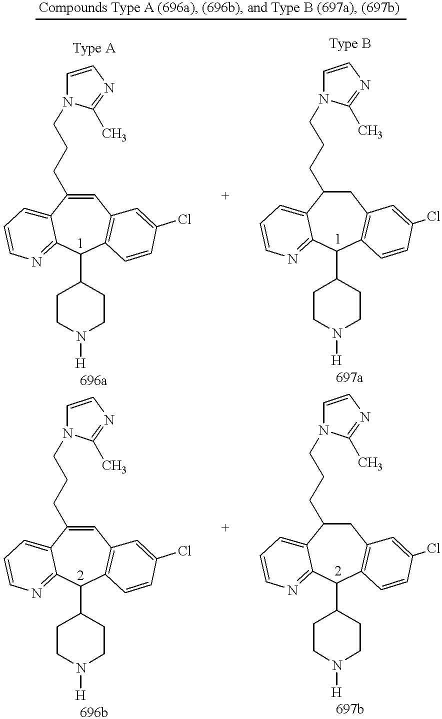

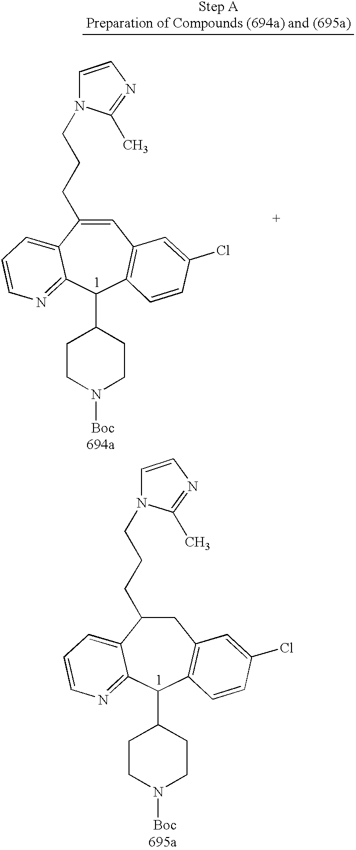

To a stirred solution of 2-methyl imidazole (1.80 g, 21.97 mmol) in anhydrous DMF (40 mL) at room temperature, was added NaH (5.3 g, 21.97 mmol) and Compound (27) from Preparative Example 4, Step E (4.0 g, 7.33 mmol). The resulting solution was stirred at room remperature for 1 hr and concentrated to dryness, followed by extraction with EtOAc-NaHCO₃. The combined organic layer was dried over Na₂SO₄, filtered and concentrated to dryness to give the mixture of single bond and double bond compounds. These compounds were further purified by column chromatography on silica gel, eluting with 2%MeOH/NH3/98%CH₂Cl₂ to yield: Pure Type A Compound (694) (0.450 g) (MH⁺=533) and a mixture of Type A (694) and Type B Compound (695) (2.55 g)(MH⁺=535).

Compounds (694) and (695) were further purified by prep HPLC, eluting with 15%IPA/85%Hexane/0.2%DEA to give: Type B Compound (695a) (isomer 1; 0.58 g, MH⁺=535.4) and Type A Compound (694a) (isomer 1; 0.61 g, MH⁺=533) and a mixture of compounds (694b) and (695b) (isomer 2 products; 0.84 g).

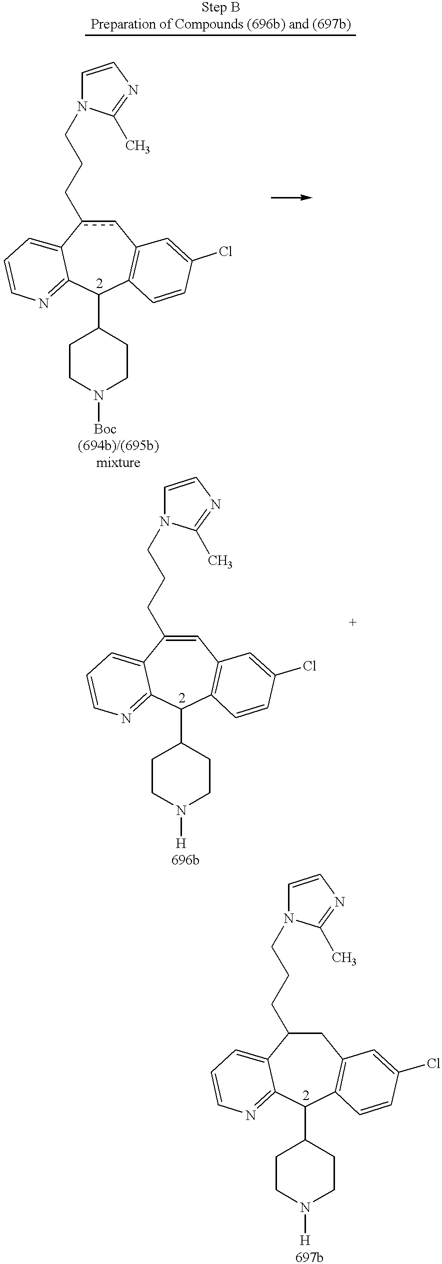

The mixture of compounds (694b/695b) from Step A above (0.8 g, 1.5 mmol) in 4N HCl/Dioxane (40 mL) was stirred at room temperature for 3 hrs and concentrated to dryness to give a mixture of deprotected compounds as product. The product was further purified by HPLC, eluting with 15%IPA/85% hexane/0.2%DEA to give the pure compound (696b) Type A (isomer 2; 0.29 g) and pure Compound (697b) Type B (isomer 2, 0.19 g).

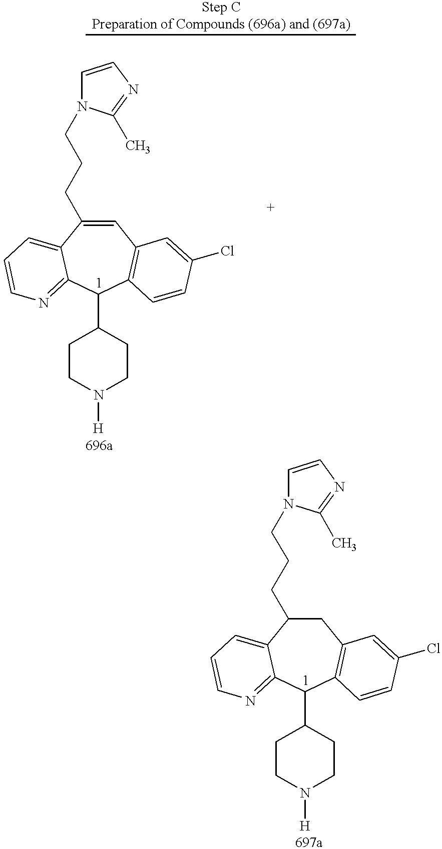

Compounds (694a) and (695a) (pure isomer 1) were individually deprotected using 4N HCl/Dioxane in essentially the same method as that of the isomer 2 products described above, to give the corresponding N-H products (696a) Type A (isomer 1) and (697a) Type B (isomer 1).

### Examples 432-437

Reacting Compound (696a) (isomer 1) in essentially the same manner as in Example 13 with the appropriate chloroformate or isocyanate, the following compounds listed in Table 13 below, were prepared.

TABLE 13
2-Methylpropylimidazole-5-Substituted Bridgehead Double bond Analogs

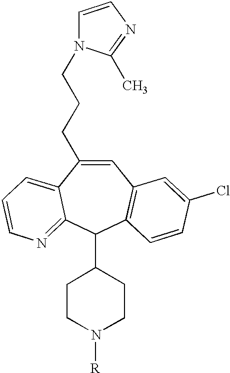

| EXAMPLE # | R | COMPOUND # | PHYSICAL DATA |
|---|---|---|---|
| 432 | 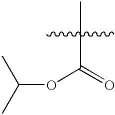 | 698 | MH+ = 519.1 |
| 433 | 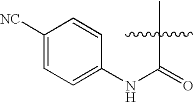 | 699 | MH+ = 577.1 |
| 434 | 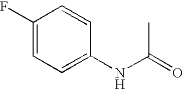 | 700 | MH+ = 570.1 |
| 435 | 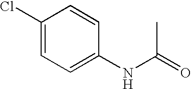 | 701 | MH+ = 585.1 |
| 436 | 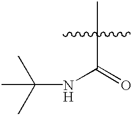 | 702 | |
| 437 | 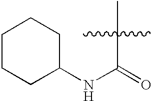 | 703 | MH+ = 558.1 |

### Examples 438-442

Reacting Compound (697a) (isomer 1) in essentially the same manner as in Example 13 with the appropriate chioroformate or isocyanate, the following compound listed in Table 14 below were prepared.

TABLE 14
2-Methylpropylimidazole-5-Substituted
Bridgehead Single bond Analogs

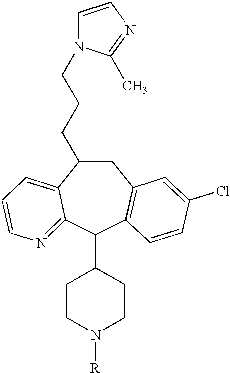

| EXAM- | | COMPOUND | PHYSICAL |
|---|---|---|---|
| PLE # | R | # | DATA |
| 438 | 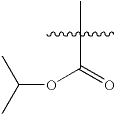 | 704 | MH+ =521.1 |
| 439 | 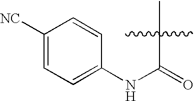 | 705 | MH+ =579.1 |
| 440 | 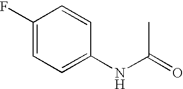 | 706 | MH+ =572.1 |
| 441 | 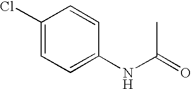 | 707 | MH+ =587.1 |
| 442 | 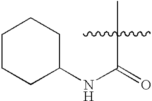 | 708 | MH+ =560.1 |

### Preparative Example 60

### Compounds (711 a), (711 b), (712a) and (712b).

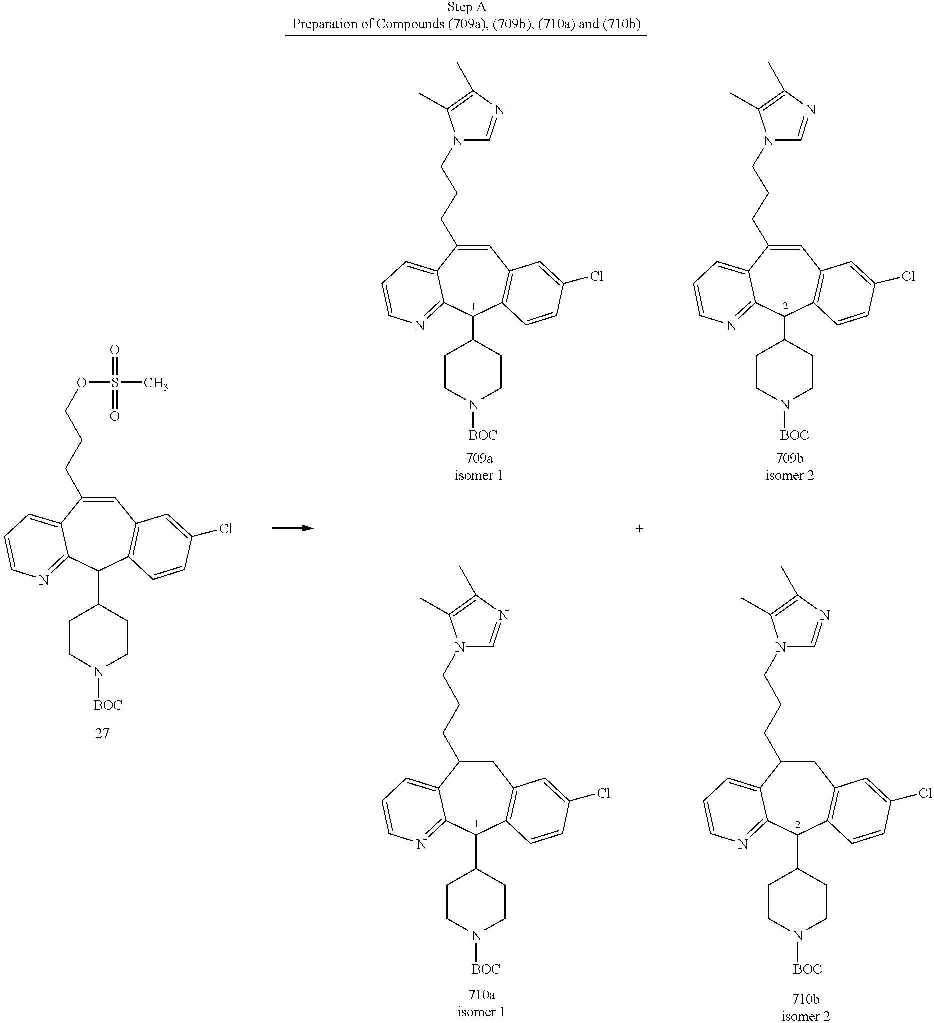

To a stirred solution of 4,5-Dimethylimidazole (1.08 g, 11.25 mmol) in anhydrous DMF (35 mL) at room temperature, was added NaH (0.27 g, 11.2 mmol) and stirred for 10 minutes, followed by the addition of Compound (27) from Preparative Example 4 Step E (4.0 g, 7.32 mmol). The resulting solution was srirred at room temperature overnight. To this solution was added the solution of 4,5-dimethylimidazole (0.35 g, 3.65 mmol) and NaH (0.088 g, 3.67 mmol) in DMF (5 mL). The resulting solution was heated at 80° C.-90° C. for 4 hrs, then cooled down to room temperature, followed by extraction with EtOAc-H₂O. The combined organic layer was washed with brine, dried over Na₂SO₄, filtered and concentrated to dryness and purified by column chromatography on silica gel, eluting with 50%EtOAc/50%hexane to 5%MeOH/CH₂Cl₂ to give the mixture of products Compound (709) Type A and Compound (710) Type B (1.2 g, MH⁺=547.3). The products were further purified by prep HPLC, using a chiral AD column, eluting with 15%IPA/85%hexane/0.2%DEA to give 4 seperate compounds:

Compound (709a) isomer 1, type A (0.291 g, MH₊=547.3), Compound (710a) isomer 1, type B (0.305 g, MH₊=549.3) and

Compound (709b) isomer 2, type A (0.280 g, MH₊=547.3), Compound (710b) isomer 2, type B (0.2 g, MH₊=549.3)

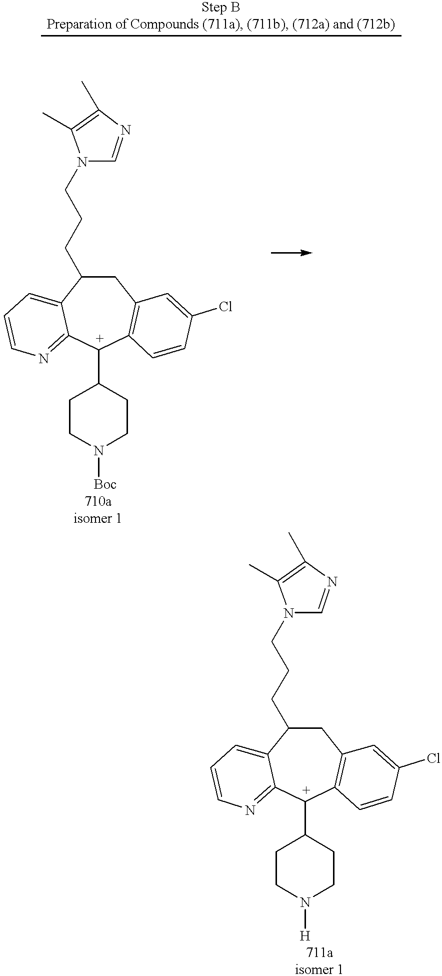

A solution of Compound (710a), isomer 1 type B (0.245 g, 0.45 mmol) in 4N HCl/Dioxane (2 mL) was stirred at room temperature for 3 hrs then concentrated to dryness to give Compound (711a) isomer I, type B product (0.184 g, 98% yield) (MH₊=455.1).

Compounds (711 b), (isomer 2; type B); (712a) (isomer 1; type A) and (712b) (isomer 2; type A) were all prepared in a similar fashion to that of Compound (711 a) isomer 1 type B in Step B above. (711 b) (0.085 g, 75% yield). (712a) (0.141 g, 75% yield), (712b) (0.106 g, 59% yield),

### Examples 443-447

Reacting Compounds (711a) and (711b) seperately following the procedure described in Example 13 with the appropriate chloroformates or isocyanates, the following compounds listed in Table 15 below were prepared.

TABLE 15
4,5-Dimethylpropylimidazole-5-Substituted
Bridgehead Single bond Analogs

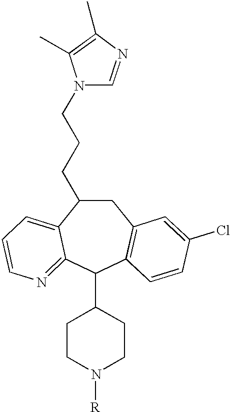

| EXAM- | | COMPOUND | PHYSICAL |
|---|---|---|---|
| PLE # | R | # | DATA |
| 443 | 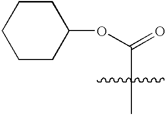 | 713 | MH+ =575.1 |
| 444 | 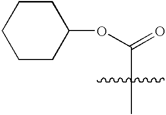 | 714 | MH+ =575.1 |
| 445 | 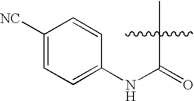 | 715 | MH+ =593.2 |
| 446 | 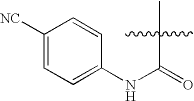 | 716 | MH+ =593.2 |
| 447 | 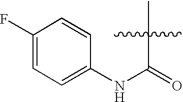 | 717 | MH+ =586.1 |

### Examples 448-454

Reacting Compounds (712a) and (712b) seperately following the procedure described in Example 13 with the appropriate chloroformates or isocyanates, the following compounds listed in Table 16 below were prepared.

TABLE 16
4,5-Dimethylpropylimidazole-5-Substituted
Bridgehead Double bond Analogs

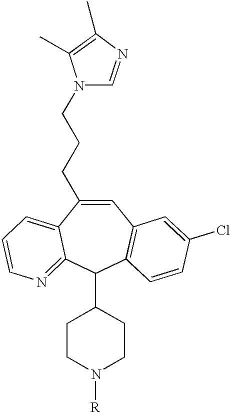

| EXAM- | | COMPOUND | PHYSICAL |
|---|---|---|---|
| PLE # | R | # | DATA |
| 448 | 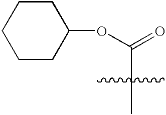 | 718 | MH+ =573.1 |
| 449 | 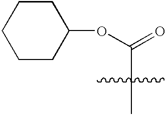 | 719 | MH+ =573.1 |
| 450 | 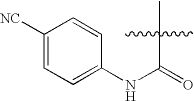 | 720 | MH+ =591.1 |
| 451 | 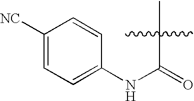 | 721 | MH+ =591.1 |
| 452 | 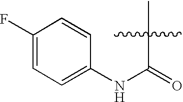 | 722 | MH+ =584.1 |
| 453 | 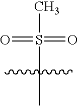 | 723 | MH+ =525.1 |
| 454 | 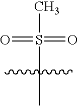 | 724 | MH+ =525.1 |

### Preparative Example 61

### Preparation of Compounds (727a), (727b), (728a) AND (728b).

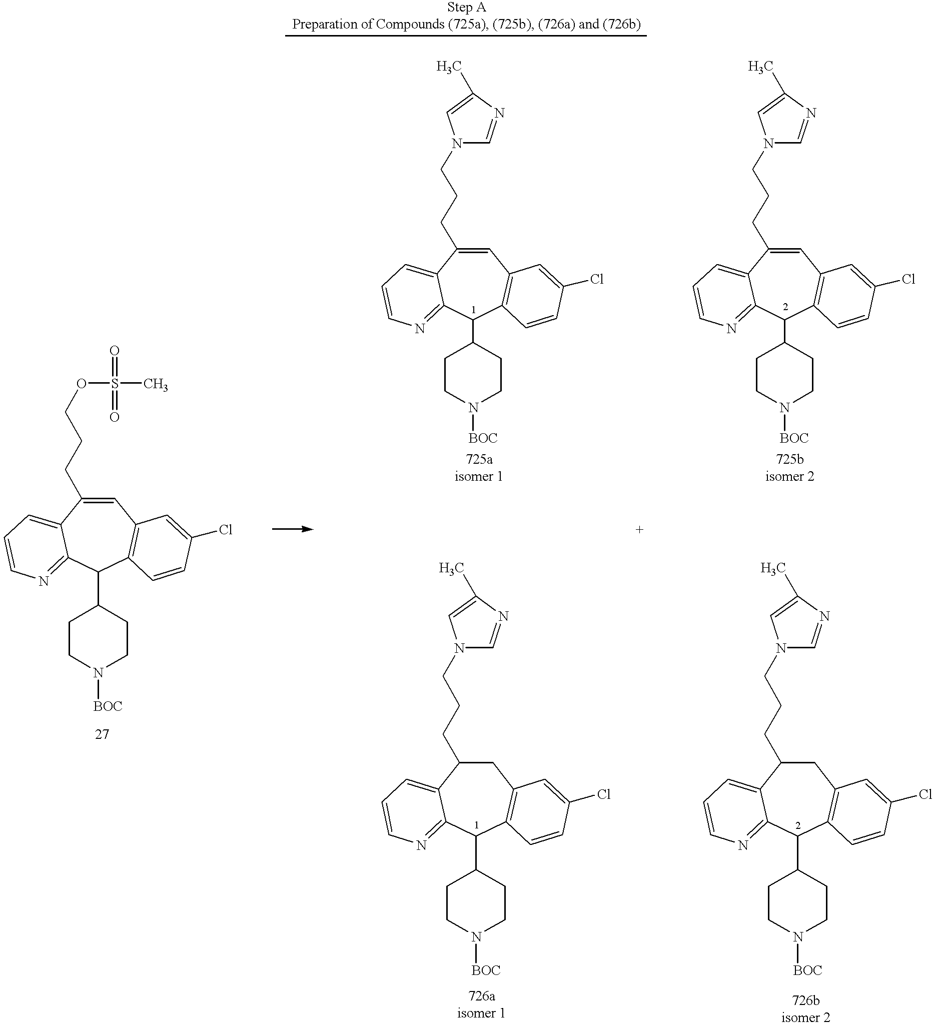

Compound (27) from Preparative Example 4, Step E was reacted in essentially the same manner as described in Preparative Example 60, Step A above substituting 4-Methylimidazole for 4,5-Dimethylimidazole to obtain four separate compounds as products. BOC derivatives

Compound (725a) isomer 1, type A (0.69 g, MH⁺=533.1)

Compound (725b) isomer 2, type A (0.10 g, MH⁺=533.1)

Compound (726a) isomer 1, type B (0.35 g, MH⁺=533.1)

Compound (726b) isomer 2, type B, (0.22 g, MH⁺=533.1)

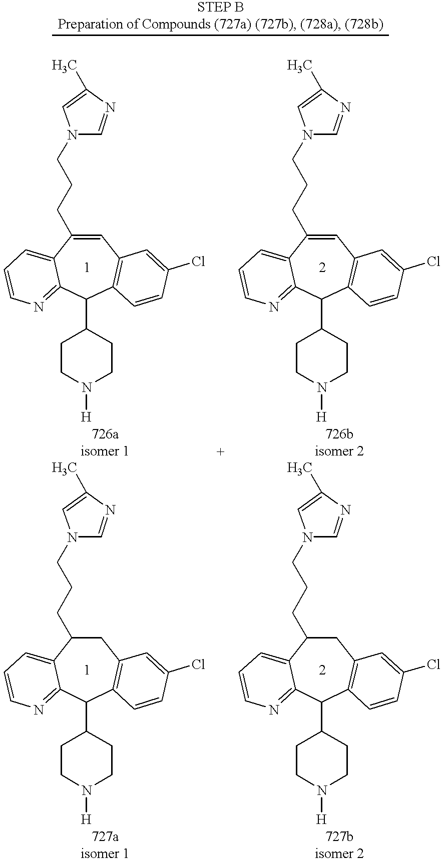

In essentially the same manner as described in Preparative Example 60, Step B, the —NH derivatives were prepared:

Compounds:

(727a) isomer 1 type B (0.3 g, 100% yield, MH₊=435.1),

(727b) isomer 2, type B;

(728a) isomer 1, type A and

(728b) isomer 2, type A.

### Examples 455-459

Reacting Compounds (727a) and (727b) separately following the procedure described in Example 13 with the appropriate chloroformate or isocyanate, the following compounds listed in Table 17 below were prepared.

TABLE 17
4-Methylpropylimidazole-5-Substituted
Bridgehead Single bond Analogs

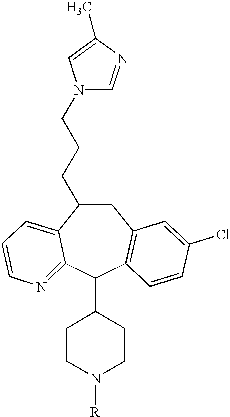

| EXAM- | | COMPOUND | PHYSICAL |
|---|---|---|---|
| PLE # | R | # | DATA |
| 455 | 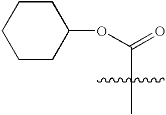 | 729 | MH+ =561.1 |
| 456 | 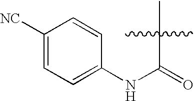 | 730 | MH+ =581.1 |
| 457 | 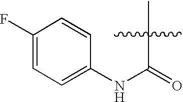 | 731 | MH+ =572.1 |
| 458 | 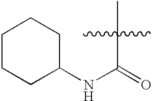 | 732 | MH+ =560.1 |
| 459 | 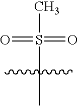 | 733 | MH+ =513.1 |

### Examples 460-469

Reacting Compounds (728a) and (728b) seperately following the procedure described in Example 13 with the appropriate chloroformates and isocyanates, the following compounds listed in Table 18 below were prepared.

TABLE 18
4-Methylpropylimidazole-5-Substituted
Bridgehead Double bond Analogs

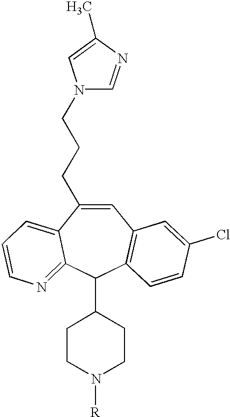

| EXAM- | | COMPOUND | PHYSICAL |
|---|---|---|---|
| PLE # | R | # | DATA |
| 460 | 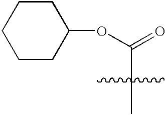 | 734 | MH+ =559.1 |
| 461 | 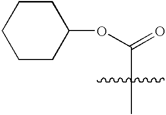 | 735 | MH+ =559.1 |
| 462 | 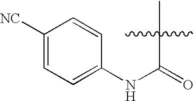 | 736 | MH+ =579.1 |
| 463 | 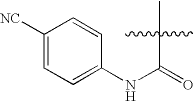 | 737 | MH+ =579.1 |
| 464 | 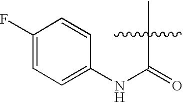 | 738 | MH+ =570.1 |
| 465 | 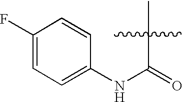 | 739 | MH+ =570.1 |
| 466 | 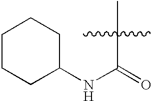 | 740 | MH+ =558.1 |
| 467 | 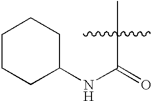 | 741 | MH+ =558.1 |
| 468 | 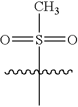 | 742 | MH+ =511.1 |
| 469 | 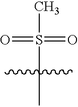 | 743 | MH+ =511.1 |

### Example 470

### Preparation of Compound (748)

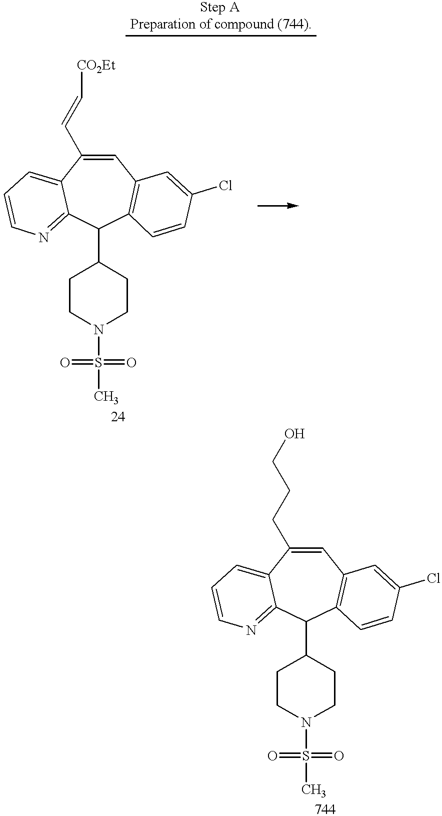

To a stirred solution of Compound (24) from Preparative Example 4, Step D (4.0 g, 8.2 mmol) under nitrogen at room temperature, was added CuCl (0.7 g, 8.2 mmol). The solution was then cooled to 0° C., followed by portion wise addition of NaBH₄ (4.66 g, 123,2 mmol). The resulting solution was stirred at 0° C. for 6 h., concentrated to dryness, then extracted with CH₂Cl₂-sat.NaHCO₃. The combined organic layer was dried over MgSO₄, filtered, concentrated and purified by column chromatography on 200 mL of normal phase silica gel, eluting with 20%EtOAc/CH₂Cl₂ to give Compound (744) (3.62 g, 99% yield, MH⁺=447).

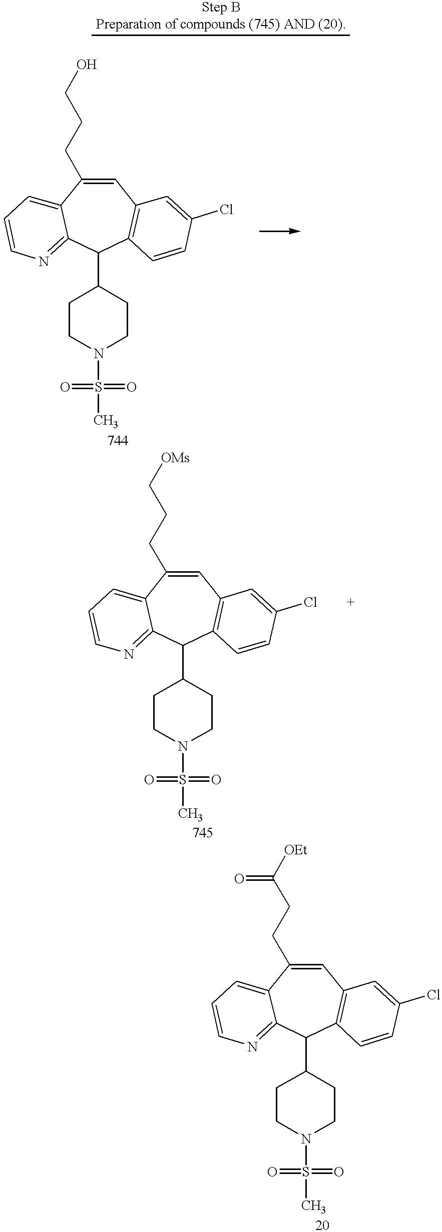

To a stirred solution of Compound (744) from Step A above (3.0 g, 5.7 mmol) in CH₂Cl₂ (100 mL) under nitrogen at room temperature, was added triethyl amine (2.4 mL, 17.1 mmol) and methanesulfonyl chloride (0.98 g, 8.7 mmol). The resulting solution was stirred at room temperature over night, then washed with saturated NaHCO₃. The combined organic layer was dried over Na₂SO₄, filtered, concentrated to dryness and purified by Biotage column chromatography, eluting with 30%EtOAc/70%CH₂Cl₂ to give Compound (745) as a white solid (1.19 g, MH⁺=525.1) and Compound (20) (1.31 g, MH⁺=489.1)

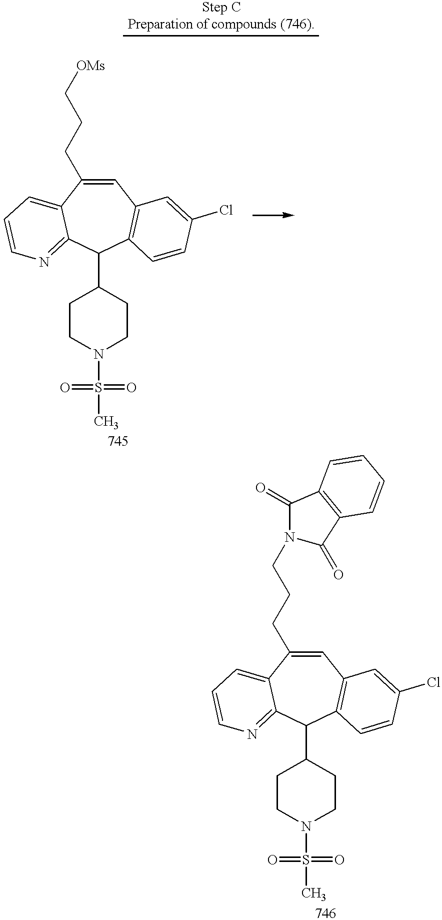

To a stirred solution of Compound (745) from Step B above (2.17 g, 4.3 mmol) in DMF (50 mL) under nitrogen at room temperature was added phthalimide potassium derivative (1.20 g, 0.5 mmol). The resulting solution was heated to 90° C. for 4 h., cooled down to room temperature, concentrated to dryness and extracted with CH₂Cl₂-sat.NaHCO₃. The combined organic layer was dried over Na₂SO₄, filtered, concentrated to dryness and purified by column chromatography on silica gel, eluting with 50%-70%EtOAc/hexane to give Compound (746) as a white solid (1.76 g, 71% yield, MH⁺=577.0).

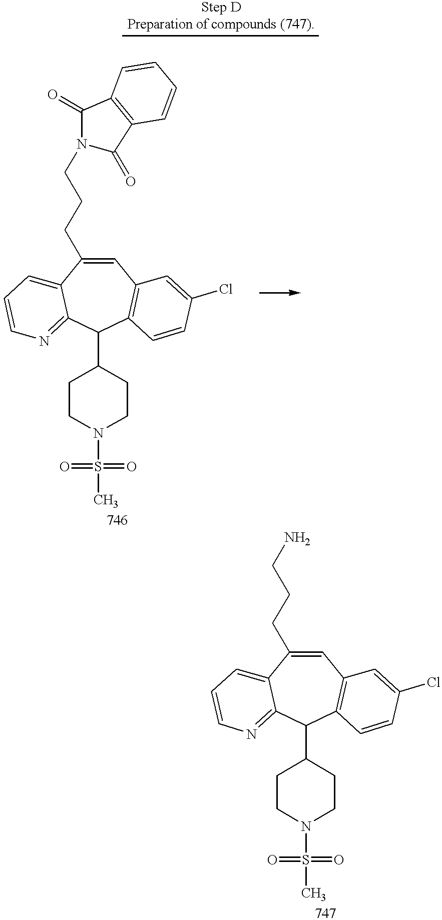

To a stirred solution of Compound (746) from Step C above (1.67 g, 2.9 mmol) in EtOH (50 mL) at room temperature, was added hydrazine monohydrate (0.29 g, 5.8 mmol). The resulting solution was heated to reflux for 4 h. cooled down to room temperature, concentrated to dryness and extracted with CH₂Cl₂-H₂O. The combined organic layer was dried over MgSO₄, filtered and concentrated to dryness to give Compound (747) as a white solid (1.23 g, 95% yield, MH⁺=446.1)

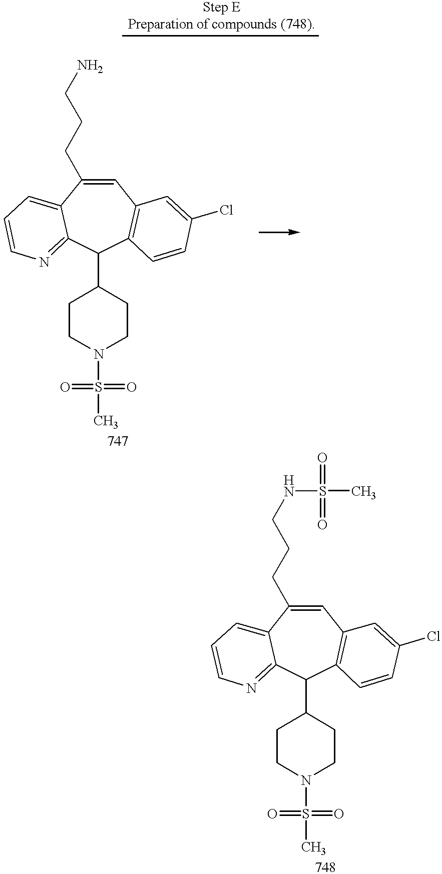

To a stirred solution of Compound (747) from Step D (0.1 g, 0.22 mmol) in CH₂Cl₂ (5 mL) under nitrogen at room temperature, was added TEA (0.06 mL, 0.45 mmol) and methanesulfonyl chloride (0.038 g, 0.34 mmol). The resulting solution was stirred at room temperature over night, then washed with sat. NaHCO₃. The combined organic layer was dried over Na₂SO₄, filtered and purified by column chromatography on silica gel, eluting with 3% MeOH—NH₃/CH₂Cl₂ to give Compound (748) as a white solid (0.087 g, 76% yield, MH⁺=524.0)

### Example 471

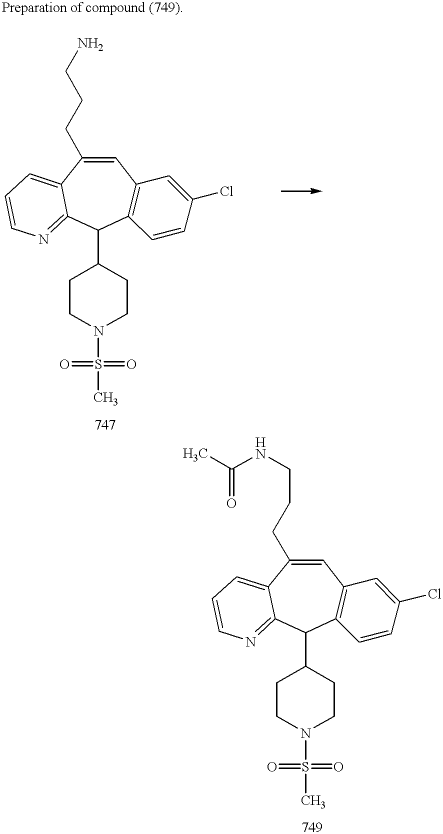

Reacting Compound (747) from Example 470 Step D above in essentially the same manner as in Step E of Example 470 substituting acetylchloride, Compound (749) was prepared.(0.048 g, 45% yield, MH₊=488.2).

### Example 472

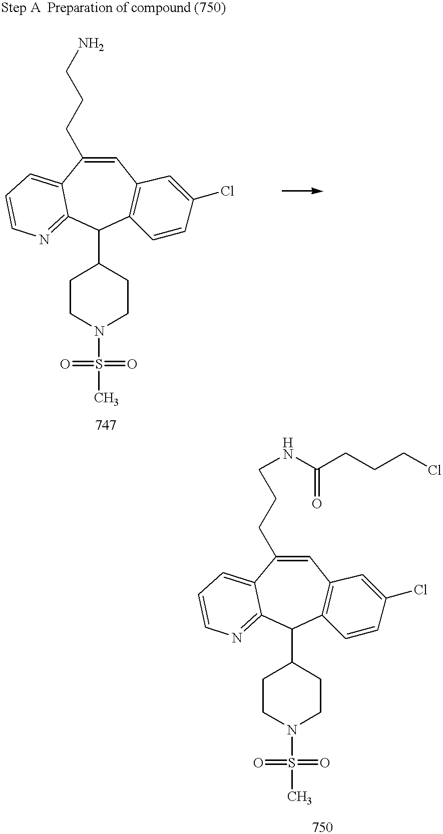

Reacting Compound (747) from Example 470 Step D above in essentially the same manner as in Step E of Example 470 substituting 4-Chlorobutyryl chloride (ACROS), Compound (750) was prepared (0.67 g, 100% yiled, MH⁺=514.1).

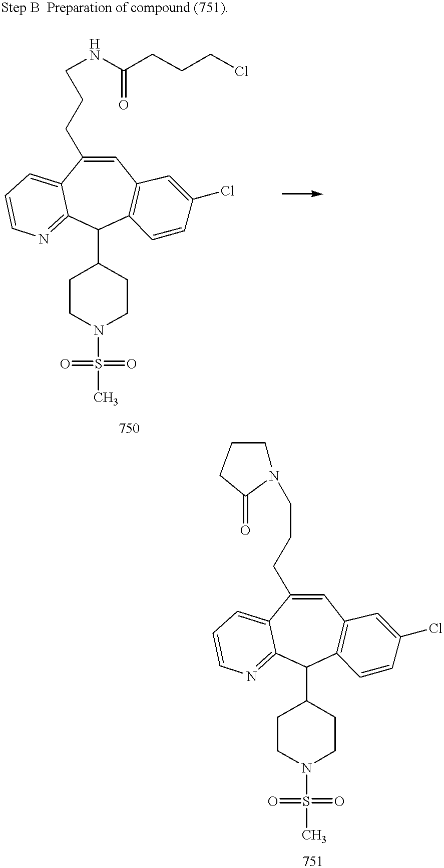

To a stirred solution of Compound (750) from Step A (0.575 g, 1.11 mmol) in toluene (15 mL) under nitrogen at room temperature, was added K₂CO₃ (0.55 g, 4.01 mmol). The resulting solution was stirred at room temperature over the weekend then heated to 55° C. for 7 h. The solution was then cooled down to room temperature, filtered, concentrated to dryness and purified by column chromatography, eluting with 1.5%MeOH—NH₃/98.5%CH₂Cl₂ to give Compound (751) as a white solid (0.15 g, 26% yield, MH⁺=524.1)

### Example 473

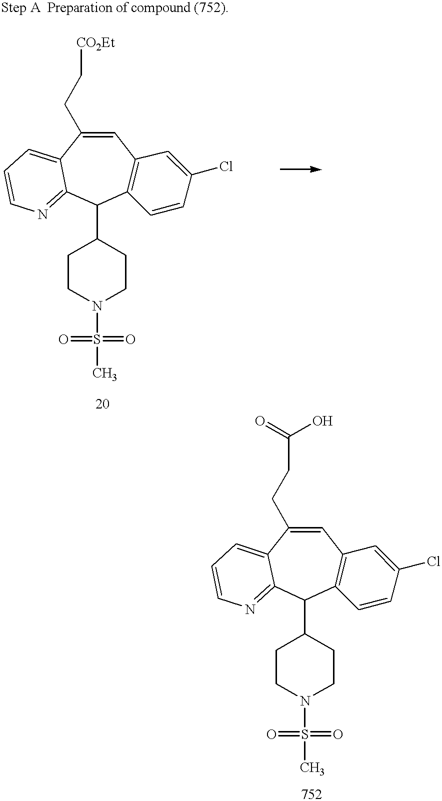

To a stirred solution of Compound (20) from Example 470, Step B (0.67 g, 1.37 mmol) in THF (5 mL), was added 1N NaOH solution (6.9 mL, 6.88 mmol). The resulting solution was stirred at room temperature overnight and concentrated to dryness. The solution was then acidified with 10% citric acid and then extracted with CH₂Cl₂. The combined organic layer was dried over MgSO₄, filtered and concentrated to dryness to give Compound (752) as a light yellow product (0.33 g, 52% yield, MH⁺=461.1)

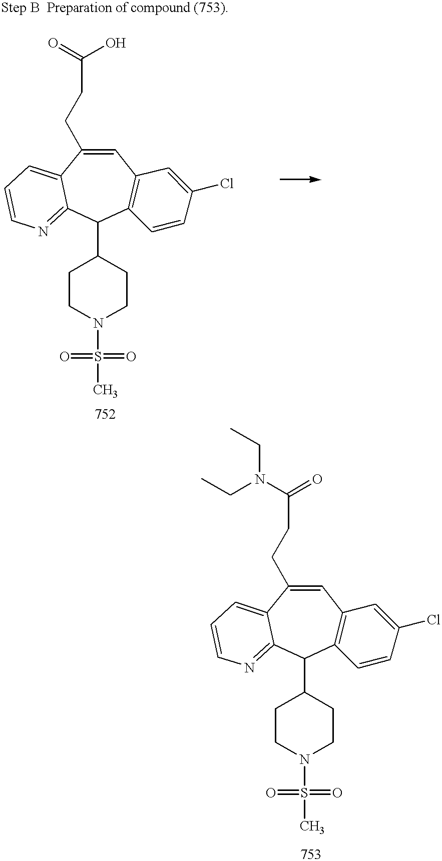

To a stirred solution of Compound (752) from Step A above (0.1 g, 0.23 mmol) in CH₂Cl₂ (5 mL) under nitrogen at room temperature, was added oxalyl chloride (0.97 g, 7.62 mmol) and diethyl amine (0.47 g, 6.43 mmol). The resulting solution was stirred at room temperature for 1 hr and concentrated to dryness. The crude product was then purified by column chromatography, eluting with 2%MeOH—NH₃/98%CH₂Cl₂ to give Compound (753) as a white solid (0.051 g, 49.5% yield, MH⁺=516.1)

### Example 474

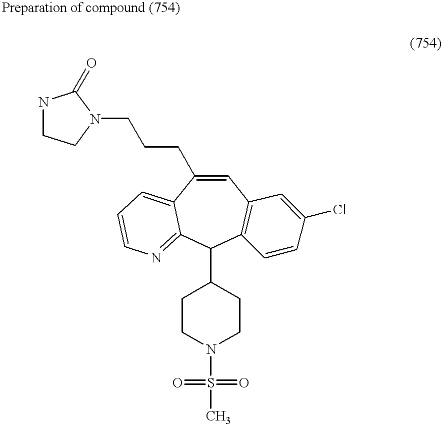

To a stirred solution of 2-imidazolidone (0.22 g, 2.0 mmol) in DMF (10 mL) was added NaH (0.28 g, 2.0 mmol). The resulting solution was stirred at room temperature for 1 hr. This solution was then added into a solution of Compound (22) from Preparative Example 3, Step C (0.67 g, 1.3 mmol) in DMF (20 mL) under nitrogen inlet at room temperature. The resulting solution was heated to 90° C. for 2 hrs, concentrated to dryness, then extracted with CH₂Cl₂-sat.NaHCO₃. The combined organic layer was then dried over MgSO₄, filtered, concentrated to dryness and purified by column chromatography on silica gel, eluting with 3% MeOH—NH₃/97% CH₂Cl₂ to give a light yellow solid (754) (0.17 g, 25% yield, MH⁺=515.1).

### Example 475

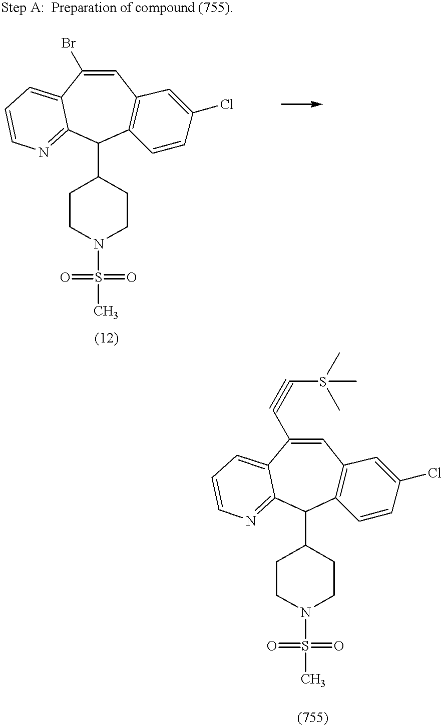

To a stirred solution of Compound (12) from Preparative Example 2, Step B (15.75 g, 0.336 mmol) in DMF (200 mL) under nitrogen inlet at room temperature, was added trimethylsilylacetalene (12.14 g, 124 mmol), bis(triphenylphosphine)palladium (II)dichloride (0.47 g, 0.67 mmol), Et₃N (13.1 mL, 94 mmol), Cul (0.89 g, 4.7 mmol) and Nal (1.53 g, 10 mmol). The resulting solution was stirred at room temperature overnight, concentrated to dryness, then extracted with CH₂Cl₂-H₂O. The combined organic layer was dried over MgSO₄, filtered, concentrated to dryness and purified by column chromatography on silica gel, eluting with 20% EtOAc/80% hexane to give the product (755) (12.35 g, M=485).

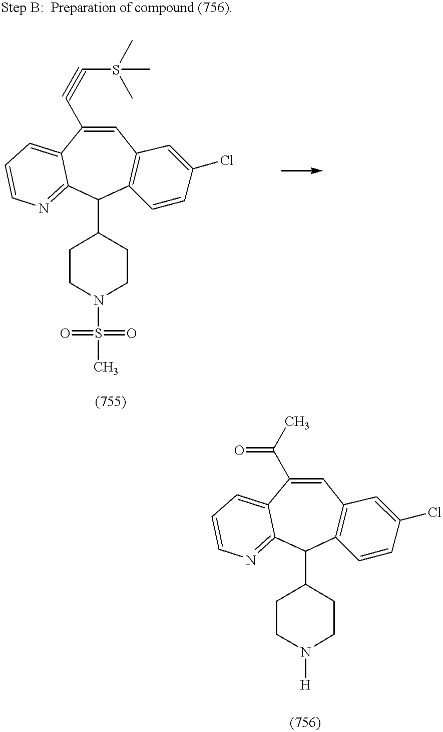

A solution of Compound (755) from Step A above (4.48 g, 9.24 mmol), in concentrated HCl (100 mL) was heated to reflux overnight. The solution was then cooled down to room temperature and basified with 50% NaOH solution (w/w) and then extracted with CH₂Cl₂. The combined organic layer was dried over MgSO₄, filtered and concentrated to dryness to give an off white solid (756) (4.40 g, 100% yield, MH⁺=353.1).

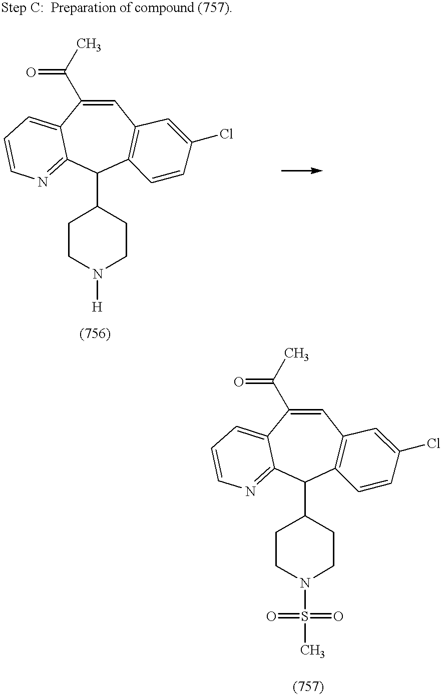

To a stirred solution of Compound (756) from step B (3.15 g, 8.93 mmol) in CH₂Cl₂ (100 mL) was added Et₃N (2.5 mL, 17.85 mmol) and methanesulfonyl chloride (0.51 g, 4.46 mmol). The resulting solution was stirred at room temperature overnight. The solution was then washed with saturated NaHCO₃ and the organic layer was dried over MgSO₄, filtered and concentrated to dryness to give a crude product (4.31 g, 100% yield, MH⁺=431.1)

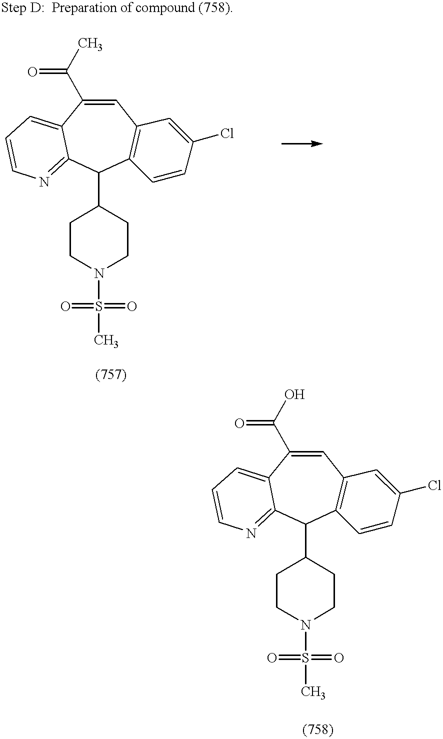

The solution of Compound (757) from Step C (3.84 g, 8.91 mmol) in 4% NaClO (150 mL) and 45% NaOH solution (15 mL) was heated to reflux for 2 hrs, then cooled down to room temperature, followed by addition of saturated sodium bisulfite solution (150 mL). The solution was then adjusted to pH=6.5 and extracted with CH₂Cl₂. The combined organic layer was dried over MgSO₄, filtered and concentrated to dryness to give a light yellow solid (3.31 g, 86% yield, MH⁺=433.1).

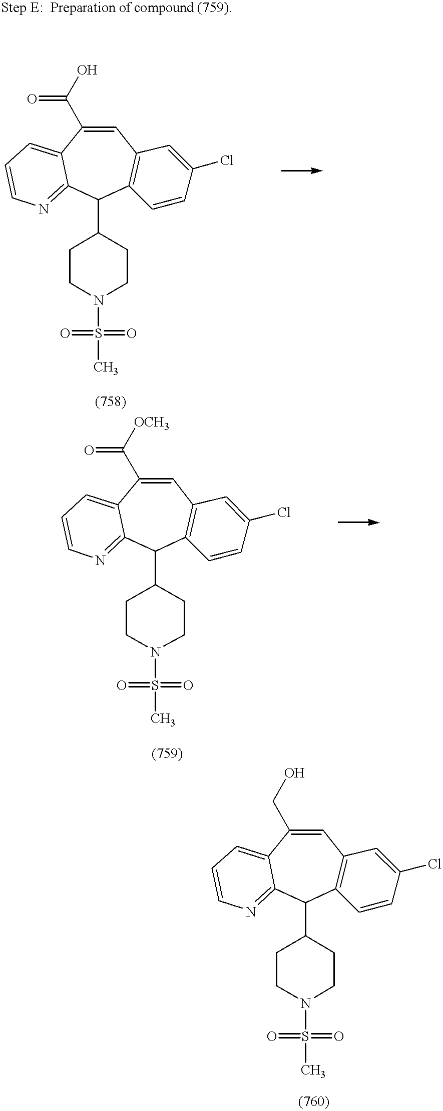

To a stirred solution of Compound (758) from step D (3.31 g, 7.65 mmol) in toluene (80 mL) and MeOH (50 mL) under nitrogen at room temperature, was added (trimethylsilyl)diazomethane (2.0M in hexane)(3.4 mL, 68.8 mmol) at 0° C., until the colorless solution turned to yellow solution. The resulting solution was stirred at 0° C. for half an hour and concentrated to dryness to give a crude product (759).

To a stirred cooling solution of the crude product (759) from above, in THF (30 mL) at 0° C. was added DIBAL (15.3 mL, 15.3 mmol). The resulting solution was stirred at 0° C. for 2hrs, followed by extraction with 10% citric acid and 1N NaOH solution. The combined organic layer was dried over MgSO₄, filtered and concentrated to dryness to give a light yellow solid (760) (2.90 g, 90% yield, MH⁺=419.1).

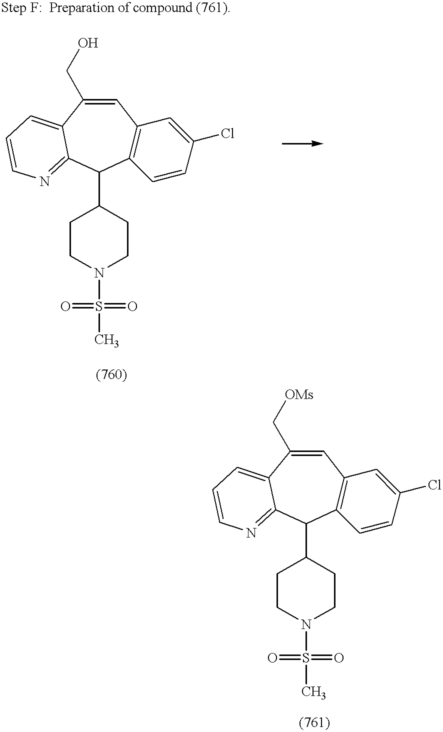

Reacting Compound (760) in essentially the same manner as Step C above, Compound (761) was prepared.

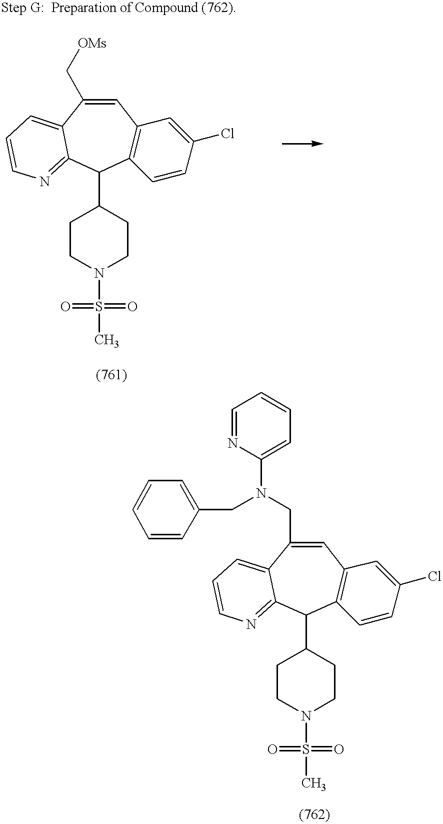

To a stirred solution of 2-benzylaminopyridine (0.115 g, 0.624 mmol) in DMF (10 mL) at room temperature, was added NaH (9.81 g, 0.41 mmol) and stirred for 0.5 hr. To a stirred solution of mesylate compound from step F (0.2 g, 0.41 mmol) in DMF (10 mL) under nitrogen inlet, was added the solution of 2-benzylaminopyridine in DMF above. The resulting solution was heated to 90° C. for 3hrs, concentrated to dryness followed by extraction with CH₂Cl₂-sat.NaHCO₃, then dried over MgSO₄, filtered, concentrated to dryness and purified by column chromatography on silica gel, eluting with 5% MeOH—NH₃/CH₂Cl₂ to give a light yellow solid (762) (0.03 g, 13% yield, MH+=585.1).

### Example 476

### Preparation of Compound (768)

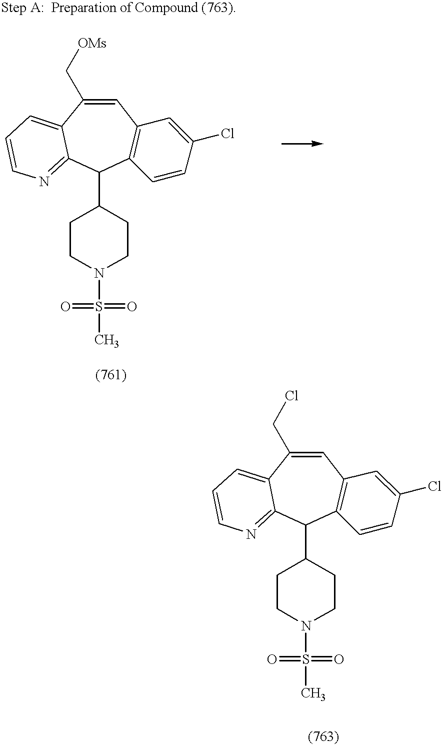

In essentially the same manner as Example 475, Step E, Compound (763) was prepared.

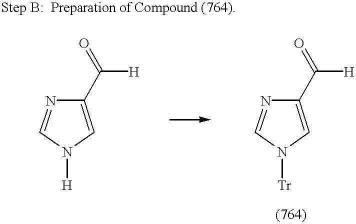

To a stirred solution of 4(5)-imidazolecarboxaldehyde (20.0 g, 0.208 mmol) in CH₂Cl₂ (200 mL), was added Et₃N (29.0 mL, 0.208 mmol). The solution was then cooled down at 0° C., followed by addition of triphenylmethylchloride (52.8 g, 0.18 mmol) at 0° C. The resulting solution was stirred at room temperature overnight and then washed it with brine, water and concentrated to dryness to give a white solid (63.0 g, 98% yield, MH⁺=339.1)

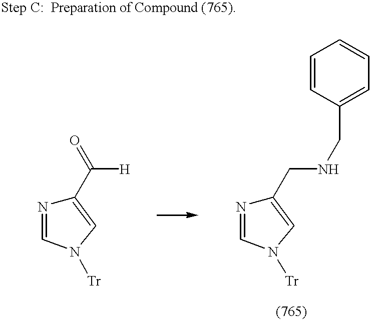

To a stirred solution of starting material benzyl amine (0.99 g, 8.87 mmol) in MeOH (50 mL) under nitrogen inlet at room temperature, was added sodium acetate (0.73 g, 8.87 mmol), 3°A molecular sieves (3.0 g) and aldehyde (3.0 g, 8.87 mmol). The resulting solution was stirred at room temperature overnight, followed by addition of NaBH₄ (0.67 g, 17.74 mmol), then stirred for 4 hrs and concentrated to dryness, followed by extraction with CH₂Cl₂-1N NaOH. The combined organic layer was dried over MgSO₄, filtered, concentrated to dryness and purified by column chromatography on silica gel, eluting with 2%MeOH—NH₃/98%CH₂Cl₂ to give light yellow oil (3.75 g, 98% yield, MH⁺=430.2)

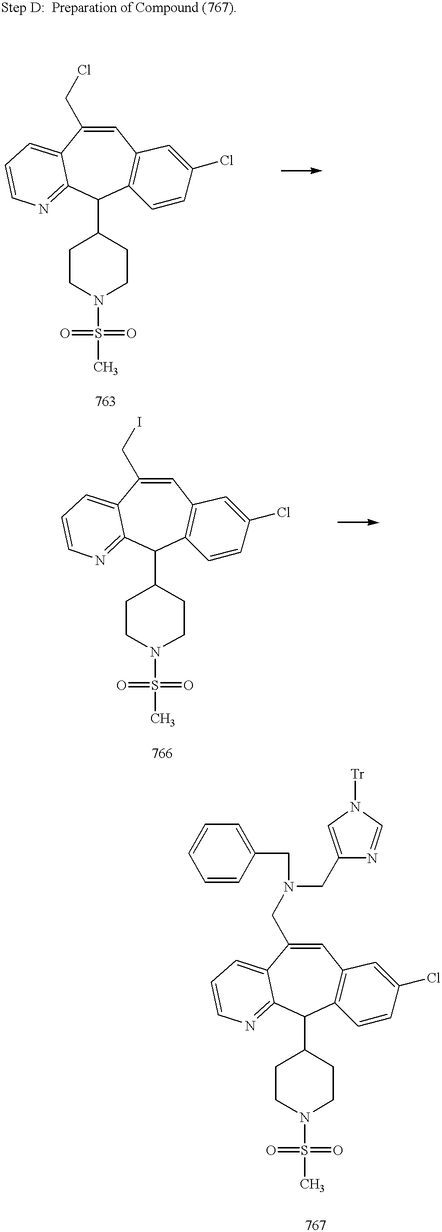

To a stirred solution of Compound (764) from step B (0.41 g, 1.14 mmol) in DMF (10 mL) under nitrogen at room temperature, was added NaH (0.02 g, 0.84 mmol). The resulting solution was stirred at room temperature for 1 hr.

To a stirred solution of Compound (763) from step A (0.4 g, 0.84 mmol) in acetone (30 mL) under nitrogen inlet at room temperature, was added Nal (0.12 g, 0.84 mmol). The resulting solution was heated to reflux for 1 hour and then concentrated to dryness to afford Compound (766). To crude Compound (766) was added, DMF (10 mL) and the solution of Compound (764) from above and NaH (0.02 g, 0.84 mmol). The resulting solution was heated to 90° C. for overnight, then concentrated to dryness and purified by column chromatography on silica gel, eluting with 2% MeOH—NH₃/98% CH₂Cl₂ to give Compound (767) as a yellow solid (0.23 g, 33% yield, MH⁺=830.4)

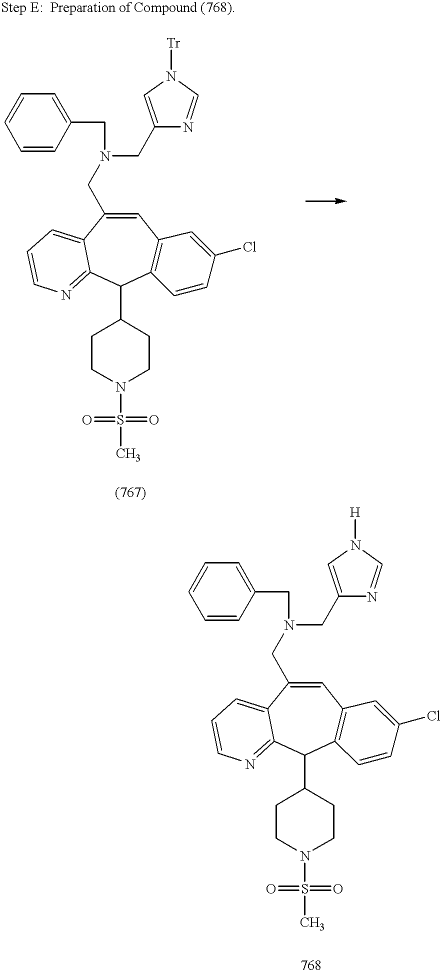

A solution of Compound (767) from step C (0.238 g, 0.29 mmol) in 80% acetic acid in H₂O was heated to reflux for 2 hrs and then concentrated to dryness, followed by extraction with CH₂Cl₂-1N NaOH. The combined organic layer was dried over MgSO₄, filtered, concentrated to dryness and purified by column chromatography on silica gel, eluting with 3% MeOH—NH₃/97%CH₂Cl₂to give white solid (0.10 g, 62% yield, M=588.2).

### Preparative Example 62

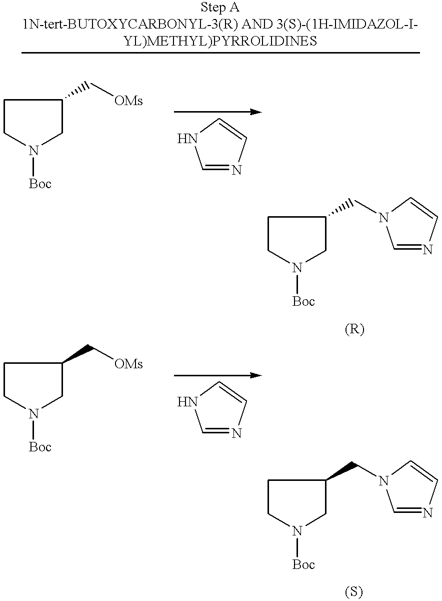

3(R)-(3-Methanesulfonyloxymethyl)pyrrolidine (J. Med. Chem. 1990, 33, 77-77) (0.993 g, 3.56 mmoles) was dissolved in anhydrous DMF (25 mL) and sodium imidazole (0.6 g, 10 mmoles) was added. The mixture was heated at 60° C. for 2h and then evaporated to dryness. The product was extracted with CH₂Cl₂ and washed with brine. CH₂Cl₂ extract was evaporated to dryness to give the titled compound (1.1409 g, 100%), ESMS: FABMS (M+1)=252; δ_{H} (CDCl₃) 1.45 (s, 9H), 1.5-1.7 (m,1H), 1.9-2.1 (m, 1H), 2.5-2.7 (m, 1H), 3.0-3.2 (m, 1H), 3.3-3.6 (m, 2H), 3.9 (dd, 2H), 6.9 (s, 1H), 7.1 (s,1H), 7.45 (s,1H)

In a similar manner, (S) isomer was prepared from 3(S)-(3-Methanesulfonyloxymethyl)pyrrolidine (0.993 g, 3.56 mmoles to give the title compound (1.1409 g, 100%).

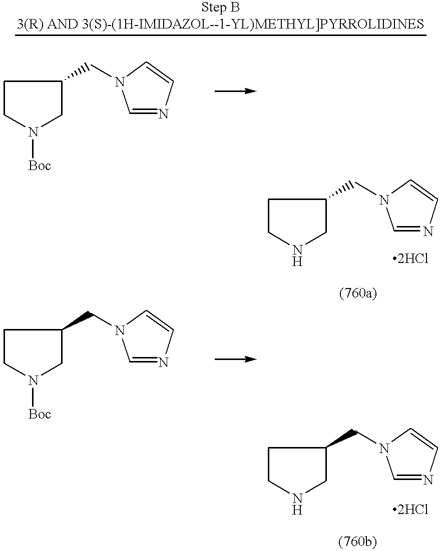

The title compound(0.48 g, 1.91 mmoles) from Step A was stirred in 4N HCl in dioxane (10 mL) for 2 h and then evaporated to dryness to give the title compound which was used to couple with the tricylic acid.

In a similar manner (S) isomer was prepared.

### Example 477

### Preparation of Compound (771)

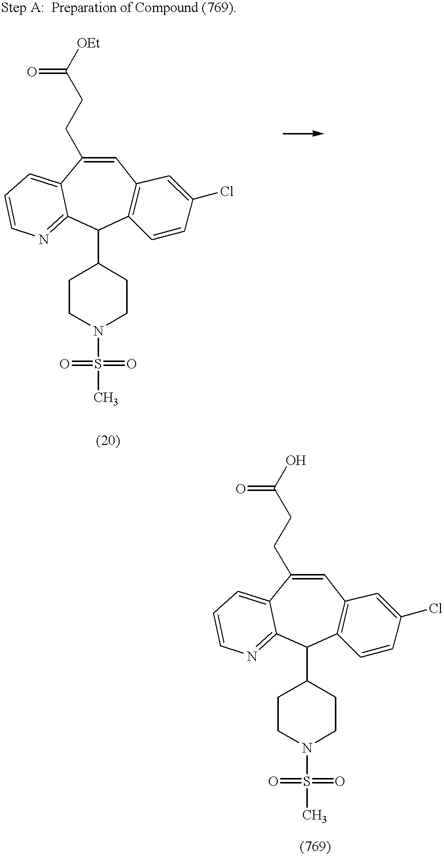

To a stirred solution of Compound (20) from preparative example 3 step B (4.86 g, 9.94 mmol) in EtOH (100 mL), was added 1N LiOH (80 mL). The resulting solution was then stirred at room temperature overnight and concentrated to dryness, followed by dissolving in CH₂Cl₂. The solution was then adjusted to pH=6.5-7.0 with 1N HCl. The aqueous layer was then separated and concentrated to dryness, then dissolved in THF to give the lithium salt (4.86 g, 100%yield,M+Li=467.1)

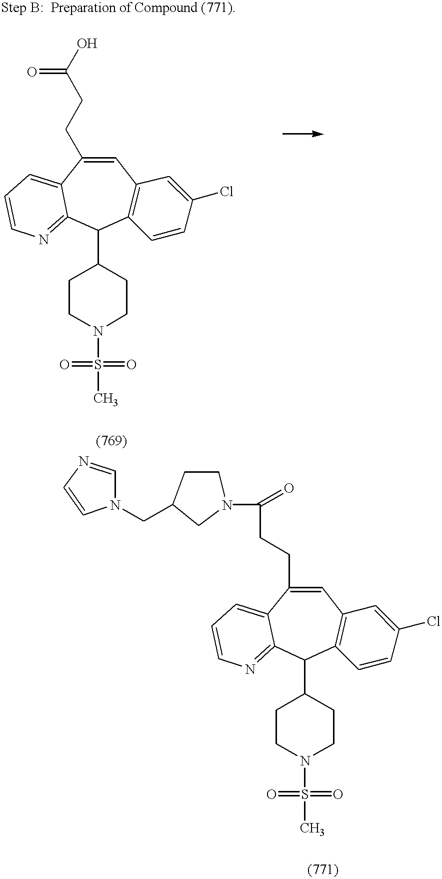

To a stirred solution of Compound (769) from step A above (0.38 g, 0.84 mmol) in DMF (10 mL) under nitrogen inlet at room temperature, was added Compound (770) from Preparative Example 62 (0.163 g, 1.09 mmol), benzotriazoyl-N-oxtris (dimethyl-amino)phosphoniumhexafluro phosphate (0.44 g, 1.01 mmol) and Et₃N (0.5 mL, 3.36 mmol). The resulting solution was stirred at room temperature overnight and concentrated to dryness, followed by extraction with CH₂Cl₂-10% Citric acid. The combined organic layer was then washed with saturated NaHCO₃, brine, dried over MgSO₄, filtered, concentrated to dryness and purified by column chromatography on silica gel, eluting with 3% MeOH—NH₃/CH₂Cl₂ to give a light yellow solid (0.12 g, M=594.2).

### Preparative Example 63

### Compound (772)

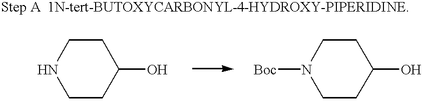

To a solution of 4-hydroxy-piperidine (2 g, 19.78 mmoles) and triethylamine (4.16 mL, 29.67 mmoles) in CH₂Cl₂ (20 mL), di-tert-butyidicarbonate (5.18 g, 23.72 mmoles) was added and stirred at room temperature for 16h. The solution was diluted with CH₂Cl₂ and washed with water, dried(MgSO₄) filtered and evaporated to give the title compound (3.95 g, 99%). FABMS (M+1)=202.

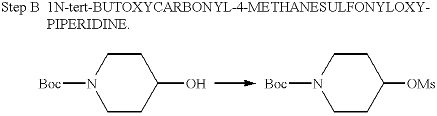

The title compound from Step A above (3.5 g, 17.39 mmoles) and triethylamine (4.85 mL, 34.79 mmoles) were dissolved in CH₂Cl₂ (30 mL) and the mixture was stirred under nitrogen at 0° C. Methanesulfonylchloride (1.62 mL, 20.88 mmoles) was added and the solution was stirred at room temperature for 2h. The solution was diluted with CH₂Cl₂ and washed with saturated aqueous sodium bicarbonate, water and dried (MgSO₄), filtered and evaporated to dryness to give the title compound (4.68 g, 96.4%). ESMS: m/z=280 (MH⁺)

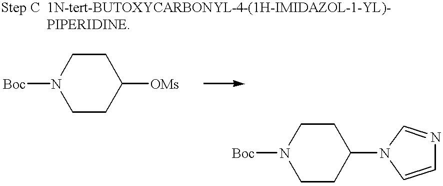

A solution of the title compound from Step B (4.0 g, 14.32 mmoles) in DMF (120 mL) was added to a stirred solution of NaH (0.52 g, 21.66 mmoles) and imidazole (1.46 g, 21.47 mmoles) in DMF (20 mL) under nitrogen atmosphere. The mixture was stirred at 60° C. for 16 h. DMF was evaporated in vacuo. The resulting crude product was extracted with CH₂Cl₂ and the extract was successively washed with water and brine, and the CH₂Cl₂ was evaporated to leave the title residue which was chromatographed on silica gel using 3% (10% conc NH₄OH in methanol)- CH₂Cl₂ as eluant to give the title compound (0.94 g, 26%). FABMS (M+1)=252; ·_{H} (CDCl₃) 1.4 (s, 9H), 1.6-1.8 (m, 2H), 2.0 (dd, 2H), 2.8 (dt, 2H), 4.05 (m, 1H), 4.2 m, 2H), 6.9 (s, 1H), 7.0 (s, 1H), 7.65 (s, 1H).

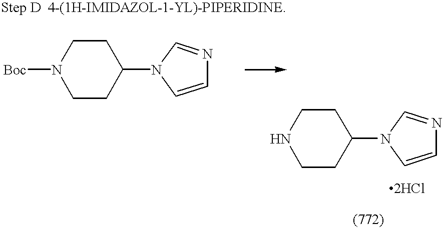

The title compound(0.21 g, 0.836 mmoles) from Step C was stirred in 4N HCl in dioxane (5 mL) for 2h and then evaporated to dryness to give the title compound (772)which was used to couple with the tricylic acid.

### Example 478

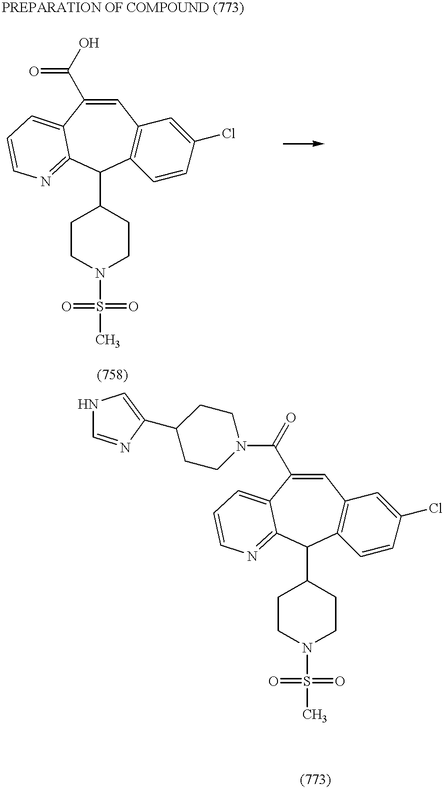

To a stirred solution of Compound (758) from Example 475 step D (0.2 g, 0.46 mmol) in CH₂Cl₂ (5 mL) under nitrogen at room temperature, was added Compound (772) from Preparative Example 63, Step D (0.19 g, 0.55 mmol), bezotriazoyl-N-oxy-tris-(dimethylamino)phosphoniumhexaflurophosphate (0.25 g, 0.55 mmol) and Et₃N (0.3 mL, 1.85 mmol). The resulting solution was stirred at room temperature overnight and concentrated to dryness, followed by extraction with CH₂Cl₂-10% citric acid. The combined organic layer was then washed with sat. NaHCO₃, brine, dried over MgSO₄, filtered concentrated to dryness and purified by column chromatography on silica gel, eluting with 3%MeOH—NH₃/CH₂Cl₂ to give a white solid (773) (0.013 g, 5% yield, M=566.2)

### Example 479

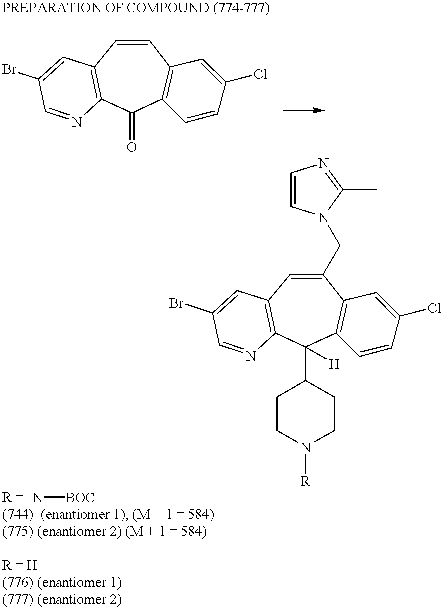

3-bromo-8-chloroazaketone (U.S. Pat. No. 5,977,128, Preparative Example 11, step A, (1999)) was reacted in essentially the same manner as in Preparative Example 23, and Example 91 to obtain the N-BOC derivatives (774) and (775). Compounds (774) and (775) were then reacted separately in essentially the same manner as in Preparative Example 19, Step D to obtain the enantiomers (776) and (777).

### Example 480

### Preparation of Compounds (778) and (779)

In essentially the same manner as in Examples (420) and (421), Compounds (778) and (779) were prepared.

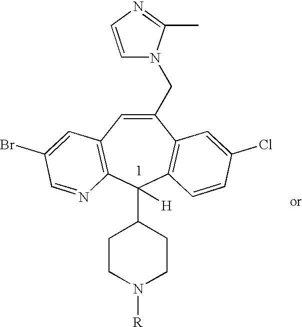

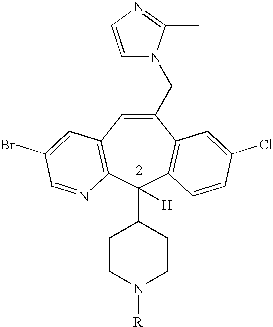

| Compound # | R = | Enantiomer | FABMS(M+1) |
|---|---|---|---|
| 778 | 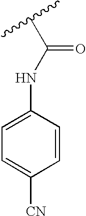 | 1 | 628 |
| 779 | 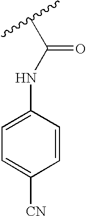 | 2 | 628 |
| Phys. Data | | | |
| (778): ¹H—NMR(Varians 400 MHz, CDCl₃, ppm): δ=8.564(1H, d, J=2 Hz), 7.784(1H, d, J=2 Hz), 7.624(1H, d, J=2 Hz), 7.51-7.37(5H, m), 7.305(1H, s), 7.267(1H, s), 6.870(1H, s), 6.867(1H, s), 6.579(1H, s), 5.282(1H, d, J=16 Hz), 5.031(1H, d, J=17 Hz), 4.576(1H, s), 3.176(4H, br ddd, J=6, 14 and 58 Hz), 2.485(3H, s), 1.950(4H, dd, J=6 and 9 Hz); MS(m/e)630(M+H), 340, 327, 293, 263, 249; HRMS | | | |
| #(Jeol JMS-HX110A) calcd for C31H27BrClN7O 628.1227(M+1), found 628.1229. | | | |

### Example 481

### Preparation of Compounds (780) and (781)

In essentially the same manner as in Example 70, Compounds (780) and (781) were prepared.

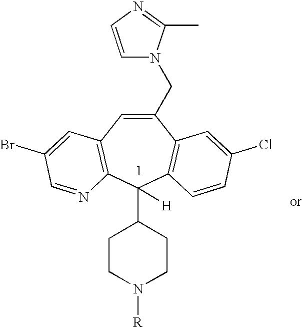

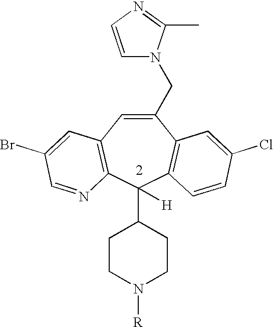

| Compound # | R = | Enantiomer | FABMS(M+1) |
|---|---|---|---|
| 780 | 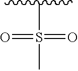 | 1 | 562 |
| 781 | 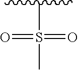 | 2 | 562 |

### Preparative Example 64

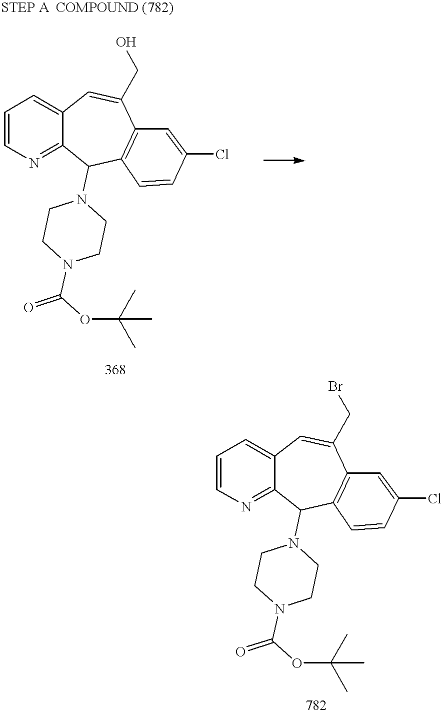

Compound (368) from Preparative Example 42, Step C (2.34 g, 5.29 mmol) was dissolved in 25 mL CH₂Cl₂ at 0° C. PPh₃ (1.66 g, 6.34 mmol) and NBS (1.03 g, 5.82 mmol) were added. After 90 mins, the reaction was diluted with CH₂Cl₂ (20 mL), washed with sat. NaHCO₃, brine and dried with MgSO₄. The crude product was purified on a silica gel column (4:1 hexanes/EtOAc to 2:1) to yield 1.8 g of Compound (782) as a light yellow solid. MS M+1 504.

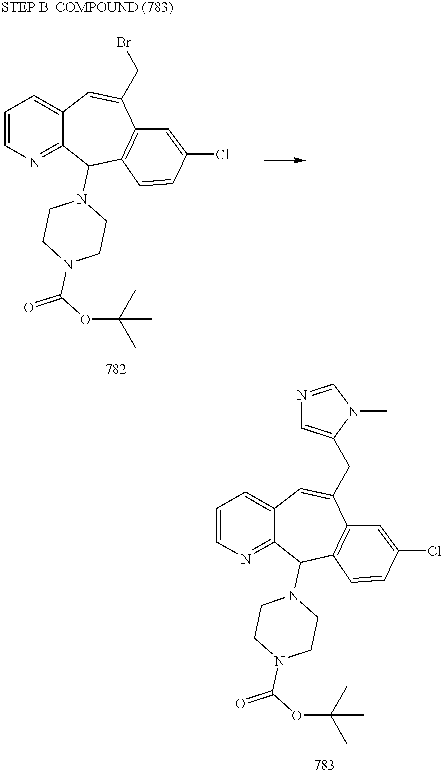

5-Iodo-1N-methyl imidazole (455 mg, 2.18 mmol) was dissolved in 10 mL THF at room temperature. EtMgBr (2.4 mL, 1.0 M in THF) was added dropwise. After 30 mins, the reaction mixture was cooled to 0° C. 10 mL THF solution of CuCN (175 mg, 1.96 mmol) and LiCl (166 mg, 3.9 mmol) was then added. 10 mins later, Compound (782) from Step A above (989 mg, 1.96 mmol, in 10 mL THF) was added. The reaction was stirred overnight. Sat. NH₄Cl solution was added to quench the reaction. The resulting emulsion was filtered through a sintered funnel and the filtrate was extracted with EtOAc twice. The organic layer was washed with NaHCO₃ solution and brine, dried over magnesium sulfate, filtered and evaporated in vivo. The resulting crude material was chromatographed on a silica gel column (using 1:1 hexanes/EtOAc then 10:1 CH₂Cl₂/MeOH) to obtain 330 mg of the title product. MS M+1=506 The enantiomers were seperated on a chiral AD column.

### Example 482

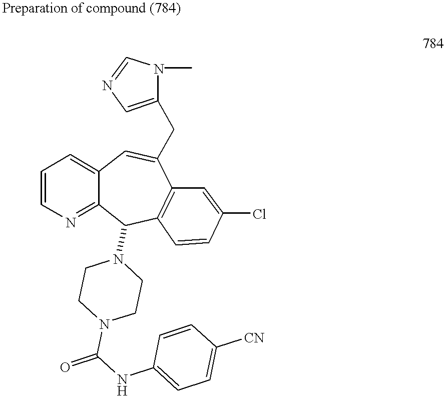

Compound (783) from Preparative Example 64, Step B above (40 mg ) was dissolved in CH₂Cl₂ (5 mL) at room temerature followed by addition of TFA (0.5 mL). After 2 hrs, the solvent was evaporated in vivo and coevaporated with PhCH₃ twice. The crude mixture was then dissolved in CH₂Cl₂ (4 mL) and Et₃N was added dropwise till the solution became basic by PH paper. 4-Cyanophenyl isocyanate (14 mg) was added. After 5 minutes, the reaction mixture was evaporated in vivo to dryness. The crude material was then purified using prep TLC plate (10:1 CH₂Cl₂/MeOH) to get 23 mg of Compound (784) as a white solid. MS M+1 550.

### Example (483)

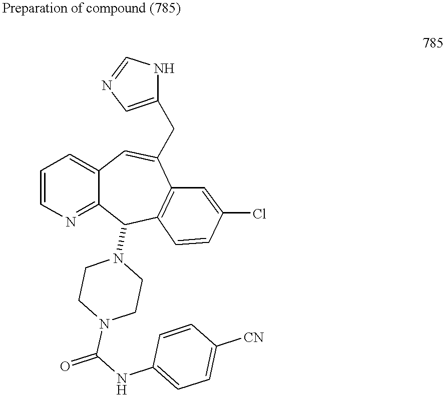

Compound (785) was prepared following essentially the same procedure as in Preparative Example 64 and Example 482, substituting 4-Iodo-1-trityl imidazole for 5-Iodo-1N-methyl imidazole.

### Example 484

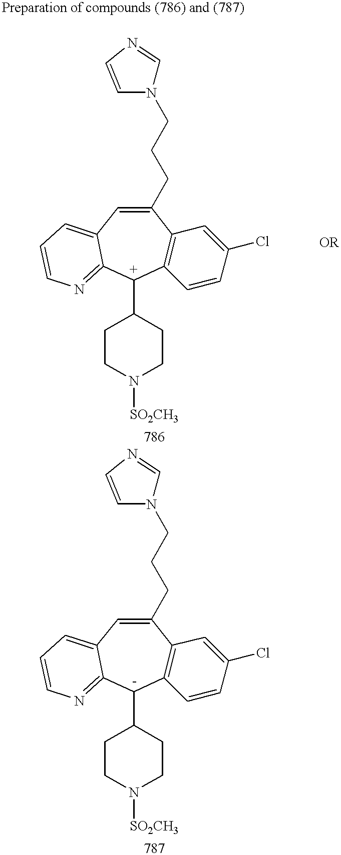

Compound (786) and (787) were prepared following essentially the same procedure as in Preparative Example 7, substituting ketones (15) and (16) from Preparative Example 2, Step D for ketones (9) and (10).

Compound (786) MH⁺=497; [α]_{D}²⁰ =+15.3;

Compound (787) MH⁺=497; [α]_{D}²⁰=−13.4.

### Example 485

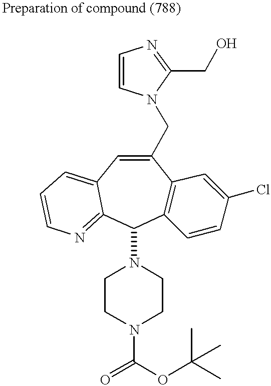

Following essentially the same procedure as in Preparative Example 33, Steps E-H, except substituting compound (365) for Compound (281) and 2-hydroxymethyl imidazole for 1-methyl imidazole, compound (788) was prepared. (788): ¹H-NMR (Varians 400 MHz, CDCl₃, ppm): δ=8.5 (1H,dd), 7.34 (1H,s), 7.59 (1H, d), 7.4 (2H, m), 7.25 (2H, m), 7.04 (1H, s), 6.9 (1H, s), 6.6 (1H, s), 5.37 (2H, dd), 4.8 (2H, dd), 4.6 (1H, s), 3.2 (5H, br s), 2.0 (2H, br s), 1.9 (2H, br s), 1.4 (9H, s).

### Preparative Example 65

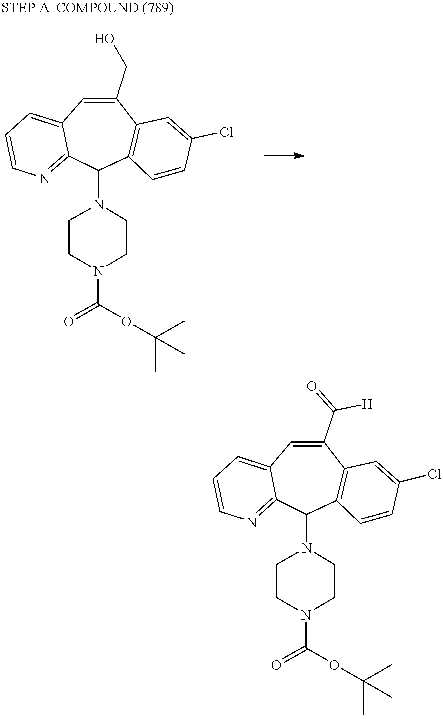

To a solution of the alcohol (3.8 g, 8.6 mmol) in CH₂Cl₂ (100 mL) under nirtogen was added MnO₂ (40 g). The resulting solution was stirred at room temperature for 4 days. The mixture was then filtered through a pad of Celite with ethyl acetate (500 mL) as the eluant. The filtrate was concentrated to yield a yellow liquid (4.0 g, MH+440.1). The crude material was separated into its pure isomers by HPLC, using a chiral AD column eluting with 20% IPA/80%Hexanes/0.2%DEA (isomer 1, 810 mg; isomer 2, 806 mg).

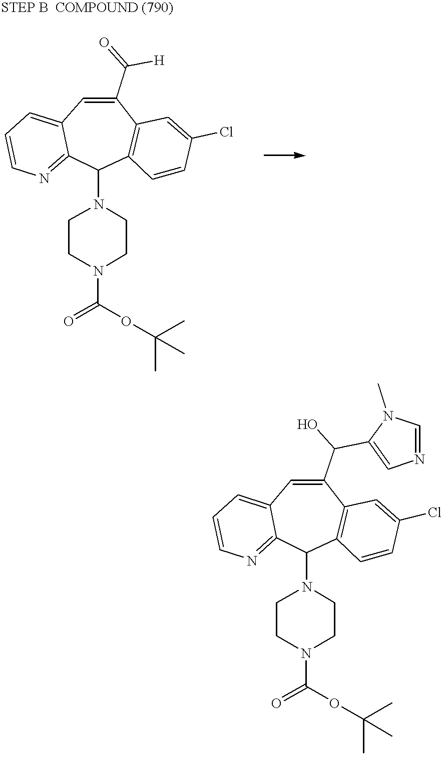

To a solution of imidazole Grignard prepared from 5-iodo-1N-methyl imidazole (312 mg, 1.5 mmol, preparative example 64 step B) was added a solution of aldehyde (791) (380 mg, 0.86 mmol) in CH₂Cl₂ (10 mL). After stirring at room temperature overnight, the mixture was heated to 40° C. for one hour. After cooling to room temperature again, saturated NH₄Cl solution was added to quench the reaction. The organic layer was dried and the solvent was evaporated. The residue was then purified by silica gel column (from 2% to 10% MeOH in CH₂Cl₂) to give the product as a brown oil (207 mg, 46% yield, MH+=522.1). The diastereomers were then separated by HPLC, using a chiral AD column eluting with 20% IPA/80%Hexanes/0.2%DEA.

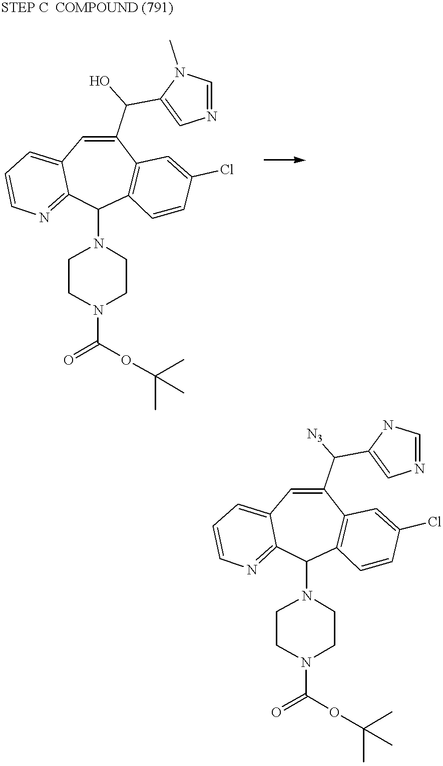

To a THF solution (5 mL) of (790) (200 mg, 0.38 mmol) at room temperature was added DPPA (210 mg, 0.76 mmol) followed by addition of DBU (120 mg, 0.76 mmol). The mixture was stirred overnight and then diluted with ethyl acetate (30 mL), washed with water twice and brine once. The organic layer was dried and the solvent was evaporated. The residue was purified by prep TLC (10% MeOH in CH₂Cl₂ with 0.2% NH₃) to give product (791) (102.8 mg, MH+547.1). Starting material (790) (58 mg) was also recovered. The diastereomers of (791) were separated on a chiral AD column.

### Example 486

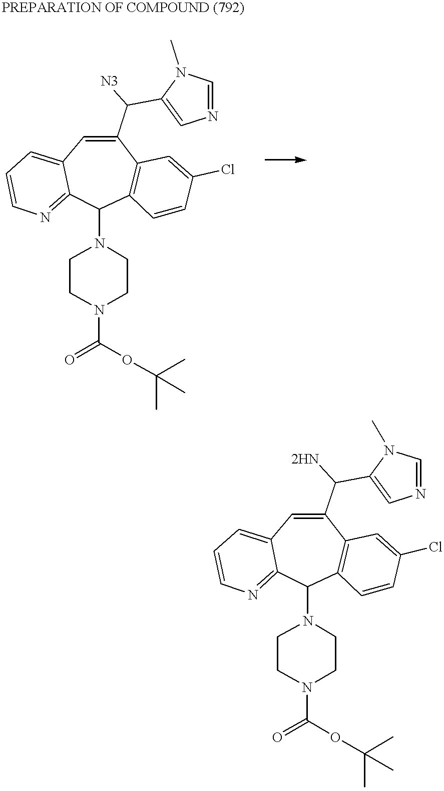

To a wet THF solution (3 mL) of (791) (48 mg, 0.09 mmol) was added PPh₃ (32 mg, 0.12 mmol) at room temperature. After stirring overnight, the reaction mixture was concentrated and the residue was purified with prep TLC (10% MeOH in CH₂Cl₂ with 0.2% NH₃) to give a white solid (24.3 mg). The white solid was then redissolved in THF/H₂O (5 mL/0.5 ml) and the mixture was heated to reflux overnight. The reaction mixture was then partitioned between ethyl acetate and water. The organic layer was dried and concentrated. The residue was purified with prep TLC (5% MeOH in CH₂Cl₂ with 0.2% NH₃) to yield a yellow solid (792) (8.3 mg, MH+521.1).

### Example 487

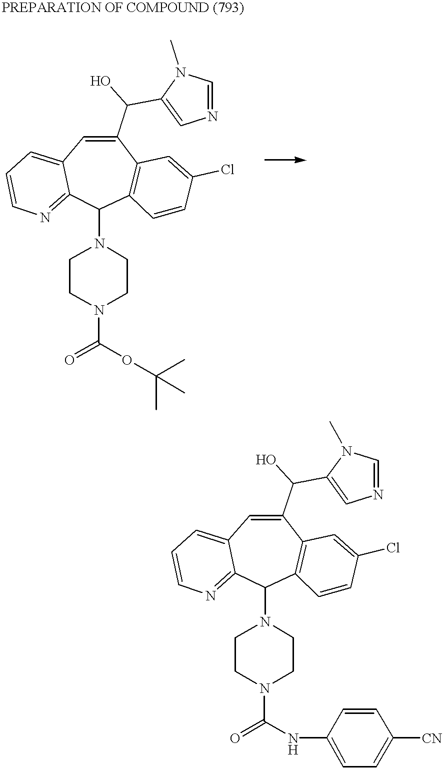

Compound (790) was converted to compound (793) following the essentially the same procedure as described in EXAMPLE 482. MS M⁺¹ 566.1.

### Example 488

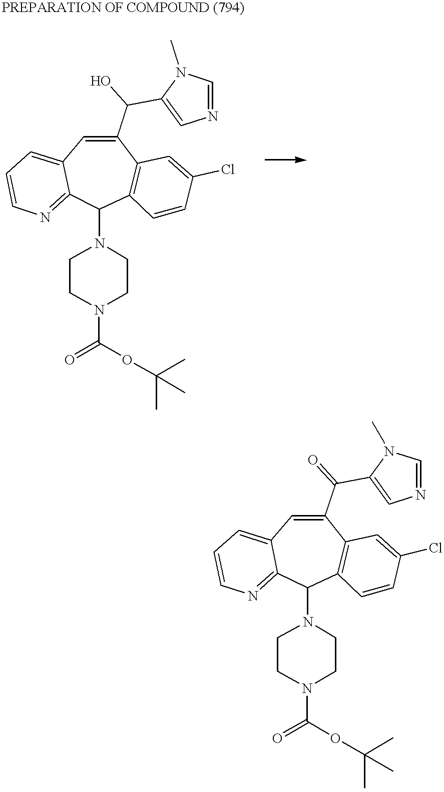

Compound (790) was converted to compound (794) following essentially the same procedure as described in PREPARATIVE EXAMPLE 65, Step A. MS M⁺¹ 520.1.

### Example 489

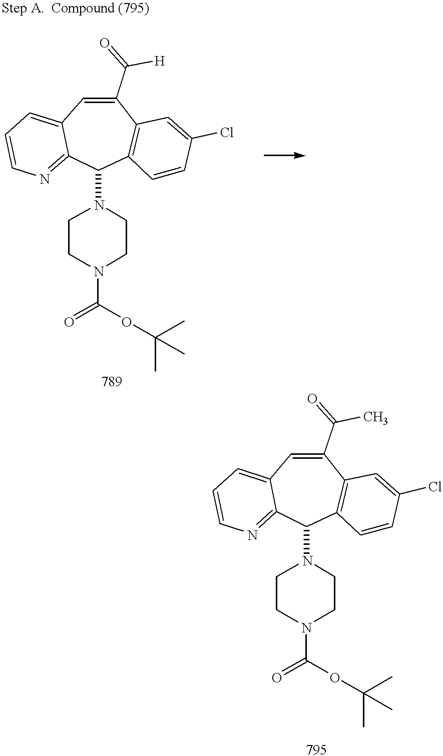

Aldehyde (789) from Preparative Example 65, Step A (150 mg, 0.34 mmol) was dissolved in THF (6 mL). To this solution was added MeMgBr (0.3 mL, 3.0M in Et₂O) dropwise. After stirring at room temperature for 4 hrs, the reaction mixture was quenched with sat. NH₄Cl solution and extracted with ethyl acetate. The organic layer was washed with brine, dried and concentrated to give a yellow solid (150 mg). The crude product was then dissolved in CH₂Cl₂ (5 mL). To this solution was added Dess-Martin Periodinane (210 mg) and a drop of water. After 1 hr, aqueous Na₂S₂O₃ solution (4 mL, 10%) was added. The mixture was stirred for 10 min. and extracted with CH₂Cl₂. The organic layer was washed with NaHCO₃, dried and concentrated. The crude material was purified using prep TLC plates (5% methanol in CH₂Cl₂) to yield the methyl ketone product (795) as a yellow solid (70 mg).

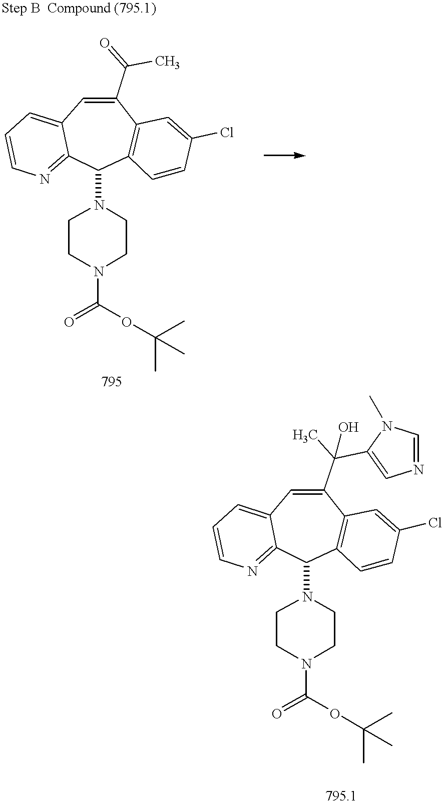

To a solution of imidazole Grignard prepared from 5-iodo-1N-methyl imidazole (624 mg, 3 mmol, see preparative example 64 step B using ClCH₂CH₂Cl as solvent instead of THF) was added a ClCH₂CH₂Cl (6 mL) solution of methyl ketone (795) (272 mg, 0.6 mmol). The mixture was heated to 60° C. for 1.5 hours. After cooling to room temperature, saturated NH₄Cl solution was added to quench the reaction. The organic layer was dried and then evaporated to dryness. The residue was then purified by silica gel column (from 2% to 10% MeOH in CH₂Cl₂) to give the product (795.1) as a brown solid (63 mg, 10:1 diastereomeric selectivity, MH+=536.1). Major diastereomer: (CDCl₃, 300 MHz) 8.47 (d, 1H), 7.66 (d,1H), 7.57 (s, 1H), 7.54 (s,1H), 7.34 (d, 1H), 7.25-7.22 (m,1H), 7.05 (s, 1H), 6.89 (s, 1H), 6.82 (s,1H), 4.61 (s, 1H), 3.84 (s, 3H), 3.24 (br s, 4H), 2.24 (m, 2H), 2.02-2.00 (m, 2H), 1.88 (s, 3H), 1.41 (s, 9H).

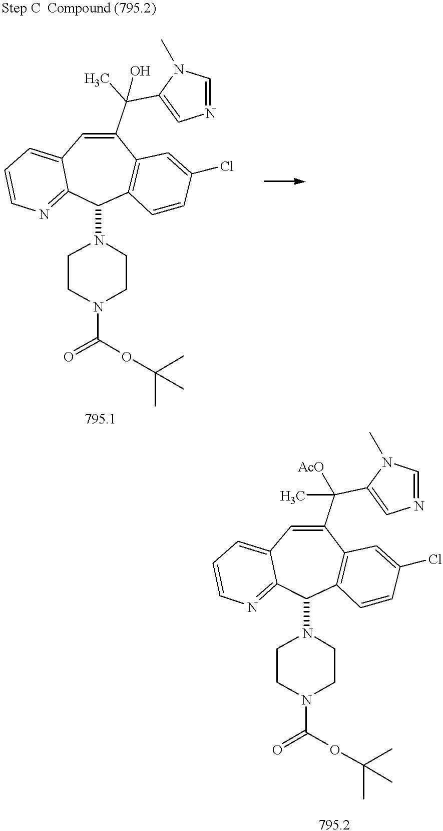

Compound (795.1) can be converted to acetate compound (795.2) by reacting it with 1 equivalent of acetic anhydride and 2 equivalents of pyridine.

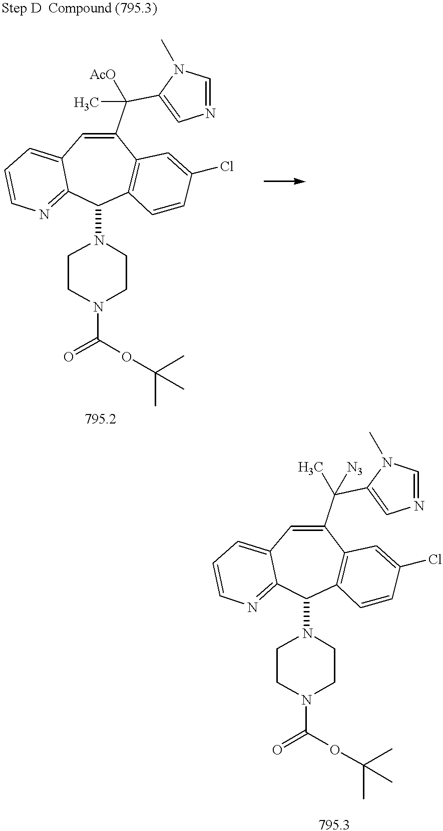

Compound (795.2) can be converted to compound (795.3) by reacting it with 1.5 equivalents of NaN₃, 15-crown-5, and a catalytic amount of Pd(dba)₂/PPh₃.

Alternatively, (795.3) can be synthesized by treating (795.1) with NaN₃, TFA followed by (Boc)₂O, and triethyl amine.

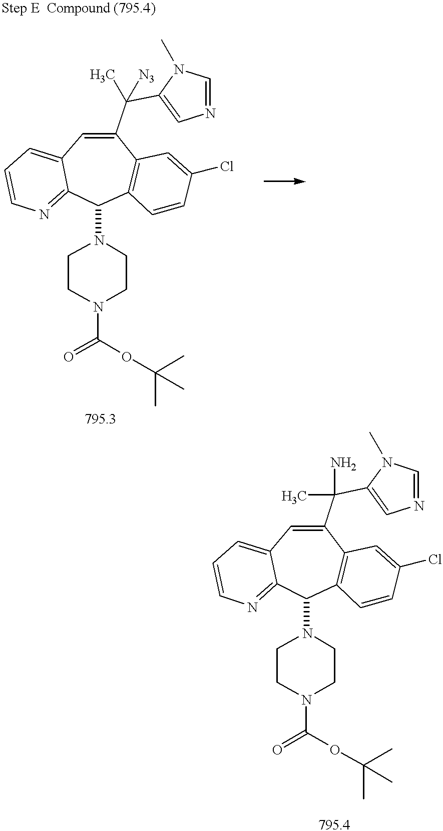

Compound (795.4) can be prepared by reacting (795.3) with P(CH₃)3/H₂O.

### Preparative Example 66

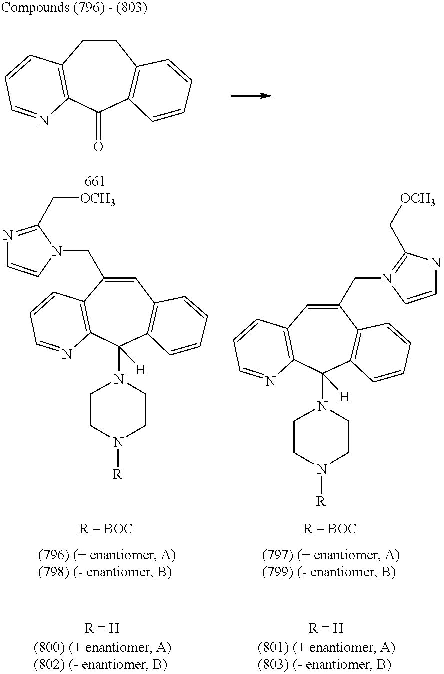

Compound 661 was reacted in essentially the same manner as in Preparative Example 23 and then Example 91 to obtain the N-BOC derivatives (796), (797), (798), and (799). Compounds (796), (797), (798), and (799) were then further reacted separately in essentially the same manner as in PREPARATIVE EXAMPLE 19, Step D to obtain the enantiomers (800), (801) (+ enantiomers, isomer A) and (802), (803) (− enantiomers, isomer B). The C5 and C-6 vinyl bromide intermediates were separated by silica gel chromatography using hexane:ethyl acetate (80:20) as described in PREPARATIVE EXAMPLE 23, Step B.

### Example 490-491

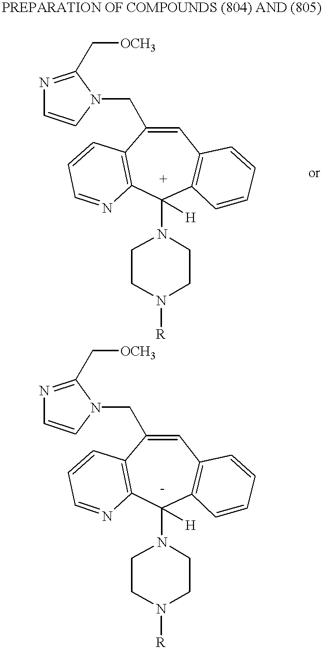

The appropriate (+) enantiomer (800) or (−) enantiomer (802) from Preparative Example 66 above, was taken up in CH₂Cl₂ treated with the corresponding isocyanate and stirred at room temperature over night. The crude product was purified directly by silica gel preparative thin layer chromatography or silica gel column chromatography to afford the following compounds in the table below:

| Example # | R | Enantiomer | Comp # | Phys. Data. |
|---|---|---|---|---|
| 490 | 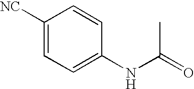 | + | (804) | Mp = 160-165° C. [α]_{D}²⁵ = +84°(0.84 mg/1 mL MeOH) MH+ = 546 |
| 491 | 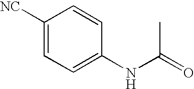 | − | (805) | Mp = 158-163° C. [α]_{D}²⁵ = −91.6°(0.84 mg/1 mL MeOH) MH+ = 546 |

### Preparative Example 67

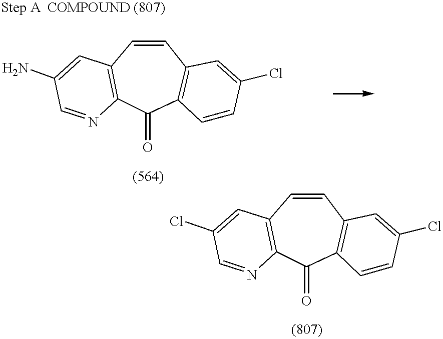

15.4 g (115 mmole) of CuCl₂ and 17 mL (144 mmol) of t-butyl nitrite was added to 400 mL of dry CH₃CN. The reaction mixture was cooled to 0° C. and 25 g of ketone (564) was added. The reaction was warmed to room temperature and stirred for two days. The mixture was concentrated under vacuum. Then 1N HCl was added to the residue until the pH was neutral, then NH₄OH was added until the pH was basic. After extraction with ethyl acetate, the organic layer was dried over MgSO₄ and concentrated under vacuum to give compound (807). Alternatively, the corresponding alcohol of 564 can be reacted as above followed by oxidation with MnO₂ in CH₂Cl₂ to give compound (807).

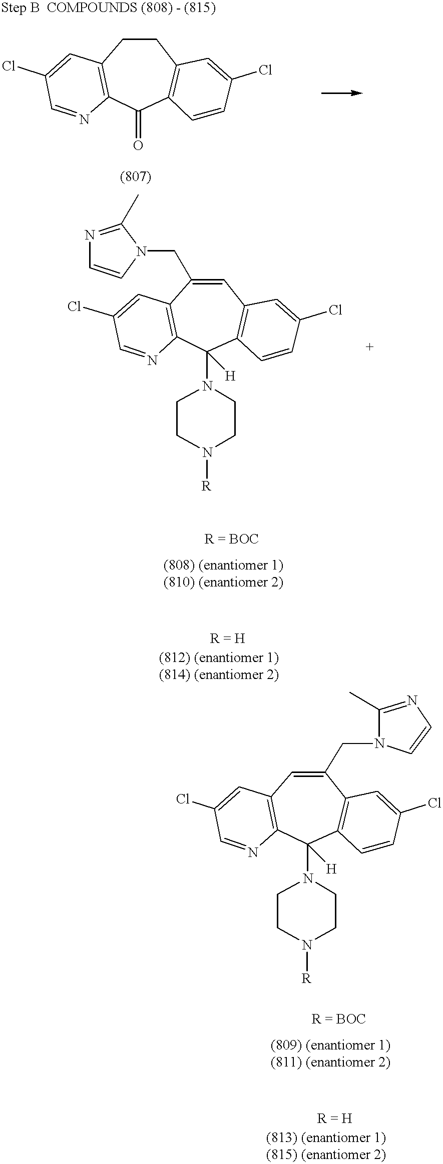

Compound (807) from step B above was reacted in essentially the same manner as in Preparative Example 23, and then Example 91 to obtain the N-BOC derivatives (808), (809), (810) and (811). These were then reacted separately in essentially the same manner as in Preparative Example 19, Step D to obtain the enantiomers (812) and (814), as well as enantiomers (813) and (815). The C5 an d C-6 vinyl bromide intermediates were separated by silica gel chromatography using hexane:ethyl acetate as described in Preparative Example 23, Step B.

### Example 493

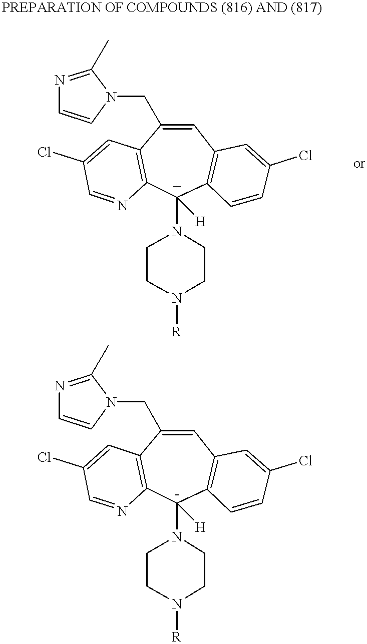

The appropriate enantiomer (812) (enantiomer 1) or (814) (enantiomer 2) from Preparative Example 67, Step B above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Starting | | | | |
|---|---|---|---|---|
| Cmp. # | R | Enantiomer | Comp # | Phys. Data. |
| (812) | 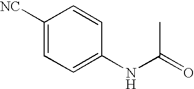 | + | 816 | Mp = 175-181° C. [α]_{D}²⁵ = +94.2°(1 mg/1 mL MeOH) |
| (814) | 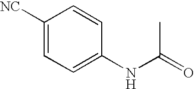 | − | (817) | Mp = 182-186° C. [α]_{D}²⁵ = −120.3°(1 mg/1 mL MeOH) |

### Example 494

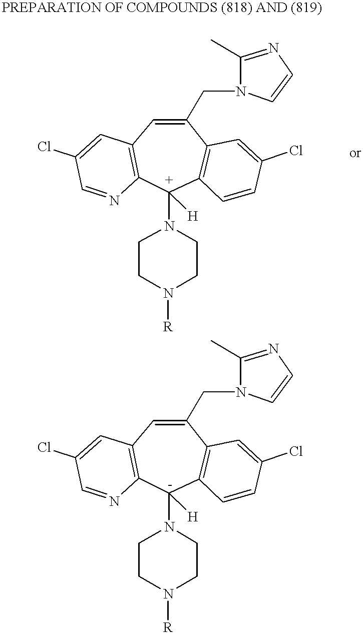

The appropriate enantiomer (813) (enantiomer 1) or (815) (enantiomer 2) from Preparative Example 67, Step B above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Starting | | | | |
|---|---|---|---|---|
| Cmp # | R | Enantiomer | Cmp # | Phys. Data. |
| (813) | 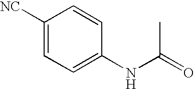 | + | (818) | Mp = 176-181° C. [α]_{D}²⁵ = +46.3°(0.79 mg/1 mL MeOH) MH+ = 584 |
| (815) | 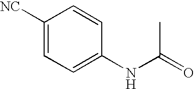 | − | (819) | Mp = 174-180° C. [α]_{D}²⁵ = −43.3°(0.94 mg/1 mL MeOH) MH+ = 584 |

### Preparative Example 68

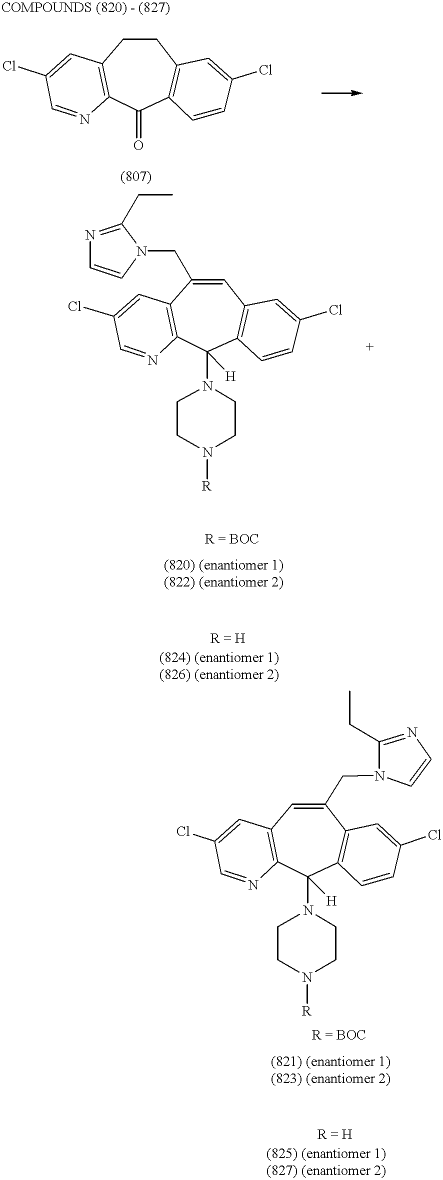

Compound (807) from Preparative Example 67, Step A above was reacted in essentially the same manner as in Preparative Example 23, and then Example 91, substituting 2-ethylimidazole for 2-methylimidazole, to obtain the N-BOC derivatives (820), (821), (822) and (823). These were then reacted seperately in essentially the same manner as in Preparative Example 19, Step D to obtain the enantiomers (824) and (826), as well as enantiomers (825) and (827). The C5 and C-6 vinyl bromide intermediates were separated by silica gel chromatography using hexane:ethyl acetate as described in Preparative Example 23, Step B.

### Example 495

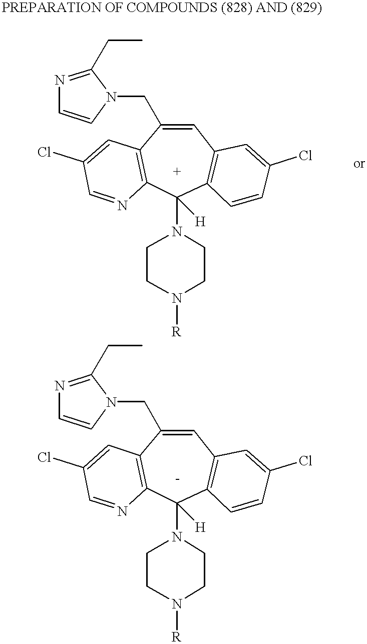

The appropriate enantiomer (824) (enantiomer 1) or (826) (enantiomer 2) from Preparative Example 68 above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Start- | | | | |
|---|---|---|---|---|
| ing | | Enan- | | |
| Cmp | | tio- | | |
| # | R | mer | Comp # | Phys. Data. |
| (824) | 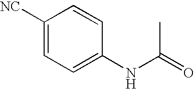 | + | (828) | Mp =176-182° C. [α]_{D}²⁵ =+84.5° (1.3 mg/1 mL MeOH) MH+ = 598 |
| (826) | 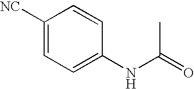 | − | (829) | Mp =175-182° C. [α]_{D}²⁵ =−88.8° (1.14 mg/1 mL MeOH) MH+ = 598 |

### Example 496

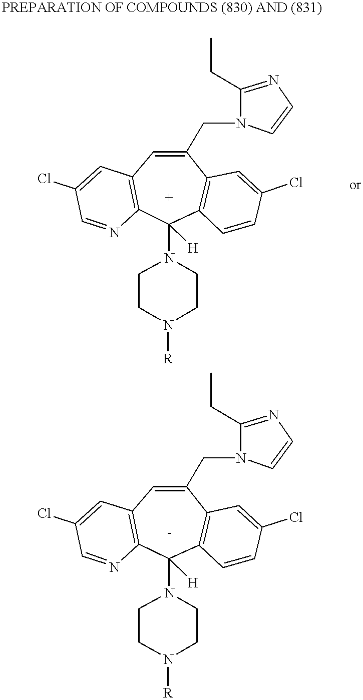

The appropriate enantiomer (825) (enantiomer 1) or (827) (enantiomer 2) from Preparative Example 68 above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Start- | | | | |
|---|---|---|---|---|
| ing | | Enan- | | |
| Cmp | | tio- | | |
| # | R | mer | Comp # | Phys. Data. |
| (825) | 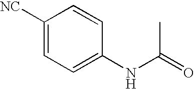 | + | (830) | Mp =170-174° C. [α]_{D}²⁵ =+39.1° (0.81 mg/1 mL MeOH) MH+ = 598 |
| (827) | 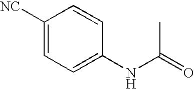 | − | (831) | Mp =170-175° C. [α]_{D}²⁵ =−36.4° (0.96 mg/1 mL MeOH) MH+ = 598 |

### Preparative Example 69

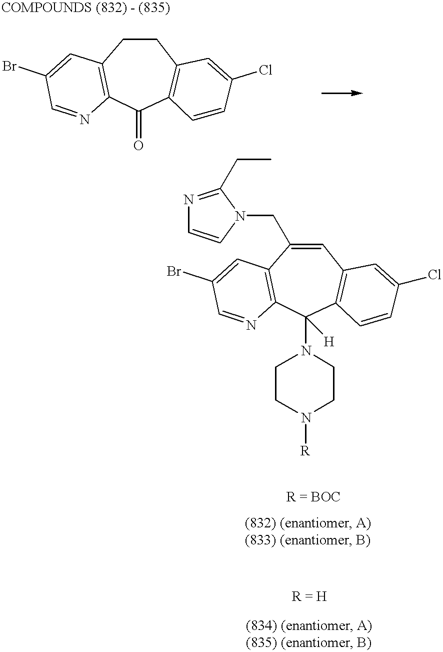

3-Bromo-8-chloroazaketone (U.S. Pat. No. 5,977,128, Preparative Example 11, Step A, (1999)) was reacted in essentially the same manner as in Preparative Example 23, and then Example 91, substituting 2-ethylimidazole for 2-methylimidazole, to obtain the N-BOC derivatives (832) and (833). These were then reacted separately in essentially the same manner as in Preparative Example 19, Step D to obtain the enantiomers (834) and (835).

### Example 497

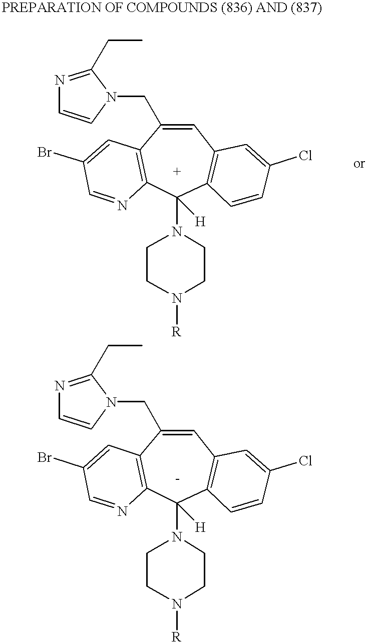

The appropriate enantiomer (834) (enantiomer 1) or (835) (enantiomer 2) from Preparative Example 69 above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Start- | | | | |
|---|---|---|---|---|
| ing | | | | |
| Cmp | | Enan- | | |
| # | R | tiomer | Comp # | Phys. Data. |
| (834) | 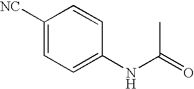 | A | (836) | Mp =172-179° C. (d) MH+ = 643 |
| (835) | 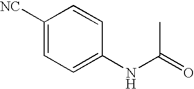 | B | (837) | Mp = 171.9-178.3° C. MH+ = 643 |

### Preparative Example 70

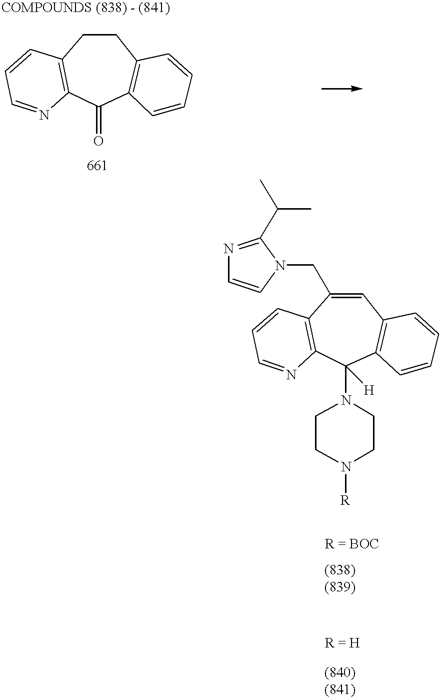

Compound 661 was reacted in essentially the same manner as in Preparative Example 23, and then Example 91, substituting 2-isopropylimidazole for 2-methylimidazole, to obtain the N-BOC derivatives (838) and (839). These were then reacted separately in essentially the same manner as in Preparative Example 19, Step D to obtain the enantiomers (840) and (841).

### Example 498

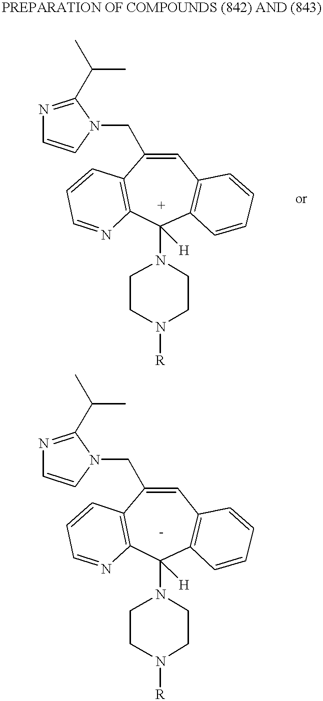

The appropriate enantiomer (840) (enantiomer 1) or (841) (enantiomer 2) from Preparative Example 70 above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Start- | | | | |
|---|---|---|---|---|
| ing | | | | |
| Cmp | | Enan- | | |
| # | R | tiomer | Comp # | Phys. Data. |
| (840) | 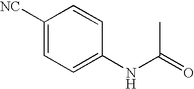 | A | (842) | Mp = 168-170° C. (d) [α]_{D}²⁵ =+64.1° (0.66 mg/1 mL MeOH) |
| (841) | 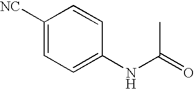 | B | (843) | Mp = 166-171° C. [α]_{D}²⁵ =−80.9° (0.85 mg/1 mL MeOH) |

### Preparative Example 71

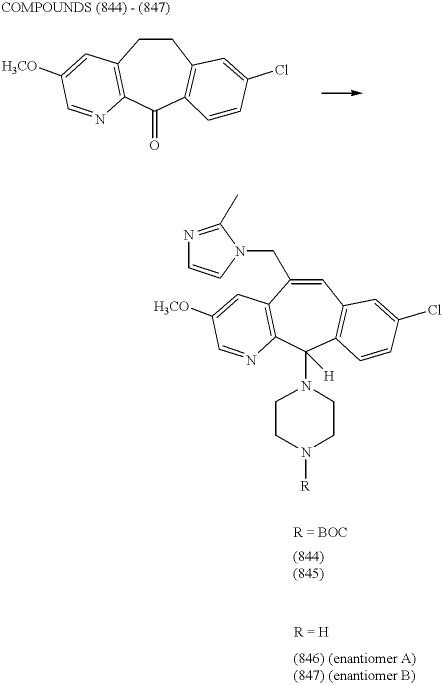

3-Methoxy-8-chloroazaketone (U.S. Pat. No. 5,977,128 (1999), Example 2, step D) was reacted in the same manner as in Preparative Example 23, and Example 91 to obtain the N-BOC derivatives (844) and (845). These compoounds were then reacted separately in essentially the same manner as in Preparative Example 19, Step D to obtain the enantiomers (846) (A) and (847) (B).

### Example 499

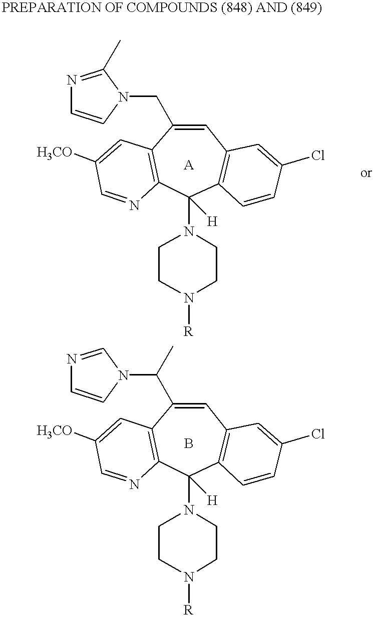

The appropriate enantiomer (846) (enantiomer A) or (847) (enantiomer B) from Preparative Example 71 above, was taken up in CH₂Cl₂, treated with 4-cyanophenyl isocyanate and stirred at room temperature over night. The crude product was purified directly by silica gel preparative thin layer chromatography or silica gel column chromatography to afford the following compounds in the table below:

| Start- | | | | |
|---|---|---|---|---|
| ing | | | | |
| Cmp | | Enan- | | |
| # | R | tiomer | Comp # | Phys. Data. |
| (846) | 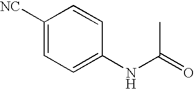 | A | (848) | Mp = 174.2-189.3° C. (d) MH+ = 580 |
| (847) | 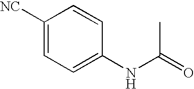 | B | (849) | Mp = 174.4-189.8° C. MH+ = 580 |

### Example 500

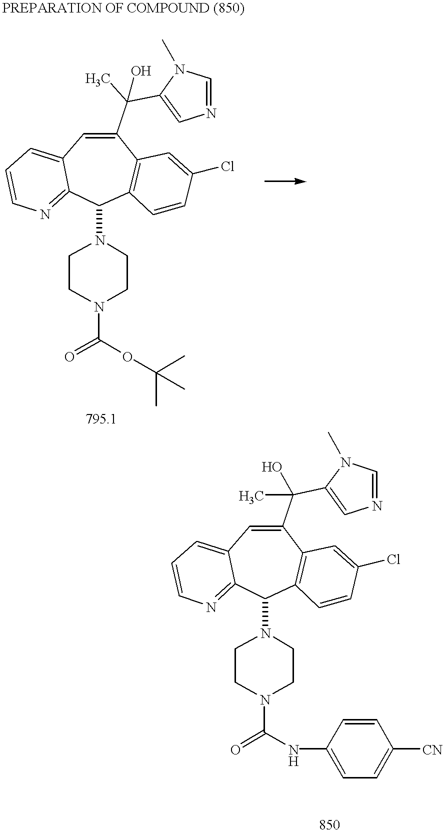

Compound (850) can be prepared by following essentially the same procedure as described in Example 482.

### Example 501

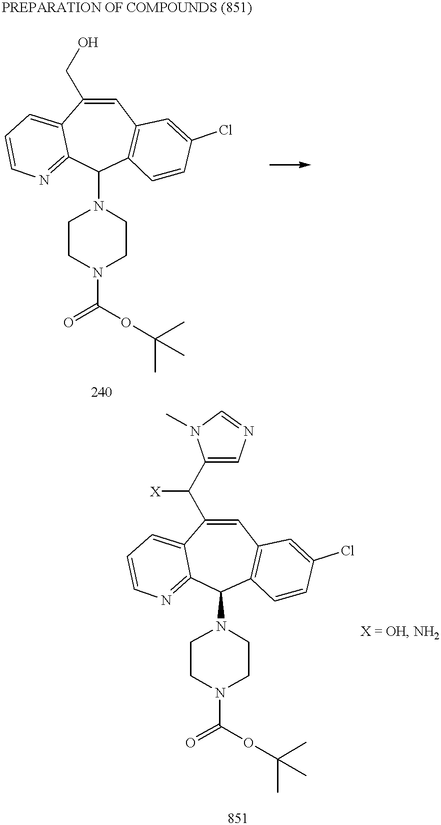

Starting with compound (240) from Preparative Example 23, Step H, compound (851) can be prepared following essentially the same procedure as described in Preparative Example 65, Steps A and B.

### Example 502

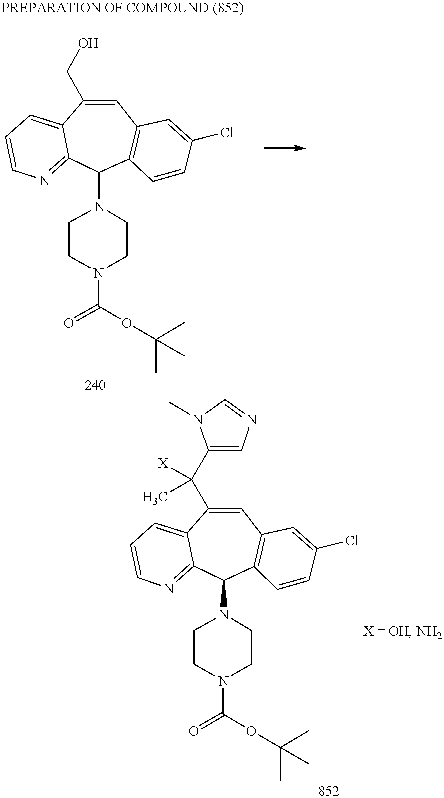

Starting with compound (240) from Preparative Example 23, Step H, compound (852) can be prepared following essentially the same procedures as described in Preparative Example 65, Step A and Example 489, Steps A-E.

### Preparative Example 72

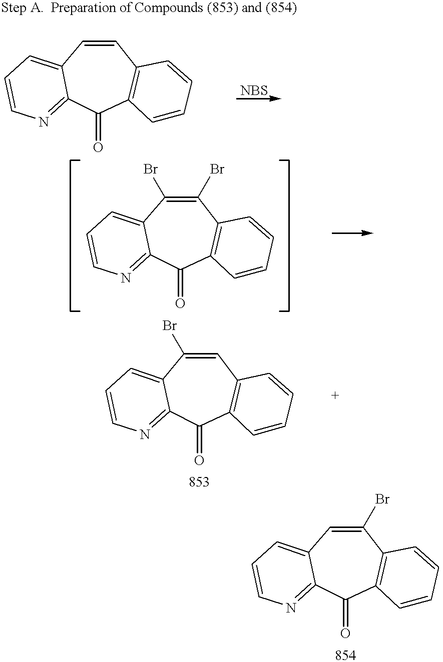

The starting tricyclic keto compound (disclosed in US Pat. No. 5,151,423) (56.5 g; 270 mmol) was combined with NBS (105 g; 590 mmol) and benzoyl peroxide (0.92 g) in CCl₄. The reaction was heated at 80° C. for 5 hr. The mixture was cooled and the resulting precipitate was filtered and treated with DBU (25.59 ml) in THF (300 mL). The resulting solution was stirred at room temperature for 24 hrs, then evaporated, followed by extraction with CH₂Cl₂-H₂O. The organic layer was dried over MgSO₄, filtered and evaporated to dryness to give a mixture of two compounds which were separated on a flash silica gel column eluting with Hexane-50% EtOAc to give the title compound (853) δ_{H} (CDCl₃) 8.8 (dd,1H), 8.45 (dd,1H), 7.99 (m,1H), 7.92 (s,1H), 7.59-7.64 (m, 3H), 7.23 (dd,1H) and (854) ⁸H (CDCl₃) 8.19 (dd,1H), 7.99 (dd,1H), 7.82 (dd, 1H), 7.25-7.65(m, 4H), 7.22 (s, 1H)

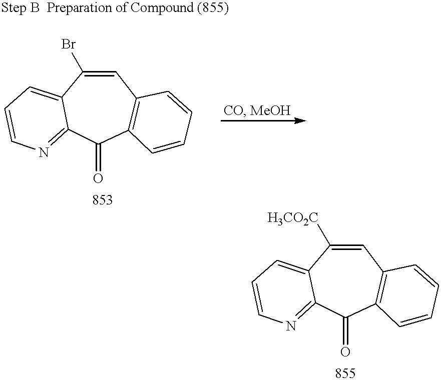

Compound (853) (25 g), triphenyl phosphine (13.75 g), and palladium chloride (1.5 g) were combine in MeOH (30 ml) and toluene (200 ml). To the mixture was added DBU (18 ml) and the mixture was sealed in a parr bomb. The mixture was stirred and subjected to 100 psi of CO at 80° C. for 5 hr. The reaction was diluted with EtOAc and washed with water. The organic layer was dried over MgSO₄, filtered and purified by flash chromatography eluting with CH₂Cl₂-10% EtOAc to give the title compound (855). δ_{H} (CDCl₃) 8.8 (dd, 1H), 8.40 (dd,1H), 8.2 (s 1H), 8.04 (dd,1H), 7.59-7.64 (m, 4H), 3.95 (s, 3H).

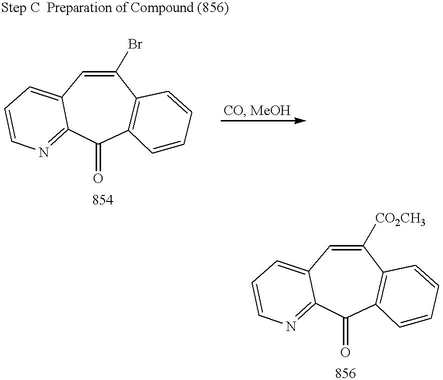

Reacting compound (854) in essentially the same manner as described in Step B above, gave the title compound (856). δ_{H} (CDCl₃) 8.85 (dd, 1H), 7.85-8.0 (m, 2H), 7.8 (s,1H), 7.25-7.31 (m, 4H)

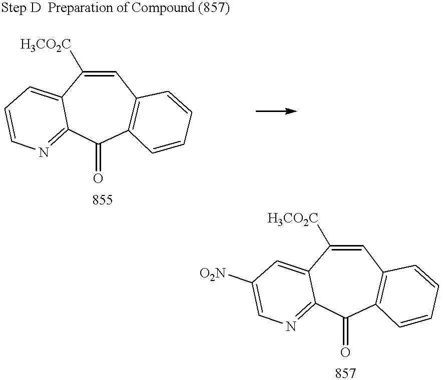

Compound (855) (19.5 g, 73.5 m mol) was dissolved in CH₂Cl₂ (100 mL) and cooled to 0° C. Tetrabutyl ammonium nitrate (31.36 g, 103 n mol) and trifluoro acetic anhydride (18.52 g, 88 m mol) were added and the mixture stirred at room temperature for 5 hrs. The reaction mixture was concentrated to dryness, followed by extraction with CH₂Cl₂—NaHCO₃. The combine organic layer was dried over MgSO₄ and concentrated to dryness and the residue was chromatographed on silica gel using CH₂Cl₂-EtOAc (25%) to give the title compound (857) (12.4 g), δ_{H} (CDCl₃) 9.45 (dd,1H), 9.05 (dd,1H), 8.28 (s 1H), 8.0 (dd, 1H), 7.65 (m, 3H), 3.98 (s, 3H).

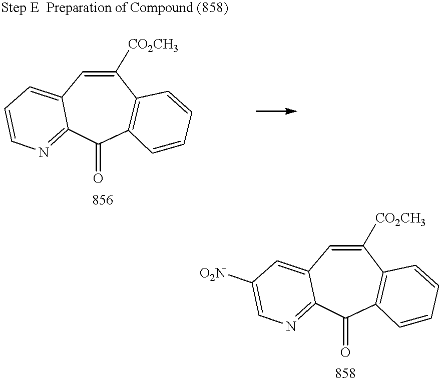

Reacting compound (856) in essentially the same manner as described in Step D above, gave the title compound (858). MH⁺=311

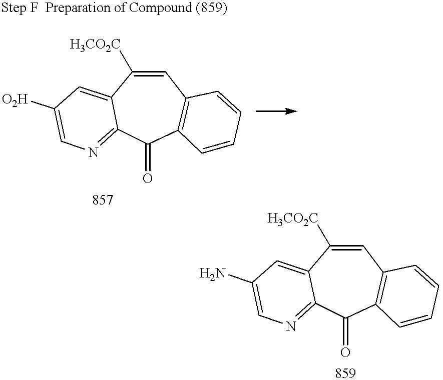

Compound (857) (6 g, ) was balloon hydrogenated in MeOH (100 mL) over Raney-Ni (4.2 g) at room temperature overnight. The catalyst was filtered off and the filtrate was evaporated to dryness to give the title compound (859) (4.66 g) MH⁺=281

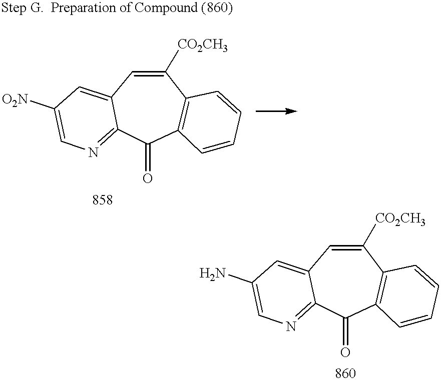

Reacting compound (858) in essentially the same manner as described in Step F above, gave the title compound (860) MH⁺=281.

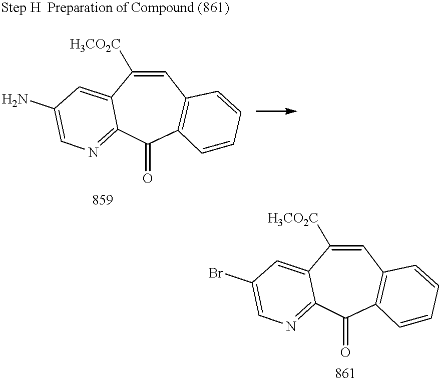

To a suspension of compound (859) (2.1 g) in 48% HBr, was added sodium nitrite (1.55 g) followed by bromine (2.11 mL) at 0° C. The mixture was stirred at room temperature overnight. Concentrated NH₄OH was then added dropwise until basic pH (to litmus paper). The reaction was extracted with CH₂Cl₂, washed with brine, dried over MgSO₄, filtered and the solvent evaporated to give the title compound (861) (1.75 g) MH⁺=345.

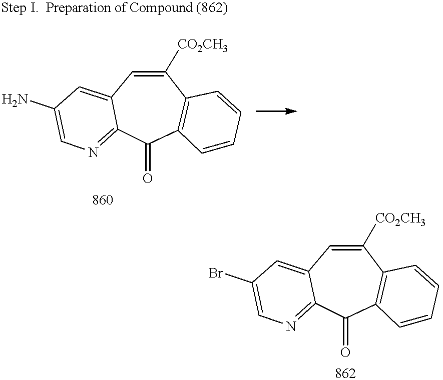

Reacting compound (861) in essentially the same manner as described in Step H above, gave the title compound (862) MH⁺345.

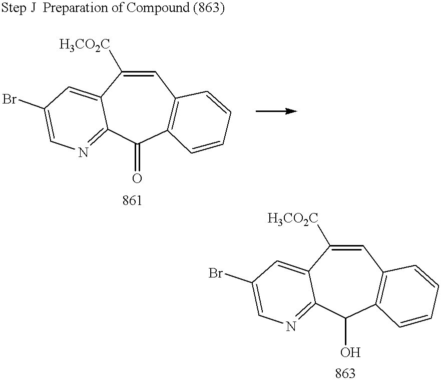

To a stirred solution of compound (861) (1.6 g, 4.64 mmole) in MeOH (30 mL) under nitrogen at 0° C. was added NaBH₄ (0.3 g, 7.9 mmole). The resulting solution was stirred at room temperature for 24 hrs, then evaporated, followed by extraction with CH₂Cl₂—H₂O. The organic layer was dried over MgSO4, filtered and evaporated to dryness to give the title compound (863) (1.58 g) MH⁺=347.

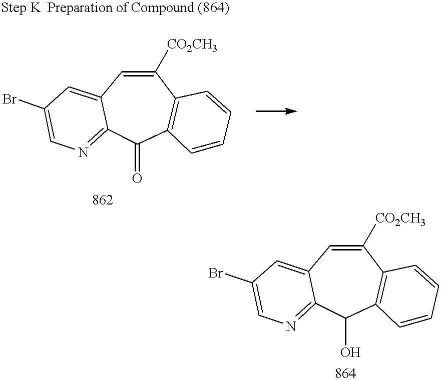

Reacting compound (862) in essentially the same manner as described in Step J above, gave the title compound (864). MH⁺=347

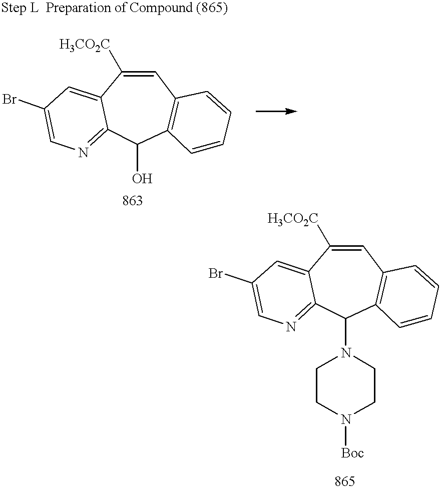

Compound 863 (1.57 g,) was stirred in thionyl chloride (10 mL) at room temperature for 4 hrs then evaporated to dryness. The resulting crude oil as taken up in acetonitrile (50 mL) and refluxed with N-Boc-piparazine (1.41 g) and triethyl amine (3.91 g) overnight. The mixture was evaporated to dryness, followed by extraction with CH₂Cl₂-NaHCO₃. The organic layer was dried over MgSO₄, filtered and evaporated to dryness to give a brown gum which was purified by column chromatography on silica gel, eluting with Hexane −20% EtOAc to give the title compound (865) (0.69 g); . MH⁺=515.

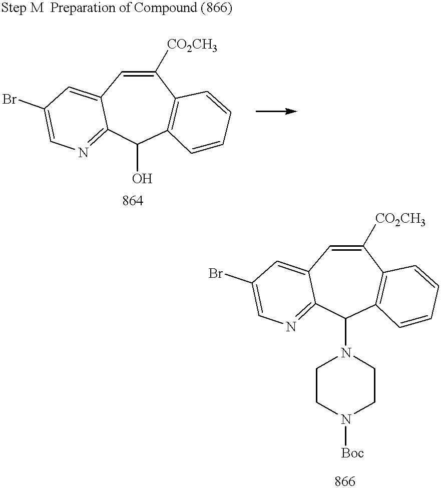

Reacting compound (864) in essentially the same manner as described in Step L above, gave the title compound (866) MH⁺=515.

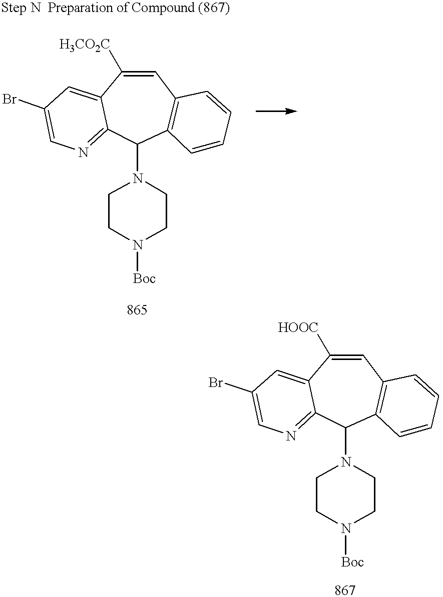

Compound (865) (0.65 g, 1.26 mmole) was refluxed with LiOH (0.45 g,, 18.79 mmole) in MeOH (15 mL) and water (1 mL) for 2 hrs. 10% aq. Citric acid was added until pH=3.5, followed by extraction with CH₂Cl₂-brine. The organic layer was dried over MgSO₄, filtered and evaporated to dryness to give a white solid (867) (0.60 g)) MH⁺=501

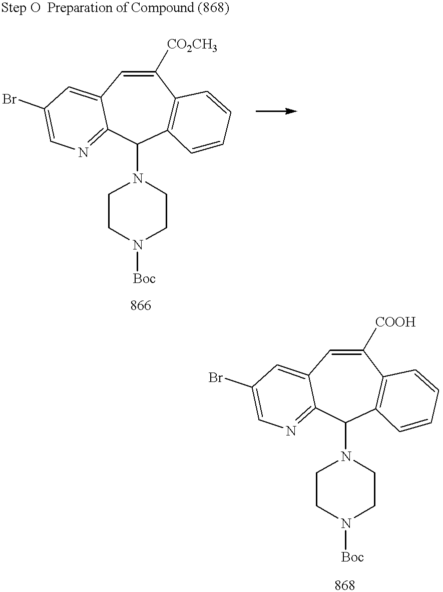

Reacting compound (866) in essentially the same manner as described in Step N above, gave the title compound (868). MH⁺=501.

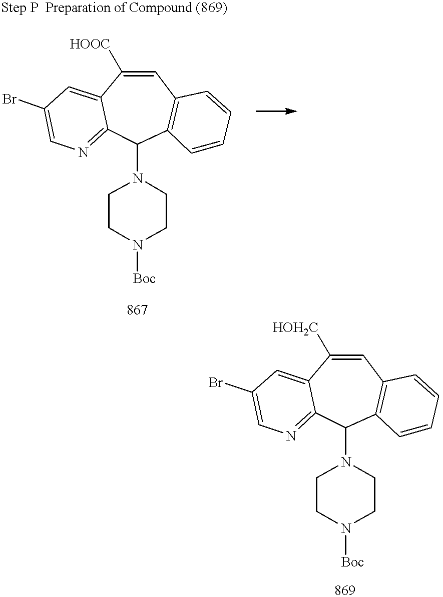

Compound (867) (0.60 g, 1.21 mmole) was stirred with carbonyl diimidazole (0.59 g, 3.63 mmole) in THF (15 mL) at at 40° C. overnight. The reaction mixture was cooled in an ice-bath then added NaBH₄ (0.28 g, 7.31 mmole) and stirred at room temperature overnight. The mixture was evaporated to dryness, followed by extraction with CH₂Cl₂-water. The organic layer was dried over MgSO₄, filtered and evaporated to give a brown gum which was purified by column chromatography on silica gel, eluting with Hexane −50% EtOAc to give the title compound (869)(0.493 g) MH⁺=487.

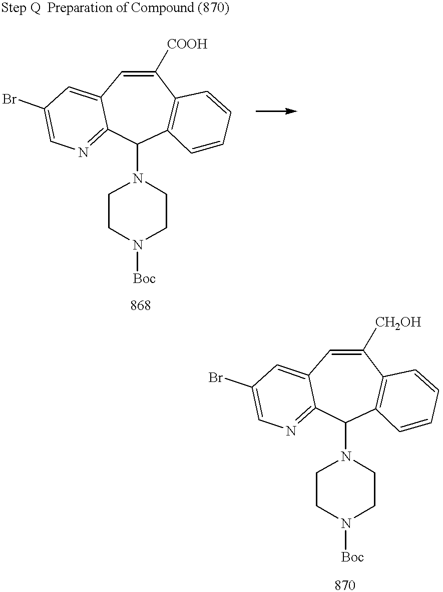

Reacting compound (868) in essentially the same manner as described in Step P above, gave the title compound (870). MH⁺=487.

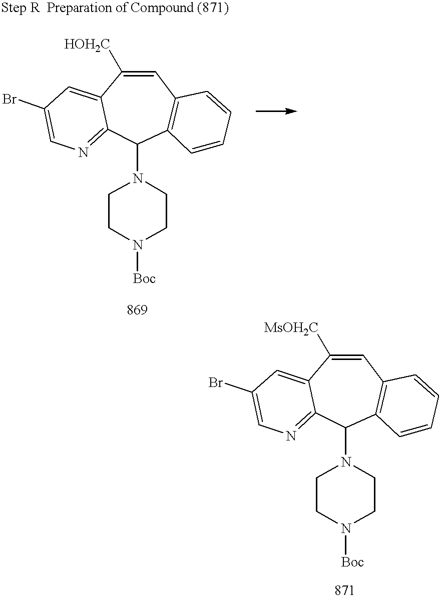

Compound (869) (0.0.38 g, 0.78 mmole) was stirred with methanesulfonyl-chloride (0.33 g, 1.296 mmole) and triethylamine (0.68 g, 6.72 mmole) in THF (10 mL) at room temperature overnight. The mixture was evaporated to dryness, followed by extraction with CH₂Cl₂-water. The organic layer was dried over MgSO₄, filtered and evaporated to dryness to give the title compound (871)(0.369 g). MH⁺=565.

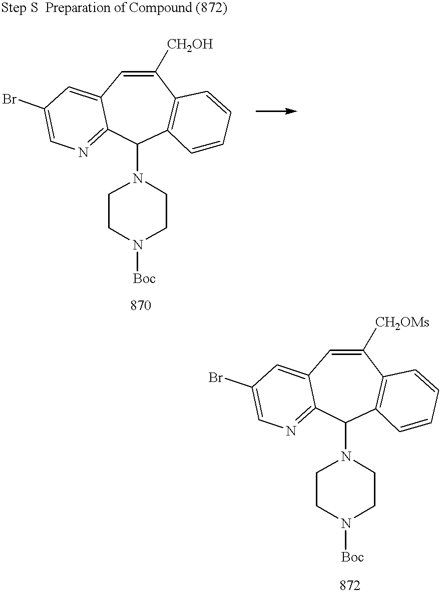

Reacting compound (870) in essentially the same manner as described in Step R above, gave the title compound (872). MH⁺=565.

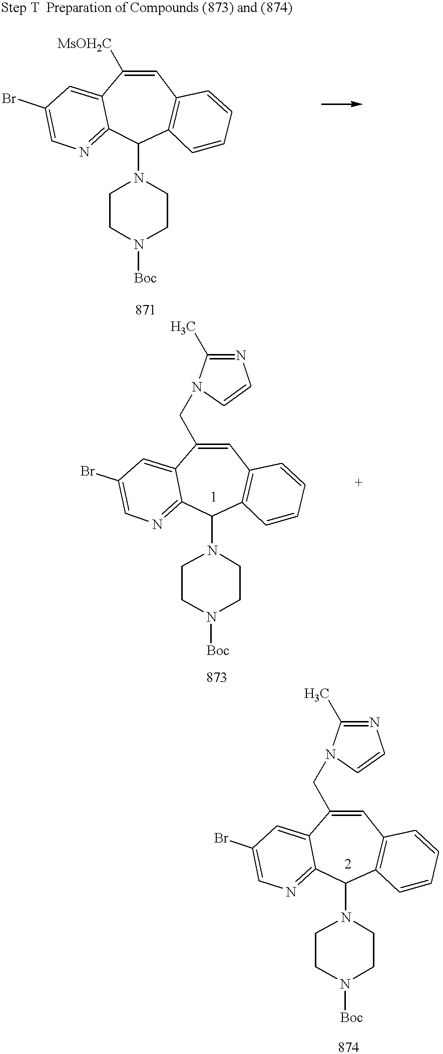

Compound (871) (0.0.369 g, 0.653 mmole) was stirred with 2-methylimidazole (0.188 g, 2.28 mmole) in DMF (5 mL) at room temperature overnight. The mixture was evaporated to dryness, followed by extraction with CH₂Cl₂-water. The organic layer was dried over MgSO₄, filtered, evaporated to dryness and then purified on silica-gel prep-plate chromatography, eluting with CH₂Cl₂-5% (MeOH-10% NH₄OH) to give the product as a mixture of isomers (1.126 g) MH+=551. Separation of the product mixture by HPLC using a prep AD column, eluting with 20% IPA/80%hexane/ 0.2%DEA (isocratic 60 ml/min.) afforded pure isomer 1 (873) (0.06 g, MH⁺=551 and isomer 2 (874) (0.0061 g) MH⁺=551.

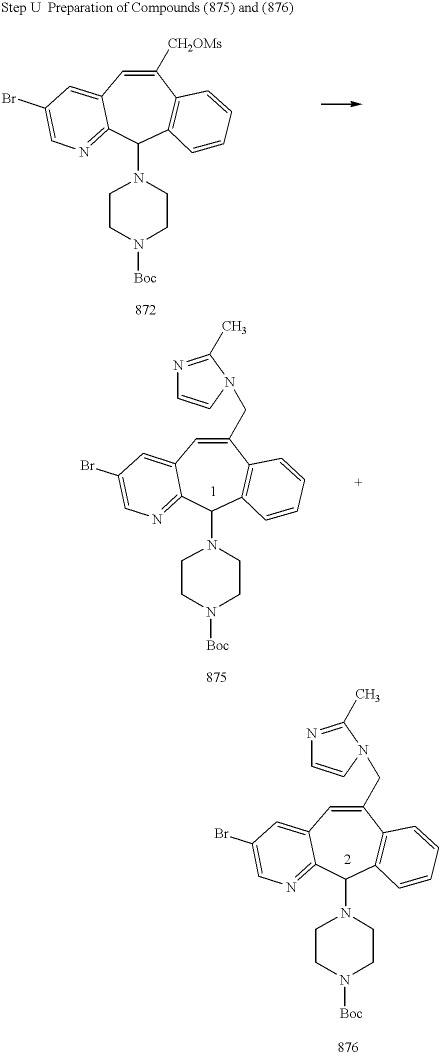

Reacting compound (872) in essentially the same manner as described in Step T above, gave the title compounds (875). MH⁺=551, and (876) MH⁺=551.

### Example 503

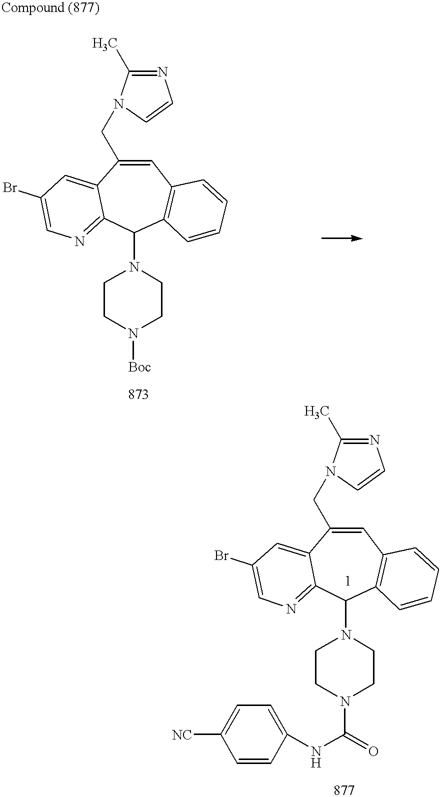

Compound (873) (0.043 g, 0.078 mmole) was stirred with TFA (5 mL) in CH₂Cl₂ (5 mL) for 4 hrs. at room temperature. The mixture was then evaporated to dryness. To the residue was added p-cyanophenylisocyanate (0.0123 g, 0.086 mmole).and triethylamine (0.5 mL) in CH₂Cl₂ (5 mL) and the mixture stirred at room temperature for 2 hrs. The mixture was evaporated to dryness, followed by extraction with CH₂Cl₂-brine. The organic layer was dried over MgSO₄, filtered and evaporated to dryness to give a brown gum which was purified by prep-plate chromatography on silica gel, eluting with CH₂Cl₂-5% (MeOH-10% NH₄OH) to give the title compound (877) (0.0394 g). MH⁺=595, δ_{H} (CDCl₃) 8.6 (1H); 8.05 (1H); 7.22-7.5 (8H); 6.99 (1H); 6.95 (1H); 6.93 (1H); 4.99-5.25 (2H); 4.6 (1H); 3.1-3.25 (4H); 2.25 (3H), 1.8-2.05 (4H).

### Example 504

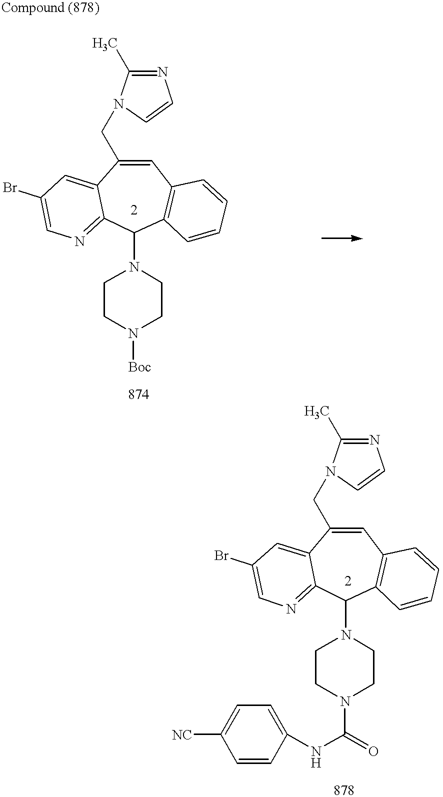

Reacting compound (874) in essentially the same manner as described in Example 503 above, gave the title compound. (878) MH⁺=595, δ_{H} (CDCl₃) 8.6 (1H); 8.05 (1H); 7.22-7.5 (8H); 6.99 (1H); 6.95 (1H); 6.93 (1H); 4.99-5.25 (2H); 4.6 (1H); 3.1-3.25 (4H); 2.25 (3H), 1.8-2.05 (4H).

### Example 505

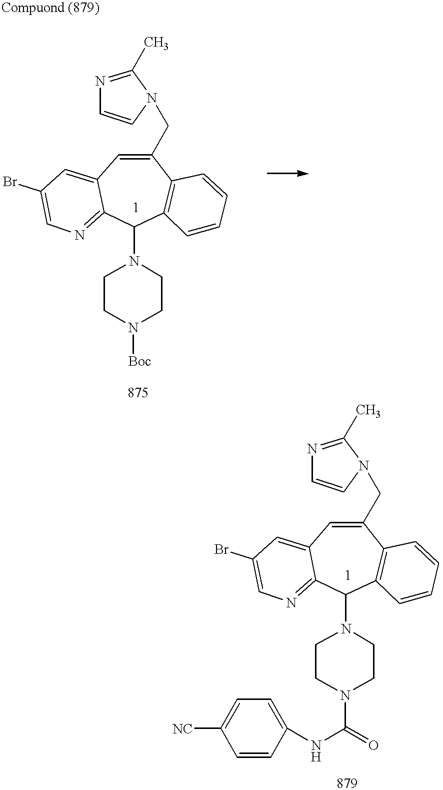

Reacting compound (875) in essentially the same manner as described in Example 503 above, gave the title compound (879). MH⁺595, δ_{H} (CDC₃) 8.55 (1H); 7.78 (1H); 7.65 (1H);7.4-7.51 (6H); 6.98 (1H); 6.9 (1H); 6.85 (1H); 5.05-5.3 ( 2H); 4.6 (1H); 3.1-3.25 (4H); 2.5 (3H), 1.8-2.00 (4H).

Assays

FPT activity was determined by measuring the transfer of [3H] farnesyl from [3H] farnesyl pyrophosphate to a biotinylated peptide derived from the C-terminus of H-ras (biotin-CVLS). The reaction mixture contains: 50 mM Tris pH7.7, 5 mM MgCl₂, 5 μM Zn⁺⁺, 5 mM DTT, 0.1% Triton-X, 0.05 μM peptide, 0.03 nM purified human farnesyl protein transferase, 0.180 μM [³H] farnesyl pyrophosphate, plus the indicated concentration of tricyclic compound or vehicle control in a total volume of 100 μl. The reaction was incubated in a Vortemp shaking incubator at 37° C., 45 RPM for 60 minutes and stopped with 150 τl of 0.25 M EDTA containing 0.5% BSA and 1.3 mg/ml Streptavidin SPA beads. Radioactivity was measured in a Wallach 1450 Microbeta liquid scintillation counter. Percent inhibition was calculated relative to the vehicle control.

COS Cell IC₅₀ (Cell-Based Assay) were determined following the assay procedures described in WO 95/10516, published Apr. 20,1995. GGPT IC₅₀ (inhibition of geranylgeranyl protein transferase, in vitro enzyme assay), Cell Mat Biochemical assay and anti-tumor activity (in vivo anti-tumor studies) could be determined by the assay procedures described in WO 95/10516. The disclosure of WO 95/10516 is incorporated herein by reference thereto.

Various tumor cells (5×10⁵ to 8×10⁶) were innoculated subcutaneously into the flank of 5-6 week old athymic nu/nu female mice. Three tumor cell models were used: mouse fibroblasts transformed with H-Ras; HTB-177 human non small cell lung cancer cells or LOX human melanoma cells. Animals were treated with beta cyclodextran vehicle only or compounds in vehicle twice a day (BID) or once a day (QD) for 7 days per week for 1 (×1), 2 (×2) or 3 (×3) weeks. The percent inhibition of tumor growth relative to vehicle controls were determined by tumor measurements.

| | | | | Average |
|---|---|---|---|---|
| Compound | | Dose | Route and | % Tumor |
| No. | Tumor | (MPK) | Schedule | Inhibition |
| (372) | H-Ras fibroblasts | 40 | po, BID, ×2 | 92 |
| ″ | H-Ras fibroblasts | 10 | po, BID, ×2 | 70 |
| ″ | H-Ras fibroblasts | 80 | po, QD, ×2 | 91 |
| ″ | H-Ras fibroblasts | 20 | po, QD, ×2 | 55 |
| ″ | H-Ras fibroblasts | 60 | po, BID, ×2 | 98 |
| ″ | H-Ras fibroblasts | 20 | po, BID, ×2 | 59 |
| ″ | H-Ras fibroblasts | 6.6 | po, BID, ×2 | 19 |
| ″ | HTB-177 | 60 | po, BID, ×3 | 87 |
| ″ | HTB-177 | 20 | po, BID, ×3 | 43 |
| ″ | HTB-177 | 120 | po, QD, ×3 | 54 |
| ″ | HTB-177 | 40 | po, QD, ×3 | 11 |
| ″ | HTB-177 | 80 | po, BID, ×3 | 96 |
| ″ | HTB-177 | 40 | po, BID, ×3 | 79 |
| ″ | HTB-177 | 20 | po, BID, ×3 | 47 |
| ″ | LOX | 15 | po, BID, ×1 | 20.9 |
| ″ | LOX | 30 | po, BID, ×1 | 54.8 |
| ″ | LOX | 60 | po, BID, ×1 | 90.3 |

(The schedule “po, BID, ×3”, for example, means orally, twice a day for 7 days (14 times per week) for 3 weeks).

Soft Agar Assay:

Anchorage-independent growth is a characteristic of tumorigenic cell lines. Human tumor cells can be suspended in growth medium containing 0.3% agarose and an indicated concentration of a farnesyl transferase inhibitor. The solution can be overlayed onto growth medium solidified with 0.6% agarose containing the same concentration of farnesyl transferase inhibitor as the top layer. After the top layer is solidified, plates can be incubated for 10-16 days at 37° C. under 5% CO₂ to allow colony outgrowth. After incubation, the colonies can be stained by overlaying the agar with a solution of MTT (3-[4,5-dimethyl-thiazol-2-yl]-2,5-diphenyltetrazolium bromide, Thiazolyl blue) (1 mg/mL in PBS). Colonies can be counted and the IC50's can be determined.

Compounds of this invention have an FPT IC₅₀ in the range of 0.001 nM to 100 nM and a Soft Agar IC₅₀ in the range of 0.01 nM to 50 nM.

The preferred compounds of the invention have an FPT IC₅₀ range of between <0.06 nM-0.44 nM and a Soft agar IC₅₀ range of between <0.05 nM-25 nM.

The most preferred compounds have an FPT IC₅₀ range of between <0.05 nM-3.0 nM and Soft agar IC₅₀ range of between 0.5 nM -5 nM.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pennsylvania.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of the compounds of the present invention in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application.

The amount and frequency of administration of the compounds of the present invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in single or divided doses, preferably, in two to four divided doses.

The chemotherapeutic agent and/or radiation therapy can be administered in association with the compounds of the present invention according to the dosage and administration schedule listed in the product information sheet of the approved agents, in the Physicians Desk Reference (PDR) as well as therapeutic protocols well known in the art. Table 1.0 below gives ranges of dosage and dosage regimens of some exemplary chemotherapeutic agents useful in the methods of the present invention. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered chemotherapeutic agents (i.e., antineoplastic agent or radiation) on the patient, and in view of the observed responses of the disease to the administered therapeutic agents.

In a preferred example of combination therapy in the treatment of pancreatic cancer, the compound of Formula (I) is administered orally in a range of from 50 to 400 mg/day, in two divided doses, in association with the antineoplastic agent, gemcitabine, which is administered at a dosage of from 750 to 1350 mg/m² weekly for three out of four weeks during the course of treatment.

In a preferred example of combination therapy in the treatment of lung cancer, the compound of Formula (I) is administered orally in a range of from 50 to 400 mg/day, in two divided doses, in association with the antineoplastic agent, paclitaxel, which is administered at a dosage of from 65 to 175 mg/m² once every three weeks.

In a preferred example of combination therapy in the treatment of gliomas, the compound of Formula (I) is administered orally in a range of from 50 to 400 mg/day, in two divided doses; in association with the antineoplastic agent, temozolomide, which is administered at a dosage of from 100 to 250 mg/m².

In another example of combination therapy, the compound of Formula (I) is administered orally in a range of from 50 to 400 mg/day, in two divided doses, in association with the antineoplastic agent, cisplatin, which is administered intravenously in a range of from 50 to 100 mg/m² once every four weeks.

In another example of combination therapy, the compound of Formula (I) is administered orally in a range of from 50 to 400 mg/day, in two divided doses, in association with the antineoplastic agent, carboplatin, which is administered intravenously in a range of from 300-360 mg/m2 once every four weeks In another example of combination therapy, the compound of Formula (I) is administered orally in a range of from 50 to 400 mg/day, in two divided doses, in association with the chemotherapeutic agent, carboplatin, which is administered intravenously in a range of from 300 to 360 mg/m2 once every four weeks and the chemotherapeutic agent, paclitaxel, which is administered at a dosage of from 65 to 175 mg/m² once every three weeks.

In yet another example of combination therapy, the compound of Formula (I) is administered orally in a range of from 50 to 400 mg/day, in two divided doses, in association with the chemotherapeutic agent, Cisplatin, which is administered intravenously in a range of from 50 to 100 mg/m2 once every four weeks and the chemotherapeutic agent, Gemcitabine, which is administered at a dosage of from 65 to 175 mg/m² once every three weeks.

The signal transduction inhibition therapy can be administered according to the dosage and administration schedule listed in the product information sheet of the approved agents, in the Physicians Desk Reference (PDR) as well as therapeutic protocols well known in the art. Table (2.0) below gives ranges of dosage and dosage regimens of some exemplary signal transduction inhibitors. It will be apparent to those skilled in the art that the administration of the signal tranduction inhibitor can be varied depending on the disease being treated and the known effects of the signal transduction inhibitor therapy on that disease. Also, in accordance with the knowledge of the skilled clinician, the therapeutic protocols (e.g., dosage amounts and times of administration) can be varied in view of the observed effects of the administered signal transduction inhibitors on the patient, and in view of the observed responses of the disease to the administered therapeutic agents.

In another example of combination therapy, the compound of Formula (I) is administered orally in a range of from 50 to 400 mg/day, in two divided doses in association with the signal tranduction inhibitor, EGF receptor kinase inhibitor, Iressa (ZD1839), which is administered orally in the range of 150-700 mg/day.

TABLE 1.0
Examplary Chemotherapeutic Agents Dosage and Dosage Regimens

| | |
|---|---|
| Cisplatin: | 50-100 mg/m² every 4 weeks (IV)* |
| Carboplatin: | 300-360 mg/m² every 4 weeks (IV) |
| Taxotere: | 60-100 mg/m² every 3 weeks (IV) |

*(IV) - intravenously

TABLE 2.0
Examplary Signal Transduction Inhibitors Dosage and Dosage Regimens

| | |
|---|---|
| Iressa (ZD1839) - EGF receptor kinase | 150-700 mg/day (oral) |
| inhibitor: | |
| OSI-774 - EGF receptor kinase | 100-1000 mg/day (oral) |
| inhibitor: | |
| Herceptin - her-2/neu antibody: | 100-250 mg/m²/week (IV)* |
| C225 - EGF receptor antibody: | 200-500 mg/m²/week (IV) |
| ABX-EGF - EGF receptor antibody: | 0.2-2 mg/kg every 2 weeks (IV) |
| Gleevec (STI-571) - bcr/abl kinase | 300-1000 mg/day (oral) |
| inhibitor: | |

*(IV) - intravenously

In the methods of the present invention, an FPT inhibitor compound of formula (I) is administered concurrently or sequentially with another therapeutic agent (i.e. a chemotherapeutic agent, a signal transduction inhibitor and/or radiation). Thus, it is not necessary that, for example, the therapeutic agent and the FPT inhibitor compound of formula (I) be administered simultaneously, just prior to or after one another.

Also, in general, the FPT inhibitor compound of formula (I), the chemotherapeutic agent, signal transduction inhibitor and/or radiation, do not have to be administered in the same pharmaceutical composition, and may, because of different physical and chemical characteristics, have to be administered by different routes. For example, the FPT inhibitor compound of formula (I) may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician .

The particular choice of the FPT inhibitor compound of formula (1), the chemotherapeutic agent, signal transduction inhibitor and/or radiation will depend upon the diagnosis of the attending physicians and their judgement of the condition of the patient and the appropriate treatment protocol.

The FPT inhibitor compound of formula (1), chemotherapeutic agent, signal transduction inhibitor and/or radiation may be administered concurrently (e.g., simultaneously, just prior to or after, or within the same treatment protocol) or sequentially, depending upon the nature of the proliferative disease, the condition of the patient, and the actual choice of chemotherapeutic agent, signal transduction inhibitor and/or radiation to be administered in conjunction (i.e., within a single treatment protocol) with the FPT inhibitor compound of formula (1).

If the FPT inhibitor compound of formula (1), chemotherapeutic agent, signal transduction inhibitor and/or radiation are not administered simultaneously, then the initial order of administration of the FPT inhibitor compound of formula (1), chemotherapeutic agent, signal transduction inhibitor and/or radiation, may not be important. Thus, the FPT inhibitor compound of formula (I) may be administered first followed by the administration of the chemotherapeutic agent, signal transduction inhibitor and/or radiation; or the chemotherapeutic agent, signal transduction inhibitor and/or radiation may be administered first followed by the administration of the FPT inhibitor compound of formula (I). This alternate administration may be repeated during a single treatment protocol. The determination of the order of administration, and the number of repititions of administration of each therapeutic agent during a treatment protocol, is well within the knowledge of the skilled physician after evaluation of the disease being treated and the condition of the patient. For example, the chemotherapeutic agent, signal transduction inhibitor and/or radiation may be administered first, especially if it is a cytotoxic agent, and then the treatment continued with the administration of the FPT inhibitor compound of formula (1), followed by, where determined advantageous, the administration of the chemotherapeutic agent, signal transduction inhibitor and/or radiation, and so on until the treatment protocol is complete.

Thus, in accordance with experience and knowledge, the practising physician can modify each protocol for the administration of a component (therapeutic agent—i.e., FPT inhibitor compound of formula (I), chemotherapeutic agent, signal transduction inhibitor or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radio-logical studies, e.g., CAT or MRI scan, and successive measure-ments can be used to judge whether or not growth of the tumor has been retarded or even reversed. Relief of disease-related symptoms such as pain, and improvement in overall condition can also be used to help judge effectiveness of treatment.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of the formula: 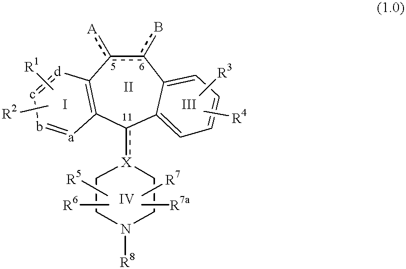
or a pharmaceutically acceptable salt or solvate thereof, wherein:
one of a, b, c and d represents N or N⁺O⁻, and the remaining a, b, c, and d groups represent carbon, wherein each carbon has an R¹ or R² group bound to said carbon; or
each of a, b, c, and d is carbon, wherein each carbon has an R¹ or R² group bound to said carbon;
the dotted lines (———) represent optional bonds;
X represents N or CH when the optional bond is absent, and represents C when the optional bond is present;
when the optional bond is present between carbon atom 5 and carbon atom 6 then there is only one A substituent bound to carbon atom 5 and there is only one B substituent bound to carbon atom 6 and A or B is other than H;
when the optional bond is not present between carbon atom 5 and carbon atom 6, then there are two A substituents bound to carbon atom 5 and two B substituents bound to carbon atom 6, wherein each A and B substituent is independently selected from:
(1) —H;
(2) —R⁹;
(3) —R⁹—C(O)—R⁹;
(4) —R⁹—CO₂—R^{9a};
(5) —(CH₂)pR²⁶;
(6) —C(O)N(R⁹)₂, wherein each R⁹ is the same or different;
(7) —C(O)NHR⁹;
(8) —C(O)NH—CH₂—C(O)—NH₂;
(9) —C(O)NHR²⁶;
(10) —(CH₂)pC(R⁹)—O—R^{9a};
(11) —(CH₂)p(R⁹)₂, wherein each R⁹ is the same or different;
(12) —(CH₂)pC(O)R⁹;
(13) —(CH₂)pC(O)R^{27a};
(14) —(CH₂)pC(O)N(R⁹)₂, wherein each R⁹ is the same or different;
(15) —(CH₂)pC(O)NH(R⁹);
(16) —(CH₂)pC(O)N(R²⁶)₂, wherein each R²⁶ is the same or different;
(17) —(CH₂)pN(R⁹)—R^{9a};
(18) —(CH₂)pN(R²⁶)₂, wherein R²⁶ is the same or different;
(19)-(CH₂)pNHC(O)R⁵⁰;
(20)-(CH₂)pNHC(O)₂R⁵⁰;
(21)-(CH₂)pN(C(O)R^{27a})₂ wherein each R^{27a} is the same or different;
(22) —(CH₂)pNR⁵¹C(O)R²⁷, or R⁵¹ and R²⁷ taken together with the atoms to which they are bound form a heterocycloalkyl ring consisting of, 5 or 6 members, provided that when R⁵¹ and R²⁷ form a ring, R⁵¹ is not H;
(23) —(CH₂)pNR⁵¹C(O)NR²⁷, or R⁵¹ and R²⁷ taken together with the atoms to which they are bound form a heterocycloalkyl ring consisting or 5 or 6 members, provided that when R⁵¹ and R²⁷ form a ring, R⁵¹ is not H;
(24) —(CH₂)pNR⁵¹C(O)N(R^{27a})₂, wherein each R^{27a} is the same or different;
(25) —(CH₂)pNHSO₂N(R⁵¹)₂, wherein each R⁵¹ is the same or different;
(26) —(CH₂)pNHCO₂R⁵⁰;
(27) —(CH₂)pNC(O)NHR⁵¹;
(28) —(CH₂)pCO₂R⁵¹;
(29) —NHR⁹; 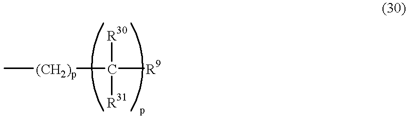
wherein R³⁰ and R³¹ are the same or different; 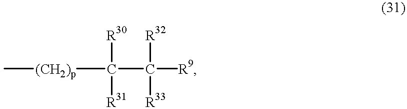
wherein R³⁰, R³¹ R³² and R³³ are the same or different;
(32) -alkenyl-CO₂R^{9a};
(33) -alkenyl-C(O)R^{9a};
(34) -alkenyl-CO₂R⁵¹;
(35) -alkenyl-C(O)-R^{27a};
(36) (CH₂)p-alkenyl-CO₂—R⁵¹;
(37) —(CH₂)pC═NOR⁵¹ and
(38) —(CH₂)p-Phthalimid;
p is 0, 1, 2, 3 or 4;
each R¹ and R² is independently selected from H, Halogen, —CF₃, —OR¹⁰, COR¹⁰, —SR¹⁰, —S(O)tR¹⁵ wherein t is 0, 1 or 2, —N(R¹⁰)2, —NO₂, —OC(O)R¹⁰, CO₂R¹⁰, —OCO₂R¹⁵, —CN, —NR¹⁰COOR¹⁵, —SR¹⁵C(O)OR¹⁵, —SR¹⁵N(R¹³)2 provided that R¹⁵in —SR¹⁵N(R¹³)2 is not —CH₂, and wherein each R¹³ is independently selected from H or —C(O)OR¹⁵, benzotriazol-1-yloxy, tetrazol-5-ylthio, or substituted tetrazol-5-ylthio, alkynyl, alkenyl or alkyl, said alkyl or alkenyl group optionally being substituted with halogen, —OR¹⁰ or —CO₂R¹⁰;
R³ and R⁴ are the same or different and each independently represent H, or any of the substituents of R and R²;
R⁵, R⁶, R7 and R^{7a} each independently represent H, —CF₃, —COR¹⁰, alkyl or aryl, said alkyl or aryl optionally being substituted with —OR¹⁰, —SR¹⁰, —S(O)ₜR¹⁵, —NR¹⁰COOR¹⁵, —N(R¹⁰)₂, —NO₂, —C(O)R¹⁰, —OCOR¹⁰, —OCO₂R¹⁵, —CO₂R¹⁰, OPO₃R¹⁰, or R⁵ is combined with R⁶ to represent ═O or ═S;
R⁸ is selected from: 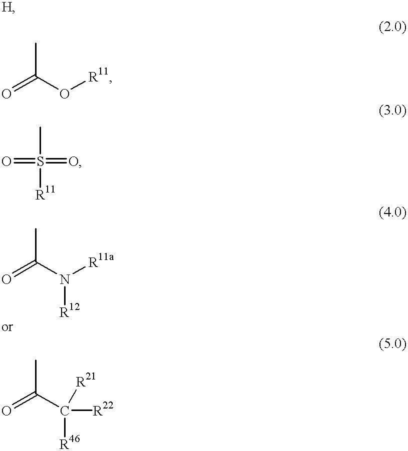
R⁹ is selected from:
(1) heteroaryl;
(2) substituted heteroaryl;
(3) arylalkoxy;
(4) substituted arylalkoxy;
(5) heterocycloalkyl;
(6) substituted heterocycloalkyl;
(7) heterocycloalkylalkyl;
(8) substituted heterocycloalkylalkyl;
(9) heteroarylalkyl;
(10) substituted heteroarylalkyl;
(11) heteroarylalkenyl;
(12) substituted heteroarylalkenyl;
(13) heteroarylalkynyl;
(14) substituted heteroarylalkynyl;
(15) arylalkyl;
(16) substituted arylalkyl;
(17) alkenyl, and
(18) substituted alkenyl;
wherein said substituted R⁹ groups are substituted with one or more substituents selected from:
(1) —OH;
(2) —CO₂R¹⁴;
(3) —CH₂OR¹⁴,
(4) halogen;
(5) alkyl;
(6) amino;
(7) trityl;
(8) heterocycloalkyl;
(9) cycloalkyl;
(10) arylalkyl;
(11) heteroaryl;
(12) heteroarylalkyl and 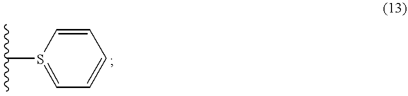
wherein R¹⁴ is independently selected from: H; alkyl; aryl, arylalkyl, heteroaryl and heteroarylalkyl;
R⁹a is selected from: alky or arylalkyl;
R¹⁰ is selected from: H; alkyl; aryl or arylalkyl;
R¹¹ is selected from:
(1) alkyl;
(2) substituted alkyl;
(3) aryl;
(4) substituted aryl;
(5) cycloalkyl;
(6) substituted cycloalkyl;
(7) heteroaryl;
(8) substituted heteroaryl;
(9) heterocycloalkyl; and
(10) substituted heterocycloalkyl;
wherein said substituted R¹¹ groups have 1, 2 or 3 substituents selected from:
(1) —OH;
(2) halogen and
(3) alkyl;
R^{11a} is selected from:
(1) H;
(2) OH;
(3) alkyl;
(4) substituted alkyl;
(5) aryl;
(6) substituted aryl;
(7) cycloalkyl;
(8) substituted cycloalkyl;
(9) heteroaryl;
(10) substituted heteroaryl;
(11) heterocycloalkyl; and
(12) substituted heterocycloalkyl;
wherein said substituted R^{11a} groups have one or more substituents selected from:
(1) —OH;
(2) —CN;
(3) —CF₃;
(4) halogen;
(5) alkyl;
(6) cycloalkyl;
(7) heterocycloalkyl;
(8) arylalkyl;
(9) heteroarylalkyl;
(10) alkenyl and
(11) heteroalkenyl;
R¹² is selected from: H, or alkyl;
R¹⁵ is selected from: alkyl or aryl;
R²¹, R²² and R⁴⁶ are independently selected from:
(1) —H;
(2) alkyl;
(3) aryl;
(4) substituted aryl,
optionally substituted with one or more substituents selected from: alkyl, halogen, CF₃ or OH;
(5) cycloalkyl;
(6) substituted cycloalkyl;
optionally substituted with one or more substituents selected from: alkyl, halogen, CF₃ or OH; 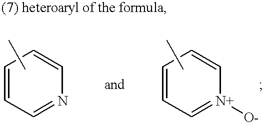 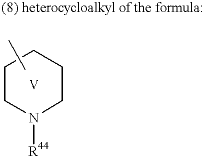
wherein R⁴⁴ is selected from:
(1) —H;
(2) alkyl;
(3) alkylcarbonyl;
(4) alkyloxy carbonyl;
(5) haloalkyl and
(6) —C(O)NH(R⁵¹);
when R², R²² or R⁴⁶ is the heterocycloalkyl of the formula above, Ring V is: 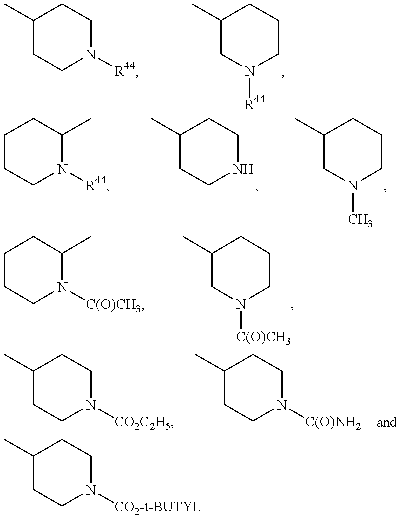
R²⁶ is selected from:
(1) —H;
(2) alkyl;
(3) alkoxyl;
(4) —CH₂—CN;
(5) R⁹;
(6) —CH₂CO₂H;
(7) —C(O)alkyl and
(8) CH₂CO₂alkyl;
R²⁷ is selected from:
(1) —H;
(2)—OH;
(3) alkyl and
(4) alkoxy;
R^{27a} is selected from:
(1) alkyl or
(2) alkoxy;
R³⁰ through R³³ is independently selected from:
(1) —H;
(2) —OH;
(3) ═O;
(4) alkyl;
(5) aryl and
(6) arylalkyl;
R⁵⁰ is selected from:
(1) alkyl;
(2) heteroaryl;
(3) substituted heteroaryl and
(4) amino;
wherein said substituents on said substituted R⁵⁰ groups are independently selected from: alkyl; halogen; or —OH;
R^{50a} is selected from:
(1) heteroaryl;
(2) substituted heteroaryl and
(3) amino;
R⁵¹ is selected from: —H, or alkyl.

2. A compound of claim 1 having the structure: 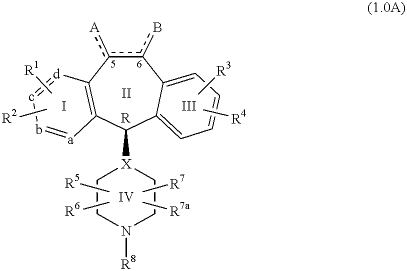
X CH or N;
B is H when the optional bond is present between C-5 and C-6, and when the optional bond between C-5 and C-6 is absent then each B is H.

3. A compound of claim 1 having the structure: 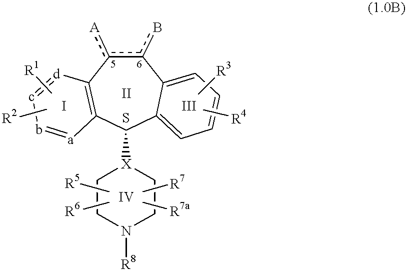
X=CH or N;
A is H when the optional bond present between C-5 and C-6, and when the optional bond between C-5 and C-6 is absent then each A is H.

4. The compound of claim 1 wherein R¹ to R⁴ are each independently selected from H or halo.

5. The compound of claim 1 wherein R⁵ to R⁷ are H.

6. The compound of claim 1 wherein a is N and the remaining b, c and d substituents are carbon.

7. The compound of claim 1 wherein a, b, c, and d are carbon.

8. The compound of claim 1 wherein the optional bond between C-5 and C-6 is present.

9. The compound of claim 1 wherein the optional bond between C-5 and C-6 is absent.

10. The compound of claim 1 wherein R⁸ is group 2.0, or 4.0.

11. The compound of claim 1 wherein R¹ to R⁴ are each independently selected from H, or halo; R⁵ to R^{7a} are H; a is N and the remaining b, c and d substituents are carbon and R⁸ is group 2.0 or 4.0.

12. The compound of claim 2 wherein R¹ to R⁴ are each independently selected from H, Br or Cl.

13. The compound of claim 1 wherein R¹ to R⁴ are each independently selected from H, Br or Cl; R⁵ to R^{7a} are H; a, b, c and d substituents are carbon and R⁸ is group 2.0 or 4.0.

14. The compound of claim 1 wherein:
(1) R¹¹ is selected from: alkyl, cycloalkyl or substituted cycloalkyl, said substituted groups are substituted with halo, alkyl or amino;
(2) R^{11a} is selected from: alkyl, aryl, substituted aryl, cycloalkyl or substituted cycloalkyl, said substituted groups are substituted with halo, —CN or CF₃;
(3) R¹², R²¹, and R²² are H; and
(4) R⁴⁶ is selected from: aryl, substituted aryl, heteroaryl of the formula: 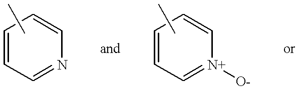
hetercycloalkyl of the formula: 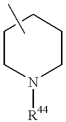
wherein, said substituted groups are substituted with alkyl, alkylcarbonyl or haloalkyl, and R⁴⁴ is selected from H or —C(O)NH₂.

15. The compound of claim 1 wherein R⁸ is selected from:
(a) group 2.0 wherein R¹¹ is selected from: t-butyl or cyclohexyl;
(b) group 3.0 wherein R¹¹ is selected from methyl or t-butyl;
(c) group 4.0 wherein, R¹² is H and R^{11a} is selected from t-butyl, cyanophenyl, chlorophenyl, fluorophenyl or cyclohexyl;
(d) group 5.0 wherein R²¹ and R²² are H and R⁴⁶ is selected from: 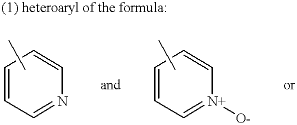 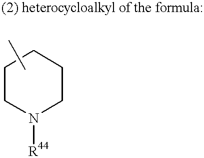
and wherein R⁴⁴ is —C(O)NH₂.

16. The compound of claim 15 wherein R⁸ is group 4.0.

17. The compound of claim 1 wherein one of A and B is H and the other is R⁹.

18. The compound of claim 17 wherein A is H and B is R⁹ wherein R⁹ is selected from:
(1) heteroaryl;
(2) substituted heteroaryl;
(3) arylalkyl;
(4) substituted arylalkyl;
(5) arylalkoxy;
(6) substituted arylalkoxy;
(7) heterocycloalkyl;
(8) substituted heterocycloalkyl;
(9) heterocycloalkylalkyl;
(10) substituted heterocycloalkylalkyl;
(11) heteroarylalkyl;
(12) substituted heteroarylalkyl;
(13) alkenyl;
(14) substituted alkenyl;
(15) heteroarylalkenyl and
(16) substituted heteroarylalkenyl,
wherein substituents for said substituted R⁹ groups are each independently selected from:
(1) —OH;
(2) —CO₂R¹⁴;
(3) —CH₂OR¹⁴,
(4) halo,
(5) alkyl;
(6) amino;
(7) trityl;
(8) heterocycloalkyl;
(9) arylalkyl;
(10) heteroaryl and
(11) heteroarylalkyl,
wherein R¹⁴ is independently selected from: H; or alkyl.

19. The compound of claim 18 wherein R⁹ is selected from:
(1) heterocycloalkylalkyl of the formula —(CH₂)ₙ-heterocycloalkyl;
(2) substituted heterocycloalkylalkyl of the formula —(CH₂)ₙ-substituted heterocycloalkyl;
(3) heteroarylalkyl of the formula —(CH₂)ₙ-heteroaryl, or
(4) substituted heteroarylalkyl of the formula —(CH₂)ₙ-substituted heteroaryl.
wherein n is 1, 2, or 3 and the substituents for said substituted R⁹ groups are each independently selected from:
(1) —OH;
(2) —CO₂R¹⁴;
(3) —CH₂OR¹⁴,
(3) halo,
(4) alkyl;
(5) amino;
(6) trityl;
(7) heterocycloalkyl;
(8) arylalkyl;
(9) heteroaryl and
(10) heteroarylalkyl.
wherein R¹⁴ is independently selected from: H; or alkyl

20. The compound of claim 19 wherein R⁹ is
(1) —(CH₂)n-imidazolyl;
(2) —(CH2)n-substituted imidazolyl;
(3) —(CH2)n-morpholinyl;
(4) —(CH2)n-substituted morpholinyl,
(5) —(CH2)n-piperazinyl, or
(6) —(CH2)n-substituted piperazinyl,
wherein n is 1, 2, or 3.

21. The compound of claim 1 wherein the optional bond is present between C-5 and C-6 and A is H and B is R⁹, or A is R⁹ and B is H; or the optional bond between C-5 and C-6 is absent and each A is H, one B is H and the other B is R⁹, or one A is H, the other A is R⁹ and each B is H; R¹ to R⁴ are independently H or halo; R⁵ to R⁷a are H; a is N and the remaining b, c, an d substituents are carbon; X is N or CH and R⁸ is group 2.0 or 4.0.

22. The compound of claim 21 wherein R⁹ is selected from:
(1) heteroaryl;
(2) substituted heteroaryl;
(3) arylalkyl;
(4) substituted arylalkyl;
(5) arylalkoxy;
(6) substituted arylalkoxy;
(7) heterocycloalkyl;
(8) substituted heterocycloalkyl;
(9) heterocycloalkylalkyl;
(10) substituted heterocycloalkylalkyl;
(11) heteroarylalkyl;
(12) substituted heteroarylalkyl;
(13) alkenyl;
(14) substituted alkenyl;
(15) heteroarylalkenyl and
(16) substituted heteroarylalkenyl,
wherein substituents for said substituted R⁹ groups are each independently selected from:
(1) —OH;
(2) —CO₂R¹⁴;
(3) —CH₂OR¹⁴,
(4) halo,
(5) alkyl;
(6) amino;
(7) trityl;
(8) heterocycloalkyl;
(9) arylalkyl;
(10) heteroaryl and
(11) heteroarylalkyl,
wherein R¹⁴ is independently selected from: H; or alkyl.

23. The compound of claim 22 wherein R⁹ is selected from:
(1) heterocycloalkylalkyl of the formula —(CH₂)ₙ-heterocycloalkyl;
(2) substituted heterocycloalkylalkyl of the formula —(CH₂)ₙ-substituted heterocycloalkyl;
(3) heteroarylalkyl of the formula —(CH₂)ₙ-heteroaryl, and
(4) substituted heteroarylalkyl of the formula —(CH₂)ₙ-substituted heteroaryl.
wherein substituents for said substituted R⁹ groups are each independently selected from:
(1) —OH;
(2) —CO₂R¹⁴;
(3) —CH₂OR¹⁴,
(3) halo,
(4) alkyl;
(5) amino;
(6) trityl;
(7) heterocycloalkyl;
(8) arylalkyl;
(9) heteroaryl and
(10) heteroarylalkyl.

24. The compound of claim 23 wherein R⁸ is group 4.0 and wherein R¹² is H and R^{11a} is selected from:
(1) alkyl;
(2) aryl;
(3) substituted aryl;
(4) cycloalkyl, and
(5) substituted cycloalkyl,
wherein said substituents of said substituted groups are selected from:
(1) halo;
(2) —CN and
(3) —CF3.

25. The compound of claim 23 wherein R⁹ is
(1) —(CH2)n-imidazolyl;
(2) —(CH2)n-substituted imidazolyl;
(3) —(CH2)n-morpholinyl;
(4) —(CH2)n-substituted morpholinyl;
(5) —(CH2)n-piperazinyl, or
(6) —(CH2)n-substituted piperazinyl,
wherein n is 1, 2, or 3.

26. The compound of claim 25 wherein the optional bond is present.

27. The compound of claim 26 wherein R⁸ is 4.0 and wherein R¹² is H and R^{11a} is selected from:
(1) alkyl;
(2) aryl;
(3) substituted aryl;
(4) cycloalkyl, and
(5) substituted cycloalkyl,
wherein said substituents of said substituted groups are selected from:
(1) halo;
(2) cyano, and
(3) CF3.

28. The compound of claim 27 wherein R⁸ is 4.0, R¹² is H and R^{11a} is substituted phenyl and wherein said substituent of said substituted group selected from:
(1) —CN or
(2) CF3.

29. The compound of claim 25 wherein the optional bond is absent.

30. The compound according to claim 1 which is selected from any one of the Examples 1-505.

31. The compound according to claim 1 which is selected from the group consisting of:

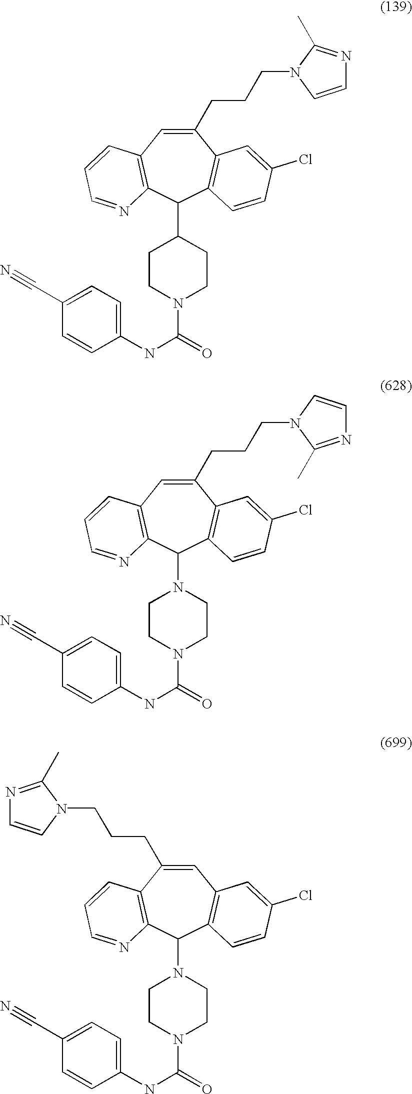

32. The compound according to claim 1 which is selected from the group consisting of:

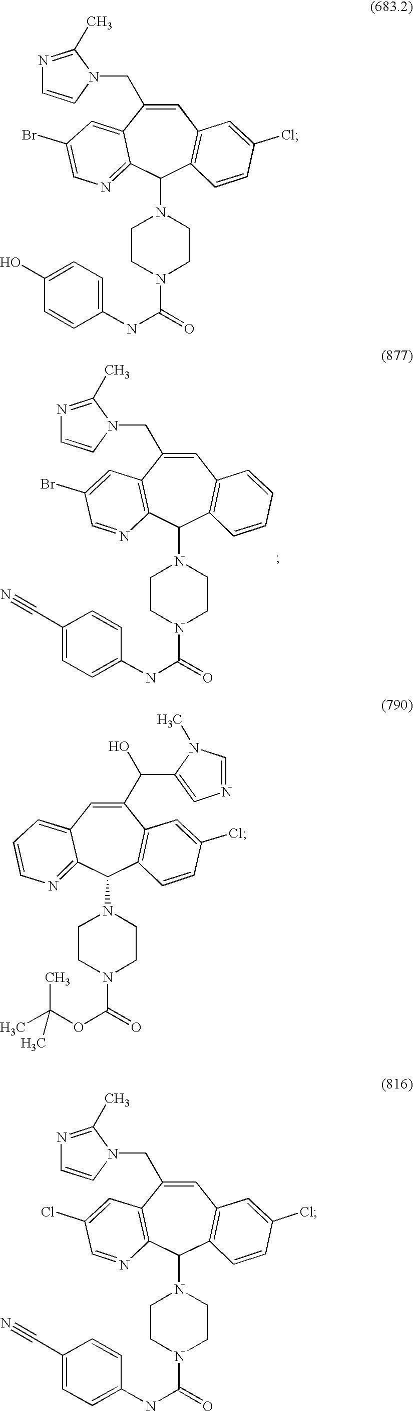

33. The compound according to claim 1 which is:

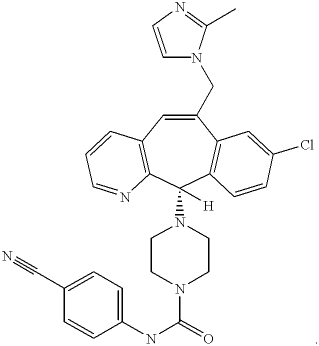

34. The compound according to claim 1 which is:

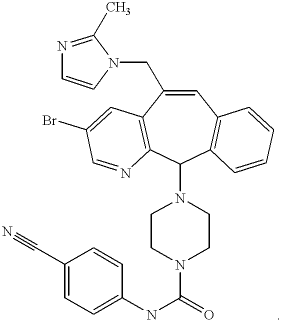

35. The compound according to claim 1 which is:

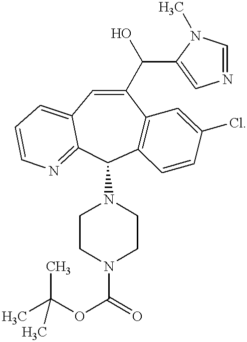

36. The compound according to claim 1 which is:

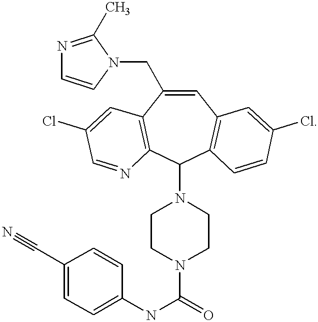

37. The compound according to claim 1 which is:

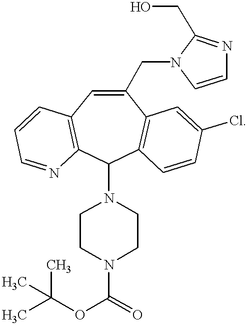

38. The compound according to claim 1 which is:

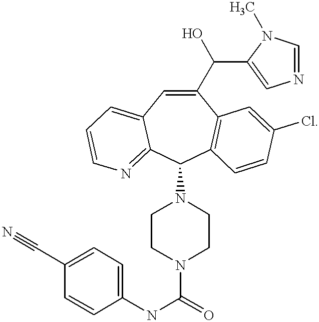

39. The compound according to claim 1 which is:

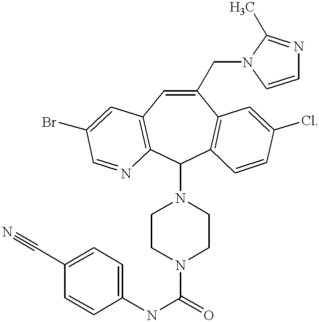

40. A pharmaceutical composition for inhibiting the abnormal growth of cells comprising an effective amount of compound of claim 1 in combination with a pharmaceutically acceptable carrier.

41. A method for inhibiting the abnormal growth of cells comprising administering an effective amount of a compound of claim 1.

42. The method of claim 41 wherein the the cells inhibited are tumor cells expressing an activated ras oncogene.

43. The method of claim 42 wherein the tumor cells inhibited are pancreatic tumor cells, lung tumor cells, myeloid leukemia tumor cells, thyroid follicular tumor cells, myelodysplastic tumor cells, head and neck tumor cells, melanoma tumor cells, breast tumor cells, prostate tumor cells, ovarian tumor cells, bladder tumor cells, glioma cells or colon tumor cells.

44. The method of claim 41 wherein the inhibition of the abnormal growth of cells occurs by the inhibition of ras farnesyl protein transferase.

45. The method of claim 41 wherein the inhibition is of tumor cells wherein the Ras protein is activated as a result of oncogenic mutation in genes other than the Ras gene.

46. A pharmaceutical composition for inhibiting the abnormal growth of cells comprising an effective amount of compound of claim 32 in combination with a pharmaceutically acceptable carrier.

47. A method for inhibiting the abnormal growth of cells comprising administering an effective amount of a compound of claim 32.

48. The method of claim 47 wherein the the cells inhibited are tumor cells expressing an activated ras oncogene.

49. The method of claim 47 wherein the cells inhibited are pancreatic tumor cells, lung tumor cells, myeloid leukemia tumor cells, thyroid follicular tumor cells, myelodysplastic tumor cells, head and neck tumor cells, melanoma tumor cells, breast tumor cells, prostate tumor cells, ovarian tumor cells, bladder tumor cells, glioma cells and colon tumor cells.

50. The method of claim 47 wherein the inhibition of the abnormal growth of cells occurs by the inhibition of ras farnesyl protein transferase.

51. The method of claim 47 wherein the inhibition is of tumor cells wherein the Ras protein is activated as a result of oncogenic mutation in genes other than the Ras gene.

52. A method of treating proliferative disease in a patient in need of such treatment, said treatment comprising administering concurrently or sequentially, an effective amount of a compound of claim 1 in combination with an effective amount of at least one chemotherapeutic agent and/or radiation.

53. The method of claim 52 wherein said proliferative disease is selected from pancreatic cancer, lung cancer, myeloid leukemia, thyroid follicular cancer, myelodysplastic syndrome, head and neck cancer, melanoma, breast cancer, prostate cancer, ovarian cancer, bladder cancer, glioma and colon cancer.

54. The method of claim 52 wherein said proliferative disease is selected from lung cancer, head and neck cancer, bladder cancer, breast cancer, prostate cancer and myeloid leukemia,

55. The method of claim 52 wherein said chemotherapeutic agent is an antineoplastic agent selected from: Uracil mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Temozolomide, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Taxol, Taxotere, Mithramycin, Deoxycoformycin, Mitomycin-C, L-Asparaginase, Interferons, Etoposide, Teniposide 17- -Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, CPT-11, Anastrazole, Letrazole, Capecitabine, Reloxafine, Droloxafine, and Hexamethylmelamine.

56. The method of claim 52 wherein said chemotherapeutic agent is a microtubule affecting agent selected from allocolchicine, Halichondrin B, colchicine, colchicine derivatives, dolastatin 10, maytansine, rhizoxin, paclitaxel, paclitaxel derivatives, Taxotere, thiocolchicine, trityl cysteine, vinblastine sulfate, vincristine sulfate, epothilone A, epothilone, discodermolide, estramustine, nocodazole and MAP4.

57. The method of claim 52 wherein said chemotherapeutic agent is selected from Gemcitabine, Cisplatin, Carboplatin, paclitaxel, paclitaxel derivatives, and Taxotere.

58. The method of claim 52 wherein the compound of claim 1 is selected from:

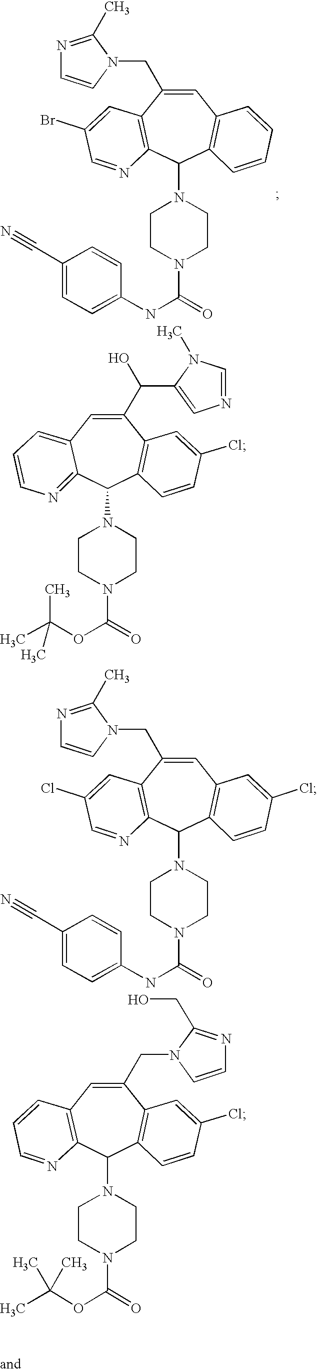

59. The method of claim 52 wherein the proliferative disease treated is selected from lung cancer, head and neck cancer, bladder cancer, breast cancer, prostate cancer and myeloid leukemia; the chemotherapeutic agent is an antineoplastic agent selected from: Uracil mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Temozolomide, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Taxol, Taxotere, Mithramycin, Deoxycoformycin, Mitomycin-C, L-Asparaginase, Interferons, Etoposide, Teniposide 17a-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, CPT-11, Anastrazole, Letrazole, Capecitabine, Reloxafine, Droloxafine, and Hexamethylmelamine and/or a microtubule affecting agent selected from:. allocoichicine, Halichondrin B, colchicine, colchicine derivatives, dolastatin 10, maytansine, rhizoxin, paclitaxel, paclitaxel derivatives, thiocoichicine, trityl cysteine, vinblastine sulfate, vincristine sulfate, epothilone A, epothilone, discodermolide estramustine, nocodazole and MAP4 and the compound of claim 1 is selected from:.

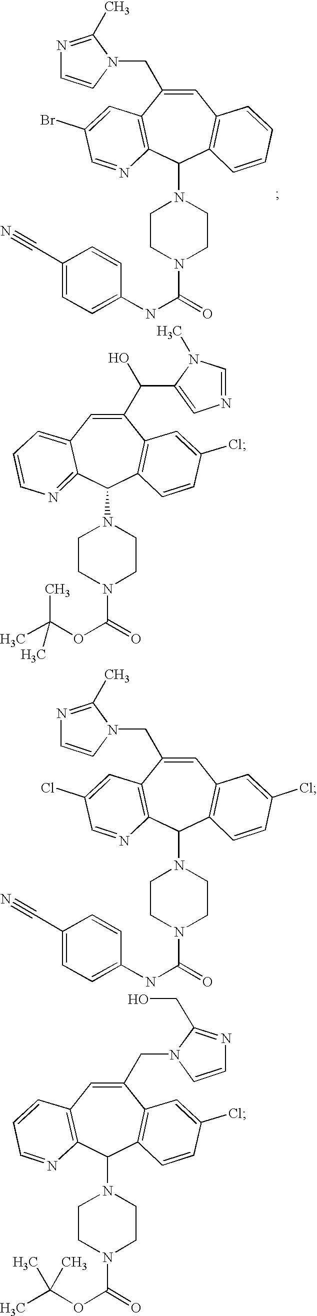

60. The method of claim 59 wherein the microtubule affecting agent is selected from Paclitaxel, a Paclitaxel derivative and Taxotere.

61. The method of claim 59 wherein the antineoplastic agent is selected from Cyclophosphamide, 5-Fluorouracil, temozolomide, Vincristine, Cisplatin, Carboplatin, and Gemcitabine.

62. The method of claim 59 wherein the antineoplastic agent is selected from Gemcitabine, Cisplatin, and Carboplatin.

63. The method of claim 59 wherein the proliferative disease treated is selected from lung cancer, head and neck cancer, bladder cancer, breast cancer, prostate cancer and myeloid leukemia; the chemotherapeutic agent is an antineoplastic agent selected from Gemcitabine, Cisplatin, and Carboplatin and/or a microtubule affecting agent selected from Taxol and Taxotere and the compound of claim 1 is selected from:

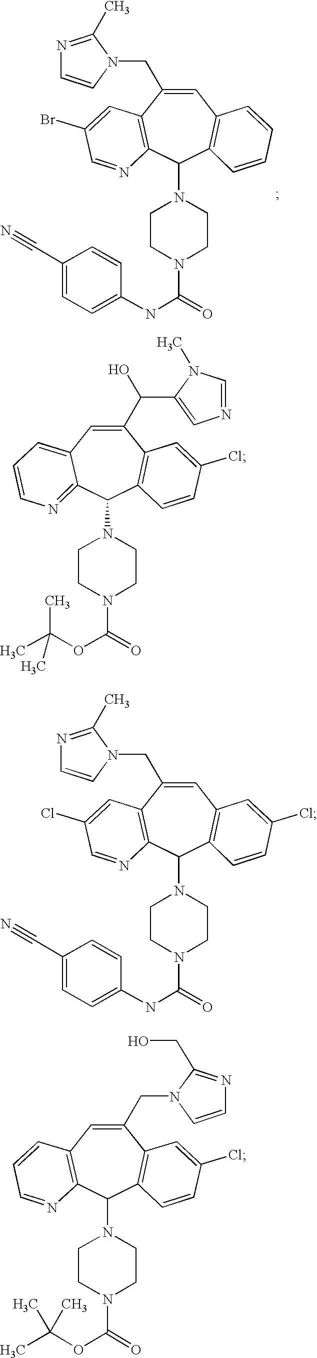

64. The method of claim 59 wherein the proliferative disease treated is lung cancer and the chemotherapeutic agent is selected from Carboplatin, Taxol and Taxotere.

65. The method of claim 59 wherein the proliferative disease treated is lung cancer and the chemotherapeutic agent is selected from Gemcitabine, and Cisplatin.

66. The method of claim 59 wherein the chemotherapeutic agent is Taxol.

67. A method of treating proliferative diseases in a patient in need of such treatment, said treatment comprising administering concurrently or sequentially, an effective amount of a compound of claim 1 in combination with an effective amount of at least one additional signal transduction inhibitor.

68. The method of claim 67 whereing the proliferative disease treated is selected from pancreatic cancer, lung cancer, myeloid leukemia, thyroid follicular cancer, myelodysplastic syndrome, head and neck cancer, melanoma, breast cancer, prostate cancer, ovarian cancer, bladder cancer, glioma and colon cancer.

69. The method of claim 67 wherein the signal tranduction inhibitor is selected from a bcr/abl kinase inhibitor, an epidermal growth factor receptor inhibitor and a her-2/neu receptor inhibitor.

70. The method of 67 wherein the signal transduction inhibitor is selected from the bcr/abl inhibitor Gleevec, the epidermal growth factor receptor inhibitors Iressa, OSI-774, Imclone C225 and Abgenix ABX-EGF; and the her-2/neu receptor inhibitor Herceptin.

71. The method of 67 wherein the proliferative disease treated is selected from lung cancer, head and neck cancer, bladder cancer, breast cancer, prostate cancer and myeloid leukemia; the signal transduction inhibitor is selected from the bcr/abi inhibitor Gleevec, the epidermal growth factor receptor inhibitors Iressa, OSI-774, Imclone C225 and Abgenix ABX-EGF; and the her-2/neu receptor inhibitor Herceptin and the compound of claim 1 is selected from [chemical structure]
